(19) United States
(12) Patent Application Publication
(10) Pub. No.: US 2014/0093903 A1
(43) Pub. Date: Apr. 3, 2014

(54) METHODS FOR DIAGNOSIS, PROGNOSIS AND METHODS OF TREATMENT

(71) Applicant: Nodality, Inc., South San Francisco, CA (US)

(72) Inventors: Jason Ptacek, Redwood City, CA (US); Racheal Hawtin, San Carlos, CA (US); Erik Evensen, Foster City, CA (US)

(73) Assignee: Nodality, Inc., South San Francisco, CA (US)

(21) Appl. No.: 14/011,715

(22) Filed: Aug. 27, 2013

Related U.S. Application Data
(60) Provisional application No. 61/693,429, filed on Aug. 27, 2012.
(60) Provisional application No. 61/720,050, filed on Oct. 30, 2012.

(51) Int. Cl. G01N 33/50

(52) U.S. Cl. CPC G01N 33/5091
USPC 435/29; 435/287.1

(57) ABSTRACT

The invention provides methods, compositions, and systems for diagnosis, prognosis, evaluation of status, and/or determination of treatment for pathological conditions.

### CROSS-REFERENCE

This application claims the benefit of U.S. Provisional Application No. 61/693,429, filed Aug. 27, 2012, and U.S. Provisional Application No. 61/720,050, filed Oct. 30, 2012, which applications are incorporated herein by reference.

This application is related to U.S. Provisional Application No. 61/693,429 filed Aug. 27, 2012 and to U.S. Ser. No. 12/748,478, filed May 20, 2010, U.S. Provisional Application No. 61/306,872, filed Feb. 22, 2010, U.S. Provisional Application No. 61/306,665, filed Feb. 22, 2010, U.S. Provisional Application No. 61/263,281, filed Nov. 20, 2009, U.S. Provisional Application No. 61/241,773, filed Sep. 11, 2009, and U.S. Provisional Application No. 61/216,825, filed May 20, 2009, U.S. Ser. No. 12/229,476, filed Aug. 21, 2008 all of which applications are incorporated herein by reference.

### BACKGROUND OF THE INVENTION

Many conditions are characterized by disruptions in cellular pathways that lead, for example, to aberrant control of cellular processes, or to uncontrolled growth and proliferation of cells. These disruptions are often caused by changes in the activity of molecules participating in cellular pathways. For example, specific signaling pathway alterations have been described for many cancers. Despite the increasing evidence that disruption in cellular pathways mediate the detrimental transformation, the precise molecular events underlying these transformations have not been elucidated. As a result, therapeutics may not be effective in treating conditions involving cellular pathways that are not well understood. Thus, the successful diagnosis of a condition and use of therapies will require knowledge of the cellular events that are responsible for the condition pathology.

In addition, patients suffering from different conditions follow heterogeneous clinical courses. For instance, tremendous clinical variability among remissions is also observed in cancer patients, even those that occur after one course of therapy. Some leukemia patients survive for prolonged periods without definitive therapy, while others die rapidly despite aggressive treatment. Patients who are resistant to therapy have very short survival times, regardless of when the resistance occurs. While various staging systems have been developed to address this clinical heterogeneity, they cannot accurately predict whether an early or intermediate stage patient will experience an indolent or aggressive course of disease.

Accordingly, there is a need for a reliable indicator of an individual predicted disease course to help clinicians to identify those patients that will respond to treatment, patients that progress to a more advanced state of the disease and patients with emerging resistance to treatment.

### SUMMARY OF THE INVENTION

As disclosed herein is a method for classifying a cell comprising contacting the cell with a modulator or an inhibitor used to determine the presence or absence of a change in activation level of an activatable element in the cell, and classifying the cell based on the presence or absence of the change in the activation level of the activatable element. In some embodiments the change in activation level of an activatable element is an increase in the activation level of an activatable element. In some embodiments the activatable element is a protein subject to phosphorylation or dephosphorylation.

In some embodiments, the classification or correlation includes classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition, such as Chronic Lymphocytic Leukemia (CLL).

In some embodiments, the tonic signaling status of a cell is correlated with a clinical outcome such as prognosis or diagnosis of the condition.

In some embodiments, the modulator is anti-IgM (also called F(ab)₂IgM or anti-μ), SDF1a, CD40L, R848 and/or a combination thereof.

In some embodiments, the activatable element is a protein. In some embodiments, the protein is selected from the group consisting of Erk1/2.

In another aspect, the invention provides methods of classifying a cell population by contacting the cell population with at least one modulator from the group F(ab)₂IgM, SDF1a, R848, anti-IgD, CD40L, thapsigargin, fludarabine, bendamustine, poly CpG, or IFNa and/or a combination thereof, determining the presence or absence of an increase in activation level of an activatable element in the cell population, and classifying the cell population based on the presence or absence of the increase in the activation of the activatable element

In another aspect, the invention provides a method of determining time to first treatment (TTFT) in a subject suffering from or suspected of suffering from Chronic Lymphocytic Leukemia (CLL) comprising (i) exposing cells from a sample obtained from the subject to at least two modulators; (ii) measuring, on a single cell basis, the level of an activated form of at least one activatable element in the cells; and (iii) determining a TTFT for the subject based on the information obtained in step (ii). In certain embodiments, the sample is a peripheral blood mononuclear cell (PBMC) sample. In certain embodiments, the two modulators comprise a BCR crosslinker, such as a BCR crosslinker comprising an anti-IgG antibody or antibody fragment, or an anti-IgD antibody or antibody fragment, for example F(Ab)₂Igm, and a chemokine, such as a chemokine selected to mimic the chemokine milieu in which B cells may be present in vivo, for example SDF1α. In certain embodiments, the cell is exposed to the modulators simultaneously for a period of 6-20 minutes. In certain embodiments, the activated form of the activatable element is selected from the group consisting of cPARP, p-AKT, p-ERK, p-LYN, p-PLCg2, p-SYK, p-H2AX, p-STAT1, p-STAT3, p-STAT5, p-STAT6, pZAP-70/pSYK, p-Lck and any combination thereof; in certain embodiments, the activated form of the activatable element is selected from the group consisting of p-AKT, p-ERK, p-LYN, p-PLCg2, p-SYK, p-H2AX, and any combination thereof; in certain embodiments, the activated form of the activatable element comprises p-ERK. In certain embodiments, the method further comprises determining basal levels in cells from the sample not exposed to modulator of an intracellular element. In certain embodiments the method further comprises gating the assay so that only healthy cells are used in the determination of step (iii), for example wherein the gating comprises exposing the cell to a detectable binding element specific for an activated form of an activatable element in the apoptosis pathway, detecting the level of the activated form of the activatable element in the cell, for example cPARP, then gating the cell as either healthy or not healthy based on the level of the activated form of the activatable element detected. In certain embodiments the method further comprises taking an action based at least in part on the TTFT determined, such as taking a later sample from the subject or initiating treatment.

In another aspect, the invention provides a method of determining the functional status of a p53 pathway in cells from a subject comprising (i) exposing cells from a sample obtained from the subject to an agent whose activity depends, at least in part, on a functional p53 pathway; (ii) measuring on a single cell basis the level of an intracellular protein whose levels increase upon induction of the p53 pathway; and (iii) from the levels measured in step (ii), determine the functional status of the p53 pathway in the cells. In certain embodiments, the subject suffers from or is suspected of suffering from CLL. In certain embodiments, the intracellular protein is p21. In certain embodiments, the method further comprises gating the assay so that only healthy cells are used in the determination of step (iii), such as exposing the cell to a detectable binding element specific for an activated form of an activatable element in the apoptosis pathway, for example cPARP, detecting the level of the activated form of the activatable element in the cell, then gating the cell as either healthy or not healthy based on the level of the activated form of the activatable element detected. In certain embodiments, the agent is an alkylating agent, such as an agent selected from the group consisting of bendamustine and fludarabine. In certain embodiments, the cells are exposed to the agent for a period of 12-36 hours. In certain embodiments, the method further comprises administering a drug to the subject, wherein the drug is a drug whose activity is dependent, at least in part, on a functional p53 pathway, such as d drug that is the same as the agent to which the cells were exposed in step (i), for example, bendamustine.

In another aspect, the invention provides a system for informing a decision by a subject and/or healthcare provider for the subject involving diagnosing, prognosing, evaluating status of, or determining a method of treatment for a condition from which the subject is suffering or is suspected of suffering, wherein the system comprises (i) the subject and the healthcare provider; (ii) a unit for analyzing a biological sample obtained from the subject by a method of analysis comprising (a) exposing cells from the sample to one or modulators, or no modulator, (b) exposing the cells to a detectable binding element that binds to a form of an activatable element in the cell, and (c) determining on a single cell basis the levels of the detectable binding element in the cell; and (iii) a unit for communicating the results of the analysis of the sample to the subject and/or healthcare provider so that a decision may be made regarding diagnosis, prognosis, state of, or treatment of the condition that the subject suffers from or is suspected of suffering from. In certain embodiments, the condition is a pathological condition selected from the group consisting of neoplastic, hematopoietic, and autoimmune conditions, such as a non-B lineage derived condition or a B-Cell or B Cell lineage derived condition, or a B-Cell or B Cell lineage derived condition, for example, CLL. In certain embodiments, the system further comprises a unit for treating the sample and transporting the sample to the analysis unit. In certain embodiments, the analysis unit comprises a flow cytometer or mass spectrometer for determining on a single cell basis the levels of the detectable binding element in the cell. In certain embodiments, the form of the activatable element detected is an activated form, and wherein the activatable element is activated by cleavage or phosphorylation. In certain embodiments, the modulator comprises a BCR crosslinker. In certain embodiments, a second modulator comprising a chemokine is also used. In certain embodiments, the form of the activatable element to which the detectable binding element binds is selected from the group consisting of cPARP, p-AKT, p-ERK, p-LYN, p-PLCg2, p-SYK, p-H2AX, p-STAT1, p-STAT3, p-STAT5, p-STAT6, pZAP-70/pSYK, p-Lck and combinations thereof. In certain embodiments, the analytical unit is configured to gate data from healthy vs. unhealthy cells, such as by determining cPARP levels in cells and gating the cells at least in part based on their cPARP levels.

In another aspect the invention provides a method of generating a report wherein the report is in a form that is transportable to an end-user comprising (i) obtaining raw data from a single cell network profile assay performed on a subject suffering from or suspected of suffering from a condition; and (ii) converting the data into a transportable report. In certain embodiments, the condition is CLL. In certain embodiments, the report is a hard copy. In certain embodiments, the report is expressed and stored on computer-readable media in the form of magnetic fields. In certain embodiments, the computer-readable media is a hard drive. In certain embodiments, the method further comprises (iii) obtaining identifying data for the identity of the subject from whom the sample was obtained and converting the data into the transportable report.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows Basal phosphorylation levels of B cell receptor signaling molecules. Intracellular phosphoflow cytometry was used to measure basal levels of phosphorylation of signaling molecules downstream of the BCR in gated B cells from peripheral blood mononuclear cells taken from CLL or healthy donors. Comparison between CLL B cells (left) and healthy B cells (right) showed greater variability in the B cells from the patient group with the exception of p-Erk and p-65 (p-values from Student's t-test comparing Arcsinh transformed MFI values from CLL and healthy B cells shown on right).

FIG. 2 shows H₂O₂ treatment segregates CLL samples into two groups based on their magnitude of BCR-mediated signaling. (A) CLL B cells were left untreated or stimulated for 10 minutes with anti-IgM or anti-IgD (10 μg/ml) alone (labeled as BCR X-link), H₂O₂ (3.3 mM) alone or the combination. Cells were fixed and permeabilized before they were incubated at 4° C. overnight with the core gating antibodies supplemented different antibody panels (Table 2). Two dimensional contour plots show exemplary samples in which CLL B cell subsets exhibit robust signaling mediated by H₂O₂ alone and some additional changes with the addition of a BCR cross-linking agent (for sample CLL014) for proximal BCR signaling molecules. Of note are distinct cell subsets with different signaling capacities within each sample. (B) Two dimensional contour plots show exemplary samples in which CLL B cell subsets show a reduced response to H₂O₂ alone as determined for proximal BCR signaling molecules. (C) STAT5, although not part of the canonical BCR network demonstrates either an increase in phosphorylation in response to H₂O₂ alone (left-hand columns) or a marginal response (right-hand columns). The two dimensional plot has SHP-2 along the X-axis as the SHP-2 antibody was in the same antibody panel as the p-STAT5 antibody. Samples CLL014 and CLL024 show distinct cell subsets with different p-STAT5 signaling capacities.

FIG. 3 shows in vitro exposure of CLL B cells to F-ara-A. Cells were exposed to vehicle or F-ara-A (1 μM) for 48 hours at 37° C. Cells were harvested and incubated with an antibody panel comprising the gating antibodies and antibodies recognizing components of the apoptotic cascade (Table 2). The two dimensional contour plots (cleaved caspase 3 (X-axis) and cleaved PARP (Y-axis)) show that samples CLL014 and CLL024 undergo apoptosis (left-hand panels, double positive for cleaved PARP and Caspase-3, left arrows) in response to F-ara-A treatment. Notably, in these samples there were also cell subsets which were refractory to F-ara-A.

FIG. 4 shows histograms comparing population distributions of all CLL and all healthy B cells based on their fluorescence intensities. (A) Arcsinh transformed fluorescence intensities from all gated CLL and healthy B cells were used to derive the histograms. CLL samples demonstrate multiple examples of bimodal activation, as revealed by modulated signaling (dashed lines) after phosphatase inhibition. See samples with arrows, third column. By contrast healthy B cells demonstrate a single cell subset (solid lines) with minimal activation of signaling. (B) Mixture models were generated from the histograms (dashed lines) of arcsinh transformed fluorescence intensities of the CLL B cells comprised of two normal distributions using the mixdistpackage (Efroni S, Schaefer C F, Buetow K H (2007) Identification of Key Processes Underlying Cancer Phenotypes Using Biologic Pathway Analysis. PLoS ONE 2(5): e425); http://icarus.math.mcmaster.ca/peter/mix/mixdist.pdf for R (http:(double slash)www.r-project.org). To determine component cell populations in a given sample, metrics were defined by computing the area under the curve for the fluorescent intensities of all cells from that sample with respect to a random sampling of 50000 events representing each mixture model-derived distribution. These metrics were termed ‘MixMod1’ and ‘MixMod2’ representing the areas under the curve for the distributions with lower (solid lines) and higher (short-dashed lines) mean fluorescent intensities, respectively. Two normal probability density populations of CLL cells that have a high and low response to signaling molecules downstream of the BCR are depicted by the arrows in the third column mediated signaling: Signaling heterogeneity observed in outlier cells.

FIG. 5 shows association between H₂O₂-mediated signaling and apoptosis induction by F-ara-A. (A) Area under the receiver operating characteristic (AUROC) curves were expressed with 95% confidence limits in order to evaluate how statistically significant H₂O₂-induced signaling is in predicting an in vitro apoptotic response to F-ara-A. The mixture model metric for H₂O₂-mediated signaling was used to calculate whether there was an association with response or lack of response to in vitro exposure to F-ara-A. A value of 0.5 for the ROC plots indicates that the association is due to chance. A value of 1.0 indicates that there is a perfect association. (B) Example of a Mixture Model showing H₂O₂-mediated increase in p-STAT5 and its ability to predict response to F-ara-A for an individual patient. An unscaled mixture model was derived from the mixture model for H₂O₂-mediated p-STAT5 signaling (top panel and FIG. 4B). Samples CLL007 and CLL021 have one population distribution of cells and are refractory to F-ara-A exposure. Samples CLL014 and CLL024 show population distributions of cells that span both subpopulations. CLL B cells in these samples are responsive to F-Ara-A exposure. CLL009 has a signaling profile predictive of apoptotic sensitivity but was refractory to in vitro F-ara-A. This latter sample does not fit the model presumably due to alternative pathways that confer refractoriness to apoptosis. Short-dashed line on the lower part of the scale—population density distribution defined by MixMod1, heavy-dashed line on the higher part of the scale—population density distribution defined by MixMod2, solid line-population density distribution for H₂O₂-mediated p-STAT5 for B cells from an individual patient.

FIG. 6 shows statistical association between H₂O₂-mediated signaling and apoptosis induction by F-ara-A (Fludarabine) in the group comprised of all CLL cells regardless of ZAP-70 or IgV_{H} mutational status compared with the group comprised of ZAP-70 positive or IgV_{H} unmutated status. (A) ROC curves from a fold change model were expressed in order to evaluate how statistically significant H₂O₂-induced signaling is in predicting an in vitro apoptotic response to F-ara-A for all CLL cells, regardless of ZAP-70 or IgV_{H} mutational status (that is, prediction of apoptotic response is based on H₂O₂-induced nodes). The fold change metric for H₂O₂-mediated signaling was used to calculate whether there was an association with response or lack of response to in vitro exposure to F-ara-A. A value of 0.5 for the ROC plots indicates that the association is due to chance. A value of 1.0 indicates that there is a perfect association. (B) ROC curves from a fold change model were expressed with 95% confidence limits to evaluate how statistically significant H₂O₂-induced signaling is in predicting in vitro apoptotic response to F-ara-A for cells with ZAP-70 positive or IgV_{H} unmutated status (that is, prediction of apoptotic response is based on H₂O₂-induced nodes in combination with ZAP-70 or IgVH status).

FIG. 7 shows the biology analyzed in Example 2.

FIG. 8 shows Kaplain-Meier curves comparing TTFT for: (A) Patients were stratified into two groups based on the log 2Fold antiIgM+SDF1a→p-ERK in CLL cells and plotted versus TTFT and (B). Patients were divided based on IgVH mutational status and plotted versus TTFT. p-values are for the log rank test. In Figure B, IgVh mutated samples are shown with the solid line and unmutated samples are shown with a dashed line.

FIG. 9 shows Signaling Nodes Associated with Unmutated IgVH.

FIG. 10 shows that SCNP Identifies Significant Relationship between p21 Induction and Probability of Having p53 Mutated B-CLL.

FIG. 11 shows that Using SCNP As Surrogate For IgVH is Promising.

FIG. 12 shows BCR and Apoptosis Signaling Show Clinical Prognostic Power: Binet Stages A & B.

FIG. 13 (A) and (B) shows the biology analyzed in Example 3.

FIG. 14 shows the Failure to Induce p21 In Response DNA Damage Evident in Donors with del17p13.

FIG. 15 shows IgVH Mutational Status Signaling Associations.

FIG. 16 shows Signaling Associated With TTFT; Comparable Performance as CD38 and IgVH Mutational Status.

FIG. 17 shows preliminary univariate and decision tree AUROC (Binet A/B only); TTFT split at 36 months.

FIG. 18 shows donors with mutated IGHV and greater αIgM+SDF1a→p-ERK have unfavorable disease course.

FIG. 19 shows mutated p53 samples have high basal p-H2AX and fail to induce p21 expression.

FIG. 20 shows that SCNP can enable models to better predict prognosis than IGVH mutational status alone.

FIG. 21 shows that SCNP has the potential to define prognosis beyond IGHV.

FIG. 22 shows SCNP enables multivariate models to better predict IGHV mutational status

FIG. 23 shows induced p21 expression is attenuated in donors with unfavorable cytogenetics.

FIG. 24 shows basal NF-kB signal and ribosomal activity increases in some CLL donors.

FIG. 25 shows ZAP-70 signaling profiles. The nodes for the pairs going from left to right (in a similar manner to FIG. 26) are anti IgM (also known as F(ab)₂IgM)>p-Lyn; anti IgM>p-PLCg2; anti IgM>p-Erk; anti IgM+anti IgD>p-Erk; anti IgM+anti IgD>p-Akt; anti IgM+SDF1a>p-Erk; anti IgD>p-S6; Thapsigargin>p-Akt; Thapsigargin>p-Erk; CpGb>IkB; and CpGb>p-Erk.

FIG. 26 shows CD38 expression profiles.

FIG. 27 shows pathways analyzed in Examples 2 and 3.

FIG. 28 shows patient characteristics for Example 2.

FIG. 29 shows CLL signaling ranges for various signaling nodes in Example 2.

FIG. 30 shows Kaplan-Meier curves of TTFT for subsets of Binet Stage A/B patients in Example 2.

FIG. 31 shows cleaved PARP values in untreated samples in patients in Example 2.

FIG. 32 shows Fludarabine-induced p-H2AX and p-53BP1 signaling was greater than bendamustine signaling at 4 hours in samples selected for low spontaneous cPARP in Example 2.

FIG. 33 shows distribution of p21 induction by bendamustine in cleaved PARP negative cells vs. all B cells at 24 hours in Example 2.

FIG. 34 shows characteristics of subjects from whom samples were obtained in Example 3.

FIG. 35 shows modulators and antibodies used in Example 3 (A) Modulators; (B) Antibodies.

FIG. 36 shows unmodulated signaling in CLL and healthy samples from Example 3 (A) Unmodulated signaling in CD19+CD5+ B-CLL cells in CLL samples and CD19+ B cells in healthy samples. The raw instrument fluorescence intensities of the signaling proteins were converted to calibrated intensity metrics (ERFs, Equivalent Number of Reference Fluorophores). IκB, S6, and STAT1 that differ in their activation status between healthy and CLL are denoted as significant by * p<0.05, **p<0.01. (B) IκB levels (ERF) in unmodulated and modulated CLL and healthy samples. CLL samples on average have lower basal IκB levels near levels observed in healthy samples after BCR modulation.

FIG. 37 shows a heatmap for modulated levels of phosphoproteins from Example 3.

FIG. 38 shows differences in signaling in between healthy and CLL in Example 3 (A) BCR signaling measured at 10 minutes within the CD19+ CD5+ B-CLL cells of CLL samples and the CD19+ B cells of the healthy samples. Data are expressed as Log 2Fold change between unmodulated to modulated levels. The nodes are grouped by signaling protein. (B) CD40L and TLR signaling is heterogeneous across CLL samples and on average is weaker in CLL samples. (C) STAT3 signaling is reduced in CLL samples. (D) p-ERK signaling induced by α-IgM, SDF1α, or the combination in CLL samples. A greater than additive p-ERK signal exists in many of the CLL samples when modulated simultaneously by B cell receptor crosslinking and the chemokine SDF1α modulated levels of phosphoproteins pAKT, pERK, and pS6 in response to various modulators, from Example 3

FIG. 39 (A) and (B) shows IgM modulation identified attenuated activation of proximal signaling proteins LYN, SYK, and PLCγ2 in B-CLL cells relative to the B cells of healthy controls indicative of broad dysfunctional signaling in CLL in Example 3.

FIG. 40 shows signaling profiles associated with IGHV mutational status in Example 3. Functional signaling analysis was performed on samples grouped by their IGHV mutational status. (A) Response to BCR engagement was expressed by the rank-based Uu metric. A Uu of 0.5 (dashed line) is represents no induced signal above unmodulated. *p<0.05, **p<0.01 Similar differences are also observed with the Log 2Fold metric. (B) Unmodulated and αIgM modulated p-ERK in M-CLL and U-CLL samples. M-CLL samples show a trend of decreasing responsiveness to αIgM with increasing basal p-ERK that is not observed in U-CLL samples. (C) Non-BCR signaling pathways including TLR(R848, CpG-B), calcium-modulation (thapsigargin), and DNA-damage (bendamustine) signaling pathways and were interrogated in M-CLL and U-CLL samples revealing significant functional differences for the two risk categories. For all nodes except IκB, an induced response results in a Uu between 0.5 and 1.0; degradation of IκB produces a Uu less than 0.5 For scaling purposes, the induced degradation of IκB is represented as 1-Uu.

FIG. 41 shows BCR modulated signaling across multiple downstream signaling proteins (p-LYN, p-SYK, p-PLCγ2, p-ERK) showed a positive correlation to unmutated IGHV as measured by both the population-based Uu metric and magnitude (Log 2Fold) in Example 3.

FIG. 42 shows signaling analysis of ZAP-70+ and ZAP-70-CLL samples in Example 3. (A) Samples were grouped using a 20% ZAP-70+ cell frequency threshold. Significant differences in BCR, calcium, and TLR9 (CpG-B) signaling are represented as *p<0.05, **p<0.01. (B) α-IgM Log 2Fold activation of p-ERK was compared between the ZAP-70+ cells and ZAP-70− cells within individual samples showing increased signaling in the ZAP-70+ fraction of cells. (C) Analysis of the levels of p-ERK quantified by the ERF metric in ZAP-70+ cells and ZAP-70− cells from unmodulated and modulated samples shows that in both conditions in ZAP-70+ express greater p-ERK.

FIG. 43 shows greater αIgM modulated signaling (p-LYN, p-PLCγ2, p-ERK) and thapsigargin modulated signaling (p-AKT, p-ERK) were identified in samples with greater than 20% ZAP-70+ cells, similar to the trends observed with U-CLL, in Example 3.

FIG. 44 shows signaling with samples stratified by CD38 expression in Example 3. Response to modulation in CD38+ and CD38-CLL samples expressed by the Uu metric. CD38+ samples associated with nodes different from those observed in U-CLL or ZAP-70 risk groups. BCR signaling was comparable between the CD38 sample groups whereas IFNα signaling and DNA damage response differed. The induced degradation of IκB is represented as 1-Uu. Significance was denoted as being not significant, ns, *p<0.05, or **p<0.01.

FIG. 45 shows CD38 positive samples showed a trend of increasing BCR signaling capacity, although these associations did not reach significance, in Example 3.

FIG. 46 shows univariate associations between signaling and TTFT, i.e., signaling nodes associated with TTFT and their predictive power, in Example 3.

FIG. 47 shows univariate associations between signaling and TTFT, i.e., signaling nodes associated with TTFT and their predictive power, in Example 3.

FIG. 48 shows, in Example 3, Kaplan-Meier analysis of TTFT for subgroups of RAI I/O patients. Signaling associates with TTFT with similar performance as IGHV mutational status and CD38 expression. (A)αIgM+SDF1α→p-ERK|Log 2Fold associates with TTFT with similar performance as IGHV mutational status (B) or CD38 expression (C) and performs better than ZAP-70 expression (D).

FIG. 49 shows intracellular proteins and modulators examined in Example 2.

FIG. 50 shows, for Example 3, signaling analysis may help define prognosis beyond IGHV mutational status. The three plots show the logistic regression model of IGHV mutational status with available TTFT data overlayed for all the CLL samples or divided by IGHV mutational status. Follow up time varied across donors with M-CLL donors having a median time of follow up of 69 months compared to 40 months for U-CLL donors.

FIG. 51 shows the trend of greater αIgM→p-ERK signaling with TTFT was observed (Uu metric, p=0.05, likelihood ratio (LR) χ2 test; log 2Fold metric p=0.07) in Example 2.

FIG. 52 (A) and (B) shows Anti-IgM+SDF1a→p-ERK|Uu plotted in IGHV mutated and unmutated samples and TTFT was depicted for patients in Example 2.

FIG. 53 shows samples with 17p deletion had impaired p21 induction in response to culturing in the presence of bendamustine in patients in Example 2.

### DETAILED DESCRIPTION OF THE INVENTION

Objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description.

All publications, patents, and patent applications mentioned in this specification are herein incorporated by reference to the same extent as if each individual publication or patent application was specifically and individually indicated to be incorporated by reference

This application incorporates by reference, in their entireties, U.S. Ser. No. 60/957,160 filed Aug. 21, 2007, U.S. Ser. No. 61/048,920 filed Apr. 29, 2008 and U.S. Ser. No. 12/229,476 filed Aug. 21, 2008.

The present invention incorporates information disclosed in other applications and texts. The following patent and other publications are hereby incorporated by reference in their entireties: Haskell et al, Cancer Treatment, 5^{th} Ed., W.B. Saunders and Co., 2001; Alberts et al., The Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7^{th} Ed., Garland, and Leroith and Bondy, Growth Factors and Cytokines in Health and Disease, A Multi Volume Treatise, Volumes 1A and 1B, Growth Factors, 1996.

Also, patents and applications that are incorporated by reference include U.S. Pat. Nos. 7,381,535, 7,393,656, 7,563,584, 7,695,924, 7,695,926, 7,939,278, 8,148,094, 8,187,885, 8,198,037, 8,206,939, 8,214,157, 8,227,202; U.S. patent application Ser Nos. 11/338,957, 11/655,789, 12/061,565, 12/125,759, 12/125,763, 12/229,476, 12/432,239, 12/432,720, 12/471,158, 12/501,274, 12/501,295, 12/538,643, 12/551,333, 12/581,536, 12/606,869, 12/617,438, 12/687,873, 12/688,851, 12/703,741, 12/713,165, 12/730,170, 12/778,847, 12/784,478, 12/877,998, 12/910,769, 13/082,306, 13/091,971, 13/094,731, 13/094,735, 13/094,737, 13/098,902, 13/098,923, 13/098,932, 13/098,939, 13/384,181; International Applications Nos. PCT/US2011/001565, PCT/US2011/065675, PCT/US2011/026117, PCT/US2011/029845, PCT/US2011/048332; and U.S. Provisional Application Ser. Nos. 60/304,434, 60/310,141, 60/646,757, 60/787,908, 60/957,160, 61/048,657, 61/048,886, 61/048,920, 61/055,362, 61/079,537, 61/079,551, 61/079,579, 61/079,766, 61/085,789, 61/087,555, 61/104,666, 61/106,462, 61/108,803, 61/113,823, 61/120,320, 61/144,68, 61/144,955, 61/146,276, 61/151,387, 61/153,627, 61/155,373, 61/156,754, 61/157,900, 61/162,598, 61/162,673, 61/170,348, 61/176,420, 61/177,935, 61/181,211, 61/182,518, 61/182,638, 61/186,619, 61/216,825, 61/218,718, 61/226,878, 61/236,281, 61/240,193, 61/240,613, 61/241,773, 61/245,000, 61/254,131, 61/263,281, 61/265,585, 61/265,743, 61/306,665, 61/306,872, 61/307,829, 61/317,187, 61/327,347, 61/350,864, 61/353,155, 61/373,199, 61/374,613, 61/381,067, 61/382,793, 61/423,918, 61/436,534, 61/440,523, 61/469,812, 61/499,127, 61/515,660, 61/521,221, 61/542,910, 61/557,831, 61/558,343, 61/565,391, 61/565,929, 61/565,935, 61/591,122, 61/640,794, 61/658,092, 61/664,426.

Some commercial reagents, protocols, software and instruments that are useful in some embodiments of the present invention are available at the Becton Dickinson Website http(double slash)www.bdbiosciences.com/features/products/, and the Beckman Coulter website, http: (double slash)www.beckmancoulter.com/Default.asp?bhfv=7. Relevant articles include High-content single-cell drug screening with phosphospecific flow cytometry, Krutzik et al., Nature Chemical Biology, 23 December (2007); Irish et al., FLt3 ligand Y591 duplication and Bcl-2 over expression are detected in acute myeloid leukemia cells with high levels of phosphorylated wild-type p53, Neoplasia, (2007), Irish et al. Mapping normal and cancer cell signaling networks: towards single-cell proteomics, Nature (2006) 6:146-155; and Irish et al., Single cell profiling of potentiated phospho-protein networks in cancer cells, Cell, (2004) 118, 1-20; Schulz, K. R., et al., Single-cell phospho-protein analysis by flow cytometry, Curr Protoc Immunol, (2007) 78:8 8.17.1-20; Krutzik, P. O., et al., Coordinate analysis of murine immune cell surface markers and intracellular phosphoproteins by flow cytometry, J. Immunol. (2005) 175(4):2357-65; Krutzik, P. O., et al., Characterization of the murine immunological signaling network with phosphospecific flow cytometry, J Immunol. (2005) 175(4):2366-73; Shulz et al., Current Protocols in Immunology (2007) 78:8.17.1-20; Stelzer et al. Use of Multiparameter Flow Cytometry and Immunophenotyping for the Diagnosis and Classification of Acute Myeloid Leukemia, Immunophenotyping, Wiley, 2000; and Krutzik, P. O. and Nolan, G. P., Intracellular phospho-protein staining techniques for flow cytometry: monitoring single cell signaling events, Cytometry A. (2003) 55(2):61-70; Hanahan D., Weinberg, The Hallmarks of Cancer, CELL (2000) 100:57-70; Krutzik et al, High content single cell drug screening with phosphospecific flow cytometry, Nat Chem. Biol. (2008) 4:132-42; and Monroe, J. G., Ligand independent tonic signaling in B-cell receptor function, Current Opinion in Immunology 2004, 16:288-295. Experimental and process protocols and other helpful information can be found at http:/proteomices.stanford.edu. The articles and other references cited below are also incorporated by reference in their entireties for all purposes.

### INTRODUCTION

In some embodiments, this invention is directed to methods and compositions for diagnosis, prognosis and to methods of treatment. In some embodiments, the physiological status of cells present in a sample (e.g. clinical sample) is used, e.g., in diagnosis or prognosis of a condition, patient selection for therapy, to monitor treatment, modify therapeutic regimens, and to further optimize the selection of therapeutic agents; which may be administered as one or a combination of agents. Hence, therapeutic regimens can be individualized and tailored according to the data obtained prior to, and at different times over the course of treatment, thereby providing a regimen that is individually appropriate.

In some embodiments, the present invention is directed to methods for classifying a sample derived from an individual having or suspected of having a condition, e.g., a neoplastic, autoimmune or a hematopoietic condition. The invention allows for identification of prognostically and therapeutically relevant subgroups of conditions and prediction of the clinical course of an individual. The methods of the invention provide tools useful in the treatment of an individual afflicted with a condition, including but not limited to methods of choosing a therapy for an individual, methods of predicting response to a therapy for an individual, methods of determining the efficacy of a therapy in an individual, methods for assigning a risk group, methods of predicting an increased risk of relapse, methods of predicting an increased risk of developing secondary complications, and methods of determining the prognosis for an individual. The present invention provides methods that can serve as a prognostic indicator to predict the course of a condition, e.g. whether the course of a neoplastic, autoimmune or a hematopoietic condition in an individual will be aggressive or indolent, thereby aiding the clinician in managing the patient and evaluating the modality of treatment to be used.

In some embodiments, the invention is directed to methods for determining the activation level of one or more activatable elements in a cell upon treatment with one or more modulators. The activation of an activatable element in the cell upon treatment with one or more modulators can reveal operative pathways in a condition that can then be used, e.g., choose a therapy for an individual, predict response to a therapy for an individual, determine the efficacy of a therapy in an individual. In some embodiments the modulators may themselves be used directly within individuals as therapeutic agents. In some embodiments the activation of an activatable agent may be used as an indicator to predict course of the condition, identify risk group, predict an increased risk of developing secondary complications, and determine the prognosis for an individual.

In some embodiments, the invention is directed to methods for classifying a cell by contacting the cell with an inhibitor, determining the presence or absence of an increase in activation level of an activatable element in the cell, and classifying the cell based on the presence or absence of the increase in the activation of the activatable element. In some embodiments, the invention is directed to methods of determining the presence or absence of a condition in an individual by subjecting a cell from the individual to a modulator and an inhibitor, determining the activation level of an activatable element in the cell, and determining the presence or absence of the condition based on the activation level upon treatment with a modulator and an inhibitor.

In some embodiments, the invention is directed to methods for classifying a cell by contacting the cell with an inhibitor, determining the presence or absence of a change in activation level of an activatable element in the cell, and classifying the cell based on the presence or absence of the change in the activation of the activatable element. In some embodiments the change is an increase. In some embodiments the change is a decrease.

In some embodiments, the invention is directed to methods of determining tonic signaling status of a cell by subjecting the cell to a modulator, determining the activation level of an activatable element that participates in a tonic signaling pathway in the cell, and determining the status of a tonic signaling pathway in the cell from the activation level. Tonic signaling in a cell may have functional consequences, for instance, to maintain certain differentiated cellular properties or functions. In some embodiments of the invention, the status of a tonic signaling pathway is used to correlate the status to differences in populations.

In some embodiments, the invention is directed to methods of determining a phenotypic profile of a population of cells by exposing the population of cells, optionally in separate cultures, to a plurality of modulators, wherein at least one of the modulators is an inhibitor, determining the presence or absence of an increase in activation level of an activatable element in the cell population from each of the separate culture and classifying the cell population based on the presence or absence of the increase in the activation of the activatable element from populations of cells in each separate culture.

In some embodiments a method for classifying a cell comprises contacting the cell with an inhibitor, determining the presence or absence of a change in an activation level of at least one activatable element in said cell, and classifying said cell based on said presence or absence of said change in the activation level of said at least one activatable element. In some embodiments the change is an increase. In some embodiments the change is a decrease.

In some embodiments the method of classifying a cell further comprises determining the level of an intracellular marker, cell surface marker or any combination thereof. For example a cell may be classified by a change in activation level of an activatable element and also by the level of one or more cell surface markers. In addition a cell may be classified by a change in activation level of an activatable element and by the level of an intracellular marker. Combinations may also be used. Serum markers are also useful in methods of diagnosis, prognosis, determining treatments effects and/or choosing a treatment.

One or more cell surface markers may also be used in the method of the invention in addition to intracellular markers (e.g. phospho-proteins). In some embodiments, the method comprises determining the level of a plurality of cell surface markers. Cell surface markers may include any cell surface molecule that is detected by flow cytometry. In some embodiments the cell surface marker is a human leukocyte differentiation antigen. In some embodiments the human leukocyte differentiation antigen is selected from the list: CD1, CD2, CD3, CD4, CD5, CD8, CD10, CD14, CD19, CD20, CD22, CD23, CD40, CD52, CD100, CD280, CD281, CD282, CD283, CD284, and CD289. In some embodiments the human leukocyte differentiation antigen is selected from the list comprising CD1 though CD300. In some embodiments the cell surface marker is any cell surface receptor or ligand. Examples of cell surface ligands and receptors include, but are not limited to, members of the TNF superfamily, interleukins, hormones, neurotransmitters, interferons, growth factors, chemokines, integrins, toll receptor ligands, prostaglandins, or leukotriene families. Other examples of cell surface markers include, but are not limited to metalloproteases. In some embodiments the cell surface marker is membrane bound IgM. In some embodiments the cell surface marker is a B-cell receptor (BCR) or a component of a BCR. In some embodiments the marker is CD45, CD5, CD14, CD19, CD20, CD22, CD23, CD27, CD37, CD40, CD52, CD79, CD38, CD96, major histocompatability antigen (MHC) Class 1 or MHC Class 2. In some embodiments the cell surface marker is membrane bound IgD. In some embodiments the cell surface marker is membrane bound IgG. In some embodiments, the method of classifying a cell comprises determining a level of at least one cell surface marker on said cell and an activation level of at least one activatable element on said cell. In some embodiments, the method of classifying a cell comprises determining the level of cell surface IgM on said cell. In some embodiments, the method comprises determining the level of cell surface IgD on said cell. In some embodiments, the method comprises determining the level of a BCR on said cell. In some embodiment the cell surface marker is associated with a disease or conditions. In some embodiments the maker is CD38 or CD96. In some embodiments the marker is CD38 and the condition is leukemia. In some embodiments the marker is CD96 and the condition is leukemia.

One or more intracellular markers may be used in the method of the invention. The levels of these markers can be determined before they are secreted and are referred to as “captured”. Examples of captured intracellular markers include, but are not limited to, TNF superfamily members, interleukins, hormones, neurotransmitters, interferons, growth factors, chemokines, integrins, prostaglandins, leukotrines and receptors for all of the above. Examples of intracellular markers also include, but are not limited to, metalloproteases. Examples of intracellular markers also include, but are not limited to, proteins involved in programmed cell death and proliferation. Examples of intracellular markers also include, but are not limited to viruses, pathogens, parasites and components or products thereof. In some embodiments, the method of classifying a cell further comprises determining the level of an intracellular pathogen or component of a pathogen. In some embodiments the intracellular pathogen is HIV. In some embodiments the intracellular pathogen is EBV. In some embodiments the intracellular component of a pathogen is a nucleic acid sequence derived from said pathogen. In some embodiments the intracellular component of a pathogen is a pathogen derived polypeptide.

The method of the invention may comprise determining the level of one or more serum markers. In some embodiments the serum marker is a marker of a condition. In some embodiments the serum marker is a marker of inflammation. In some embodiments the serum marker is a soluble cytokine, TNF superfamily member, interleukin, hormone, neurotransmitter, interferon, growth factor, chemokine, integrin, prostaglandin, leukotriene or any soluble receptor thereof. In some embodiments the serum marker is a marker of a specific disease or condition. In some embodiments the serum marker is a cancer marker. In some embodiments the serum marker is a leukemia marker. In some embodiments the serum marker is beta-2-microglobulin, calcitonin, CD20, CD23, CD52, IL6, IL2R, ICAM-1, CD14, IgG, thymidine kinase or ferritin. In some embodiments the serum marker is a pharmaceutical drug, pathogen, virus, parasite, small compound or toxin. Therefore, in some embodiments, the methods described herein are for diagnosis, prognosis or determining a method of treatment for a subject or patient. In some embodiments the methods comprise classifying a cell or population of cells. In certain embodiments, the methods of diagnosis, prognosis or determining a method of treatment comprise determining the level of at least one serum marker derived from the subject or patient. In some embodiments the serum marker is a cytokine, chemokine, soluble receptor, growth factor, antibody or binding protein. In some embodiments the serum marker is a pathogen. In some embodiments the serum marker is a pharmaceutical compound or drug.

In one embodiment, the present invention can distinguish between responders and non-responder cells from patients after those cells are treated with an anti-cancer agent, such as 9-β-D-arabinosyl-2-fluoroadenine (F-ara-A), the free nucleoside of fludarabine. In an embodiment of the invention, CLL cells are contacted with modulators, such as F(ab)₂ IgM (also called anti-μ) and H₂O₂ alone or combined together. Activatable elements such as phosphorylated Lyn, Syk, PLCγ2, BLNK, STAT5, Erk, p65/RelA, Akt (Akt1, Akt2, Akt3), S6, Chk2, cleaved PARP, cleaved caspase 3, cleaved caspase 8, cytosolic cytochrome C and Bcl-2 expression are analyzed to assist in the correlation between responses in cells and clinical outcomes.

The subject invention also provides kits for use in determining the physiological status of cells in a sample, the kit comprising one or more specific binding elements for signaling molecules, and may additionally comprise one or more therapeutic agents. The kit may further comprise a software package for data analysis of the physiological status, which may include reference profiles for comparison with the test profile.

As disclosed herein is a method for classifying a cell comprising contacting the cell with a modulator or an inhibitor used to determine the presence or absence of a change in activation level of an activatable element in the cell, and classifying the cell based on the presence or absence of the change in the activation level of the activatable element. In some embodiments the change in activation level of an activatable element is an increase in the activation level of an activatable element. In some embodiments the activatable element is a protein subject to phosphorylation or dephosphorylation.

In some embodiments, one aspect of the invention is tyrosine phosphatase inhibitor (e.g. peroxide) mediated STAT5 or AKT phosphorylation to segregate or stratify patients. In another embodiment, the invention relates to measuring in vitro apoptosis in response to F-ara-A into separate classes of patients who are apoptosis competent or refractory. Another aspect of the invention relates to the use of classification and modeling methods such as logistic regression (including regularized, penalized, and shrinkage methods including lasso and ridge), decision trees, random forests, support vector machines, boosting, etc. to generate univariate and multivariate models associating tyrosine phosphatase inhibitor (e.g. hydrogen peroxide (H₂O₂)) or B-cell receptor cross linking induced changes in phosphorylation with the ability of cells to undergo apoptosis. Another aspect of the invention is the detection of ZAP-70 to increase the predictability of the area under the ROC curve or the use of the ROC curve to determine the suitability of a classification and modeling method. Another aspect of the invention relates to the use of mixture models to represent data for the uses disclosed herein. In another embodiment, detection of ZAP-70, IGVH and/or CD38 can be used as clinical covariates that can be combined with phosphorylation and/or signaling readouts, in multivariate models of the methods described throughout the specification.

In some embodiments of the methods, the invention provides a method for classifying a cell by contacting the cell with an inhibitor; determining the activation levels of a plurality of activatable elements in the cell; and classifying the cell based on the activation level. In some embodiments, the inhibitor is a kinase or phosphatase inhibitor, such as adaphostin, AG 490, AG 825, AG 957, AG 1024, aloisine, aloisine A, alsterpaullone, aminogenistein, API-2, apigenin, arctigenin, AY-22989, BAY 61-3606, bisindolylmaleimide IX, chelerythrine, 10-[4′-(N,N-Diethylamino)butyl]-2-chlorophenoxazine hydrochloride, dasatinib, 2-Dimethylamino-4,5,6,7-tetrabromo-1H-benzimidazole, 5,7-Dimethoxy-3-(4-pyridinyl)quinoline dihydrochloride, edelfosine, ellagic acid, enzastaurin, ER 27319 maleate, erlotinib, ET18OCH3, fasudil, flavopiridol, gefitinib, GW 5074, H-7, H-8, H-89, HA-100, HA-1004, HA-1077, HA-1100, hydroxyfasudil, indirubin-3′-oxime, 5-Iodotubercidin, kenpaullone, KN-62, KY12420, LFM-A13, lavendustin A, luteolin, LY-294002, LY294002, mallotoxin, ML-9, NSC-154020, NSC-226080, NSC-231634, NSC-664704, NSC-680410, NU6102, olomoucine, oxindole I, PD-153035, PD-98059, PD-169316, phloretin, phloridzin, piceatannol, picropodophyllin, PKI, PP1, PP2, purvalanol A, quercetin, R406, R788, rapamune, rapamycin, Ro 31-8220, roscovitine, rottlerin, SB202190, SB203580, sirolimus, sorafenib, SL327, SP600125, staurosporine, STI-571, SU-11274, SU1498, SU4312, SU6656, 4,5,6,7-Tetrabromotriazole, TG101348, Triciribine, Tyrphostin AG 490, Tyrphostin AG 825, Tyrphostin AG 957, Tyrphostin AG 1024, Tyrphostin SU1498, U0126, VX-509, VX-667, VX-680, W-7, wortmannin, XL-019, XL-147, XL-184, XL-228, XL-281, XL-518, XL-647, XL-765, XL-820, XL-844, XL-880, Y-27632, ZD-1839, ZM-252868, ZM-447439, H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenyarsine oxide, Pyrrolidine Dithiocarbamate, or Aluminum fluoride. In some embodiments the phosphatase inhibitor is a tyrosine phosphatase inhibitor, such as H₂O₂.

In some embodiments the cell or cell population (hereinafter called a “cell”) is a hematopoietic-derived cell. In some embodiments, the hematopoietically derived cell is selected from the group consisting of pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells. In some embodiments, the hematopoietic derived cell is a B-lymphocyte lineage progenitor and derived cell, e.g., an early pro-B cell, late pro-B cell, large pre-B cell, small pre-B cell, immature B cell, mature B cell, plasma cell and memory B cell, a CD5+ B cell, a CD38+ B cell, a B cell bearing a mutated or non mutated heavy chain of the B cell receptor, or a B cell expressing ZAP-70.

In some embodiments, the classification or correlation includes classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition, such as Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibrosis, or atypical immune lymphoproliferations, systemic lupus erythematosis (SLE), rheumatoid arthritis (RA). In some embodiments, the neoplastic, autoimmune or hematopoietic condition is non-B lineage derived, such as acute myeloid leukemia (AML), Chronic Myeloid Leukemia (CML), non-B cell acute lymphocytic leukemia (ALL), non-B cell lymphomas, myelodysplastic disorders, myeloproliferative disorders, myelofibrosis, thrombocythemias, or non-B atypical immune lymphoproliferations. In some embodiments, the neoplastic, autoimmune or hematopoietic condition is a B-Cell or B cell lineage derived disorder, such as Chronic Lymphocytic Leukemia (CLL), B-cell lymphoma, B lymphocyte lineage leukemia, B lymphocyte lineage lymphoma, Multiple Myeloma, acute lymphoblastic leukemia (ALL), B-cell pro-lymphocytic leukemia, precursor B lymphoblastic leukemia, hairy cell leukemia or plasma cell disorders, e.g., amyloidosis or Waldenstrom's macroglobulinemia, B cell lymphomas including but not limited to diffuse large B cell lymphoma, follicular lymphoma, mucosa associated lymphatic tissue lymphoma, small cell lymphocytic lymphoma and mantle cell lymphoma. In some embodiments, the condition is CLL. In some embodiments, the CLL is defined by a monoclonal B cell population that co-expresses CD5 with CD19 and CD23 or CD5 with CD20 and CD23 and by surface immunoglobulin expression.

In some embodiments, the clinical outcome is the staging or grading of a neoplastic, autoimmune or hematopoietic condition. Examples of staging in methods provided by the invention include aggressive, indolent, benign, refractory, Roman Numeral staging, TNM Staging, Rai staging, Binet staging, WHO classification, FAB classification, IPSS score, WPSS score, limited stage, extensive stage, staging according to cellular markers such as ZAP-70 and CD38, occult, including information that may inform on time to progression, progression free survival, overall survival, or event-free survival.

In some embodiments of the invention, the activation level of the plurality of activatable elements in the cell is selected from the group consisting of cleavage by extracellular or intracellular protease exposure, novel hetero-oligomer formation, glycosylation level, phosphorylation level, acetylation level, methylation level, biotinylation level, glutamylation level, glycylation level, hydroxylation level, isomerization level, prenylation level, myristoylation level, lipoylation level, phosphopantetheinylation level, sulfation level, ISGylation level, nitrosylation level, palmitoylation level, SUMOylation level, ubiquitination level, neddylation level, citrullination level, deamidation level, disulfide bond formation level, proteolytic cleavage level, translocation level, changes in protein turnover, multi-protein complex level, oxidation level, multi-lipid complex, and biochemical changes in cell membrane. In some embodiments, the activation level is a phosphorylation level. In some embodiments, the activatable element is selected from the group consisting of proteins, carbohydrates, lipids, nucleic acids and metabolites. In some embodiments, the activatable element is a protein. In some embodiments, the activatable element is a change in metabolic state, temperature, or local ion concentration. In embodiments where the activatable element is a protein, in some embodiments the protein is a protein subject to phosphorylation or dephosphorylation, such as kinases, phosphatases, adaptor/scaffold proteins, ubiquitination enzymes, adhesion molecules, contractile proteins, cytoskeletal proteins, heterotrimeric G proteins, small molecular weight GTPases, guanine nucleotide exchange factors, GTPase activating proteins, caspases and proteins involved in apoptosis (e.g. PARP), ion channels, molecular transporters, molecular chaperones, metabolic enzymes, vesicular transport proteins, hydroxylases, isomerases, transferases, deacetylases, methylases, demethylases, proteases, esterases, hydrolases, DNA binding proteins or transcription factors. In some embodiments, the protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHPT, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tpl2, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP-70, Lyn, Cbl, SLP-76, PLCγ, PLCγ 2, transcription factor, STAT1, STAT3, STAT4, STAT5, STATE, FAK, p130CAS, PAKs, LIMK1/2, Hsp90, Hsp70, Hsp27, SMADs, Rel-A (p65-NFKB), CREB, Histone H2B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, PARP, IAPB, Smac, Fodrin, Actin, Src, Lyn, Fyn, Lyn, NIK, IκB, p65(RelA), IKK□, PKA, PKC□, PKC□, PKC□, PKC□, CAMK, Elk, AFT, Myc, Egr-1, NFAT, ATF-2, Mdm2, p53, DNA-PK, Chk1, Chk2, ATM, ATR, β-catenin, CrkL, GSK3β, GSK3β, and FOXO. In some embodiments, the protein selected from the group consisting of Erk, Syk, ZAP-70, Lyn, Btk, BLNK, Cbl, PLC□2, Akt, RelA, p38, S6. In some embodiments the protein is S6.

In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lyn, Fgr, Yes, Csk, Abl, Btk, ZAP-70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3a, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phospholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon □, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, PARP, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPB, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Glycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-catenin, FOXO transcription factor, STAT1, STAT2, STAT3, STAT4, STAT5a, STAT5b, STATE, p53, WT-1, HMGA, pS6, 4EPB-1, eIF4E-binding protein, RNA polymerase, initiation factors, elongation factors.

In some embodiments of the methods of the invention, the modulator to which the cell is subjected is an activator or an inhibitor. In some embodiments, the modulator is, e.g., a growth factor, cytokine, adhesion molecule modulator, hormone, small molecule, polynucleotide, antibodies, natural compounds, lactones, chemotherapeutic agents, immune modulator, carbohydrate, proteases, ions, reactive oxygen species, or radiation. In some embodiments, the modulator is a B cell receptor modulator, e.g., a B cell receptor activator such as a cross-linker of the B cell receptor complex or the B-cell co-receptor complex. In some embodiments of the invention, the cell is subjected to a modulator and a separate B cell receptor modulator (such as a B cell receptor cross-linker). In some embodiments, the cross-linker is an antibody, or molecular binding entity. In some embodiments, the cross-linker is an antibody, such as a multivalent antibody. In some embodiments, the antibody is a monovalent, bivalent, or multivalent antibody made more multivalent by attachment to a solid surface or tethered on a nanoparticle surface to increase the local valency of the epitope binding domain. In some embodiments, the cross-linker is a molecular binding entity, such as an entity that acts upon or binds the B cell receptor complex via carbohydrates or an epitope in the complex. In some embodiments, the molecular binding entity is a monovalent, bivalent, or multivalent binding entity that is made more multivalent by attachment to a solid surface or tethered on a nanoparticle surface to increase the local valency of the epitope binding domain. In some embodiments where the modulator is a B cell receptor modulator, e.g., a B cell receptor activator such as a cross-linker of the B cell receptor complex or the B-cell co-receptor complex, cross-linking includes binding of an antibody or molecular binding entity to the cell and then causing its crosslinking via interaction of the cell with a solid surface that causes crosslinking of the BCR complex via antibody or molecular binding entity. In some embodiments, the crosslinker is selected from the group consisting of F(ab)₂ IgM, IgG, IgD, polyclonal BCR antibodies, monoclonal BCR antibodies, and Fc receptor derived binding elements. The Ig may be derived from a species selected from the group consisting of mouse, goat, rabbit, pig, rat, horse, cow, shark, chicken, or llama. In some embodiments, the crosslinker is F(ab)₂ IgM, Polyclonal IgM antibodies, Monoclonal IgM antibodies, Biotinylated F(ab)₂ IgG/M, Biotinylated Polyclonal IgM antibodies, Biotinylated Monoclonal IgM antibodies and/or a combination thereof.

In some embodiments of the methods of the invention, the cell is subjected to a B cell receptor activator and a phosphatase inhibitor or kinase inhibitor, such as F(ab)₂IgM or biotinylated F(ab)₂IgM and a phosphatase inhibitor (e.g., H₂O₂).

In some embodiments, the invention provides a method of determining a tonic signaling (ligand independent) status of a cell by subjecting the cell to a modulator, determining the activation level of an activatable element that participates in a tonic signaling pathway in the cell, and determining the status of a tonic signaling pathway in the cell from the activation level. In some embodiments, a condition of an individual is determined based on tonic signaling status of a cell. In some embodiments, the condition is a neoplastic, autoimmune and/or hematopoietic condition as discussed above.

In some embodiments, the tonic signaling status of a cell is correlated with a clinical outcome such as prognosis or diagnosis of the condition.

In some embodiments, the correlation is determining the individual's response to a treatment, e.g., normal responder, hyper responder, poor responder, having emerging resistance, non-compliant, and adverse reaction.

In some embodiments of this aspect, the invention provides a method of correlating an activation level of a B-lymphocyte lineage derived cell with a neoplastic, autoimmune or hematopoietic condition in an individual by subjecting the B-lymphocyte lineage derived cell from the individual to a modulator; determining the activation levels of a plurality of activatable elements that participate in a tonic signaling pathway in the B-lymphocyte lineage derived cell; and identifying a pattern of the activation levels of the plurality of activatable elements in the tonic signaling pathway in the cell that correlates with a clinical outcome, such as the prediction of outcome for a particular treatment, a prognosis or diagnosis of a certain condition (e.g., a neoplastic condition).

In some embodiments of the methods of the invention, the cell is further subjected to a second modulator, e.g., the cell may be subjected to a B cell receptor activator and a phosphatase inhibitor, such as F(ab)₂IgM or biotinylated F(ab)₂IgM and a phosphatase inhibitor (e.g., H₂O₂).

In addition to determining the activation level of an activatable protein, in some embodiments the methods for classifying a cell further comprise determining the level of an additional intracellular marker and/or a cell surface marker. In some embodiments the methods for classifying a cell comprise determining the level of an additional intracellular marker. In some embodiments the intracellular marker is a captured intracellular cytokine. In some embodiments the methods for classifying a cell comprise determining the level of an additional cell surface marker. In some embodiments the cell surface marker is a cell surface ligand or receptor. In some embodiments the cell surface marker is a component of a B-cell receptor complex. In some embodiments the cell surface marker is CD45, CD5, CD19, CD20, CD22, CD23, CD27, CD37, CD40, CD52, CD79, CD38, CD96, major histocompatability antigen (MHC) Class 1 or MHC Class 2.

In some embodiments the methods of the invention for prognosis, diagnosis, or determination of treatment further comprise determining the level of an additional serum marker. In some embodiments the serum marker comprises a protein. In some embodiments the serum marker is a cytokine, growth factor, chemokine, soluble receptor, small compound, or pharmaceutical drug. In some embodiments the serum marker comprises a component or product of a pathogen or parasite. In some embodiments the serum marker is selected from a group consisting of beta-2-microglobulin, calcitonin, thymidine kinase and ferritin.

In some embodiments, the invention provides a method of correlating an activation level of B-lymphocyte lineage derived cells with a neoplastic, autoimmune or hematopoietic condition in an individual by subjecting the B-lymphocyte lineage derived cell from the individual to a modulator; determining the activation levels of a plurality of activatable elements in the B-lymphocyte lineage derived cell; and identifying a pattern of the activation levels of the plurality of activatable elements in the cell that correlates with the neoplastic condition. In some embodiments, the activatable element is selected from the group consisting of elements selected from the group consisting of Erk, Syk, ZAP-70, Lyn, Btk, BLNK, Cbl, PLCγ2, Akt, RelA, p38, S6 (which can be phosphorylated). In some embodiments, the activatable element is selected from the group consisting of Cbl, PLCγ2, and S6. In some embodiments, the activatable element is S6. In some embodiments, the B-lymphocyte lineage progenitor or derived cell is selected from the group consisting of early pro-B cell, late pro-B cell, large pre-B cell, small pre-B cell, immature B cell, mature B cell, plasma cell and memory B cell, a CD5+ B cell, a CD38+ B cell, a B cell bearing a mutilated or non mutated heavy chain of the B cell receptor, or a B cell expressing ZAP-70. In some embodiments, the invention provides methods for correlating and/or classifying an activation state of a CLL cell with a clinical outcome in an individual by subjecting the CLL cell from the individual to a modulator, where the CLL cell expresses a B-Cell receptor (BCR), determining the activation levels of a plurality of activatable elements, and identifying a pattern of the activation levels of the plurality of activatable elements to determine the presence or absence of an alteration in signaling proximal to the BCR, wherein the presence of the alteration is indicative of a clinical outcome.

In some embodiments the method comprises identifying a pattern of said activation levels of said plurality of activatable elements in said cell, wherein said pattern is correlated to a disease or condition.

In some embodiments, the correlation is determining the individual's response to a specific treatment, e.g., normal responder, hyper responder, poor responder, having emerging resistance, non-compliant, and adverse reaction.

In some embodiments of the invention, the modulator to which the cell is subjected is an activator or an inhibitor. In some embodiments, the modulator is, e.g., a growth factor, cytokine, adhesion molecule modulator, hormone, small molecule, polynucleotide, antibody, natural compound, lactone, chemotherapeutic agent, immune modulator, carbohydrate, protease, ion, reactive oxygen species, or radiation. In some embodiments, the modulator is an antibody, e.g. anti-CD20 (such as rituximab), anti-CD22 (such as epratuzumab), anti-CD23 (such as lumiliximab) or anti-CD52 (such as alemtuzumab), that recognize antigens on the cell surface. Newer generation antibodies have been generated to the above cell surface antigens. In some embodiments, the modulator is a B cell receptor complex modulator, e.g., anti-CD20, which recognizes a component of the B cell receptor co-complex, or a B cell receptor activator such as a cross-linker of the B cell receptor complex or the B-cell co-receptor complex. In some embodiments, the cross-linker is an antibody, or molecular binding entity. In some embodiments, the cross-linker is an antibody, such as a multivalent antibody. In some embodiments, the antibody is a monovalent, bivalent, or multivalent antibody made more multivalent by attachment to a solid surface or tethered on a nanoparticle surface to increase the local valency of the epitope binding domain. In some embodiments, the cross-linker is a molecular binding entity, such as an entity that acts upon or binds the B cell receptor complex via carbohydrates or an epitope in the complex. In some embodiments, the molecular binding entity is a monovalent, bivalent, or multivalent binding entity that is made more multivalent by attachment to a solid surface or tethered on a nanoparticle surface to increase the local valency of the epitope binding domain. In some embodiments where the modulator is a B cell receptor modulator, e.g., a B cell receptor activator such as a cross-linker of the B cell receptor complex or the B-cell co-receptor complex, cross-linking includes binding of an antibody or molecular binding entity to the cell and then causing its crosslinking via interaction of the cell with a solid surface that causes crosslinking of the BCR complex via antibody or molecular binding entity. In some embodiments, the crosslinker is selected from the group consisting of F(ab)₂ IgM, IgG, IgD, polyclonal BCR antibodies, monoclonal BCR antibodies, Fc receptor derived binding elements and/or a combination thereof. In some embodiments, the Ig is derived from a species selected from the group consisting of mouse, goat, rabbit, pig, rat, horse, cow, shark, chicken, or llama. In some embodiments, the crosslinker is F(ab)₂ IgM, Polyclonal IgM antibodies, Monoclonal IgM antibodies, Biotinylated F(ab)₂ IgG/M, Biotinylated Polyclonal IgM antibodies, Biotinylated Monoclonal IgM antibodies and/or a combination thereof.

In some embodiments of the methods of the invention, the cell is further subjected to a second modulator, e.g., the cell may be subjected to a B cell receptor activator and a kinase inhibitor Such as a PI3 kinase inhibitor or a JAK inhibitor (see U.S. Nos. 61/226,878 and 61/157,900 which are hereby incorporated by reference) or a phosphatase inhibitor. In some embodiments, the second modulator is F(ab)₂IgM or F(ab)₂IgM and H₂O₂.

In some embodiments, the modulator is selected from the group F(ab)₂IgM, SDF1a, R848, anti-IgD, CD40L, thapsigargin, fludarabine, bendamustine, poly CpG, or IFNa and/or a combination thereof.

In some embodiments, the activatable element is a protein. In some embodiments, the protein is selected from the group consisting of Akt1, Akt2, Akt3, SAPK/JNK1,2,3, p38s, Erk1/2, Syk, ZAP-70, Btk, BLNK, Lyn, PLCγ, PLCγ 2, STAT1, STAT3, STAT4, STAT5, STATE, CREB, Lyn, p-S6, Cbl, NF-κB, GSK3β, CARMA/Bcl10 and Tcl-1. In some embodiments, the activatable element is STAT5, PLC□□□Syk, Erk, or Lyn. In some embodiments, these markers are used to predict response to fludarabine.

In some embodiments, tonic signaling (ligand independent signaling) is shown in a subset of CLL patients by using H₂O₂ alone or in combination with a crosslinker, such as F(ab)₂IgM. In some embodiments, if the cell demonstrates evidence of tonic signaling after treatment with H₂O₂, then that is one embodiment of a predictive response to a drug, such as fludarabine as one example.

In one embodiment of the invention, tonic signaling is shown by measuring canonical B cell signaling molecules such as p-Lyn, p-Syk, p-BLNK, p-PLCγ2, p-Erk, p-Akt, p-S6, p-65/RelA, as well as non-canonical signaling markers such as p-STAT5.

In some embodiments, ZVAD is used as a modulator to analyze cell death pathways to investigate whether a therapeutic agent affects caspase independent or caspase dependent pathways. ZVAD will block caspase dependant cleavage and it can be used to distinguish caspase-dependent from caspase-independent cell death. This analysis is useful to determine if test substances or drugs will affect either apoptotic pathway and whether both caspase-dependent and caspase-independent pathways are necessary for a therapeutic agent to effectively promote cell death.

In another embodiment, mixture models are used to assess response to treatment. A sample signaling profile may be compared to a standard signaling profile and a result determined. In one embodiment, data generated from the tests described herein are compared to a standard profile defined by a mixture model derived from measurements from one or a plurality of samples. Data can be used to create a profile of results for patients in order to predict who will respond to a particular therapeutic regimen, those who will not, and variations thereof. Test results may be compared to a standard profile once it is created and correlations to responses may be derived. A test may be structured so that an individual patient sample may be viewed with these populations in mind and allocated to one population or the other, or a mixture of both and subsequently to use this correlation to patient management, therapy, prognosis, etc.

In another aspect, the invention provides methods of classifying a cell population by contacting the cell population with at least one modulator, where the modulator is from the group F(ab)₂IgM, SDF1a, R848, anti-IgD, CD40L, thapsigargin, fludarabine, bendamustine, poly CpG, or IFNa and/or a combination thereof, determining the presence or absence of an increase in activation level of an activatable element in the cell population, and classifying the cell population based on the presence or absence of the increase in the activation of the activatable element.

### CLL

Chronic lymphocytic leukemia (CLL) is the most common adult leukemia in the Western world and is characterized by aberrant accumulation of CD5+ B lymphocytes in the peripheral blood, bone marrow and secondary lymphoid organs. Clinical presentation, natural course of the disease and response to treatment are all extremely variable with survival ranging from months to decades. Although the biological mechanisms to account for this unpredictable clinical course are unknown, several biological indicators have been linked to CLL. However, there is often discordance between their predictive value for disease outcome. Thus predisposing factors in determining the clinical benefit of these markers include whether the disease is at early or late stage as well as the treatment that the patient may have undergone. A variety of cytogenetic abnormalities including del(17p13.1), del(11q22.3), trisomy 12 are associated with poor prognosis while del(13q.14.3) is associated with a more favorable clinical course (Hallek 2008, Hamblin 2007 Ghia et al., 2007). In addition, at least two predominant subtypes of CLL have been identified based on the presence or absence of somatic mutations within the immunoglobulin heavy chain variable region (IgV_{H}). The outcome of patients with leukemic cells that express unmutated IgV_{H} is poor compared to that of patients in which leukemic cells express mutated IgV_{H} ((Hamblin 1999), Ghia et al., Crit. Rev. Oncol. Hematol. 2007, Hallek, 2008). The latter is commonly but not invariably linked to positive expression of ZAP-70 and CD38 (Damle 1999). Moreover, considerable molecular heterogeneity between leukemic samples has been identified through gene array technology (Rodriguez et al 2007). Given the availability of newly approved and investigational therapeutic agents, a greater understanding of CLL disease biology is needed to predict disease progression and assist in selecting optimal therapeutic agents on an individual patient basis.

Studies in leukemia have described a new approach in which single cell network profiling in response to extracellular inputs (such as growth factors and cytokines) can be used to distinguish healthy from diseased cells (Irish Cell (2004), Irish Blood (2006), Irish et al., Nat Rev Cancer (2006), Kotecha et al., (2008)). Induced protein phosphorylation rather than the frequently measured basal phospholylation state of a protein is more informative as it takes into account signaling dysregulation that is the consequence of numerous cytogenetic, epigenetic and molecular changes characteristic of transformed cells, Multiparameter flow cytometry at the single cell level not only measures multiple phospho-signaling proteins but also delineates cell sub-sets within complex primary cell populations. No prior sorting of cell subpopulations is required before challenge with an extracellular modulator.

Central to B development is the role of the B cell receptor signal complex composed of a surface immunoglobulin molecule non-covalently associated with the signal transducing-CD79/CD79 heterodimer. In normal B cells, stimulation of the BCR by antigen leads to phosphorylation of immunoreceptor tyrosine-based activation motifs (ITAMs) within the cytoplasmic tails of CD79 and CD79, Subsequent recruitment of Syk to these motifs propagates a signal through activation of downstream signaling molecules such as BLNK, phosphatidylinositol-3-Kinase, phospholipase C-γ and the Ras/Raf/Erk pathways (Brezski and Monroe 2008, Efremov et al., Autoimmunity Reviews 2007 Kurosaki et al., J. Exp Med 182, p1815, 1995, Takata et al., J. Exp Med 184, p31 1996), During normal B cell maturation, signals from the B cell receptor lead to remarkably different biochemical responses depending on the developmental stage in which the B-cell resides (Gauld et al). Additionally, there is essential fine tuning of BCR signaling for the survival and proliferation of healthy B cells, which has been shown to involve phosphatase(s) whose activity is regulated by NADPH-oxidase-generated H₂O₂ (Reth M. Nat Immunol. 2002; 3:1129-1134 and Irish J M, J Immunol. 2006; 177:1581-1589).

Recently, it has been recognized that in conjunction with antigen-driven responses, ligand-independent signaling, (termed tonic signaling) by the pre-B cell receptor and BCR has an important role in B cell development and in mature B cells respectively. In addition to the recognized role of CD79□ and CD79□, tyrosine phosphatases are also likely to impact on tonic signaling. This is based on a study of B cells in which inhibition of these phosphatases by H₂O₂ or vanadate revealed tyrosine phosphorylation of signaling proteins associated with the BCR (Wienands, J., Larbolette, O. & Reth, M. PNAS (1996), Reth Nat Rev. Immunol 2002, Monroe (2006) Irish et al., J. Immunol. 2006),

As mentioned above, significant associations have been observed between clinical course of CLL and certain features of the BCR, indicating that antigen-dependent and independent stimulation and signaling may play an important role in the pathogenesis of the disease. Most CLL B cells express a BCR comprised of IgM with or without somatic mutations. In addition, the biased usage of V_{H} genes, the preferential usage of kappa or lambda light-chains, as well as aberrant expression of BCR signaling mediators suggest a central role for the B cell receptor signaling network in CLL. This is corroborated by in vitro studies in which significant differences in BCR signaling were found in CLL primary patient samples (Chen et al. Blood 2007, Gobessi Blood 2007 Deglesne et al., Can Res 66, p7158, 2006, Muzio et al., Blood 2008 112, p188 Guarini et al., Blood 2008 112 p782).

Under normal physiological conditions, apoptosis proceeds from sensors that monitor cell stress and damage to effectors that relay the signals to activate programmed cell death pathways. In cancer, cells have co-opted a variety of mechanisms to evade apoptosis for the purpose of survival and disease progression and also to over-ride any benefit from a therapeutic agent (Hanahan and Wienberg, Cell 2000). CLL is no different in that it may show inactivated p53 signaling (17p deletion) in a subset of patients. In other patient subsets, different mechanisms over-riding apoptosis have evolved resulting in refractoriness or resistance to therapies. The current study was undertaken to determine whether and how ligand dependent and ligand independent (tonic) BCR signaling was associated with subverted apoptotic pathways in patient samples exposed in vitro to fludarabine, a drug at the core of many CU treatment regimens.

Based on prior studies in leukemic samples, modulated SCNP using flow cytometry was applied to determine whether there were subsets of cells between samples and within the same sample that showed: a) alterations in BCR responses b) differences in fludarabine-induced apoptosis c) associations between BCR signaling and in vitro chemosensitivity to fludarabine. The data presented here suggest tonic BCR signaling may play a role in the response mediated by agents that induce apoptosis.

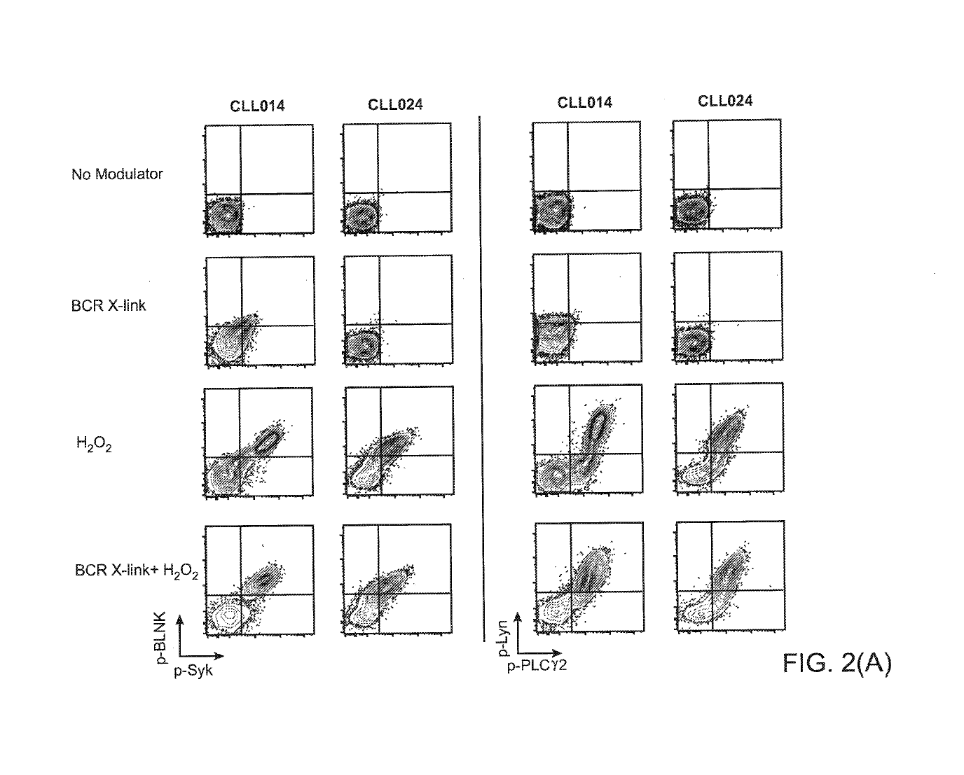

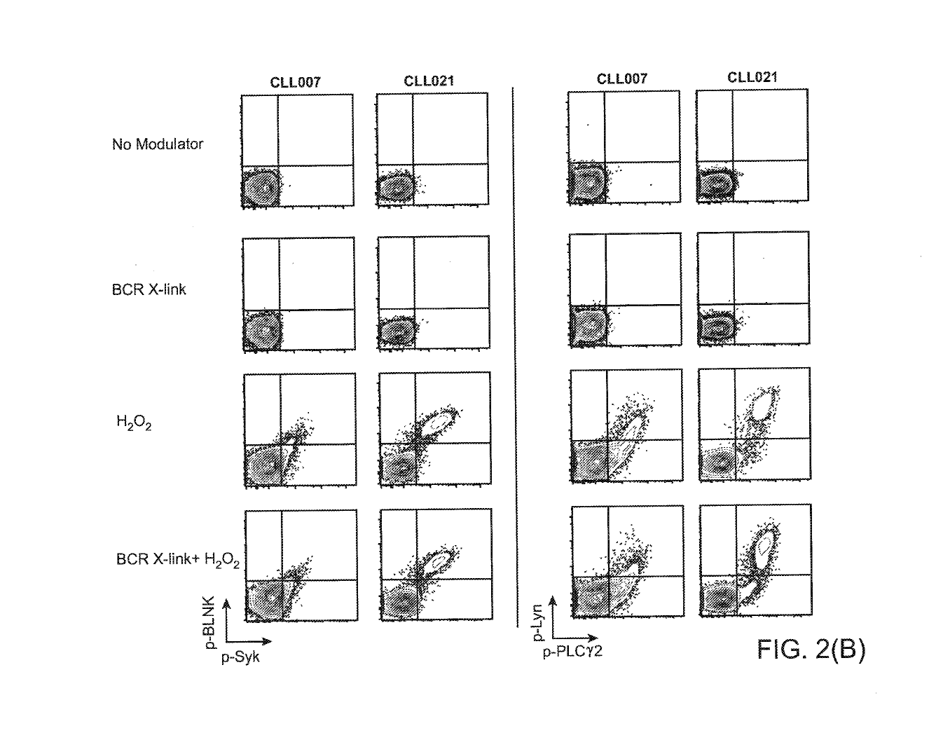

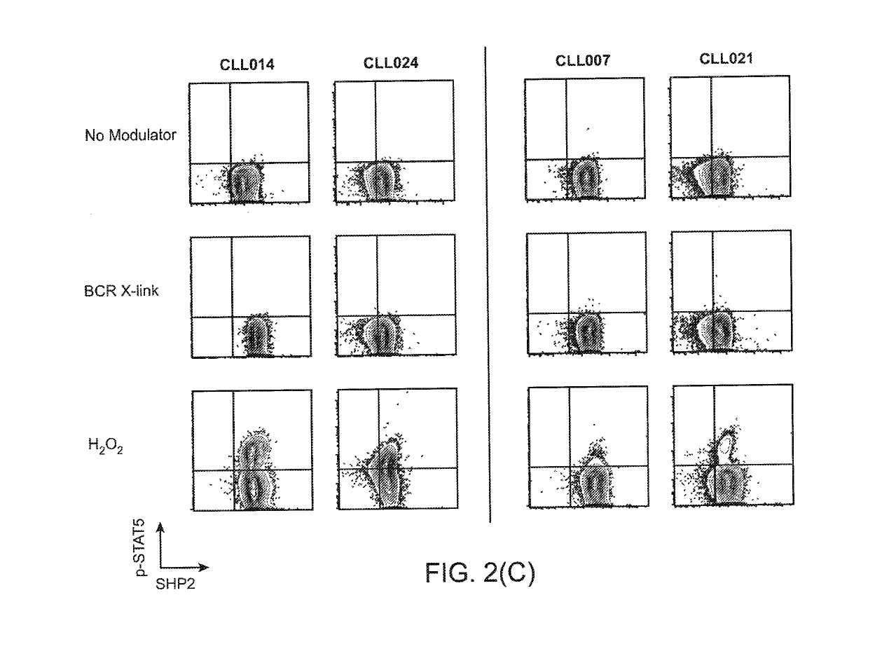

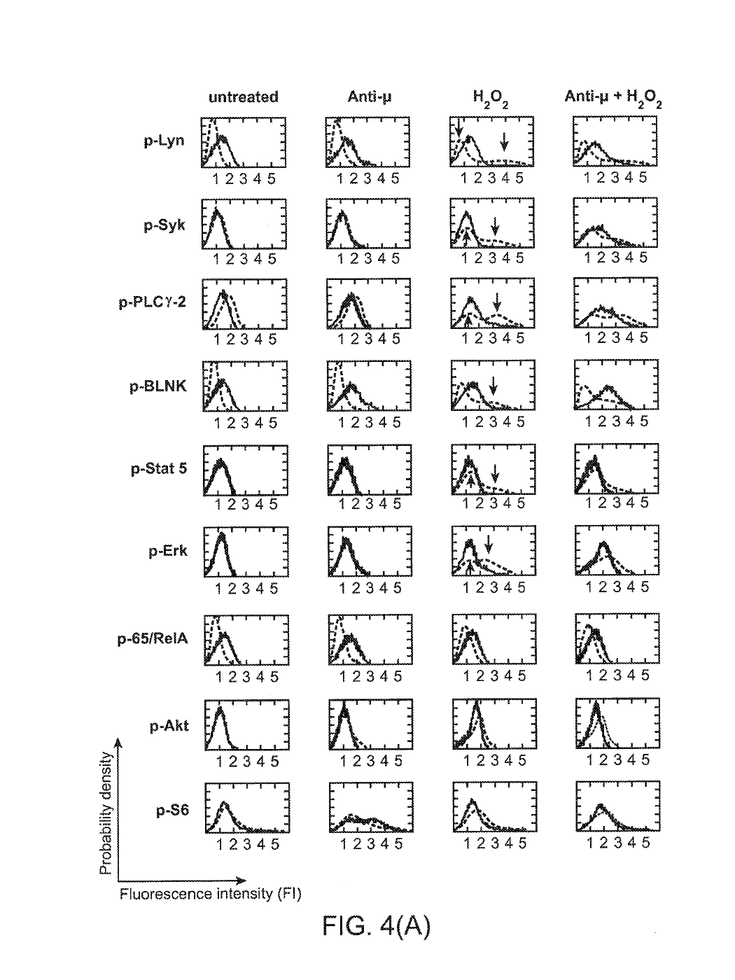

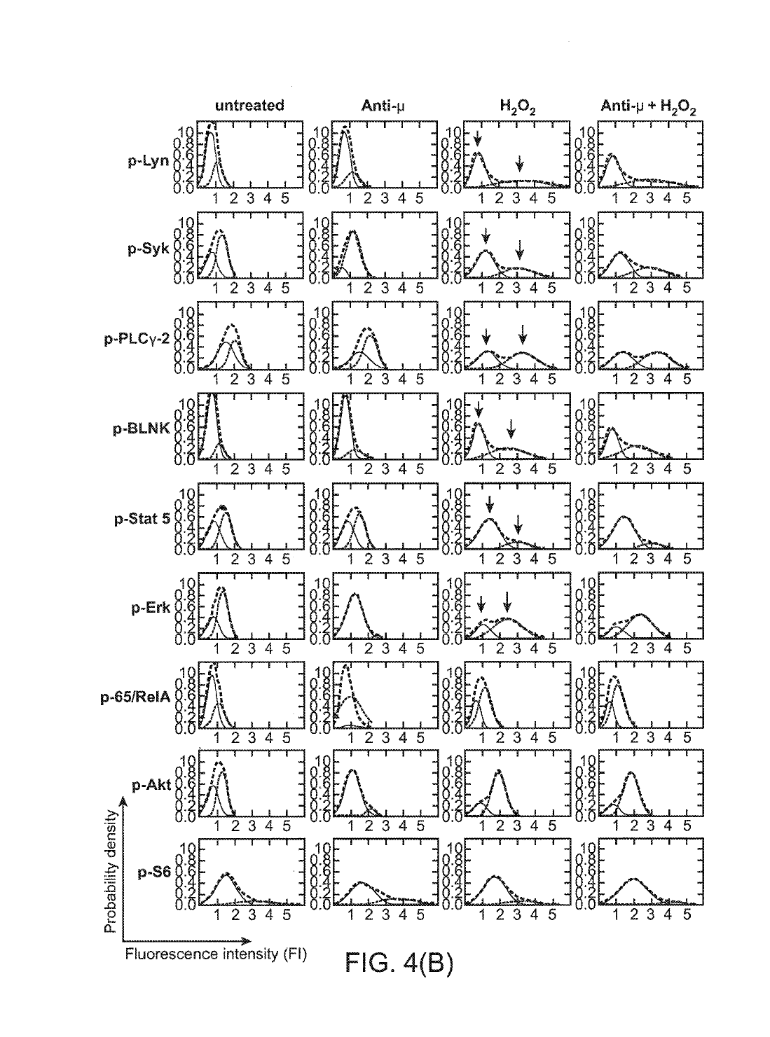

In one aspect of the invention, the range of basal signaling in CLL B cells from patient samples is very broad compared to B cells from healthy donors. Mixture models show the distribution of different signaling subpopulations within a sample. A mixture model is created by making a virtual sample by looking at the distribution of signaling subpopulations in an entire cohort of samples (FIG. 4A, B). Heterogeneity of signaling is seen within cell subsets in individual samples. The heterogeneity is revealed by treatment of the sample with a modulator including, but not limited to H₂O₂. Such heterogeneity is not observed by monitoring the basal phosphorylation state in the absence of a modulator. This heterogeneity could have therapeutic implications. One or more cell subsets in a sample with a differential signaling response could be, for example a therapeutically resistant clone. See FIG. 2A patient sample CLL014 treated with H₂O₂.

In some CLL samples, subpopulations of B cells undergo an increase in p-STAT5 in response to modulators, including but not limited to phosphatase inhibitors such as H₂O₂. See FIG. 2C.

Another embodiment of the invention is detection of B cell subsets within CLL patient samples (that are refractory or competent) to undergo apoptosis induced by in vitro treatment of therapeutic agents including, but not limited to fludarabine. This drug forms the core of many CLL patient treatment regimens.

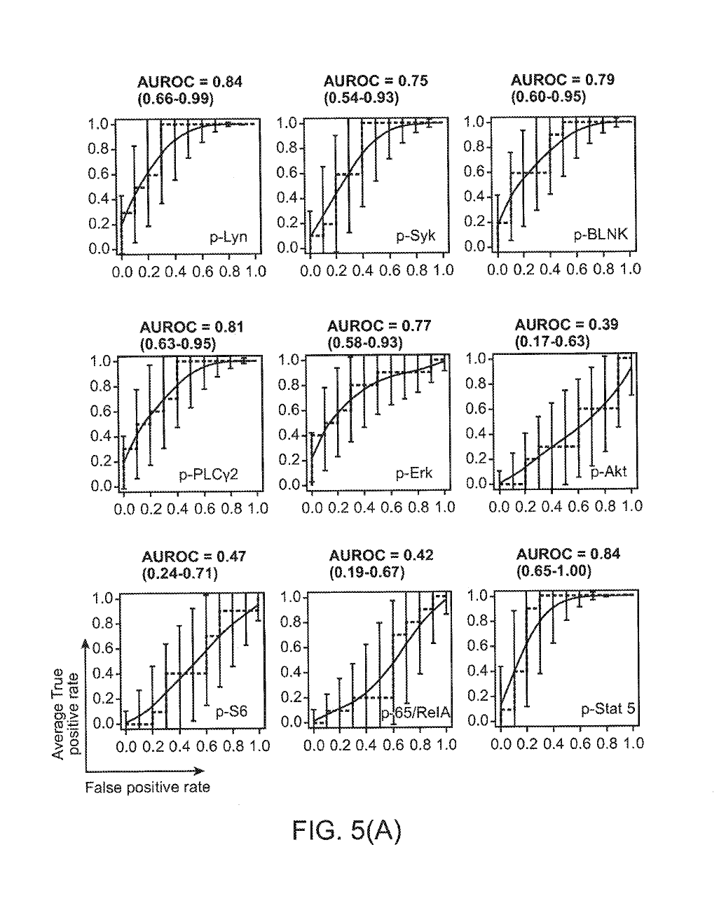

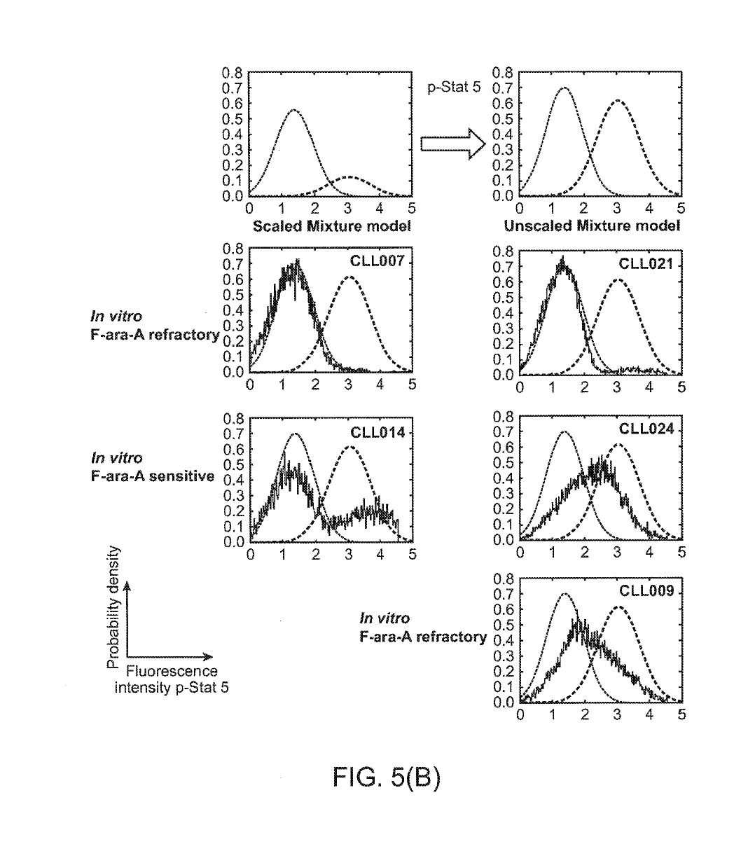

Patients can be stratified by the modulated signaling responses in their CLL samples. Patients can also be stratified by the apoptotic response of their CLL samples exposed in vitro to therapeutic agents such as fludarabine. Apoptotic responses stratify the patient samples into those that are competent versus those that are refractory. Also, the level of signaling stratifies patient samples during basal and/or modulated signaling states. The present data shows that there is an association between increased signaling responses and an ability to undergo apoptosis. Another embodiment of the invention relates to the statistical methods used to demonstrate these biological pathway associations. These statistical methods include, but are not limited to Area Under the Receiver Operating Characteristic (AUROC) (see FIG. 5A) using metrics including, but not limited to the mixture models shown in FIGS. 4A and 4B. The AUROC curves show that increased phosphorylation of Lyn, Syk, BLNK, PLCγ2, Erk, and STAT5 are highly predictive of cell subpopulations competent to undergo apoptosis in vitro. See FIG. 5A. An AUROC value greater than 0.5 can indicate an improved predictive value as opposed to chance association. An AUROC value of one indicates that the predictive value is perfect. An AUROC which has a value>0.65>0.70>0.75>0.8>0.85>0.9>0.95>0.97 can form the basis for a predictive test for patient management. In some embodiment, the methods of the invention determine the presence or absence of a change in activation level of at least two activatable elements of Lyn, Syk, BLNK, PLCγ2, Erk 1/2 or STAT5 in a cell.

In some embodiments of the invention, detection using mixture models and univariate or multivariate analysis described above can be used in a predictive test for diagnosis and/or patient management, for example, using classification and modeling methods such as logistic regression (including regularized, penalized, and shrinkage methods including lasso and ridge), decision trees, random forests, support vector machines, boosting, etc. to generate univariate and multivariate models. In some embodiments, analysis can be done using univariate and multivariate models associating hydrogen peroxide (H₂O₂) or B-cell receptor cross linking induced changes in phosphorylation with the ability of cells to undergo apoptosis.

Another embodiment of the invention allows a user to understand whether the signaling data for an intracellular signaling molecule is predictive for the apoptotic response of a sample from an individual patient. As such, it can be the basis of a test to determine whether a patient's CLL disease will respond to a therapeutic agent including, but not limited to fludarabine. See FIG. 5B.

### Methods

In some embodiments, the invention provides methods, including methods to determine the physiological status of a cell, e.g., by determining the activation level of an activatable element upon contact with one or more modulators. In some embodiments, the invention provides methods, including methods to classify a cell according to the status of an activatable element in a cellular pathway. The information can be used in prognosis and diagnosis, including susceptibility to disease(s), status of a diseased state and response to changes, in the environment, such as the passage of time, treatment with drugs or other modalities. The physiological status of the cells provided in a sample (e.g. clinical sample) may be classified according to the activation of cellular pathways of interest. The cells can also be classified as to their ability to respond to therapeutic agents and treatments.

One or more cells, or samples containing one or more cells, can be isolated from body samples, such as, but not limited to, smears, sputum, biopsies, secretions, cerebrospinal fluid, bile, blood, lymph fluid, urine and feces, a lavage of a tissue or organ (e.g. lung) or tissue which has been removed from organs, such as breast, lung, intestine, skin, cervix, prostate, and stomach. For example, a tissue sample can comprise a region of functionally related cells or adjacent cells. Such samples can comprise complex populations of cells, which can be assayed as a population, or separated into sub-populations. Such cellular and acellular samples can be separated by centrifugation, elutriation, density gradient separation, apheresis, affinity selection, panning, FACS, centrifugation with Hypaque, etc. By using antibodies specific for markers identified with particular cell types, a relatively homogeneous population of cells may be obtained. Alternatively, a heterogeneous cell population can be used. Cells can also be separated by using filters. For example, whole blood can also be applied to filters that are engineered to contain pore sizes that select for the desired cell type or class. Rare pathogenic cells can be filtered out of diluted, whole blood following the lysis of red blood cells by using filters with pore sizes between 5 to 10 μm, as disclosed in U.S. patent application Ser. No. 09/790,673. Other devices can separate tumor cells from the bloodstream, see Demirci U, Toner M., Direct etch method for microfluidic channel and nanoheight post-fabrication by picoliter droplets, Applied Physics Letters 2006; 88 (5), 053117; and Irimia D, Geba D, Toner M., Universal microfluidic gradient generator, Analytical Chemistry 2006; 78: 3472-3477. Once a sample is obtained, it can be used directly, frozen, or maintained in appropriate culture medium for short periods of time. Methods to isolate one or more cells for use according to the methods of this invention are performed according to standard techniques and protocols well-established in the art.

Suitable cells include those cell types associated in a wide variety of disease conditions, even while in a non-diseased state. Accordingly, suitable eukaryotic cell types include, but are not limited to, tumor cells of all types (e.g. melanoma, myeloid leukemia, carcinomas of the lung, breast, ovaries, colon, kidney, prostate, pancreas and testes), cardiomyocytes, dendritic cells, endothelial cells, epithelial cells, lymphocytes (T-cell and B cell), mast cells, eosinophils, vascular intimal cells, macrophages, natural killer cells, erythrocytes, hepatocytes, leukocytes including mononuclear leukocytes, stem cells such as hematopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include primary disease state cells, such as primary tumor cells. Suitable cells also include known research cells, including, but not limited to, Jurkat T cells, NIH3T3 cells, CHO, COS, etc. See the ATCC cell line catalog, hereby expressly incorporated by reference.

In some embodiments, the cells are cultured post collection in a media suitable for revealing the activation level of an activatable element (e.g. RPMI, DMEM) in the presence, or absence, of serum such as fetal bovine serum, bovine serum, human serum, porcine serum, horse serum, or goat serum. When serum is present in the media it could be present at a level ranging from 0.0001% to 100%. In some embodiments serum is present in the media at a level ranging from 0.0001% to 90%. In some embodiments serum is present in the media at a level ranging from 0.01% to 30%. In some embodiments serum is present in the media at 1, 2, 3, 4, 5, 6, 7, 8, 9 or 10%. In some embodiments, serum is present in the media at any suitable level.

In some embodiments, the cell is a hematopoietic cell. Examples of hematopoietic cells include but are not limited to pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells.

In some embodiments, the cells used in the present invention are taken from a patient. Cells used in the present invention can be purified from whole blood by any suitable method.

The term “patient” or “individual” as used herein includes humans as well as other mammals. The methods generally involve determining the status of an activatable element. The methods also involve determining the status of a plurality of activatable elements.

In some embodiments, the invention provides a method of classifying a cell by determining the presence or absence of a change in activation level of an activatable element in the cell upon treatment with one or more modulators, and classifying the cell based on the presence or absence of the change in the activation of the activatable element. In some embodiments the change is a decrease. In some embodiments the change is an increase. In some embodiments of the invention, the activation level of the activatable element is determined by contacting the cell with a binding element that is specific for an activation state of the activatable element. In some embodiments, a cell is classified according to the activation level of a plurality of activatable elements after the cell have been subjected to a modulator. In some embodiments of the invention, the activation levels of a plurality of activatable elements are determined by contacting a cell with a plurality of binding element, where each binding element is specific for an activation state of an activatable element.

The classification of a cell according to the status of an activatable element can comprise classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition such as those conditions shown in the Summary and under the section marked Conditions.

Modulators include compounds or conditions capable of impacting cellular signaling networks. Modulators can include single or multiple agents. For example, anti-μ (also called F(ab)₂ IgM, anti-IgM or αIgM) and H₂O₂ act together in healthy bone marrow cells. A modulator can be an activator or an inhibitor. Modulators can take the form of a wide variety of environmental inputs. Examples of modulators include but are not limited to growth factors, cytokines, chemokines, soluble receptors, Toll-like receptor ligands, pathogens, parasites, components of pathogens or parasites, adhesion molecule modulators, pharmaceutical compounds, drugs, hormones, small molecules, polynucleotides, antibodies, natural compounds, lactones, chemotherapeutic agents, immune modulators, carbohydrates, proteases, ions, reactive oxygen species, radiation, physical parameters such as heat, cold, UV radiation, peptides, and protein fragments, either alone or in the context of cells, cells themselves, viruses, and biological and non-biological complexes (e.g. beads, plates, viral envelopes, antigen presentation molecules such as major histocompatibility complex). Examples of modulators include, but are not limited to, F(ab)₂IgM, SDF1a, R848, anti-IgD, CD40L, thapsigargin, fludarabine, bendamustine, poly CpG, or IFNa and/or combinations thereof. Additional modulators, inhibitors and activators are disclosed in U.S. 61/085,789 which is hereby incorporated by reference in its entirety. Fludarabine is shown in V. Gandhi and W. Plunkett (2002) Clin. Pharmacokinet 41:93-103, which is hereby incorporated by reference in its entirety. R848 is Resiquimod, a drug that acts as an immune response modifier, and has antiviral and antitumour activity. It is used as a topical cream in the treatment of skin lesions such as those caused by herpes simplex virus, and as an adjuvant to increase the effectiveness of vaccines. It has several mechanisms of action, being both an agonist for toll-like receptor 7 and 8, and an upregulator of the opioid growth factor receptor.

In some embodiments, the modulator is an activator. In some embodiments the modulator is an inhibitor. In some embodiments, the invention provides methods for classifying a cell by contacting the cell with an inhibitor, determining the presence or absence of a change in activation level of an activatable element in the cell, and classifying the cell based on the presence or absence of the change in the activation of the activatable element. In some embodiments the change is a decrease. In some embodiments the change is an increase. In some embodiments, a cell is classified according to the activation level of a plurality of activatable elements after the cell have been subjected to an inhibitor. In some embodiments, the inhibitor is an inhibitor of a cellular factor or a plurality of factors that participates in a signaling cascade in the cell. In some embodiments, the inhibitor is a kinase or phosphatase inhibitor. Examples of kinase inhibitors include adaphostin, AG 490, AG 825, AG 957, AG 1024, aloisine, aloisine A, alsterpaullone, aminogenistein, API-2, apigenin, arctigenin, AY-22989, BAY 61-3606, bisindolylmaleimide IX, chelerythrine, 10-[4′-(N,N-Diethylamino)butyl]-2-chlorophenoxazine hydrochloride, dasatinib, 2-Dimethylamino-4,5,6,7-tetrabromo-1H-benzimidazole, 5,7-Dimethoxy-3-(4-pyridinyl)quinoline dihydrochloride, edelfosine, ellagic acid, enzastaurin, ER 27319 maleate, erlotinib, ET18OCH₃, fasudil, flavopiridol, gefitinib, GW 5074, H-7, H-8, H-89, HA-100, HA-1004, HA-1077, HA-1100, hydroxyfasudil, indirubin-3′-oxime, 5-Iodotubercidin, kenpaullone, KN-62, KY12420, LFM-A13, lavendustin A, luteolin, LY-294002, LY294002, mallotoxin, ML-9, NSC-154020, NSC-226080, NSC-231634, NSC-664704, NSC-680410, NU6102, olomoucine, oxindole I, PD-153035, PD-98059, PD 169316, phloretin, phloridzin, piceatannol, picropodophyllin, PK1, PP1, PP2, purvalanol A, quercetin, R406, R788, rapamune, rapamycin, Ro 31-8220, roscovitine, rottlerin, SB202190, SB203580, sirolimus, sorafenib, SL327, SP600125, staurosporine, STI-571, SU-11274, SU1498, SU4312, SU6656, 4,5,6,7-Tetrabromotriazole, TG101348, Triciribine, Tyrphostin AG 490, Tyrphostin AG 825, Tyrphostin AG 957, Tyrphostin AG 1024, Tyrphostin SU1498, U0126, VX-509, VX-667, VX-680, W-7, wortmannin, XL-019, XL-147, XL-184, XL-228, XL-281, XL-518, XL-647, XL-765, XL-820, XL-844, XL-880, Y-27632, ZD-1839, ZM-252868, ZM-447439, Examples of phosphatase inhibitors include, but are not limited to H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenyarsine oxide, Pyrrolidine Dithiocarbamate, and Aluminum fluoride. In some embodiments, the phosphatase inhibitor is H₂O₂.

In some embodiments, the methods of the invention provide methods for determining the presence or absence of a condition in an individual by subjecting a cell from the individual to a modulator and an inhibitor, determining the activation level of an activatable element in the cell, and determining the presence or absence of a condition based on the activation level. In some embodiments, the activation level of a plurality of activatable elements in the cell is determined. The inhibitor can be an inhibitor as described herein. In some embodiments, the inhibitor is a phosphatase inhibitor. In some embodiments, the inhibitor is H₂O₂. The modulator can be any modulator described herein. In some embodiments, the modulator is a B cell receptor modulator. In some embodiments, the B cell receptor modulator is a B cell receptor activator. An example of B cell receptor activator is a cross-linker of the B cell receptor complex or the B-cell co-receptor complex. In some embodiments, cross-linker is an antibody or molecular binding entity. In some embodiments, the cross-linker is an antibody. In some embodiments, the antibody is a multivalent antibody. In some embodiments, the antibody is a monovalent, bivalent, or multivalent antibody made more multivalent by attachment to a solid surface or tethered on a nanoparticle surface to increase the local valency of the epitope binding domain.

The cross-linker can be a molecular binding entity. In some embodiments, the molecular binding entity acts upon or binds the B cell receptor complex via carbohydrates or an epitope in the complex. In some embodiments, the molecular is a monovalent, bivalent, or multivalent is made more multivalent by attachment to a solid surface or tethered on a nanoparticle surface to increase the local valency of the epitope binding domain.

The cross-linking of the B cell receptor complex or the B-cell co-receptor complex can comprise binding of an antibody or molecular binding entity to the cell and then causing its crosslinking via interaction of the cell with a solid surface that causes crosslinking of the BCR complex via antibody or molecular binding entity.

The crosslinker can be F(ab)₂ IgM, IgG, IgD, polyclonal BCR antibodies, monoclonal BCR antibodies, Fc receptor derived binding elements and/or a combination thereof. The Ig can be derived from a species selected from the group consisting of mouse, goat, rabbit, pig, rat, horse, cow, shark, chicken, llama or human. The Ig or binding element can be fully human or partially human and can be generated by any suitable method known in the art. In some embodiments, the crosslinker is F(ab)₂ IgM, Polyclonal IgM antibodies, Monoclonal IgM antibodies, Biotinylated F(ab)₂ IgG/M, Biotinylated Polyclonal IgM antibodies, Biotinylated Monoclonal IgM antibodies and/or a combination thereof.

In some embodiments, the methods of the invention provides for the use of more than one modulator. In some embodiments, the methods of the invention utilize a B cell receptor activator and a phosphatase inhibitor. In some embodiments, the methods of the invention utilize F(ab)₂IgM or biotinylated F(ab)₂IgM and H₂O₂.

In some embodiments, the methods of the invention provides for methods of classifying a cell population, or determining a phenotypic profile of a population of cells, by exposing the cell population in separate cultures to a plurality of modulators and determining the status of activatable elements in the cell populations. In some embodiments, the status of a plurality of activatable elements in the cell population, or the phenotypic profile, is determined. In some embodiments, at least one of the modulators of the plurality of modulators is an inhibitor. The modulator can be any modulators described herein. In some embodiments, the modulator is selected from the group consisting of F(ab)₂IgM, SDF1a, R848, anti-IgD, CD40L, thapsigargin, fludarabine, bendamustine, poly CpG, or IFNa and a combination thereof. In some embodiments of the invention, the status of an activatable element is determined by contacting the cell population with a binding element that is specific for an activation state of the activatable element. In some embodiments, the status of a plurality of activatable elements is determined by contacting the cell population with a plurality of binding elements, where each binding element is specific for an activation state of an activatable element.

In some embodiments, the methods of the invention provide for methods for classifying a cell population by contacting the cell population with at least one modulator, where the modulator is from the group F(ab)₂IgM, SDF1a, R848, anti-IgD, CD40L, thapsigargin, fludarabine, bendamustine, poly CpG, or IFNa and/or a combination thereof, and determining the status of an activatable element in the cell population. In some embodiments, the status of a plurality of activatable elements in the cell population is determined. In some embodiments of the invention, the status of an activatable element is determined by contacting the cell population with a binding element that is specific for an activation state of the activatable element. In some embodiments, the status of a plurality of activatable elements is determined by contacting the cell population with a plurality of binding elements, where each binding element is specific for an activation state of an activatable element.

In some embodiments, the invention provides a method for classifying a B-lymphocyte progenitor or derived cell as described herein by contacting the cell with a modulator, determining the presence or absence of a change in activation level of an activatable element in the cell, and classifying the cell based on the presence or absence of the change in the activation of the activatable element. In some embodiments the change is a decrease. In some embodiments the change is an increase. In some embodiments, the presence or absence of a change in the activation level of an activatable element is determined by contacting the cell with a binding element that is specific for an activation state of the activatable element. In some embodiments, a B-lymphocyte progenitor or derived cell is classified according to the activation level of a plurality of activatable elements after the cells have been subjected to a modulator. In some embodiments, the presence or absence of a change in the activation levels of a plurality of activatable elements is determined by contacting the cell population with a plurality of binding elements, where each binding elements is specific for an activation state of an activatable element. In some embodiments, the method for classifying a B-lymphocyte progenitor or derived cell further comprises determining the level of at least one cell-surface marker. In some embodiments, the method for classifying a B-lymphocyte progenitor or derived cell further comprises determining the level of at least one intracellular marker, for example a captured intracellular cytokine In some embodiments, the B-lymphocyte progenitor or derived cell is associated with a condition such a neoplastic, autoimmune or hematopoietic condition. Thus, in some embodiments, the invention provides methods for classifying a B-lymphocyte progenitor or derived cell associated with a condition (e.g. neoplastic, autoimmune or hematopoietic condition) by contacting the cell with a modulator, determining the presence or absence of a change in activation level of one or more activatable elements in the cell, and classifying the cell based on the presence or absence of the change in the activation of the one or more activatable elements. In some embodiments the change is a decrease. In some embodiments the change is an increase.

In some embodiments, the invention provides methods for correlating and/or classifying an activation state of a CLL cell with a clinical outcome in an individual by subjecting the CLL cell from the individual to a modulator, wherein the CLL cell expresses B-Cell receptor (BCR), determining the activation levels of a plurality of activatable elements, and identifying a pattern of the activation levels of the plurality of activatable elements to determine the presence or absence of an alteration in signaling proximal to the BCR, wherein the presence of the alteration is indicative of a clinical outcome. In some embodiments, the activation levels of a plurality of activatable elements are determined by contacting the cell with a plurality of binding elements, where each binding element is specific for an activation state of an activatable element. The clinical outcome can be any clinical outcome described herein.

In some embodiments, the methods of the invention provide methods for determining tonic signaling status of a cell by subjecting the cell to a modulator, determining the activation level of an activatable element that participates in a tonic signaling pathway in the cell, and determining the status of a tonic signaling pathway in the cell from the activation level. In some embodiments, the status of a plurality of activatable elements in the cell population is determined. In some embodiments, the activation level of an activatable element is determined by contacting the cell with a binding element that is specific for an activation state of the activatable element. In some embodiments, the activation level of a plurality of activatable elements is determined by contacting the cell with a plurality of binding elements, where each binding element is specific for an activation state of an activatable element. In some embodiments, the tonic signaling is mediated by a cellular receptor. In some embodiments, the tonic signaling is mediated by a T-cell receptor (TCR). In some embodiments, the tonic signaling is mediated by the B-cell receptor (BCR). In some embodiments, the tonic signaling status in the cell is used to classify the cell as described herein. Patterns and profiles of one or more activatable elements are detected using the methods known in the art including those described herein. In some embodiments, patterns and profiles of activatable elements that are cellular components of a cellular pathway are detected using the methods described herein. In some embodiments, patterns and profiles of activatable elements that are cellular components of a signaling pathway are detected using the methods described herein. In some embodiments, patterns and profiles of activatable elements that are cellular components of a tonic signaling pathway are detected using the methods described herein. For example, patterns and profiles of one or more phosphorylated polypeptide are detected using methods known in art including those described herein.

In some embodiments of the methods described herein, cells (e.g. normal non-transformed cells) other than the cells associated with a condition (e.g. cancer cells) can be used to make clinical decisions. Cells, other than cells associated with a condition (e.g. cancer cells), are in fact reflective of the condition. Normal cells (e.g. healthy cells or non-transformed cells) can be used, e.g., in assigning a risk group, predicting an increased risk of relapse, predicting an increased risk of developing secondary complications, choosing a therapy for an individual, predicting response to a therapy for an individual, determining the efficacy of a therapy in an individual, and/or determining the prognosis for an individual. For instance, in the case of cancer, infiltrating immune cells can determine the outcome of the disease. In another aspect, a combination of information from a cancer cell plus responding immune cells in the blood of a cancer patient can be used for diagnosis or prognosis of the cancer.

### Conditions

The methods of the invention are applicable to any condition in an individual involving, indicated by, and/or arising from, in whole or in part, altered physiological status in a cell. The term “physiological status” includes mechanical, physical, and biochemical functions in a cell. In some embodiments, the physiological status of a cell is determined by measuring characteristics of cellular components of a cellular pathway. Cellular pathways are well known in the art. In some embodiments the cellular pathway is a signaling pathway. Signaling pathways are also well known in the art (see, e.g., Hunter T., Cell (2000) 100(1): 113-27; Pawson T, Kofler M Curr Opin Cell Biol. 2009 April; 21(2):147-53 Cell Signaling Technology, Inc., 2002 Catalogue, Pathway Diagrams pgs. 232-253). A condition involving or characterized by altered physiological status may be readily identified, for example, by determining the state in a cell of one or more activatable elements, as taught herein. See also the patent applications cited herein, such as U.S. Pat. No. 8,227,202.

In some embodiments, the condition is CLL. In some embodiments, CLL is defined by a monoclonal B cell population that may co-express the following markers alone or in all possible combinations: CD5, CD20, CD19, CD22, CD23, CD38, and CD45. Other arrangements include CDCD5 with CD19 and CD23 or CD5 with CD20 and CD23 and by surface immunoglobulin expression. In some embodiments, CLL is defined by a monoclonal B cell population that co-expresses CD5 with CD19 and CD23 or CD5 with CD20 and CD23 and dim surface immunoglobulin expression. In some embodiments, the level of expression of the B cell receptor is also measured including its components such as IgM, IgG, IgI, IgD, kappa chain, lambda chain, Igα (CD79α)/Igβ (CD79β).

CLL is a clonal B cell disorder with an incidence of about 15,000 cases/yr and is the most common leukemia in western countries. The disease is first suspected by presence of lymphocytosis greater than 4,000/μl wbcs. Its phenotypic characterization shows CD5+, CD19+, CD20+, and CD23+. Clonality is determined by mutually exclusive expression of lambda or kappa light chains. Disease staging systems introduces by Rai and Binet are based on clinically determinable features. Cytogenetic changes associated with poor clinical outcome include 11q22-23 deletion, 17p deletion, trisomy 12, and p53 dysfunction which is through 17p deletion as one dominant mechanism. Cytogenetic changes associated with benign clinical course include the 13q14 deletion. Molecular markers include IgVH, CD 38, and ZAP-70.

One embodiment of the invention is directed to tumors and autoimmune diseases generally. Another embodiment of the invention relates to solid tumors and hematopoietic tumors. Other conditions within the scope of the present invention include, but are not limited to, cancers such as gliomas, lung cancer, colon cancer and prostate cancer. Specific signaling pathway alterations have been described for many cancers, including loss of PTEN and resulting activation of Akt signaling in prostate cancer (Whang Y E. Proc Natl Acad Sci USA Apr. 28, 1998; 95(9):5246-50), increased IGF-1 expression in prostate cancer (Schaefer et al., Science Oct. 9, 1998, 282: 199a), EGFR over expression and resulting ERK activation in glioma cancer (Thomas C Y. Int J Cancer Mar. 10, 2003; 104(1):19-27), expression of HER2 in breast cancers (Menard et al. Oncogene. Sep. 29, 2003, 22(42):6570-8), and APC mutation and activated Wnt signaling in colon cancer (Bienz M. Curr Opin Genet Dev 1999 October, 9(5):595-603).

Diseases other than cancer involving altered physiological status are also encompassed by the present invention. For example, it has been shown that diabetes involves underlying signaling changes, namely resistance to insulin and failure to activate downstream signaling through IRS (Burks D J, White M F. Diabetes 2001 February; 50 Suppl 1:S140-5). Similarly, cardiovascular disease has been shown to involve hypertrophy of the cardiac cells involving multiple pathways such as the PKC family (Malhotra A. Mol Cell Biochem 2001 September; 225 (1-):97-107). Inflammatory diseases, such as rheumatoid arthritis, are known to involve the chemokine receptors and disrupted downstream signaling (D'Ambrosio D. J Immunol Methods 2003 February; 273 (1-2):3-13). The invention is not limited to diseases presently known to involve altered cellular function, but includes diseases subsequently shown to involve physiological alterations or anomalies.

In some embodiments, the present invention is directed to methods for classifying one or more cells in a sample derived from an individual having or suspected of having condition. In some embodiments, the invention allows for identification of prognostically and therapeutically relevant subgroups of the conditions and prediction of the clinical course of an individual. In some embodiments, the invention provides method of classifying a cell according to the activation level of one or more activatable element in a cell from an individual having or suspected of having condition. In some embodiments, the classification includes classifying the cell as a cell that is correlated with a clinical outcome. The clinical outcome can be the prognosis and/or diagnosis of a condition, and/or staging or grading of a condition. In some embodiments, the classifying of the cell includes classifying the cell as a cell that is correlated to a patient response to a treatment. In some embodiments, the classifying of the cell includes classifying the cell as a cell that is correlated with minimal residual disease or emerging resistance.

### Activatable Elements

The methods and compositions of the invention may be employed to examine and profile the status of any activatable element in a cellular pathway, or collections of such activatable elements. Single or multiple distinct pathways may be profiled (sequentially or simultaneously), or subsets of activatable elements within a single pathway or across multiple pathways may be examined (again, sequentially or simultaneously).

As will be appreciated by those in the art, a wide variety of activation events can find use in the present invention. In general, the basic requirement is that the activation results in a change in the activatable protein that is detectable by some indication (termed an “activation state indicator”), preferably by altered binding of a labeled binding element or by changes in detectable biological activities (e.g., the activated state has an enzymatic activity which can be measured and compared to a lack of activity in the non-activated state). What is important is to differentiate, using detectable events or moieties, between two or more activation states (e.g. “off” and “on”).

The activation state of an individual activatable element is either in the on or off state. As an illustrative example, and without intending to be limited to any theory, an individual phosphorylatable site on a protein can activate or deactivate the protein. The terms “on” and “off,” when applied to an activatable element that is a part of a cellular constituent, are used here to describe the state of the activatable element, and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations. In some embodiments, the basis for classifying cells is that the distribution of activation levels for one or more specific activatable elements will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to classify cells in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may not contain activatable elements, of cell or a population of cells may be used to classify a cell or a population of cells into a class. Once the activation level of intracellular activatable elements of individual single cells is known they can be placed into one or more classes, e.g., a class that corresponds to a phenotype. A class encompasses a class of cells wherein every cell has the same or substantially the same known activation level, or range of activation levels, of one or more intracellular activatable elements. For example, if the activation levels of five intracellular activatable elements are analyzed, predefined classes that encompass one or more of the intracellular activatable elements can be constructed based on the activation level, or ranges of the activation levels, of each of these five elements. It is understood that activation levels can exist as a distribution and that an activation level of a particular element used to classify a cell may be a particular point on the distribution but more typically may be a portion of the distribution.

In addition to activation levels of intracellular activatable elements, expression levels of intracellular or extracellular biomolecules, e.g., proteins can be used alone or in combination with activation states of activatable elements to classify cells. Further, additional cellular elements, e.g., biomolecules or molecular complexes such as RNA, DNA, carbohydrates, metabolites, and the like, may be used in conjunction with activatable states or expression levels in the classification of cells encompassed here.

In some embodiments, other characteristics that affect the status of a cellular constituent may also be used to classify a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

Additional elements may also be used to classify a cell, such as the expression level of extracellular or intracellular markers, nuclear antigens, enzymatic activity, protein expression and localization, cell cycle analysis, chromosomal analysis, cell volume, and morphological characteristics like granularity, size and size of nucleus or other distinguishing characteristics. For example, B cells can be further subdivided based on the expression of cell surface markers such as B-cell receptor comprised of a membrane-bound form of a ligand binding moiety such as IgM, IgH, IgD, heavy chains non-covalently linked with kappa and lambda light chains and a signal transduction moiety which is heterodimer called Ig-α/Ig-β (CD79), bound together by disulfide bridges. Each member of the dimer spans the plasma membrane and has a cytoplasmic tail bearing an immunoreceptor tyrosine-based activation (ITAM) motif transduction moiety. Additional elements that are BCR regulators, such as the following can be used to classify the cell: CD45, CD5, CD19, CD20, CD22, CD23, CD27, CD37, CD40, CD52, CD38, CD96, major histocompatability antigen (MHC) Class 1 or MHC Class 2.

Alternatively, predefined classes of cells can be classified based upon shared characteristics that may include inclusion in one or more additional predefined class or the presence of extracellular and/or intracellular markers, a similar gene expression profile, mutational status, epigenetic silencing, nuclear antigens, enzymatic activity, protein expression and localization, cell cycle analysis, chromosomal analysis, cell volume, and morphological characteristics like granularity and size of nucleus or other distinguishing characteristics.

In some embodiments, the physiological status of one or more cells is determined by examining and profiling the activation level of one or more activatable elements in a cellular pathway. In some embodiments, a cell is classified according to the activation level of a plurality of activatable elements. In some embodiments, a hematopoietic cell is classified according to the activation levels of a plurality of activatable elements. In some embodiments, the activation level of one or more activatable elements of a hematopoietic cell is correlated with a condition. In some embodiments, the activation level of one or more activatable elements of a hematopoietic cell is correlated with a neoplastic, autoimmune or hematopoietic condition as described herein. Examples of hematopoietic cells include but are not limited to pluripotent hematopoietic stem cells, myeloid progenitors, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells. In some embodiments, the hematopoietic cell is a B-lymphocyte lineage progenitor or derived cell as described herein.

In some embodiments, the activation level of one or more activatable elements in single cells within the sample is determined. Cellular constituents that may include activatable elements include without limitation, proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation from one part of the cell to another, change in conformation (due to, e.g., change in pH or ion concentration), by proteolytic cleavage, and the like. Upon activation, a change occurs to the activatable element, such as covalent modification of the activatable element (e.g., binding of a molecule or group to the activatable element, including but not limited to, phosphorylation, acetylation, methylation, ubiquitination) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of activation of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. In some embodiments, at least about 2, 3, 4, 5, 6, 7, 8, 9, 10 or more than 10 intracellular activatable elements are determined.

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis (e.g. PARP), cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and U.S. Pat. No. 7,393,656 entitled “Methods and compositions for risk stratification” the content of which are incorporate here by reference.

In some embodiments, the activatable element that is a protein is selected from the group consisting Exemplary signaling proteins include, but are not limited to, kinases, HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lyn, Fgr, Yes, Csk, Abl, Btk, ZAP-70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks (1 and 2 for example), IKKs, GSK3β, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, lipid signaling, phosphoinositide kinases, phospholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon □, interferon □, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, □-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, PARP, proteins involved in apoptosis, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPB, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Glycoprotein, nucleoside transporters, transcription factors/DNA binding proteins, Ets, Elk, SMADs, Rel-A (p65-NFKB), CREB, NFAT, ATF-2, AFT, Myc, Fos, Sp1, Egr-1, T-bet, HIFs, FOXOs, E2Fs, SRFs, TCFs, Egr-1,13-catenin, FOXO STAT1, STAT3, STAT4, STAT5, STATE, p53, WT-1, HMGA, regulators of translation, pS6, 4EPB-1, eIF4E-binding protein, regulators of transcription, RNA polymerase, initiation factors, and elongation factors.

In some embodiments the protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHPT, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP-70, Lyn, Cbl, SLP-76, PLCγ1, PLCγ2, STAT1, STAT2, STAT3, STAT4, STAT5a, STAT5b, STATE, FAK, p130CAS, PAKs, LIMK1/2, Hsp90, Hsp70, Hsp27, SMADs, Rel-A (p65-NFKB), CREB, Histone H2B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, PARP, IAPB, Smac, Fodrin, Actin, Src, Lyn, Fyn, Lyn, NIK, IκB, p65(RelA), IKKβ, PKA, PKCγ, PKC□, PKC□, PKC□, CAMK, Elk, AFT, Myc, Egr-1, NFAT, ATF-2, Mdm2, p53, DNA-PK, Chk1, Chk2, ATM, ATR, β-catenin, CrkL, GSK3β, GSK3β, and FOXO.

In some embodiments, the classification of a cell according to activation level of an activatable element, e.g., in a cellular pathway comprises classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition. In some embodiments, the clinical outcome is the staging or grading of a neoplastic, autoimmune or hematopoietic condition. Examples of staging include, but are not limited to, aggressive, indolent, benign, refractory, Roman Numeral staging, TNM Staging, Rai staging, Binet staging, WHO classification, FAB classification, IPSS score, WPSS score, limited stage, extensive stage, staging according to cellular markers such as ZAP-70, IgV_{H} mutational status and CD38, occult, including information that may inform on time to progression, progression free survival, overall survival, or event-free survival.

In some embodiments, methods and compositions are provided for the classification of a cell according to the activation level of an activatable element, e.g., in a cellular pathway wherein the classification comprises classifying a cell as a cell that is correlated to a patient response to a treatment. In some embodiments, the patient response is selected from the group consisting of complete response, partial response, nodular partial response, no response, progressive disease, stable disease and adverse reaction.

In some embodiments, methods and compositions are provided for the classification of a cell according to the activation level of an activatable element, e.g., in a cellular pathway wherein the classification comprises classifying the cell as a cell that is correlated with minimal residual disease or emerging resistance.

In some embodiments, methods and compositions are provided for the classification of a cell according to the activation level of an activatable element, e.g., in a cellular pathway wherein the classification comprises selecting a method of treatment. Example of methods of treatments include, but are not limited to, chemotherapy, biological therapy, radiation therapy, bone marrow transplantation, Peripheral stem cell transplantation, umbilical cord blood transplantation, autologous stem cell transplantation, allogeneic stem cell transplantation, syngeneic stem cell transplantation, surgery, induction therapy, maintenance therapy, and watchful waiting.

Generally, the methods of the invention involve determining the activation levels of an activatable element in a plurality of single cells in a sample.

### Signaling Pathways

In some embodiments, the methods of the invention are employed to determine the status of an activatable element in a signaling pathway. In some embodiments, a cell is classified, as described herein, according to the activation level of one or more activatable elements in one or more signaling pathways. Signaling pathways and their members have been extensively described. See (Hunter T. Cell (2000) 100(1): 13-27). Exemplary signaling pathways include the following pathways and their members: The MAP kinase pathway including Ras, Raf, MEK, ERK and elk; the PI3K/Akt pathway including PI-3-kinase, PDK1, Akt and Bad; the canonical and non-canonical NF-□B pathway including Nik, IKKs, IkB and NF-κB and the Wnt pathway including frizzled receptors, beta-catenin, APC and other co-factors and TCF (see Cell Signaling Technology, Inc. 2002 Catalog pages 231-279 and Hunter T., supra.). In some embodiments of the invention, the correlated activatable elements being assayed (or the signaling proteins being examined) are members of the MAP kinase, Akt, NFkB, WNT, STAT and/or PKC signaling pathways. The methods of the invention also comprise the methods, signaling pathways and signaling molecules disclosed in U.S. 61/085,789 which is hereby incorporated by reference in its entirety.

In some embodiments, the methods of the invention are employed to determine the status of a signaling protein in a signaling pathway known in the art including those described herein. Exemplary types of signaling proteins within the scope of the present invention include, but are not limited to, kinases, kinase substrates (i.e. phosphorylated substrates), phosphatases, phosphatase substrates, binding proteins (such as 14-3-3), receptor ligands and receptors (cell surface receptor tyrosine kinases and nuclear receptors)). Kinases and protein binding domains, for example, have been well described (see, e.g., Cell Signaling Technology, Inc., 2002 Catalogue “The Human Protein Kinases” and “Protein Interaction Domains” pgs. 254-279). Signaling proteins are identified above as activatable elements. See also, U.S. Pat. No. 8,227,202.

MAP Kinase Pathway:

In some embodiments, the methods of the invention are employed to determine the status of an activatable element in the MAP kinase pathway. Without intending to be limited to any theory, the MAP Kinase pathway is a signal transduction pathway that couples intracellular responses to the binding of growth factors to cell surface receptors. This pathway is very complex and includes many protein components. In many cell types, activation of this pathway promotes cell division.

PI3K/Akt Pathway:

In some embodiments, the methods of the invention are employed to determine the status of an activatable element in a PI3K/Akt pathway. Without intending to be limited to any theory, the PI3K/Akt pathway plays a role in effecting alterations in abroad range of cellular functions in response to extracellular signals. A downstream effector of PI3K is the serine-threonine kinase Akt which in response to PI3K activation phosphorylates and regulates the activity of a number of targets including kinases, transcription factors and other regulatory molecules. The serine/threonine kinase Akt functions intracellularly as a nodal point for a constellation of converging upstream signaling pathways, which involve stimulation of receptor tyrosine kinases such as IGF-1R, HER2/Neu, VEGF-R, PDGF-R), and an assembly of membrane-localized complexes of receptor-PI3K and activation of Akt through the second messenger PIP3. The integration of these intracellular signals at the level of Akt and its kinase activity, regulates the phosphorylation of its several downstream effectors, such as NF-B, mTOR, Forkhead, Bad, GSK-3 and MDM-2. These phosphorylation events, in turn, mediate the effects of Akt on cell growth, proliferation, protection from pro-apoptotic stimuli, and stimulation of neoangiogenesis. Akt and its upstream regulators are deregulated in a wide range of solid tumors and hematologic malignancies. The Akt pathway is the central cell survival pathway that is activated by such oncogenic events as over expression of an upstream receptor tyrosine kinase such as EGFR (ibid) or loss of an upstream regulatory protein such as PTEN (ibid).

NE-κB Pathway:

In some embodiments, the methods of the invention are employed to determine the status of an activatable element in a NF-κB pathway. Without intending to be limited to any theory, the NF-κB pathway is involved in regulating many aspects of cellular activity, in stress, injury and especially in pathways of the immune response. Some examples are the response to and induction of IL-2, the induction of TAP1 and MHC molecules by NF-κB, and many aspects of the inflammatory response, e.g. induction of IL-1 (alpha and beta), TNF-alpha and leukocyte adhesion molecules (E-selectin, VCAM-1 and ICAM-1). Moreover, NF-κB is involved in many aspects of cell growth, differentiation and proliferation via the induction of certain growth and transcription factors (e.g. c-myc, ras and p53). The NF-κB signal transduction pathway is misregulated in a variety of human cancers, especially those of lymphoid cell origin. Several human lymphoid cancer cells are reported to have mutations or amplifications of genes encoding NF-κB transcription factors. In most cancer cells NF-κB is constitutively active and resides in the nucleus. In some cases, this may be due to chronic stimulation of the IKK pathway, while in others the gene encoding IkBa may be defective. Such continuous nuclear NF-κB activity not only protects cancer cells from apoptotic cell death, but may even enhance their growth activity. Designing anti-tumor agents to block NF-κB activity or to increase their sensitivity to conventional chemotherapy may have great therapeutic value.

WNT Pathway:

In some embodiments, the methods of the invention are employed to determine the status of an activatable element in a WNT pathway. Without intending to be limited to any theory, the Wnt signaling pathway describes a complex network of proteins most well known for their roles in embryogenesis and cancer, but also involved in normal physiological processes in adult animals. The canonical Wnt pathway describes a series of events that occur when Wnt proteins bind to cell-surface receptors of the Frizzled family, causing the receptors to activate Disheveled family proteins and ultimately resulting in a change in the amount of β-catenin that reaches the nucleus. Disheveled (DSH) is a key component of a membrane-associated Wnt receptor complex which, when activated by Wnt binding, inhibits a second complex of proteins that includes axin, GSK-3, and the protein APC. The axin/GSK-3/APC complex normally promotes the proteolytic degradation of the β-catenin intracellular signaling molecule. After this “β-catenin destruction complex” is inhibited, a pool of cytoplasmic β-catenin stabilizes, and some β-catenin is able to enter the nucleus and interact with TCF/LEF family transcription factors to promote specific gene expression.

PKC Pathway:

In some embodiments, the methods of the invention are employed to determine the status of an activatable element in a PKC pathway. Without intending to be limited to any theory, PKC pathway is associated with cell proliferation, differentiation, and apoptosis. At least eleven closely related PKC isozymes have been reported that differ in their structure, biochemical properties, tissue distribution, subcellular localization, and substrate specificity. They are classified as conventional, novel, and atypical isozymes. Conventional PKC isozymes are Ca2+-dependent, while novel and atypical isozymes do not require Ca2+ for their activation. All PKC isozymes, with the exception of and, are activated by diacylglycerol (DAG). PKC isozymes negatively or positively regulate critical cell cycle transitions, including cell cycle entry and exit and the G1 and G2 checkpoints. Altered PKC activity has been linked with various types of malignancies. Higher levels of PKC and differential activation of various PKC isozymes have been reported in breast tumors, adenomatous pituitaries, thyroid cancer tissue, leukemic cells, and lung cancer cells. Down regulation of PKC□ is reported in the majority of colon adenocarcinomas and in the early stages of intestinal carcinogenesis. Thus, PKC inhibitors have become important tools in the treatment of cancers. The involvement of PKC in the regulation of apoptosis adds another dimension to the effort to develop drugs that will specifically target PKC. PKC pathway activation is thought to also play a role in diseases such as cardiovascular disease and diabetes.

In some embodiments of the invention, the methods described herein are employed to determine the status of an activatable element in a signaling pathway. Methods and compositions are provided for the classification of a cell according to the status of an activatable element in a signaling pathway. The cell can be a hematopoietic cell. Examples of hematopoietic cells are described above.

In some embodiments, the classification of a cell according to the status of an activatable element in a signaling pathway comprises classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition. In some embodiments, the clinical outcome is the staging or grading of a neoplastic, autoimmune or hematopoietic condition. Examples of staging include, but are not limited to, aggressive, indolent, benign, refractory, Roman Numeral staging, TNM Staging, Rai staging, Binet staging, WHO classification, FAB classification, IPSS score, WPSS score, limited stage, extensive stage, staging according to cellular markers such as ZAP-70, IgV_{H} mutational status and CD38, occult, including information that may inform on time to progression, progression free survival, overall survival, or event-free survival.

In some embodiments, methods and compositions are provided for the classification of a cell according to the status of an activatable element in a signaling pathway wherein the classification comprises classifying a cell as a cell that is correlated to a patient response to a treatment. In some embodiments, the patient response is selected from the group consisting of complete response, partial response, nodular partial response, no response, progressive disease, stable disease and adverse reaction.

In some embodiments, methods and compositions are provided for the classification of a cell according to the status of an activatable element in a signaling pathway wherein the classification comprises classifying the cell as a cell that is correlated with minimal residual disease or emerging resistance.

The invention is not limited to presently elucidated signaling pathways and signal transduction proteins, and encompasses signaling pathways and proteins subsequently identified.

### B-Cell Receptor Pathway

In some embodiments, the methods and compositions of the invention may be employed to examine and profile the status of any activatable element in B-Cell Receptor (BCR) signaling, or collections of such activatable elements in a B-lymphocyte lineage progenitor or derived cell. In some embodiments, the physiological status of one or more B-lymphocyte lineage progenitor or derived cell is determined by examining and profiling the status of one or more activatable element in BCR signaling. In some embodiments, a B-lymphocyte lineage progenitor or derived cell is classified, as described herein, according to the activation level of one or more activatable elements in BCR signaling. Examples of B-lymphocyte lineage derived cell include, but are not limited to, B-lymphocyte lineage early pro-B cell, late pro-B cell, large pre-B cell, small pre-B cell, immature B cell, mature B cell, plasma cell, memory B cell, a CD5+ B cell, a CD38+ B cell, a B cell bearing a mutated or non mutated heavy chain of the B cell receptor and a B cell expressing ZAP-70. In some embodiments, the B-lymphocyte lineage progenitor or derived cell is a cell associated with a condition as described herein.

Without intending to be limited to any theory, BCR cross-linking triggers phosphorylation of tyrosines within the ITAM motif domains of Ig□ and Ig□ by Src family member tyrosine kinases (e.g., Lyn, Lyn, Blk, Fyn). The phosphorylated ITAMs of Ig□□ recruit and enhance phosphorylation of Syk (directly) and Btk (via Syk). BCR cross-linking also brings together numerous regulator and adapter molecules (e.g., SLP-65/BLNK, Grb2, CD22, SHP-1) and compartmentalizes the BCR in lipid rafts with coreceptors CD19 and CD21. Following Syk and Btk activation, the enzymes phospholipase-Cγ2 (PLCγ2) and PI3K propagate BCR signaling. PLCγ 2 activation generates calcium flux, inositol-1,4,5-triphosphate, and diacylglycerol, and results in activation of protein kinase C and NF-κB. Syk interacts with PLCγ2 via adapters, whereas Btk can interact directly, and each is required for PLCγ 2 activity following BCR cross-linking. Both Syk and Btk can activate PI3K following BCR cross-linking Activation of PI3K enables Akt-mediated survival signaling, and PI3K is required for BCR-mediated survival during B cell development. PLCγ 2 and PI3K also initiate kinase cascades that result in phosphorylation of the MAPK family proteins ERK1/2 and p38. Activation of the Ras-Raf-ERK1/2 signaling cascade is considered a central event in BCR signaling, and decreased Ras activation due to RasGRP1 and RasGRP3 loss in mouse impairs B cell proliferation. In contrast, p38 is a stress response protein that interacts with p53 and regulates cell cycle checkpoints. Differential activation of ERK1/2 and p38 might enable the BCR to drive diverse cellular outcomes, but the question arises whether a given B cell activates these two pathways simultaneously or favors one pathway depending on additional signaling context.

Efficient activation of BCR signaling depends on generation of H₂O₂ and inactivation of negative regulatory protein tyrosine phosphatases (PTPs). Following BCR cross-linking, recruitment and activation of calcium-dependent NADPH oxidases (NOX) proteins, such as NOX5, enables production of H₂O₂ and lowers the signaling threshold for the BCR. BCR-induced H₂O₂ transiently inactivates membrane proximal PTPs, including SHP-1, via reversible oxidation of the catalytic cysteine to sulfenic acid. Elegant work reconstituting the BCR signaling pathway in insect cells has suggested a model of redox feedback loops where H₂O₂ inactivates PTPs and enables amplification of early signaling events, such as Syk phosphorylation and ITAM binding. Recent work characterized endogenously generated H₂O₂ as the primary redox species generated by BCR signaling and indicated that NOX-dependent production of H₂O₂ was critical to initiate a wave of BCR signaling in mouse A20 B cells.

In some embodiments, the invention provides a method for classifying a B-lymphocyte lineage progenitor or derived cell upon treatment with a modulator and/or inhibitor. Examples of B-lymphocyte lineage progenitor or derived cells include, but are not limited to an early pro-B cell, late pro-B cell, large pre-B cell, small pre-B cell, immature B cell, mature B cell, plasma cell and memory B cell, a CD5+ B cell, a CD38+ B cell, a B cell bearing a mutated or non mutated heavy chain of the B cell receptor, or a B cell expressing ZAP-70.

In some embodiments, the classification includes classifying the cell according to the status of an activatable element in a BCR pathway as a cell that is correlated with a clinical outcome. In some embodiments, the invention provides methods for classifying a B-lymphocyte lineage progenitor or derived cell based on an alteration in signaling proximal to the BCR. In some embodiments, the clinical outcome is the prognosis and/or diagnosis of a condition. In some embodiments, the clinical outcome is the presence or absence of a neoplastic, autoimmune or a hematopoietic condition, such as Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia. In some embodiments, the condition is CLL. In some embodiments, the invention provides methods for classifying a CLL cell based on an alteration in signaling proximal to the BCR. The presence of the alteration is indicative of a clinical outcome. In some embodiments, CLL is defined by a monoclonal B cell population that may co-express the following markers alone or in all possible combinations: CD5, CD 20, CD19, CD22, CD23, CD38, and CD45. Other arrangements include CDCD5 with CD19 and CD23 or CD5 with CD20 and CD23 and by surface immunoglobulin expression. In some embodiments, CLL is defined by a monoclonal B cell population that co-expresses CD5 with CD19 and CD23 or CD5 with CD20 and CD23 and dim surface immunoglobulin expression. Additional B-cell markers can be used to identify or classify a B-lymphocyte lineage progenitor or derived cell. Non-limiting examples such as the following can be used to classify the cell: CD45, CD5, CD19, CD20, CD22, CD23, CD27, CD37, CD40, CD52, CD38, CD96, major histocompatability antigen (MHC) Class 1 or MHC Class 2.

In some embodiments of the methods of the invention, the classifying of the B-lymphocyte lineage progenitor or derived cell based on activation level of an activatable element in BCR pathway includes classifying the cell as a cell that is correlated to a patient response to a treatment, as defined above.

In some embodiments of the methods of the invention, the classifying of the B-lymphocyte lineage progenitor or derived cells based on activation of an activatable element in BCR pathway includes classifying the cell as a cell that is correlated with minimal residual disease or emerging resistance.

### Tonic Signaling

In some embodiments, the methods and compositions of the invention may be employed to determine the status of a tonic signaling pathway in a cell. In some embodiments, the methods and compositions of the invention may be employed to examine and profile the status of any activatable element in a tonic signaling pathway, or collections of such activatable elements in a cell. In some embodiments, the physiological status of a cell is determined by examining and profiling the status of one or more activatable elements in a tonic signaling pathway. In some embodiments, a cell is classified, as described herein, according to the status of one or more activatable elements in a tonic signaling pathway. The term “tonic signaling” includes ligand-independent signaling, antigen independent signaling, basal signaling, signaling in the resting state, and non-induced or ligand-independent signaling.

Without intending to be limited to any theory, recent evidence supports the notion that in most signal transduction systems regulated by cellular receptors some basal level of signaling occurs continuously in a ligand-independent manner, although the flux through such systems may vary considerably. The basal, tonic, or the steady state level of signaling in unstimulated cells is the result of equilibrium of positive and negative regulators within a signaling pathway. Thus, the balanced actions of positive and negative regulators of signal transduction set the steady state equilibrium. The steady state level of signaling in the unstimulated state may itself have functional consequences, for instance, to maintain certain differentiated cellular properties or functions.

In some embodiments, the invention provides for methods of determining tonic signaling status of a cell. Methods and compositions are provided for the classification of a cell according to the status of an activatable element in a tonic signaling pathway. The cell can be a hematopoietic cell. Examples of hematopoietic cells are described above.

In some embodiments, the classification of a cell according to the status of an activatable element in a tonic signaling pathway comprises classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition. Examples of neoplastic, autoimmune or hematopoietic conditions include, but are not limited to, such as Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia. In some embodiments, the condition is CLL. In some embodiments, CLL is defined by a monoclonal B cell population that co-expresses CD5 with CD19 and CD23 or CD5 with CD20 and CD23 and by surface immunoglobulin expression.

In some embodiments, the clinical outcome is the staging or grading of a neoplastic, autoimmune or hematopoietic condition. Examples of staging are defined above.

In some embodiments, the invention provides methods for classifying a CLL cell based on an alteration in signaling proximal to the BCR that is indicative of the presence of tonic signaling. The presence of the alteration is indicative of a clinical outcome, where the clinical outcome is as described herein.

In some embodiments, methods and compositions are provided for the classification of a cell according to the status of an activatable element in a tonic signaling pathway wherein the classification comprises classifying a cell as a cell that is correlated to a patient response to a treatment. In some embodiments, the patient response is selected from the group consisting of complete response, partial response, nodular partial response, no response, progressive disease, stable disease and adverse reaction.

In some embodiments, methods and compositions are provided for the classification of a cell according to the status of an activatable element in a tonic signaling pathway wherein the classification comprises classifying the cell as a cell that is correlated with minimal residual disease or emerging resistance.

In some embodiments, methods and compositions are provided for the classification of a cell according to the status of an activatable element in a tonic signaling pathway wherein the classification comprises selecting a method of treatment. Examples of methods of treatments are described above.

### Binding Element

In some embodiments of the invention, the activation level of an activatable element is determined by contacting a cell with a binding element that is specific for an activation state of the activatable element. The term “Binding element” includes any molecule, e.g., peptide, polypeptide (including an antibody) nucleic acid, small organic molecule which is capable of detecting an activation state of an activatable element over another activation state of the activatable element. See U.S. Ser. No. 12/229,476 which is incorporated by reference in its entirety.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and norleucine are considered amino acids for the purposes of the invention. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999, both of which are expressly incorporated by reference herein.

Methods of the present invention may be used to detect any particular activatable element in a sample that is antigenically detectable and antigenically distinguishable from other activatable element which is present in the sample. For example, as demonstrated (see, e.g., the Examples) and described herein, the activation state-specific antibodies of the present invention can be used in the present methods to identify distinct signaling cascades of a subset or subpopulation of complex cell populations; and the ordering of protein activation (e.g., kinase activation) in potential signaling hierarchies. Hence, in some embodiments the expression and phosphorylation of one or more polypeptides are detected and quantified using methods of the present invention. In some embodiments, the expression and phosphorylation of one or more polypeptides that are cellular components of a cellular pathway are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of an activatable element. Also preferably, the binding of the activation state-specific antibody is indicative of a specific activation state of a specific activatable element.

In some embodiments, the binding element is an antibody. In some embodiment, the binding element is an activation state-specific antibody. In some embodiment, the binding element is a phospho-specific antibody.

As pointed out above, activation state specific antibodies can be used to detect kinase activity, however additional means for determining kinase activation are provided by the present invention. For example, substrates that are specifically recognized by protein kinases and phosphorylated thereby are known. Antibodies that specifically bind to such phosphorylated substrates but do not bind to such non-phosphorylated substrates (phospho-substrate antibodies) may be used to determine the presence of activated kinase in a sample.

Many antibodies, many of which are commercially available (for example, see Cell Signaling Technology, see cellsignal.com, Millipore, eBioscience, Caltag, Santa Cruz Biotech, Abcam, BD Biosciences, Sigma and Anaspec) the contents which are incorporated herein by reference) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lyn, Fgr, Yes, Csk, Abl, Btk, ZAP-70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3β, GSK3□, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, lipid signaling, phosphoinositide kinases, phospholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferonα, interferon γ, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, □-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, PARP, proteins involved in apoptosis, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPB, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Glycoprotein, nucleoside transporters, transcription factors/DNA binding proteins, Ets, Elk, SMADs, Rel-A (p65-NFKB), CREB, NFAT, ATF-2, AFT, Myc, Fos, Sp1, Egr-1, T-bet, □-catenin, HIFs, FOXOs, E2Fs, SRFs, TCFs, Egr-1, β-catenin, FOXO STAT1, STAT3, STAT4, STAT5, STATE, p53, WT-1, HMGA, regulators of translation, pS6, 4EPB-1, eIF4E-binding protein, regulators of transcription, RNA polymerase, initiation factors, elongation factors. In some embodiments, the protein is S6.

Non-activation state antibodies may also be used in the present invention. In some embodiments, non-activation state antibodies bind to epitopes in both activated and non-activated forms of an element. Such antibodies may be used to determine the amount of non-activated plus activated element in a sample. In some embodiments, non-activation state antibodies bind to epitopes present in non-activated forms of an element but absent in activated forms of an element. Such antibodies may be used to determine the amount of non-activated element in a sample. Both types of non-activation state antibodies may be used to determine if a change in the amount of activation state element, for example from samples before and after treatment with a candidate bioactive agent as described herein, coincide with changes in the amount of non-activation state element. For example, such antibodies can be used to determine whether an increase in activated element is due to activation of non-activation state element, or due to increased expression of the element, or both.

### Labels

The methods and compositions of the instant invention provide binding elements comprising a label or tag. By label is meant a molecule that can be directly (i.e., a primary label) or indirectly (i.e., a secondary label) detected; for example a label can be visualized and/or measured or otherwise identified so that its presence or absence can be known. A compound can be directly or indirectly conjugated to a label which provides a detectable signal, e.g. radioisotopes, fluorescers, enzymes, antibodies, particles such as magnetic particles, chemiluminescers, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes. See U.S. Ser. No. 12/229,476 which is incorporated by reference in its entirety.

In some embodiments, one or more binding elements are uniquely label. Using the example of two activation state specific antibodies, by “uniquely labeled” is meant that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels fall into four classes: a) isotopic labels, which may be radioactive or heavy isotopes; b) magnetic, electrical, thermal labels; c) colored, optical labels including luminescent, phosphorous and fluorescent dyes or moieties; and d) binding partners. Labels can also include enzymes (horseradish peroxidase, etc.) and magnetic particles. In some embodiments, the detection label is a primary label. A primary label is one that can be directly detected, such as a fluorophore.

Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 1996 Molecular Probes Handbook by Richard P. Haugland, hereby expressly incorporated by reference. Suitable fluorescent labels also include, but are not limited to, green fluorescent protein (GFP; Chalfie, et al., Science 263(5148):802-805 (Feb. 11, 1994); and EGFP; Clontech—Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), .beta.-galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558). All of the above-cited references are expressly incorporated herein by reference.

In some embodiments, labels for use in the present invention include: Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes) (Eugene, Oreg.), FITC, Rhodamine, and Texas Red (Pierce, Rockford, Ill.), Cy5, Cy5.5, Cy7 (Amersham Life Science, Pittsburgh, Pa.). Tandem conjugate protocols for Cy5PE, Cy5.5PE, Cy7PE, Cy5.5APC, Cy7APC are known in the art. Quantitation of fluorescent probe conjugation may be assessed to determine degree of labeling and protocols including dye spectral properties are also well known in the art. In some embodiments the fluorescent label is conjugated to an aminodextran linker which is conjugated to a binding element or antibody. Additional labels listed in and are available through the on-line and hard copy catalogues of BD Biosciences, Beckman Coulter, AnaSpec, Invitrogen, Cell Signaling Technology, Millipore, eBioscience, Caltag, Santa Cruz Biotech, Abcam and Sigma, the contents of which are incorporated herein by reference.

In some embodiments, the activatable elements are labeled with tags suitable for Inductively Coupled Plasma Mass Spectrometer (ICP-MS) as disclosed in Tanner et al. Spectrochimica Acta Part B: Atomic Spectroscopy, 2007 March; 62(3):188-195; Ornatsky et al, mRNA Detection in Leukemia Cell lines by Novel Metal-Tagged in situ Hybridization using Inductively Coupled Plasma Mass Spectometry, Translational Oncogenomics (2006):1, 1-9; Ornatsky et al, Multiple Cellular Antigen Detection by ICP-MS, J. 1 mm. Methods 308 (2006) 68-76; and Lou et al., Polymer-Based Elemental Tags for Sensitive Bioassays, Angew. Chem. Int. Ed., (2007) 46, 6111-6114.

Production of antibody-embedded substrates is well known; see Slinkin et al., Bioconj. Chem., 2:342-348 (1991); Torchilin et al., supra; Trubetskoy et al., Bioconj. Chem. 3:323-327 (1992); King et al., Cancer Res. 54:6176-6185 (1994); and Wilbur et al., Bioconjugate Chem. 5:220-235 (1994) (all of which are hereby expressly incorporated by reference), and attachment of or production of proteins with antigens is described above. Calmodulin-embedded substrates are commercially available, and production of proteins with CBP is described in Simcox et al., Strategies 8:40-43 (1995), which is hereby incorporated by reference in its entirety.

As will be appreciated by those in the art, tag-components of the invention can be made in various ways, depending largely upon the form of the tag. Components of the invention and tags are preferably attached by a covalent bond.

### Alternative Activation State Indicators

An alternative activation state indicator useful with the instant invention is one that allows for the detection of activation by indicating the result of such activation. For example, phosphorylation of a substrate can be used to detect the activation of the kinase responsible for phosphorylating that substrate. Similarly, cleavage of a substrate can be used as an indicator of the activation of a protease responsible for such cleavage. Methods are well known in the art that allow coupling of such indications to detectable signals, such as the labels and tags described above in connection with binding elements. For example, cleavage of a substrate can result in the removal of a quenching moiety and thus allowing for a detectable signal being produced from a previously quenched label.

### Modulators

In some embodiments, the methods and composition utilize a modulator. A modulator can be an activator, an inhibitor or a compound capable of impacting a cellular pathway. Modulators can take the form of environmental cues and inputs.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments any suitable amount of serum is used. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments, modulators produce different activation states depending on the concentration of the modulator, duration of exposure or whether they are used in combination or sequentially with other modulators.

In some embodiments the modulator is selected from the group consisting of growth factor, cytokine, adhesion molecule modulator, drugs, hormone, small molecule, polynucleotide, antibodies, natural compounds, lactones, chemotherapeutic agents, immune modulator, carbohydrate, proteases, ions, reactive oxygen species, peptides, and protein fragments, either alone or in the context of cells, cells themselves, viruses, and biological and non-biological complexes (e.g. beads, plates, viral envelopes, antigen presentation molecules such as major histocompatibility complex). In some embodiments, the modulator is a physical stimuli such as heat, cold, UV radiation, and radiation. Examples of modulators, include but are not limited to, F(ab)₂IgM, SDF1a, R848, anti-IgD, CD40L, thapsigargin, fludarabine, bendamustine, poly CpG, or IFNa and/or combinations thereof.

In some embodiments, the modulator is an activator. In some embodiments the modulator is an inhibitor. In some embodiments, cells are exposed to one or more modulator. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the modulator is a B cell receptor modulator. In some embodiments, the B cell receptor modulator is a B cell receptor activator. An example of B cell receptor activator is a cross-linker of the B cell receptor complex or the B-cell co-receptor complex. In some embodiments, cross-linker is an antibody or molecular binding entity. In some embodiments, the cross-linker is an antibody. In some embodiments, the antibody is a multivalent antibody. In some embodiments, the antibody is a monovalent, bivalent, or multivalent antibody made more multivalent by attachment to a solid surface or tethered on a nanoparticle surface to increase the local valency of the epitope binding domain.

In some embodiments, the cross-linker is a molecular binding entity. In some embodiments, the molecular binding entity acts upon or binds the B cell receptor complex via carbohydrates or an epitope in the complex. In some embodiments, the molecular is a monovalent, bivalent, or multivalent is made more multivalent by attachment to a solid surface or tethered on a nanoparticle surface to increase the local valency of the epitope binding domain.

In some embodiments, the cross-linking of the B cell receptor complex or the B-cell co-receptor complex comprises binding of an antibody or molecular binding entity to the cell and then causing its crosslinking via interaction of the cell with a solid surface that causes crosslinking of the BCR complex via antibody or molecular binding entity.

In some embodiments, the crosslinker is F(ab)₂ IgM, IgG, IgD, polyclonal BCR antibodies, monoclonal BCR antibodies, Fc receptor derived binding elements and/or a combination thereof. The Ig can be derived from a species selected from the group consisting of mouse, goat, rabbit, pig, rat, horse, cow, shark, chicken, or llama. In some embodiments, the crosslinker is F(ab)₂ IgM, Polyclonal IgM antibodies, Monoclonal IgM antibodies, Biotinylated F(ab)₂ IgG/M, Biotinylated Polyclonal IgM antibodies, Biotinylated Monoclonal IgM antibodies and/or combination thereof.

In some embodiments, the inhibitor is an inhibitor of a cellular factor or a plurality of factors that participates in a cellular pathway (e.g. signaling cascade) in the cell. In some embodiments, the inhibitor is a kinase or phosphatase inhibitor. Examples of kinase inhibitors are recited above.

In some embodiments H₂O₂ is administered as an inhibitor. In some embodiments H₂O₂ is administered at between 0.01 and 50 mM. In some embodiments H₂O₂ is administered at between 0.1 and 10 mM. In some embodiments H₂O₂ is administered at between 1 and 10 mM. In some embodiments H₂O₂ is administered at between 1 and 5 mM. In some embodiments H₂O₂ is administered at 0.5, 1, 1.5, 2, 2.5, 3, 3.5, 4, 4.5, 5, 5.5, 6, 6.5, 7, 7.5, 8, 8.5, 9, 9.5 or 10 mM. In certain embodiments, H₂O₂ is administered at 3.0 mM. In certain embodiments, H₂O₂ is administered at 3.3 mM. In some embodiments the duration of exposure of H₂O₂ is between 0.01 and 360 minutes. In some embodiments the duration of exposure of H₂O₂ is between 0.1 and 240 minutes. In some embodiments the duration of exposure of H₂O₂ is between 0.5 and 180 minutes. In some embodiments the duration of exposure of H₂O₂ is between 0 and 120 minutes. In some embodiments the duration of exposure to H₂O₂ is between 5 and 15 minutes. In some embodiments the duration of exposure to an inhibitor, such as H₂O₂ as one example (and used below) is 1, 2, 3, 4, 5, 10, 15, 20, 25, 30, 35, 40, 45, 50, 55, 60, 70, 80, 90, 100, 110, 120, 140, 160 or 180 minutes. In some embodiments the duration of exposure of H₂O₂ is 10 minutes. In some embodiments H₂O₂ is administered as an inhibitor with at least one other modulator. In some embodiments H₂O₂ is administered as an inhibitor with F(ab)₂ IgM or any suitable BCR agonist. In some embodiments H₂O₂ is administered before administration of F(ab)₂ IgM. In some embodiments H₂O₂ is administered simultaneously with F(ab)₂ IgM. In some embodiments H₂O₂ is administered after F(ab)₂ IgM.

In some embodiments, the activation level of an activatable element in a cell is determined after contacting the cell with at least 2, 3, 4, 5, 6, 7, 8, 9, or 10 modulators. In some embodiments, the activation level of an activatable element in a cell is determined after contacting the cell with at least 2, 3, 4, 5, 6, 7, 8, 9, or 10 modulators where at least one of the modulators is an inhibitor. In some embodiments, the activation level of an activatable element in a cell is determined after contacting the cell with an inhibitor and a modulator, where the modulator can be an inhibitor or an activator. In some embodiments, the activation level of an activatable element in a cell is determined after contacting the cell with an inhibitor and an activator. In some embodiments, the activation level of an activatable element in a cell is determined after contacting the cell with two or more modulators.

In some embodiments, a phenotypic profile of a population of cells is determined by measuring the activation level of an activatable element when the population of cells is exposed to a plurality of modulators in separate cultures. In some embodiments, the modulators include from the group F(ab)₂IgM, SDF1a, R848, anti-IgD, CD40L, thapsigargin, fludarabine, bendamustine, poly CpG, or IFNa and/or a combination thereof.

### Detection

In practicing the methods of this invention, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention is performed according to standard techniques and protocols well-established in the art. See U.S. Ser. No. 12/229,476 and Ser. No. 12/460,029 which is incorporated by reference in its entirety.

One or more activatable elements can be detected and/or quantified by any method that can detect and/or quantitate the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters.

In some embodiments, the present invention provides methods for determining an activatable element's activation profile for a single cell. The methods may comprise analyzing cells by flow cytometry on the basis of the activation level of at least two activatable elements. Binding elements (e.g. activation state-specific antibodies) are used to analyze cells on the basis of activatable element activation level, and can be detected as described below. Alternatively, non-binding elements systems as described above can be used in any system described herein.

When using fluorescent labeled components in the methods and compositions of the present invention, it will recognized that different types of fluorescent monitoring systems, e.g., Cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., U.S. Pat. Nos. 6,455,263; 6,821,740; 6008,052; 6,897,954; 7,381,535, and 7,393,656 as well as U.S.P. Publication 20100197512 each expressly incorporated herein by reference). Other flow cytometers that are commercially available include the LSR II and the Canto II both available from Becton Dickinson others are available from Attune Acoustic Cytometer (Life Technologies, Carlsbad, Calif.) and the CyTOF (DVS Sciences, Sunnyvale, Calif.). See Shapiro, Howard M., Practical Flow Cytometry, 4th Ed., John Wiley & Sons, Inc., 2003 for additional information on flow cytometers.

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of a change in activation level in an activatable element in response to a modulator. In some embodiments the change is a decrease. In some embodiments the change is an increase.

In some embodiments, the cells are first contacted with fluorescent-labeled activation state-specific binding elements (e.g. antibodies) directed against specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic filed while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyze the population of cells by flow cytometry. Preferably, cells are analyzed on the basis of the activation level of at least two activatable elements. In some embodiments, a multiplicity of activatable element activation-state antibodies is used to simultaneously determine the activation level of a multiplicity of elements.

In some embodiment, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

As will be appreciated, the present invention also provides for the ordering of element clustering events in signal transduction. Particularly, the present invention allows the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

The present invention provides a valuable method of determining the presence of cellular subsets within cellular populations. Ideally, signal transduction pathways are evaluated in homogeneous cell populations to ensure that variances in signaling between cells do not qualitatively nor quantitatively mask signal transduction events and alterations therein. As the ultimate homogeneous system is the single cell, the present invention allows the individual evaluation of cells to allow true differences to be identified in a significant way.

Thus, the invention provides methods of distinguishing cellular subsets within a larger cellular population. As outlined herein, these cellular subsets often exhibit altered biological characteristics (e.g. activation levels, altered response to modulators) as compared to other subsets within the population. For example, as outlined herein, the methods of the invention allow the identification of subsets of cells from a population such as primary cell populations, e.g. peripheral blood mononuclear cells that exhibit altered responses (e.g. response associated with presence of a condition) as compared to other subsets. In addition, this type of evaluation distinguishes between different activation states, altered responses to modulators, cell lineages, cell differentiation states, etc.

As will be appreciated, these methods provide for the identification of distinct signaling cascades for both artificial and stimulatory conditions in complex cell populations, such a peripheral blood mononuclear cells, or naive and memory lymphocytes.

When necessary, cells are dispersed into a single cell suspension (e.g. by enzymatic digestion with a suitable protease, collagenase, dispase, etc; and the like). An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 min in acetone at −200 C; and the like as known in the art and according to the methods described herein.

In some embodiments, one or more cells are contained in a well of a 96 well plate or other commercially available multi-well plate. In an alternate embodiment, the reaction mixture or cells are in a cytometric measurement device. Other multi-well plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activatable element. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, (2007), 62(3):188-195.).

As will be appreciated by one of skill in the art, the instant methods and compositions find use in a variety of other assay formats in addition to flow cytometry analysis. For example, a chip analogous to a DNA chip can be used in the methods of the present invention. Arrayers and methods for spotting nucleic acid to a chip in a prefigured array are known. In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell.

In some embodiments, a chip comprises a multiplicity of the “second set binding elements,” in this case generally unlabeled. Such a chip is contacted with sample, preferably cell extract, and a second multiplicity of binding elements comprising element activation state specific binding elements is used in the sandwich assay to simultaneously determine the presence of a multiplicity of activated elements in sample. Preferably, each of the multiplicity of activation state-specific binding elements is uniquely labeled to facilitate detection.

In some embodiments confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2, which is hereby incorporated in its entirety. Data obtained by scanning of the multi-well plate can be analyzed and activation profiles determined as described below.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiment, the methods of the invention include the use of liquid handling components. The liquid handling systems can include robotic systems comprising any number of components. In addition, any or all of the steps outlined herein may be automated; thus, for example, the systems may be completely or partially automated. See U.S. Ser. Nos. 12/679,448 and 12/606,869.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation. Additional examples of automation, automated sample collection and analysis are disclosed in U.S. Ser. Nos. 12/432,239 and 12/606,869 which are hereby incorporated by reference in their entireties.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradeable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc.

### Analysis

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g. microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. Methods for the analysis of multiple parameters are well known in the art. In some embodiments flow cytometry applications require software for different phases of operation and analysis, see 12/501,274; 12/501,295; 12/293,081; 12/538,643; 12/460,029; and 13/566,991 which are hereby incorporated by reference in their entireties.

In some embodiments where flow cytometry is used, flow cytometry experiments are arrayed and the results are approximated as fold changes using a heat map to facilitate evaluation. Generally speaking, arrayed flow cytometry experiments simplify multidimensional flow cytometry data based on experimental design and observed differences between flow cytometry samples. One common way of comparing changes in a set of flow cytometry samples is to overlay histograms of one parameter on the same plot. Arrayed flow cytometry experiments ideally contain a reference sample against which experimental samples are compared. This reference sample is placed in the first position of the array, and subsequent experimental samples follow the control in the sequence. Reference samples can include normal and/or cells associated with a condition (e.g. tumor cells).

In some embodiments where flow cytometry is used, prior to analyzing of data the populations of interest and the method for characterizing these populations are determined. For instance, there are at least two general ways of identifying populations for data analysis: (i) “Outside-in” comparison of Parameter sets for individual samples or subset (e.g., patients in a trial). In this more common case, cell populations are homogenous or lineage gated in such a way as to create distinct sets considered to be homogenous for targets of interest. An example of sample-level comparison would be the identification of signaling profiles in tumor cells of a patient and correlation of these profiles with non-random distribution of clinical responses. This is considered an outside-in approach because the population of interest is pre-defined prior to the mapping and comparison of its profile to other populations. (ii) “Inside-out” comparison of Parameters at the level of individual cells in a heterogeneous population. An example of this would be the signal transduction state mapping of mixed hematopoietic cells under certain conditions and subsequent comparison of computationally identified cell clusters with lineage specific markers. This could be considered an inside-out approach to single cell studies as it does not presume the existence of specific populations prior to classification. A major drawback of this approach is that it creates populations which, at least initially, require multiple transient markers to enumerate and may never be accessible with a single cell surface epitope. As a result, the biological significance of such populations can be difficult to determine. The main advantage of this unconventional approach is the unbiased tracking of cell populations without drawing potentially arbitrary distinctions between lineages or cell types.

Each of these techniques capitalizes on the ability of flow cytometry to deliver large amounts of multiparameter data at the single cell level. For cells associated with a condition (e.g. neoplastic, autoimmune or hematopoetic condition), a third “meta-level” of data exists because cells associated with a condition (e.g. cancer cells) are generally treated as a single entity and classified according to historical techniques. These techniques have included organ or tissue of origin, degree of differentiation, proliferation index, metastatic spread, and genetic or metabolic data regarding the patient.

In some embodiments, the present invention uses variance mapping techniques for mapping condition signaling space. These methods represent a significant advance in the study of condition biology because it enables comparison of conditions independent of a putative normal control. Traditional differential state analysis methods (e.g., DNA microarrays, subtractive Northern blotting) generally rely on the comparison of cells associated with a condition from each patient sample with a normal control, generally adjacent and theoretically untransformed tissue. Alternatively, they rely on multiple clusterings and re-clusterings to group and then further stratify patient samples according to phenotype. In contrast, variance mapping of condition states compares condition samples first with themselves and then against the parent condition population. As a result, activation states with the most diversity among conditions provide the core parameters in the differential state analysis. Given a pool of diverse conditions, this technique allows a researcher to identify the molecular events that underlie differential condition pathology (e.g., cancer responses to chemotherapy), as opposed to differences between conditions and a proposed normal control.

In some embodiments, when variance mapping is used to profile the signaling space of patient samples, conditions whose signaling response to modulators is similar are grouped together, regardless of tissue or cell type of origin. Similarly, two conditions (e.g. two tumors) that are thought to be relatively alike based on lineage markers or tissue of origin could have vastly different abilities to interpret environmental stimuli and would be profiled in two different groups.

When groups of signaling profiles have been identified it is frequently useful to determine whether other factors, such as clinical responses, presence of gene mutations, and protein expression levels, are non-randomly distributed within the groups. If experiments or literature suggest such a hypothesis in an arrayed flow cytometry experiment, it can be judged with simple statistical tests, such as the Student's t-test and the □² test. Similarly, if two variable factors within the experiment are thought to be related, the r² correlation coefficient from a linear regression is used to represent the degree of this relationship.

In one embodiment of the invention, several metrics have been developed that compare distributions of per cell fluorescent intensities. These metrics may operate with one or more readouts (i.e., in one or more dimensions). The metrics may compare fluorescent intensities between cells (possibly from gated populations) from a single sample in basal and modulated states or compare the fluorescent intensities of cells from a given sample to a reference distribution of intensities; the fluorescent intensities may be untransformed compensated data or transformed using functions such as logarithm of base 2, natural logarithm, logarithm of base 10, arcsinh, etc. The reference distribution may be derived from a cohort of samples in a current experiment or from historical data and may comprise cells in one or more states including basal and modulated with one or more modulators; in addition, the reference distribution may be from a gated population, which may or may not be the same population as the population for which the metric is calculated (e.g., the metric may be computed comparing B-cells to T-cells). The reference distribution may be treated as discrete cell events, as a histogram of cell events (representing frequencies of intensities) or as one of a plurality of distribution functions (e.g., normal, beta, gamma, exponential, Dirichlet, non-uniform rational B-splines, etc.) The parameters for the distribution functions describing the distributions of cell events may be derived via methods including expectation maximization [REF: Hastie, Tibshirani, and Friedman, The Elements of Statistical Learning, pp 236-243, 2001.], Markov-chain Monte Carlo, or spectral methods [e.g., FFT]. Comparison of curves may be performed using a variety of metrics including Area Under the Curve (AUC), Cohen's D (Jacob Cohen (1988), Statistical Power Analysis for the Behavioral Sciences (second ed.)), Chi-Square, Kolmogorov-Smirnov, or other statistics.

One embodiment of the invention uses Uᵤ. This metric is designed to estimate the overlap between one and multi-dimensional distributions of cells that have been treated with a modulator and those that have not been treated with a modulator. Cells from both the modulated and unmodulated wells are ranked in decreasing order of intensity values for an antibody-fluorochrome conjugate. These rankings are then converted to an Receiver operating characteristic (ROC) curve, with the fraction of unmodulated cells on the x-axis and the fraction modulated cells on the y-axis. As one moves down the ranked list, an empirical ROC curve can be plotted by either moving parallel to the y-axis by 1/N_{modulated} if one encounters a modulated cell or the x-axis by 1/N_{unmodulated} if one encounters a unmodulated cell. The Uᵤ metric is then computed as a area under the ROC curve. The Uu metric may also be considered as the scaled Mann-Whitney U statistic. If one encounters only modulated cells before any unmodulated cells, the Uᵤ metric will equal 1.0. One the other hand, if all modulated cells are ranked lower than the modulated cells, AUCᵤₛ will equal 0.0. Finally, a perfect overlap between the two distributions, with the chance of encounter a modulated or unmodulated cells at a given intensity is about the same, Uᵤ will be close to 0.5.

Examples of analysis for activatable elements are described in US publication number 20060073474 entitled “Methods and compositions for detecting the activation state of multiple proteins in single cells” and US publication number 20050112700 entitled “Methods and compositions for risk stratification” and U.S. Ser. Nos. 61/085,789 and 12/229,976, which are hereby incorporated by reference in their entirety.

### Adjustments to Account for Unhealthy Cells in Analysis

Gating may be performed so that only data from healthy cells is used in analyses. In some embodiments, the health of the cells is determined by using cell markers that indicate cell health. In some embodiments, cells that are dead or undergoing apoptosis are removed from the analysis. In some embodiments, cells are stained with apoptosis and/or cell death markers such as labeled anti-cPARP antibodies or Aqua dyes. Scatter characteristics may also be used. Cells undergoing apoptosis and/or cells that are dead can be gated out of the analysis. In other embodiments, apoptosis is monitored over time before and after treatment. For example, in some embodiments, the percentage of healthy cells can be measured at time zero and then at later time points and conditions such as, for example: 24 h with no modulator, and 24 h with treatment with an agent, such as fludarabine or bendamustine. In some embodiments, the measurements of activatable elements are adjusted by measurements of sample quality for the individual sample, such as the percent of healthy cells present.

In some embodiments, a regression equation will be used to adjust raw node readout scores for the percentage of healthy cells at 24 hours post-thaw. In some embodiments, means and standard deviations will be used to standardize the adjusted node readout scores.

Before applying the SCNP classifier, raw node-metric signal readouts (measurements) for samples can adjusted for the percentage of healthy cells and then standardized. The adjustment for the percentage of healthy cells and the subsequent standardization of adjusted measurements is applied separately for each of the node-metrics in the SCNP classifier.

The following formula can be used to calculate the adjusted, normalized node-metric measurement (z) for each of the node-metrics of each sample. z=((x−(b₀+b₁.times.pcthealthy))-residual_mean)/residual_sd, where x is the raw node-metric signal readout, b₀ and b₁ are the coefficients from the regression equation used to adjust for the percentage of healthy cells (pcthealthy), and residual_mean and residual_sd are the mean and standard deviation, respectively, for the adjusted signal readouts in the training set data. The values of b₀, b₁, residual_mean, and residual_sd for each node-metric are included in the embedded object below, with values of the latter two parameters stored in variables by the same name. The values of the b₀ and b₁ parameters are contained on separate records in the variable named “estimate”. The value for b₀ is contained on the record where the variable “parameter” is equal to “Intercept” and the value for b₁ is contained on the record where the variable “parameter” is equal to “percenthealthy24 Hrs”. The value of pcthealthy will be obtained for each sample as part of the standard assay output. The SCNP classifier will be applied to the z values for the node-metrics to calculate the continuous SCNP classifier score and the binary induction response assignment (pNR or pCR) for each sample.

In some embodiments, the measurements of activatable elements are adjusted by measurements of sample quality for the individual cell populations or individual cells, based on markers of cell health in the cell populations or individual cells. Examples of analysis of healthy cells can be found in U.S. application Ser. No. 61/374,613 filed Aug. 18, 2010, the content of which is incorporated herein by reference in its entirety for all purposes.

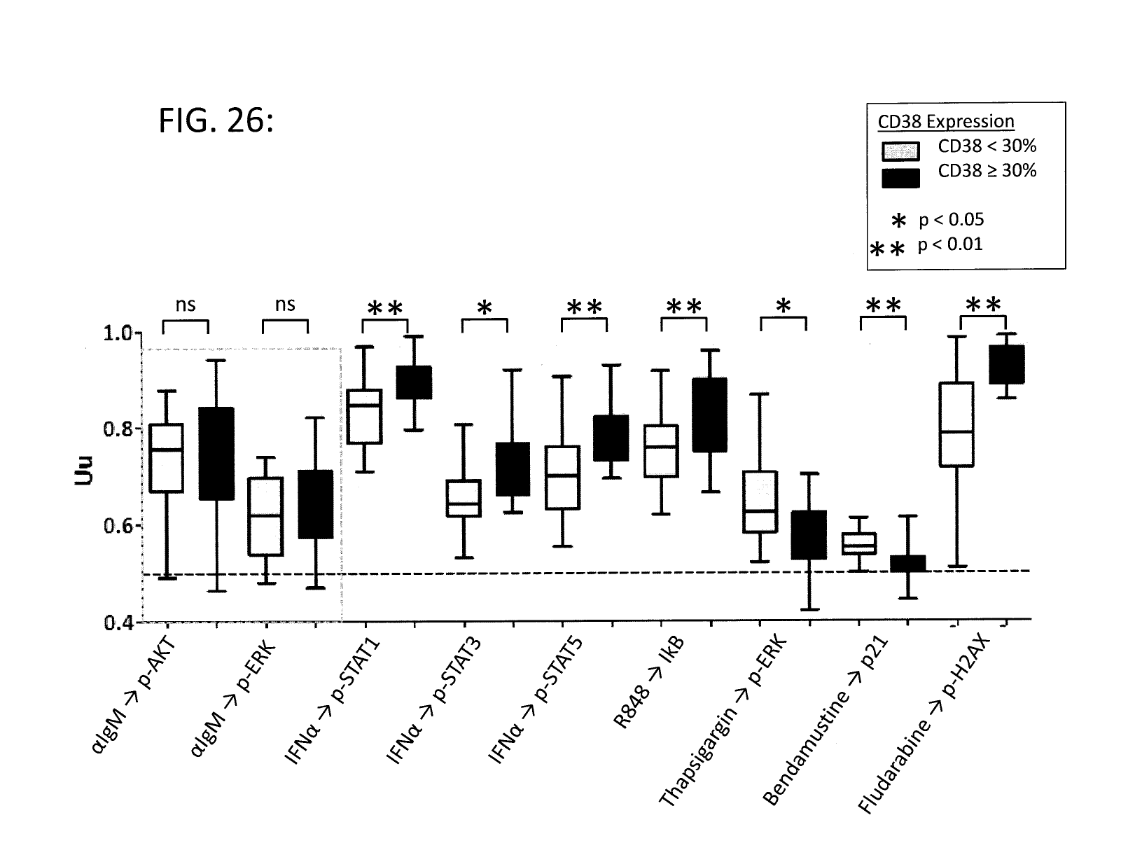

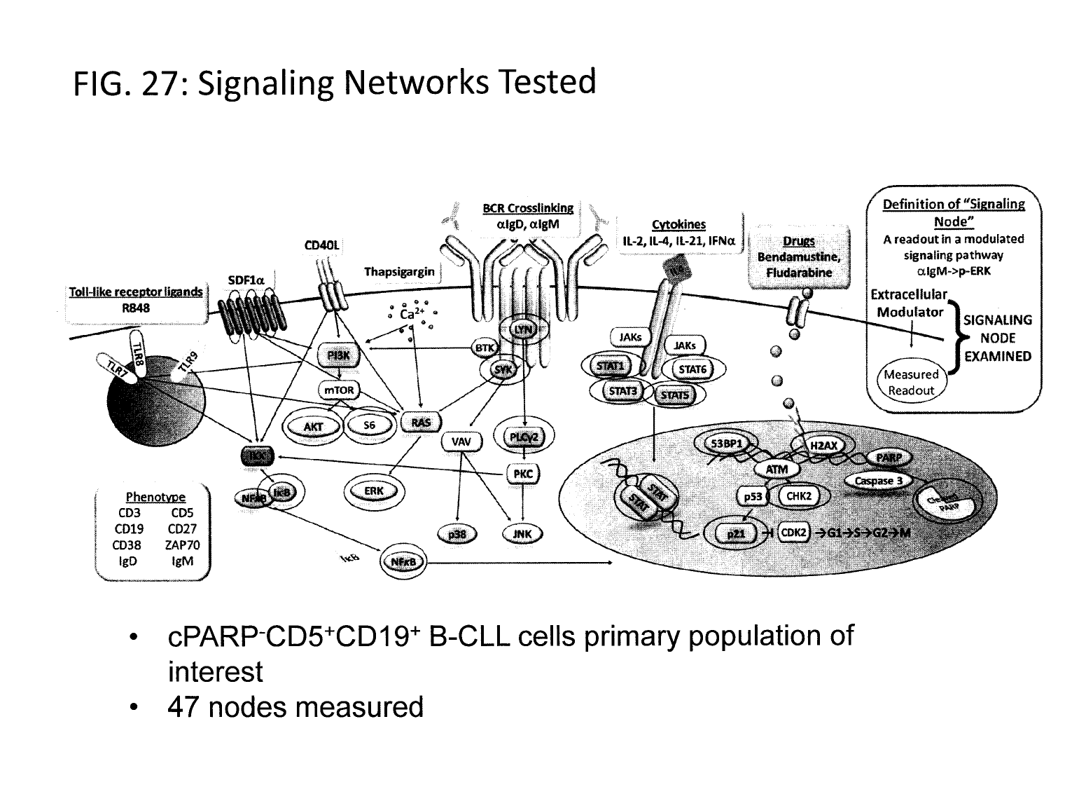

CLL serves as an example of the methods of the invention. The data shown in FIGS. 26, 27 and 28 of U.S. Ser. No. 12/229,976 ('976) is a heat map comparing the activation states of multiple activatable elements in 22 CLL patients and 4 control patients. This data demonstrates that B-cells from various CLL patients display distinguishable patterns of activatable elements as visualized by a heat map. An inhibitor or inhibitor plus another modulator further define additional patterns of activatable elements that allow identification, classification and grouping of cryptic or aberrant hematopoietic populations (i.e. patient clustering). In FIGS. 26, 27 & 28 patient samples are indicated at the top of the heat map. Each column represents a single patient. CLL indicates that the sample was obtained from a patient diagnosed with CLL. CON indicates that the sample was obtained from a control patient. The heat map legend is indicated at the top of the figure and uses a shaded scale based on the log 10-fold increase, or decrease, in mean fluorescence intensity (MFI), relative to the unstimulated control (0 min).

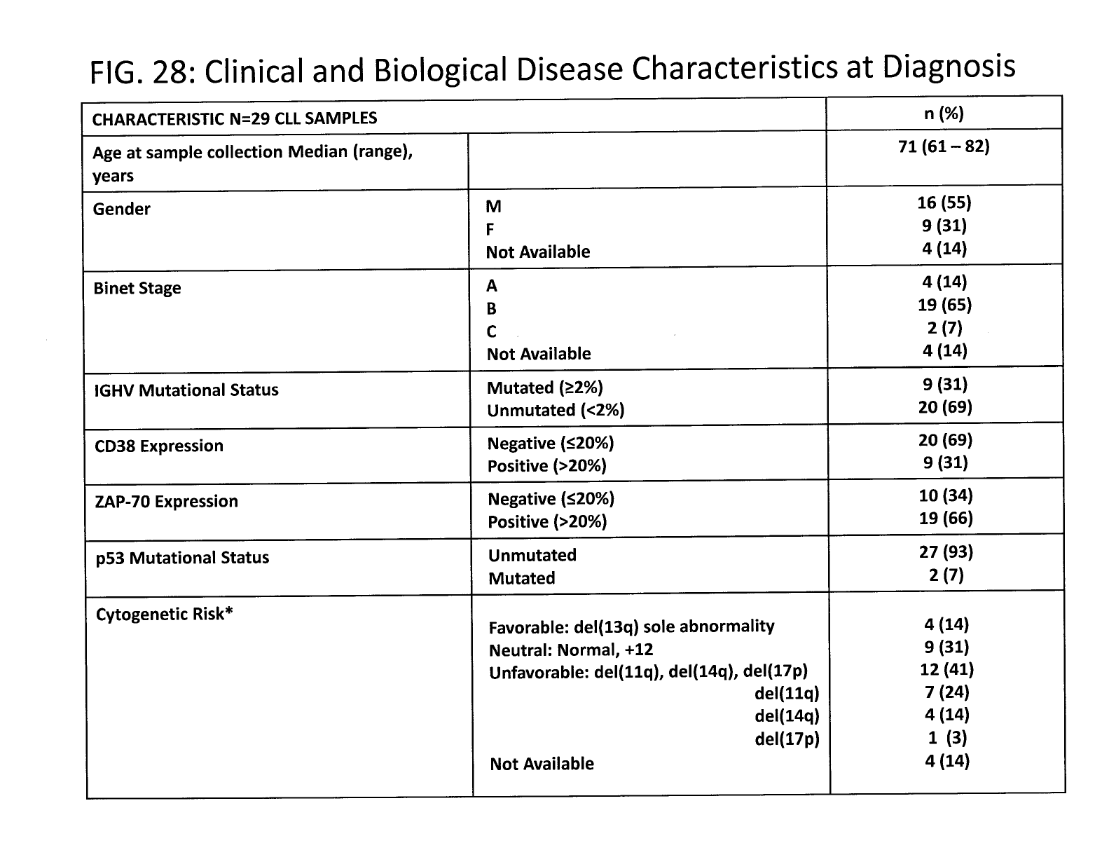

The heat map depicts the activation state of various activatable elements by denoting a change, or lack thereof, in the level of an activatable element revealed by the presence of an inhibitor and/or additional modulator. Thus, the heat map can depict the presence or absence of an increase in the activation level of a plurality of activatable elements in a cell upon contacting said cell with an inhibitor or a modulator. Labels to the right of the heat map indicate the activatable element detected, e.g. a phospho-protein. Labels to the right also indicate the modulator or inhibitor treatment for that row. “US” indicates unstimulated or untreated. FIG. 28 of '976 illustrates a pattern of activation levels of a plurality of activatable elements in a cell. FIG. 28 further illustrates the identification of patient clustering groups (i.e. clustering groups). A patient clustering group is comprised of samples from patients that display similar or distinct patterns of activation levels in one or more activatable elements in response to one or more modulators (e.g., an inhibitor, or an inhibitor and another modulator). FIG. 28 of '976 illustrates a clustering group comprised of samples from patients in which the activation levels of p-PLCγ2, p-SyK/ZAP-70, p-BLNK and p-Lyn are similar in response to the same stimulus. Some patient clustering groups are revealed upon modulation or treatment with an inhibitor as illustrated by the boxed regions. Treatment with H₂O₂ reveals a patient clustering group defined by the levels of p-PLCγ2, p-Syk/ZAP-70, p-BLNK and p-Lyn (FIG. 28, bottom right boxed area) that are similar to those of the four control patients (FIG. 28, bottom center box). Treatment with H₂O₂ further reveals a patient clustering group that is distinct from the controls (FIG. 28, 9 patients to the left of bottom boxed area). Modulation with H₂O₂ and BCR crosslinking defines another patient clustering group comprised of samples from patients that display the activation levels of p-BLNK, p-Syk and p-PLCγ2 (FIG. 28, top left boxed area) that are similar to the control patients (top center box). In addition, modulation with H₂O₂ and BCR crosslinking further reveals another clustering group distinct from the controls (10 patients to the right of top boxed area).

Thus, also provided herein is a method of deriving a classification. Deriving a classification involves defining a clustering group. A clustering group is defined by determining the activation state of a plurality of activatable elements from a plurality of cells wherein each cell is derived from an individual with a known conditions and/or known clinical outcome. A clustering group may define a pattern that associated with a known condition or known clinical outcome. Any suitable activatable element can be used wherein the activation level of said activatable element provides useful information regarding a known condition or clinical outcome of a patient. A cell derived from a patient with an unknown condition and/or unknown clinical outcome may be classified depending upon which clustering group it is identified with. This can further lead to diagnosis, prognosis, and/or evaluation or choice of treatment for the patient.

In another embodiment, measurements of expression or induced change in expression combinations of two activatable elements treated with one or more modulators may be used as inputs to algorithms such as logistic regression modeling or generally known classification methods to produce a score. The score may, for example, indicate the likelihood of response to fludarabine. Examples of modulator and activatable element (written as modulator→activatable element) combinations are: H₂O₂→p-Erk+anti-IgM or anti-IgD→p-STAT5, anti-IgM or anti-IgD→p-STAT5+H₂O₂→p-S6, H₂O₂→p-Lyn+Fludarabine→Caspase8, H₂O₂→p-PLCγ2+Unstim→CD5, H₂O₂→p-65-RelA+Unstim→CD5, H₂O₂→p-Erk+Unstim→CD5, Fludarabine→Caspase8+H₂O₂→p-S6, H₂O₂→p-BLNK+Fludarabine→Caspase8, H₂O₂→p-Lyn+Unstim→CD5, H₂O₂→p-Syk+Fludarabine→Cytochrome-C, H₂O₂→p-S6+Unstim→CD5, Unstim→SHP2+Fludarabine→Caspase8, H₂O₂→p-PLCγ2+Fludarabine→Caspase8, H₂O₂→p-Syk+Fludarabine→Caspase8, H₂O₂→p-BLNK+Unstim→CD5, H₂O₂→p-Lyn+Fludarabine→Cytochrome-C, Fludarabine→Cytochrome-C+H₂O₂→p-STAT5, H₂O₂→p-Syk+Unstim→CD5, H₂O₂→p-STAT5+Unstim→CD5, Unstim→CD20+Fludarabine→Caspase8, Staurosporine→Cytochrome-C+H₂O₂→p-65-RelA, H₂O₂→p-BLNK+Fludarabine→Cytochrome-C, Fludarabine→Caspase8+H₂O₂→p-Erk, H₂O₂→p-Erk+Fludarabine→Cytochrome-C, H₂O₂→p-S6+Staurosporine→Cytochrome-C, H₂O₂→p-PLCγ2+Fludarabine→Cytochrome-C, Staurosporine→Cytochrome-C+Unstim→IgM, H₂O₂→p-Syk+Unstim→IgM, H₂O₂→p-STAT5+Unstim→CD22, Unstim→CD5+Unstim→CD38, Unstim→CD20+Unstim→CD5, anti-IgM or anti-IgD→p-STAT5+Unstim→CD38, H₂O₂→p-Lyn+Unstim→CD22, Unstim→SHP2+Unstim→CD5, Unstim→CD20+anti-IgM or anti-IgD→p-PLCγ2.

In certain embodiments of the invention, combinations of modulators (or absence of modulator) and readouts may be used to provide information. See, e.g., Examples 2, 3, and 4 and the Figures referenced therein. A “readout” may be a measure of the activation state of an activatable element or a measure of the level of a protein; an example of the former is that the response to anti-IgM modulation can be measured using p-ERK as a readout (p-ERK is an activated form of ERK) and an example of the latter is the response to bendamustine can be measured using p21 levels (p21 acts through expression levels, not activation). Modulators useful in these embodiments include BCR crosslinkers, e.g anti IgM antibody such as F(ab)₂IgM and anti IgD antibody; chemokines, e.g. SDF1a; TLR modulators, e.g., R848 and CpG-B; other modulators such as CD40L, TCR crosslinkers, and CCL17; cytokines, e.g., IL-4, IL-2, IL-21, and IFNa; drugs, e.g. alkylating agents such as bendamustin, DNA synthesis inhibitors, such as fludarabine, and thapsigargin; In certain embodiments, markers, such as proteins are used to provide additional information, such as cell phenotype, and include cell surface proteins, such as cell surface proteins specific to B cells or classes of B cells; examples of markers that provide additional information include CD3, CD5, CD19, CD 27, CD38, ZAP 70, IgD, IgM. Readouts include IKB, NFKB, ERK (p-ERK), AKT (p-AKT), s6 (p-s6), LYN (p-LYN), SYK (p-SYK), PLcg2 (p-PLcg2), STAT1 (p-STAT1), STAT3 (p-STAT3), STAT5 (p-STAT5), STAT6 (p-STAT6), 538BP1, H2AX (p-H2AX), PARP (cleaved PARP, cPARP), S1p76 (p-S1p76), and p21. Other useful readouts include Lck (p-Lck). In certain embodiments, the methods and compositions of the invention utilize a combination of readouts, e.g., the readouts in certain embodiments include both activation states of activatable elements, e.g., proteins, and expression levels of certain proteins, e.g., p21.

### Methods and Compositions for CLL

In certain embodiments the invention provides methods and compositions useful in diagnosis, prognosis, evaluation, or prediction, such as time to first treatment (TTFT), predicting response to a drug, predicting status of pathways, such as the p53 pathway, for CLL.

B-cell chronic lymphocytic leukemia (B-CLL or CLL) is a disorder that with a highly variable clinical course. Some patients experience indolent disease and don't require treatment for several years, often surviving for over a decade, while others have a more aggressive form that requires early treatment. Current prognostic factors available to stratify patients include IGHV mutational status, ZAP70 expression, cytogenetic risk profile, and CD38 expression. While these can help assess disease risk, no reliable method currently exists to predict when treatment will be needed (time to first treatment, TTFT) or to guide clinical management of individual patients. The Rai and Binet clinical staging systems are widely used and correlate with survival for CLL patients at the population level, however, they lack the ability to individually distinguish patients with early stage B-CLL who will progress to aggressive disease from those with indolent disease.

Prognostic factors such as the immunoglobulin heavy chain variable region (IGHV) mutational status, cytogenetics, fluorescence in-situ hybridization (FISH), and expression of surface markers CD38 and ZAP 70 have been used, both individually and in combination, to improve prognostic accuracy and to define a course of treatment. B-CLL cells which express unmutated IGHV (U-CLL) have a more rapidly progressive clinical course than those patients whose cells express a mutated IGHV gene (M-CLL). At the time of diagnosis, 80% of CLL patients will have chromosomal abnormalities identified using fluorescence in-situ hybridization (FISH) with those who express 17p- having a particularly poor outcome associated with impaired p53 pathway signaling. CD38 has been linked to the proliferation of B-CLL cells and the presence of high numbers of CD38⁺ B-CLL cells in the blood is associated with a poor prognosis. ZAP-70 is expressed in most cases of U-CLL and less frequently in M-CLL, and while it correlates with more rapid disease progression in both IGHV gene mutation subtypes, the lack of assay standardization limits its clinical utility.

There is now strong evidence that B-cell receptor (BCR) signaling is a driving event in disease onset and progression, with U-CLL cells displaying a higher degree of BCR activity than M-CLL and correlating with more aggressive disease. ZAP-70 expression has also been linked to greater BCR activation, although likely in a kinase independent mechanism, acting as a scaffold or by competing for inhibitors of SYK.

BCR stimulation induces an increase of intracellular calcium, global protein tyrosine phosphorylation, and activation of proteins downstream of the BCR signaling pathways, i.e., spleen tyrosine kinase (SYK), extracellular signal-regulated kinase (ERK), and serine/threonine-protein kinase AKT. Signaling events downstream of the BCR are heterogeneous among B-CLL patients there is an association between increased anti-IgM→p-ERK signaling and a shorter time to first treatment (TTFT) in B-CLL.

In addition, patients with CLL that carry p53 mutations represent a small, but therapeutically challenging patient subgroup. These mutations are found in B-CLL cells in 5 to 8% of patients receiving first line treatment, and patients with disease cells carrying these mutations respond poorly to conventional fludarabine or alkylating agent-based chemotherapy regimens. Without being bound by theory, this may be due to the fact that both these chemotherapeutic drugs require functional p53-dependent pathways in order to induce cell death, although some reports suggest a p53-independent induced death by the more recently approved alkylating agent bendamustine. Mutations in the p53 gene are commonly acquired during the course of disease through clonal evolution and expand under therapeutic pressure, to an approximate incidence of 20% of all B-CLL at disease relapse and of 40% to 50% of fludarabine-refractory B-CLL. Progression free and overall survival are significantly decreased in patients with B-CLL carrying p53 mutations and p53 mutations have been identified as the strongest prognostic marker for overall survival in B-CLL patients.

Thus in certain embodiments the invention provides methods, compositions, and systems to prognose CLL, e.g., determine TTFT in patients diagnosed with CLL, and/or to determine potential response to treatment in subjects diagnosed with CLL.

In a first embodiment, the invention provides methods to determine TTFT in a subject suffering from or suspected of suffering from CLL comprising exposing cells from a sample obtained from the subject to at least two modulators and detecting, on a single cell basis, the level of an activated form of at least one intracellular activatable element, such as a protein, and from this information determining a TTFT for the subject. Detecting the level may be a relative term, and does not necessarily mean finding an actual concentration; it includes, for example, detecting for a single cell a fluorescence intensity for a fluorophore bound to an antibody that binds to the activated form, and using the fluorescence intensity as a basis for determining a level. The sample may be any suitable sample, such as a PBMC sample. The level of the activated form may be measured by any suitable technique, as described herein, such as flow cytometry or mass cytometry. In certain embodiments, the activatable element is a protein. In certain embodiments, the activated form is phosphorylated or cleaved. The cells in the treated sample may be gated so that only healthy cells are included in the analysis. Gating criteria may include scatter data, data from staining for dead cells (e.g., Aqua blue), and/or data from staining for cells exhibiting characteristics of apoptosis (e.g., cPARP levels), as described herein. In some cases the method may further include informing the subject and/or a clinician, e.g., by means of a report generated from the analysis of the sample, who may then decide on a course of action, based at least in part on the information from the analysis. The action may involve taking a later sample from the subject at a time determined, at least in part, by the TTFT information gained in the method. Action may also involve initiation of treatment, and giving the subject the treatment, such as administering a drug to the subject, for example at a time determined at least in part using the analysis of the invention.

In certain of these embodiments, the two modulators comprise a BCR crosslinker and a chemokine The BCR crosslinker may be any suitable BCR crosslinker as described herein, such as an anti-IgM antibody or antibody fragment, or an anti-IgG antibody or antibody fragment. In certain embodiment the BCR crosslinker may be F(ab)₂IgM. The chemokine may be any suitable chemokine The chemokine may be a chemokine selected to mimic the chemokine milieu in which B cells may be present in vivo. In certain embodiments the chemokine is SDF1□. The cell may be exposed to the modulators sequentially or simultaneously. The time of exposure may be any suitable time, for example a selected from the range of 1-120 min, or 1-60 min, or 1-30 min, or 1-20 min, or 2-30 min, or 2-20 min, or 4-30 min, or 4-20 min, or 4-15 min, or 6-30 min, or 6-20 min, or 6-15 min, or the time of exposure may be 1, 2, 3, 4, 5, 6, 7, 8, 9, 10, 11, 12, 13, 14, 15, 16, 17, 18, 19, 20, 21, 22, 23, 24, or 25 min. The exposure may be terminated by fixing the cells by any suitable method such as the methods described herein.

In certain embodiments, other cells may be exposed to other modulators, and levels of an activated form of one or more activatable element may be measured. Examples of other modulators include BCR crosslinker alone, such as F(ab)₂IgM, chemokine alone, such as SDF1α, CD40L, α-IgD, IL-21, IFNα, bendamustine, CpG-B, a combination of α-IgM and α-IgD, R848, IL-4, IL-2, Fludarabine, or Thapsigargin.

Cells may be permeabilized and exposed to a labeled binding element, e.g., a labeled antibody, to an activated form of an activated element, as described elsewhere herein. The activated form of the activatable element may be cPARP, p-AKT, p-ERK, p-LYN, p-PLC□2, p-SYK, p-H2AX, p-STAT1, p-STAT3, p-STAT5, p-STAT6, pZAP-70/pSYK, or any combination thereof. In certain embodiments, the activated form of the activatable element is p-AKT, p-ERK, p-LYN, p-PLCγ2, p-SYK, p-H2AX, or any combination thereof. In certain embodiments, the activated form of the activatable element is p-ERK. The levels of IκB may also be measured, either alone or in combination with other elements listed here.

In certain embodiments, one or more nodes (modulator and readout), are examined. As exemplified in this embodiment, one node is α-IgM+SDF1α→pERK. Other nodes may also include αIgM→p-AKT, αIgM→p-ERK, αIgM→p-LYN, αIgM→p-PLCg2, αIgM→p-SYK, αIgM+aIgD→p-AKT, αIgM+aIgD→p-ERK, αIgM+SDF1a→p-AKT, aIgD→p-AKT, aIgD→p-AKT, aIgD→p-AKT, R848→p-ERK, CD40L→p-AKT, CD40L→p-AKT, Fludarabine→p-H2AX, and any combination thereof.

Additional data from basal levels, either expression levels or activation levels if the element is an activatable element, of certain elements in cells not exposed to modulator may be included in the analysis. Such elements can include one or more of p-S6, p-STAT1, IκB, p-ERK, p-LYN, p-PLCγ2, p-STAT3, p-STAT5, p-STAT6, or p-SYK, or any combination thereof. In certain embodiments, the element includes p-S6, p-STAT1, IκB, or any combination thereof. In certain embodiments, the element comprises p-S6. In certain embodiments, the element comprises p-STAT1. In certain embodiments, the element comprises IκB. In certain embodiments analysis may be performed based solely on basal level data, without use of data from modulated cells and activatable elements in response to modulation. In certain of these embodiments, data from the activation level of an activatable element such as cPARP may be used in gating, as described herein, but no modulation need be used.

Additional data from indicators of relevant characteristics may also be included in the analysis. These may include one or more of immunoglobulin heavy chain variable region (IGHV) mutational status, cytogenetics, fluorescence in-situ hybridization (FISH), and expression of surface markers CD38 and ZAP 70.

Further data may also be included in the analysis, including one or more of patient age, gender, race, and the like.

In certain of these embodiments in which samples are gated for healthy cells, the gating criteria may include one or more of scatter data, Amine aqua dye staining data, and data from an indicator of apoptosis, for example an activated form of an activatable element involved in the apoptosis pathway, such as cPARP. In the case of an indicator of apoptosis, such as cPARP, cells may be exposed to not only labeled binding element, e.g., antibody, specific for at least one intracellular activatable element, but an additional labeled binding element, e.g., antibody, specific for the indicator of apoptosis, such as cPARP (in the case of cPARP, it is itself an additional activatable element). A cutoff for the indicator of apoptosis may be established and only data from cells on the side of the cutoff indicating no apoptosis or apoptosis not progressed beyond a certain point may be used. Similar cutoffs may be established for scatter data and/or Amine aqua blue staining intensity

In certain embodiments where the detection technique is flow cytometry, the data collection may be optimized by use of rainbow beads, as described in U.S. Pat. No. 8,187,885, and U.S. Patent Application Publication No. 20130096948, both of which are incorporated herein by reference in their entirety.

In certain of these embodiments the data for analysis is gated based on markers, such as surface markers or intracellular markers. In certain embodiments these markers include one or more of CD3, CD5, CD19, CD27, CD38, ZAP70, IgD, IgM, or any combination thereof. In certain embodiments these markers include CD3, CD5, CD19, CD27, CD38, or any combination thereof. In certain embodiments these markers include CD3, CD5, CD19, or any combination thereof.

In a second embodiment, the invention provides methods to determine functional status of the p53 pathway, for example in cells from a subject suffering from or suspected of suffering from CLL, comprising exposing cells from a sample obtained from the subject, e.g., a subject suffering from or suspected of suffering from CLL to an agent whose activity depends, at least in part, on a functional p53 pathway and measuring, on a single cell basis, the level of at least one intracellular protein whose levels increase upon induction of p53 activity, and from this information determining the functional status of the p53 pathway in the cells. In this embodiment, the protein is not an activatable element and it is the levels of the protein that are measured, not levels of an activated form of the protein. In certain of these embodiments, the mutational status of p53 is determined. The sample may be any suitable sample, such as a PBMC sample. The levels in single cells may be measured by any suitable technique, as described herein, such as flow cytometry or mass cytometry. In certain embodiments, the levels of p21 are measured. The cells in the treated sample may be gated so that only healthy cells are included in the analysis. Gating criteria may include scatter data, data from staining for dead cells (e.g., Aqua blue), and data from staining for cells exhibiting characteristics of apoptosis (e.g., cPARP levels), as described herein. In certain of this second embodiment, the information may be used in combination with other information, e.g., information obtained in analysis described for the first embodiment, to, e.g., prognose a condition, such as CLL, in the subject, e.g., to predict TTFT. In certain of this second embodiment, the information may be used to determine if the subject is a likely responder or non-responder to certain treatment agents, such as alkylating agents, e.g., bendamustine, and/or DNA synthesis inhibitors, e.g., fludarabine. In some cases the method may further include informing the subject and/or the subject's clinician, e.g., by means of a report generated from the analysis of the sample, who may then decide on a course of action, based at least in part on the information from the analysis. The action may involve treating the patient by administering a drug whose action is dependent, at least in part, on a functional p53 pathway. Action may also involve initiation of treatment, and giving the patient the treatment, at a time determined at least in part using the analysis of the invention.

In certain of these embodiments in which samples are gated for healthy cells, the gating criteria may include one or more of scatter data, Amine aqua dye staining data, and data from an indicator of apoptosis, such as cPARP. In the case of an indicator of apoptosis, such as cPARP, cells may be exposed to not only labeled binding element, e.g., antibody, specific for at least one protein whose expression depends on functional p53 pathway, but an additional labeled binding element, e.g., antibody, specific for the indicator of apoptosis, such as cPARP (in the case of cPARP, it is itself an additional activatable element). A cutoff for the indicator of apoptosis may be established and only data from cells on the side of the cutoff indicating no apoptosis may be used. Similar cutoffs may be established for scatter data and/or Amine aqua blue staining intensity.

In certain embodiments where the detection technique is flow cytometry, the data collection may be optimized by use of rainbow beads, as described in U.S. Pat. No. 8,187,885, incorporated herein by reference in its entirety.

In certain of these embodiments, the agent whose activity depends, at least in part, on a functional p53 pathway is selected from the group consisting of bendamustine and fludarabine. In certain of these embodiments, the agent is bendamustine. The cell may be exposed to the agent for a time sufficient to observe activation of the p53 pathway, for example 6-48 hours, or 12-36 hours, or 18-32 hours, or 20-28 hours, or 24 hours. The exposure may be terminated by fixing the cells by any suitable method such as the methods described herein.

Cells may be permeabilized and exposed to a labeled binding element, e.g., a labeled antibody, to an element whose levels are to measured, as described elsewhere herein. The element whose levels are to be measured may be, e.g., p21.

In certain embodiments the activation levels of one or more activatable elements, e.g., activatable elements that indicate DNA double strand break response, may also be measured. Such elements may include any suitable element, e.g., p-Chk2, p-H2AX, p-53BP1, or any combination thereof.

Additional data from basal levels, either expression levels or activation levels if the element is an activatable element, of certain elements in cells not exposed to modulator may be included in the analysis. Such elements can include one or more of p-s6, p-STAT1, IκB, p-ERK, p-LYN, p-PLCγ2, p-STAT3, p-STAT5, p-STAT6, or p-SYK, or any combination thereof. In certain embodiments, the element includes p-s6, p-STAT1, IκB, or any combination thereof. In certain embodiments, the element comprises p-s6. In certain embodiments, the element comprises p-STAT1. In certain embodiments, the element comprises IκB.

Additional data from indicators of relevant characteristics may also be included in the analysis. These may include one or more of immunoglobulin heavy chain variable region (IGHV) mutational status, cytogenetics, fluorescence in-situ hybridization (FISH), and expression of surface markers CD38 and ZAP 70.

Further data may also be included in the analysis, including one or more of patient age, gender, race, and the like.

In certain of these embodiments the data for analysis is gated based on markers, such as surface markers or intracellular markers. In certain embodiments these markers include one or more of CD3, CD5, CD19, CD27, CD38, ZAP70, IgD, IgM, or any combination thereof. In certain embodiments these markers include CD3, CD5, CD19, CD27, CD38, or any combination thereof. In certain embodiments these markers include CD3, CD5, CD19, or any combination thereof.

In certain embodiments the method further comprises administering a drug to the subject, wherein the drug is a drug whose activity is dependent, at least in part, on a functional p53 pathway. In certain embodiments the drug is the same as the agent to which cells are exposed in a sample obtained from the subject, e.g., bendamustine.

In a third embodiment, the invention provides methods to determine response to a drug in a subject suffering from or suspected of suffering from CLL, comprising exposing a first portion of cells from a sample obtained from the subject to the drug and a second portion of the sample to no drug, and measuring, on a single cell basis, the activation level of at least one intracellular protein related to the initiation of apoptosis, comparing the activation levels in the treated cells with the activation level in the untreated cells, and from the results of the comparison, determining whether or not the subject will respond to the drug. The embodiment may also include administering the drug to the subject. The method of this third embodiment may be carried out in conjunction with the method of the first embodiment and/or the second embodiment to provide additional information, e.g., for prognosis or prediction for the subject. The sample may be any suitable sample, such as a PBMC sample. The activation levels in single cells may be measured by any suitable technique, as described herein, such as flow cytometry or mass cytometry. In certain embodiments, the activatable element is a protein. In certain embodiments, the activation is phosphorylation or cleavage. In some cases the method may further include informing the subject and/or a clinician, e.g., by means of a report generated from the analysis of the sample, who may then decide on a course of action, based at least in part on the information from the analysis.

Any suitable drug thought to act through apoptosis may be tested. In certain embodiments, the drug is an alkylating agent. In certain embodiments, the drug is bendamustine

The cells exposed to the drug may be exposed to the drug for a time sufficient to observe initiation of apoptosis as reflected in the activation level of the activatable element, for example 6-48 hours, or 12-36 hours, or 18-32 hours, or 20-28 hours, or 24 hours. The exposure time may be terminated by fixing the cells by any suitable method such as the methods described herein.

In certain embodiments where the detection technique is flow cytometry, the data collection may be optimized by use of rainbow beads, as described in U.S. Pat. No. 8,187,885, incorporated herein by reference in its entirety.

Cells may be permeabilized and exposed to a labeled binding element, e.g., a labeled antibody, to an activatable element whose activation level is to be measured, as described elsewhere herein. The element whose activation level is to be measured may be, e.g., cPARP.

Additional data from basal levels, either expression levels or activation levels if the element is an activatable element, of certain elements in cells not exposed to modulator may be included in the analysis. Such elements can include one or more of p-s6, p-STAT 1, IκB, p-ERK, p-LYN, p-PLCγ2, p-STAT3, p-STAT5, p-STAT6, or p-SYK, or any combination thereof. In certain embodiments, the element includes p-s6, p-STAT1, IκB, or any combination thereof. In certain embodiments, the element comprises p-s6. In certain embodiments, the element comprises p-STAT 1. In certain embodiments, the element comprises IκB.

Additional data from indicators of relevant characteristics may also be included in the analysis. These may include one or more of immunoglobulin heavy chain variable region (IGHV) mutational status, cytogenetics, fluorescence in-situ hybridization (FISH), and expression of surface markers CD38 and ZAP 70.

Further data may also be included in the analysis, including one or more of patient age, gender, race, and the like.

In certain of these embodiments the data for analysis is gated based on markers, such as surface markers or intracellular markers. In certain embodiments these markers include one or more of CD3, CD5, CD19, CD27, CD38, ZAP70, IgD, IgM, or any combination thereof. In certain embodiments these markers include CD3, CD5, CD19, CD27, CD38, or any combination thereof. In certain embodiments these markers include CD3, CD5, CD19, or any combination thereof.

### Systems

The invention also provides systems.

In certain embodiments, the invention provides a system for informing a decision by a subject and/or healthcare provider for the subject involving diagnosing, prognosing, evaluating status of, or determining a method of treatment for a condition from which the subject is suffering or is suspected of suffering, wherein the system comprises 1) the subject and the healthcare provider; 2) a unit for analyzing a biological sample obtained from the subject by a method of analysis comprising a) exposing cells from the sample to one or modulators, or no modulator, b) exposing the cells to a detectable binding element that binds to a form of an activatable element in the cell, and c) determining on a single cell basis the levels of the detectable binding element in the cell and 3) a unit for communicating the results of the analysis of the sample to the subject and/or healthcare provider so that a decision may be made regarding diagnosis, prognosis, state of, or treatment of the condition that the subject suffers from or is suspected of suffering from. The system may further comprise a unit for treating and transporting the sample from the patient to the analysis unit.

The subject can be a human who suffers from, or is suspected of suffering from, a condition, where the condition can be any condition as described herein. In some cases, the condition is a pathological condition such as a neoplastic, hematopoietic, or autoimmune condition, such as Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibrosis, or atypical immune lymphoproliferations, systemic lupus erythematosis (SLE), rheumatoid arthritis (RA).

In certain embodiments, the neoplastic, autoimmune or hematopoietic condition is non-B lineage derived. In certain embodiments the non-B lineage derived condition is selected from the group consisting of acute myeloid leukemia (AML), Chronic Myeloid Leukemia (CML), non-B cell acute lymphocytic leukemia (ALL), non-B cell lymphomas, myelodysplastic disorders, myeloproliferative disorders, myelofibrosis, thrombocythemias, or non-B atypical immune lymphoproliferations. In some embodiments, the neoplastic, autoimmune or hematopoietic condition is a B-Cell or B cell lineage derived disorder. In certain embodiments the B-Cell or B cell lineage derived disorder is selected from the group consisting of Chronic Lymphocytic Leukemia (CLL), B-cell lymphoma, B lymphocyte lineage leukemia, B lymphocyte lineage lymphoma, Multiple Myeloma, acute lymphoblastic leukemia (ALL), B-cell pro-lymphocytic leukemia, precursor B lymphoblastic leukemia, hairy cell leukemia or plasma cell disorders, e.g., amyloidosis or Waldenstrom's macroglobulinemia, B cell lymphomas including but not limited to diffuse large B cell lymphoma, follicular lymphoma, mucosa associated lymphatic tissue lymphoma, small cell lymphocytic lymphoma and mantle cell lymphoma. In some embodiments, the condition is AML or CLL. In certain embodiments, the condition is CLL. In some embodiments, the CLL is defined by a monoclonal B cell population that co-expresses CD5 with CD19 and CD23 or CD5 with CD20 and CD23 and by surface immunoglobulin expression.

The sample may be any sample as described herein. In certain embodiments, the sample is a blood sample. In certain embodiments, the sample is a bone marrow aspirate sample. The sample may be a sample obtained previously, or it may be a sample that the subject or healthcare provider requests to be made based on information that makes one or both suspect the presence of a condition, or on diagnosis of the condition and the desire to obtain relevant information regarding prognosis, course of treatment or progression of the condition, prediction of effectiveness of a particular treatment for this subject. Thus, in general, the subject and/or healthcare provider order the obtaining of the sample and the use of the system to obtain the desired information.

In certain embodiments, the system also includes a unit for treating the sample and transporting the sample to the analysis unit. Treatment includes any necessary treatment to allow the sample to be transported to the analysis unit without significant degradation of relevant characteristics. Various methods of treatment which may be used in this unit are as described herein. In certain embodiments, the treatment includes cryopreservation.

The analysis unit carries out SCNP as described herein. The modulator or modulators can be any modulator or modulators as described herein. In certain embodiments, no modulator is used (e.g. embodiments in which the analysis determines basal levels of activatable or other elements in cells). In certain embodiments, only modulators are used. In certain embodiments in which the condition is CLL, and a prognosis is to be determined, the modulator or modulators may include a BCR crosslinker. In certain embodiments in which the condition is CLL, the modulator or modulators may include a BCR crosslinker, e.g. αIgM such as F(ab)₂IgM or αIgD, and a chemokine, such as SDF1α. Other modulators useful in CLL are as described herein. Exemplary modulators for CLL include BCR crosslinker alone, such as F(ab)₂IgM, chemokine alone, such as SDF1α, CD40L, α-IgD, IL-21, IFNα, bendamustine, CpG-B, a combination of α-IgM and α-IgD, R848, IL-4, IL-2, Fludarabine, or Thapsigargin. Sets of modulators for determination of the functionality of the p53 pathway and determination of treatment are as described herein, such as an agent whose action is dependent on activation of the p53 pathway, such as an alkylating agent, or such as bendamustine or fludarabine. It will be apparent that the modulator or modulators used in the analysis unit may be tailored to the condition examined, of which CLL is merely exemplary.

In the methods used in the analytical unit, a form of an activatable element is detected by exposing the cell to a detectable binding element and detecting the element. Activatable elements are described herein. In certain embodiments, the activated form is the form detected. Activated forms may be, e.g., phosphorylated or cleaved. In certain embodiments the element is a protein and the form detected is a phosphorylated form or a cleaved form. Detectable binding elements are as described herein, for example antibodies specific to a specific form of an activatable element, e.g., antibodies specific to a phosphorylated form or antibodies specific to a cleaved form. The component of the analytical unit for detection may be any suitable component as described herein, such as flow cytometer or mass spectrometer. In certain embodiments the element detected does not exist as activated and non-activated forms, in which case the total level of the element is detected using a detectable binding element specific to the element to be detected. In embodiments in which the condition is CLL, and a prognosis is to be made, detectable binding elements may be any element or set of elements as described herein, e.g., binding elements for cPARP, p-AKT, p-ERK, p-LYN, p-PLCg2, p-SYK, p-H2AX, p-STAT1, p-STAT3, p-STAT5, p-STAT6, pZAP-70/pSYK, or any combination thereof; p-AKT, p-ERK, p-LYN, p-PLCg2, p-SYK, p-H2AX, or any combination thereof, or p-ERK. The levels of IκB may also be measured, either alone or in combination with other elements listed here. Similar additional sets of binding elements, for prognosis and for determination of status of p53 pathway, and for determination of treatment, are as described herein. As with modulators, these binding elements are exemplary for CLL and other conditions will have their own sets of binding element

The analytical unit may also be configured to analyze the raw data obtained from the detection of the detectable binding elements in single cells, or it may transmit the data to a separate data manipulation unit or units.

The analytical unit may also be configured to gate data from healthy cells vs unhealthy cells, also as described herein, e.g., by scatter, Amine Aqua staining, and/or cPARP determinations. The analytical unit may be manually controlled or automated or a combination thereof, also as described herein.

The unit for communicating the results of the analysis of the sample to the subject and/or healthcare provider so that a decision may be made regarding diagnosis, prognosis, state of, or treatment of the condition that the subject suffers from or is suspected of suffering from, may be any suitable unit. For example, the unit may generate a hard copy of a report of the results which may be physically transported to the patient and/or healthcare provider. Alternatively, the results may be electronically communicated, and displayed in a format suitable for communicating the results to the subject and/or healthcare provider, e.g., on a screen, or as a printed report.

The system allows the subject and/or the healthcare provider to receive information to assist in the diagnosis, prognosis, evaluation of status, or determining a method of treatment for the condition. For the patient, the additional information and the extra certainty it provides can provide emotional comfort and the greater probability of a successful outcome. For the physician, the system allows for greater ability to diagnose, prognose, evaluate, or determine treatment for the patient, and to subsequently receive payment. In the case of CLL, in certain embodiments the system allows, at least in part, the determination of a TTFT, or a determination of the functionality of the p53 pathway, or a determination of the likelihood of a method of treatment. In general in these embodiments, the subject will already have been diagnosed with CLL, and the system allows greater certainty as to the probable course of the disease and a more informed choice of, e.g., intervals for subsequent testing, as well as evaluation of subsequent samples. For subjects in whom the disease has progressed to the point of treatment, the system allows greater certainty for the patient and provider in knowing whether or not to pursue a particular treatment, such as treatment with a particular drug, e.g., an alkylating agent such as bendamustine, or more generally a drug that is dependent on a functional p53 pathway. For example, in CLL there is a possibility that a mutation in the p53 pathway will occur during the disease course and the system allows subject and healthcare provider to make a decision regarding treatment based on the probable presence or absence of the mutation and thus obtain a more favorable treatment outcome. Again, CLL is merely exemplary, but in all cases the subject and/or healthcare provider achieve a greater degree of certainty and comfort by using the system.

### Methods of Generating Reports

The invention also provides methods of generating reports based on the results of one or more single cell network profile (SCNP) assays. The report is in a form suitable for transport to an end user. The report may be in any suitable form, such as a hard (paper) copy or in electronic form, such as a data file or files stored in an electronically readable media, such as expressed and stored on computer readable medium in the form of magnetic fields on a hard drive or etchings on a CDROM. The transport may be physical transport or it may be electronic transport, or any other suitable transport so long as the report arrives at its destination in substantially the same form as it started, though it may converted at its destination into other forms

The report contains information generated by a SCNP assay, for example, an assay on a sample from a subject suffering from or suspected of suffering from a condition, such as CLL. In the case of CLL, in certain embodiments the report contains information relevant to determination of TTFT, determination of the functionality of the p53 pathway, determination of likely effect of a treatment, e.g., drug, or a combination thereof, as described elsewhere herein. The SCNP assay generates raw data, and in its most basic form a report may contain just the raw data; one of the simplest reports is a report of raw data from detection of a specific form of one activatable element in one cell; one or more such reports may be transported together or separately to one or more end-users. In more sophisticated forms, the report may contain the results of manipulation of the raw data, such as control corrections, gating, calibrations, application of one or more statistical models, construction of a classifier, and the like. The report may include diagnosis, prognosis, treatment, or other relevant information. The report may include recommendations for action, such as a recommendation regarding use, dosage, timing, and other aspects of treatment of a condition with a particular agent, e.g., drug. In addition the report will contain identifier information for the sample or samples on which the SCNP assay was run. At the other end of the spectrum from a report of raw data is a report that includes merely the final prognosis, diagnosis, treatment recommendation, etc., for the particular subject from whom a sample that was run in a SCNP assay was obtained. However, a report of the invention may include any or all aspects from raw data to final recommendations

Thus, a method of generating a report may include 1) obtaining raw data from a SCNP assay on a sample, or data produced by manipulation of raw data from an SCNP assay, e.g., an SCNP assay performed on a sample obtained from a subject suffering from or suspected of suffering from CLL; and 2) converting the data into a transportable report. In certain embodiments, the transportable report is a hard copy such as a paper report, and the conversion of the data is accomplished by methods well-known in the art for producing hard copies, such as printing the report at a printer connected to a computer. In certain embodiments, the transportable report is expressed and stored on computer-readable media in the form of magnetic fields, e.g., on a hard drive or etching on a CD. Methods for expressing and storing data on computer-readable media in the form of magnetic fields are also well-known in the art, see, e.g., U.S. Pat. Nos. 7,714,933 and 7,082,426, and U.S. Patent Applications Nos. 20130096948, 20050009078, and 20030100995, all of which are incorporated by reference herein in their entirety. In certain embodiments, the method includes 3) obtaining identifying data for the identity of the subject from whom the sample was obtained and converting the data into the transportable report. Such identifying data does not necessarily need to identify the personal identity of the subject, e.g., name, but does need to convey enough information so that the data in the report can be matched to a subject from whom the sample on which the report is based was obtained.

The invention also provides compositions comprising a report as described above in electronically readable medium, in addition to the methods of producing them.

### Kits

In some embodiments the invention provides kits. Kits provided by the invention may comprise one or more of the state-specific binding element described herein, such as phospho-specific antibodies. In some embodiments, the kit comprises one or more of the phospho-specific antibodies specific for the proteins selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHPT, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP-70, Lyn, Cbl, SLP-76, PLC□□, PLCγ 2, STAT1, STAT2, STAT3, STAT4, STAT5, STAT6, FAK, p130CAS, PAKs, LIMK1/2, Hsp90, Hsp70, Hsp27, SMADs, Rel-A (p65-NFKB), CREB, Histone H2B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, PARP, IAPB, Smac, Fodrin, Actin, Src, Lyn, Fyn, Lyn, NIK, IκB, p65(RelA), IKKβ, PKA, PKCγ, PKC□, PKC□, PKC□, CAMK, Elk, AFT, Myc, Egr-1, NFAT, ATF-2, Mdm2, p53, DNA-PK, Chk1, Chk2, ATM, ATR, β-catenin, CrkL, GSK3β, GSK3β, and FOXO. In some embodiments, the kit comprises one or more of the phospho-specific antibodies specific for the proteins selected from the group consisting of Erk, Syk, ZAP-70, Lyn, Btk, BLNK, Cbl, PLCγ2, Akt, RelA, p38, S6. In some embodiments, the kit comprises one or more of the phospho-specific antibodies specific for the proteins selected from the group consisting of Akt1, Akt2, Akt3, SAPK/JNK1,2,3, p38s, Erk1/2, Syk, ZAP-70, Btk, BLNK, Lyn, PLCγ, PLCγ 2, STAT1, STAT3, STAT4, STAT5, STAT6, CREB, Lyn, p-S6, Cbl, NF-κB, GSK3β, CARMA/Bcl10 and Tcl-1.

Kits provided by the invention may comprise one or more of the modulators described herein. In some embodiments, the kit comprises one or more modulators selected from the group consisting of F(ab)₂IgM, SDF1a, R848, anti-IgD, CD40L, thapsigargin, fludarabine, bendamustine, poly CpG, or IFNa as modulators, and detection elements, such as antibodies, directed to CD3, CD5, CD19, CD20 for external cell surface markers, as well as one or more of antibodies directed to cPARP, p-AKT, p-ERK, p-LYN, p-PLCg2, p-SYK, p-H2AX, p-STAT1, p-STAT3, p-STAT5, p-STATE, pZAP-70/pSYK, or any combination thereof; or antibodies directed to one or more of p-AKT, p-ERK, p-LYN, p-PLCg2, p-SYK, p-H2AX. Optionally, controls such as Ramos cells or peripheral blood mononuclear cells (PBMCs) from healthy donors can be included in the kit. These cells may be fresh, frozen, lyophilized or in any other appropriate state. In one embodiment, the kit comprises modulators such as H₂O₂ and anti-μ, as well as detection elements directed to one or more of the following: p-Lyn, p-Syk, p-BLNK, p-PLCγ2, p-Erk, p-Akt, p-S6, p-65/RelA, as well as non-canonical signaling markers such as p-STAT5. Inclusion of fludarabine into a kit will be useful to analyze cell responses to that drug. Kits may also contain labels that are detectable by flow cytometers or mass spectrometers. In addition the invention encompasses kits that contain the modulators F(Ab)₂IgM and SDF1a and labeled antibodies to p-ERK; as well as kits that contain bendamustine and/or fludarabine and labeled antibodies to p-21. Either of these kits may also contain antibodies to cPARP. It will be appreciated that a “kit” includes the elements bundled as one package as well as the elements provided separately if the intent, e.g., through instruction or other communication, is to use them together at the end point for a specific assay.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such kits may also include instructions to access a database such as described in U.S. Ser. No. 61/087,555 for selecting an antibody specific for the pathway of interest. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are expressly incorporated by reference in their entirety.

### EXAMPLES

### Example 1

### Signaling Pathways in CLL Samples

### Cell Preparation

Intracellular network responses of CLL patient samples subjected to modulators of signaling, were analyzed using flow cytometry-based Single Cell Network Profiling (SCNP). Of the many signaling modulators studied, H₂O₂ treatment (a general inhibitor of tyrosine phosphatase activity) stratified CU, patients into two subsets, one showing augmented BCR signaling and the second with little or no response. These data suggest that differential phosphatase activity with consequent aberrations in tonic (ligand independent) signaling proximal to BCR signaling was driving the biology of these two patient groups. Importantly, signaling in patients was reflected in all the measured components of the canonical B cell receptor network. Thus, p-Lyn, p-Syk, p-BLNK, p-PLCγ2, p-Erk and p-Akt showed parallel phosphorylation responses and were either augmented in unison, or not activated at all. In vitro F-Ara-A-exposure of samples from the same group of CLL patients identified patients with a significant number of apoptosis competent cells, and other patients that were refractory to apoptotic induction in vitro. Statistical analysis of the two data sets revealed that the capacity of patient samples to show peroxide-mediated augmented BCR signaling was highly associated with the ability of cells in these patients to exhibit apoptotic proficiency to F-Ara-A in vitro. This potential link between mechanisms governing apoptosis, phosphatase activity and BCR signaling in B cells provides a means of identifying patient samples that are either responsive or refractory to a given therapy By extrapolation, such a test based on these separation criteria can play an informative role in the choice of therapeutic agent for patients,

Cryopreserved PBMCs, 23 from CLL patients and 7 from healthy donors, were rapidly thawed in a 37° C. water bath. 1 mL of pre-warmed thawing media (PBS 1% FBS, 2 mM EDTA) was added dropwise to each of the cryovials. Thawed cells were transferred to a tube containing 8 mL thawing media. Tubes were inverted and centrifuged at 200×g for 8 minutes at room temperature. Supernatant was decanted, cell pellets were resuspended in 1 mL RPMI 1640 1% FCS and filtered over 70 um nylon mesh (BD Falcon) to remove cell clumps and debris. 11 mL of additional RPMI 1640 1% FCS was added to the samples to wash. A 20 uL aliquot was removed from each sample and placed into a solution of PBS 4% FCS CD45 Alexa Flour 700 and Propidium Iodide for viability and counting on a BDLSRII cytometer. The remaining volume of cell suspension was centrifuged at 200×g for 8 minutes at room temperature

Supernatant was aspirated and each sample was resuspended in 4 mL of PBS. 4 mL of 2× Amine Aqua (Invitrogen) was added to each sample and incubated at 37° C. for 15 minutes. 10 mL of RPMI 1640 1% FCS was added to each sample to neutralize the Amine Aqua staining and samples were centrifuged at 200×g for 8 minutes at room temperature. The supernatant was decanted and all samples were resuspended at a density of 2.4×10⁶ cells/mL, using a volume specific to the number of cells determined from the cell counting procedure. Samples were arrayed in a 96-well deep-well “mother” plate according to donor ID.

“Daughter” plates designated for treatment with modulators for or apoptosis inducing agents were generated from the “mother” plate with the use of a Liquidator 96-well pipettor (Rainin) Plates designated for treatment with modulators for phospho-readouts received 250 uL (6.0×10⁵ cells) of cell suspension per well, and plates designated for treatment with apoptosis inducing agents received 333 uL (8.0×10⁵ cells) of cell suspension per well. The “daughter” plates were prepared in duplicate and allowed to rest for 1 hour in an incubator at 37° C., 5% CO₂ before treatment.

### Cell Counting

A 20 uL cell suspension from each sample was incubated in individual wells of a 96-well u-bottom plate (BD Falcon) in 180 uL of PBS 4% FCS, CD45 Alexa Fluor700, and 1 μg/mL Propidium Iodide for 10 minutes at room temperature, shielded from light. After 10 minutes 25 μL of each sample was run on a BDLSRII cytometer (BDIS, San Jose, Calif.) equipped with a high throughput sampler (HTS). Events were gated on CD45+, PI−. Counts of events in the CD45+, PI− gate were exported in a CSV file and total cell numbers were calculated in Microsoft Excel.

### Ramos Cell Line Control

Ramos cell line controls were acquired from ATCC and cultured according to the manufacturer's protocol.

### Phenotypic Staining on Unfixed PBMCs

Three panels of fluorochrome conjugated antibodies were incubated with each sample. All three panels contained four common mAbs: CD3 Pacific Blue, CD20PerCP Cy5.5, CD5 biotin, and CD19 Alexa Flour 700. The varying combinations of mAbs in each of the panels was listed: Panel 1: IgM FITC, IgD PE, IgG APC; Panel 2: λ-light chain FITC, κ-light chain PE, CD38 APC; Panel 3: CD45 FITC, CD7913 PE, CD22 APC.

All antibodies for each panel were cocktailed in pre-titered saturating concentrations. 50 μL of each cocktail were aliquoted and arrayed in a deep-well 96-well plate. 42 μL of each sample from the prepared “mother” plate was added to the wells containing antibody cocktail. Cells were incubated for 30 minutes at room temperature, shielded from light.

After 30 minutes the cells were washed with 1 mL PBS and centrifuged at 400×g. After centrifugation the supernatant was aspirated. The 2° staining cocktail was prepared by adding 0.254 of streptavidin Qdot605 (Invitrogen) to 9.754 of PBS. 104 of 2° staining cocktail was added to each sample and incubated for 30 minutes at room temperature, shielded from light.

After 30 minutes the cells were washed with 1 mL FACS Buffer (PBS, 0.5% BSA, 0.05% NaN₃) and centrifuged at 400×g. After centrifugation the supernatant was aspirated and 100 μL of FACS Buffer was added to each well. The total volume of each well was transferred to a standard depth u-bottom 96-well plate (BD Falcon) for acquisition on a BD FACSCantoII equipped with the HTS unit.

### Modulation of Cells

Each sample was treated in bulk for 10 minutes at 37° C. with goat polyclonal IgM or IgG (F(ab′)₂, (Southern Biotech), final concentration 10 μg/ml, Phorbol Myristate Acetate ((PMA) Sigma), final concentration 400 nM, and H₂O₂, final concentration 3.3 mM. For the combination of anti-μ and H₂O₂, anti-μ was added first followed by H₂O₂ within 30 seconds.

For single agent modulators 150 μL of 10× solutions were arrayed into wells of a 96-well v-bottom plate (Nunc). Corresponding daughter plates were taken from the 37° C. incubator, and 200 mL of RPMI1640 1% FCS was added to each well using the Liquidator 96-well pipettor. Using a Hydra, 504 of 10× modulator solution was aspirated from the 10× plate and delivered to the daughter plates. The daughter plates were pulse vortexed for 5 seconds and placed into a 37° C. water bath for 10 minutes. At the end of the 10 minute incubation 2000 μL 5.6% PFA (final 1.6%) was added to each well to fix the cells.

For BCR x-linking in combination with phosphatase inhibitor H₂O₂, a “BCR X-link” 96-well deep-well plate was loaded with 500 μL of RPMI1640 1% FCS or 25 μg/mL IgM F(ab′)₂ in the appropriate wells. Another “H₂O₂” standard 96-well v-bottom plate was prepared with RPMI1640 1% FCS or 33 mM H₂O₂. Corresponding daughter plates were taken from the 37° C. incubator. Using the Liquidator 96-well pipettor (Rainin) 200 uL was aspirated from the “BCR X-link” plate and added to the daughter plates with cells. Not allowing more than 30 seconds to pass, 50 uL of 10×H₂O₂ was aspirated from the “H₂O₂” plate and added to the daughter plate using the Hydra 96-well pipettor (Matrix). The plate was pulse vortexed for 5 seconds and incubated in a 37° C. water bath for ten minutes. At the end of the 10 minute incubation 200 uL 5.6% PFA (final 1.6%) was added to each well to fix the cells.

After 10 minute incubation at 37° C. all plates were centrifuged at 1000×g for 5 minutes at room temperature. Supernatant was aspirated and plates were vortexed for 30 seconds to disrupt cell pellet. Cells were permeabilized by adding 6004 of ice cold 100% methanol to each well using the Costar 96-channel pipettor (Costar). Plates were covered with adherent foil seals and placed in a −80° C. freezer for at least 24 hours.

Treatment of Cells with Apoptosis Inducing Agents

5× solutions of apoptosis inducing agents were prepared: staurosporine at 25 μM and Fludarabine at 5 μM. ZVAD was prepared at 5× as well at a concentration of 500 μM. Combination preparations of staurosporine or fludarabine plus ZVAD were also prepared at 5×.

Solutions of apoptosis inducing agents, ZVAD, and media controls were arrayed into a 96-well deep well plate. The corresponding daughter plates for apoptosis conditions were removed from the 37° C. incubator. Facilitating the use of the Liquidator, 140 μL of 5× drug was aspirated from the 96-well deep well plate and added to the cells in the apoptosis daughter plate. The daughter plate was pulse-vortexed for 5 seconds and placed into a 37° C., 5% CO₂ incubator for 48 hours.

After 48 hours the cells were washed twice with PBS and centrifuged at 4000×g for 8 minutes at room temperature. Plates were pulse-vortexed to disrupt cell pellet and 200 μL of 1× Amine Aqua (Invitrogen) was added to the cells. Plates were placed in 37° C., 5% CO₂ incubator for 15 minutes.

After 15 minute incubation 1 mL RPMI1640 1% FCS was added to each well and plates were centrifuged at 1200 RPM for 8 minutes at room temperature. Supernatant was aspirated and 200 □L of RPMI1640 1% FCS was added to each well followed by 200 □L of 3.2% PFA to fix the cells. Plates were incubated in at 37° C. water bath for ten minutes then centrifuged at 1000×g for 8 minutes at room temperature. Plates were pulse-vortexed to disrupt cell pellets and 600 □L of ice-cold methanol was added to each well to permeabilize the cells. Plates were sealed with adhesive foil covers and placed at −80° C. overnight.

### Intracellular Staining of Cells

100 uL of sample suspension from apoptosis plates were aliquoted into 3 separate deep-well 96-well plates. 1 mL of FACS Buffer (PBS 0.5% BSA, 0.05% NaN₃) was added to each well and plates were centrifuged at 1000×g for 8 minutes at room temperature. The supernatant was aspirated and samples were washed again with 1 mL FACS Buffer and centrifuged at 1000×g for 8 minutes at room temperature. Antibody cocktails for signaling and apoptosis readouts were prepared in FACS Buffer. All cocktails contained a common panel of fluorochrome conjugated mAbs against cell surface antigens: CD3—Pacific Blue, CD20—PerCPCy5.5, CD5—biotin. Antibodies for each of the signaling panels are as follows: panel 1: pAkt—Alexa Fluor 488, pSyk—(Phycoerythrin) PE, pBLNK—Alexa Fluor 647; panel 2: pS6—Alexa Fluor 488, pPLC□₂—PE, pLyn—Alexa Fluor 647; panel 3: pErk—Alexa Fluor 488, SHP-1 purified; panel 4: SHP-2 purified, pSTAT5—PE, p-65/RelA—Alexa Fluor 647. Antibody cocktails were aliquoted into corresponding deep-well 96-well plates for staining

TABLE 1
Antibody panels used for measurements of signaling downstream of BCR, apoptosis
and delineation of cell subsets.

| | Ax488 | PE | Ax647 |
|---|---|---|---|
| Signaling Panel 1 | p-Akt(S473)* | pSyk(Y352)/ | p-BLNK(Y84) |
| | | p-ZAP-70(Y319) | |
| Signaling Panel 2 | p-S6(S235/S236)* | p-PLCg2(Y759) | p-Lyn(Y505) |
| Signaling Panel 3 | p-Erk(T202/Y204) | Empty | SHP-1** (2° Goat-anti- |
| | | | rabbit-Ax647) |
| Signaling Panel 4 | SHP-2* (2° Goat-anti- | p-STAT5(Y694) | p65/RelA(S529) |
| | rabbitAx488) | | |
| | FITC | PE | Ax647 |
| Apoptosis Panel 1 | Cleaved Caspase 3 | Cleaved PARP | Cytochrome C |
| Apoptosis Panel 2 | Empty | Cleaved PARP | p-Chk2(T68)* (2° GaR- |
| | | | Ax647) |
| | FITC | PE | APC |
| Phenotypic Panel 1 | IgM | IgD | IgG |
| Phenotypic Panel 2 | □-light chain | □-light chain | CD38 |
| Phenotypic Panel 3 | CD45 | CD79□ | CD22 |

All antibodies are from Becton Dickinson Biosciences (San Jose, Calif.) unless otherwise noted.

500 μL of cell suspensions in methanol from the signaling plates were aliquoted into a separate deep-well 96-well plate. Plates were centrifuged at 1000×g for 8 minutes at room temperature.

Supernatant was aspirated and 1 mL of FACS Buffer (PBS 0.5% BSA, 0.05% NaN₃) was added to wash the cells. Plates were centrifuged at 1000×g for 8 minutes, supernatant aspirated and 1 mL wash repeated. After the second wash, samples were resuspended in 400 μL of FACS Buffer. 100 μL from each signaling plate was delivered to each of the staining plates with pre-aliquoted antibody cocktail. Plates were sealed with adhesive foil, pulse-vortexed for 7 seconds, and placed in 4° C. shielded from light for 16 h.

Antibody cocktails for apoptosis readouts were prepared in FACS Buffer. All cocktails contained a common panel of fluorochrome conjugated mAbs against cell surface antigens: CD3—Pacific Blue, CD20—PerCPCy5.5, CD5—biotin. Antibodies for each of the apoptosis panels are as follows: panel 1: Cleaved Caspase 3—FITC, Cleaved PARP—PE, Cytochrome C—Alexa Fluor 647; panel 2: BCL-2—FITC, Cleaved PARP—PE, Cleaved Caspase 8 purified, panel 3: Cleaved PARP—PE, pChk2 purified. Antibody cocktails were aliquoted into corresponding deep-well 96-well plates for staining

500 μL of cell suspensions in methanol from the apoptosis plates were aliquoted into a separate deep-well 96-well plates. Plates were centrifuged at 1000×g for 8 minutes at room temperature. Supernatant was aspirated and 1 mL of FACS Buffer (PBS 0.5% BSA, 0.05% NaN₃) was added to wash the cells. Plates were centrifuged at 1000×g for 8 minutes, supernatant aspirated and 1 mL wash repeated. After the second wash samples were resuspended in 3004 of FACS Buffer. 1004 from each signaling plate was delivered to each of the staining plates with pre-aliquoted antibody cocktail. Plates were sealed with adhesive foil, pulse-vortexed for 7 seconds, and placed in 4° C. shielded from light for 16 h.

After 16 h incubation both the signaling and apoptosis plates were processed similarly. 1 mL of PBS was added to each well and plates were centrifuged at 1000×g for 8 minutes at room temperature. Supernatant was aspirated and 10 μL of 2° staining cocktail was added to the residual volume in the wells. Every well received streptavidin Qdot605 (0.25 μl in 10 μL). For wells stained with SHP2 2° goat anti-rabbit Alexa Fluor 488 (1:5000) was added in addition to streptavidin Qdot605. For wells stained with SHPT or pChk2, goat anti-rabbit Alexa Fluor 647 (1:5000) was added in addition to streptavidin Qdot605 (the combination comprises the secondary stain). Samples were incubated with secondary stain was incubated at room temperature for 30 mins shielded from light.

After incubation with secondary stain, plates were washed with 1 mL of FACS Buffer and centrifuged at 1000×g for 8 minutes at room temperature. Supernatant was aspirated and plates were pulse-vortexed for 7 seconds to disrupt the pellet. 80 μL of FACS Buffer was added to the residual volume in the wells. Using the Liquidator 96-well pipettor, samples were transferred from deep-well 96-well plates to standard profile 96-well u-bottom plates.

Samples were acquired on a BD FACSCantoII flow cytometer.

Surface marker studied were CD3−/CD20+/CD5+ (gating); IgM/IgD/IgG/λLC/κLC/CD79b/CDE19 (BCR); and CD38/CD22/CD45. Modulators used were anti-μ with or without H₂O₂, or anti-γ with or without H₂O₂, and PMA. Signaling and phosphatase expression molecules analyzed included four panels: 1) p-Akt/p-Syk/p-BLNK; 2) p-S6/p-PLC γ2/p-Lyn; 3) p-Erk/SHP-1; and 4) SHP-2/p-STAT5/p-p65.

Evaluation of DNA damage and apoptosis was made after exposure of samples to the following modulators: F-ara-A (Fludarabine) alone, staurosporine alone, ZVAD alone or the combination of F-ara-A with ZVAD and staurosporine and ZVAD and involved 3 panels of antibodies that recognized activated proteins within these pathways: 1) c-Caspase 3/c-PARP/cyt C; 2) Bcl-2/c-Caspase 8/c-PARP; and 3) p-Chk2/c-PARP.

Each determination was made in duplicate and correlation coefficients (r2 values) were greater than 0.8 in most cases.

Cells are deemed sensitive or responsive to F-ara-A as measured by apoptosis markers cleaved caspase and PARP.

Evaluating the physical consequences of apoptosis on the cells (namely plasma membrane blebbing and breakdown) showed a modest increase in cell death by amine aqua in F-ara-A treated or untreated cells.

Of the 110 nodes measured per sample, 106 had R²>0.8. The four nodes where R² was less could be accounted for by outliers.

Basal levels of phosphorylation are more variable in CLL samples than in healthy B cells

### Results

Surface marker characterization and basal phosphorylation states in the CLL cohort

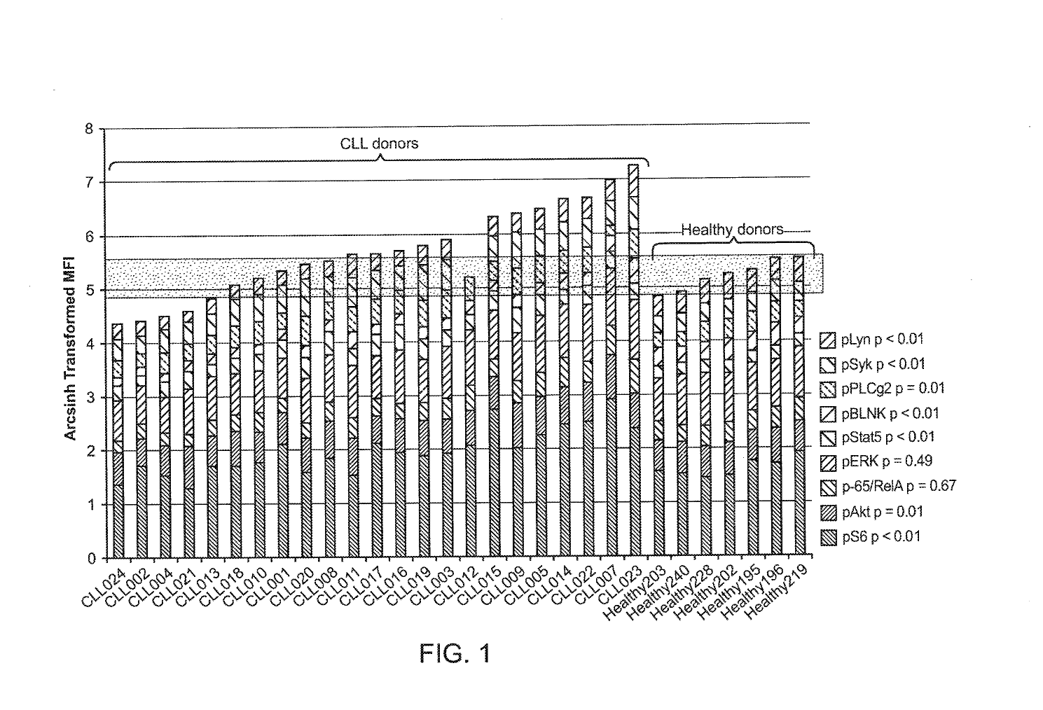

Comparison of MFI values of BCR signaling molecules in their basal phosphorylation states showed greater variability in CLL versus healthy B cells (FIG. 1). MFI values for p-Akt and p-Lyn spanned a range of 16 and 17 respectively among healthy B cells and 63 and 66 in CLL B cells. p-Erk and p-65/RelA showed no significant differences between healthy and CLL samples, indicating that at their basal level the activation state of these molecules did not reflect a CLL-dependent phenotype. Expression of markers (determined as MFIs) associated with the B cell lineage, and tyrosine phosphatases (CD45, SHP-1 and SHP-2), were compared between healthy and CLL B cells. Surface marker expression was homogeneous in B cells from healthy donors whereas in CLL B-cells surface marker expression was more heterogeneous. The expected 2:1 kappa/lambda ratio was evident in healthy B cells, and contrasted with the distorted ratios observed in CLL samples indicating clonal expansion of a malignant cell. A subset (13/23) of CLL samples expressed kappa chain exclusively, and a further subset of 7/23 CLL samples expressed only lambda chain. In 3 samples no light chain was detected suggesting clonal expansion of an immature B cell. Expected hallmarks of CLL were seen in the low expression of IgM and CD79β□ in individual patient samples. No statistical classification of CLL samples into distinct subgroups could be made based on expression levels of the measured markers and tyrosine phosphatases.

### Modulated Signaling Responses Distinguish Subgroups of CLL Patient Samples

To test whether phenotypic characterization of CLL physiology could be discerned based on responses of cells to extracellular stimuli, modulated BCR intracellular signaling was determined either in response to anti-μ (ligand-dependent) or post H₂O₂ treatment to evaluate the contribution of tonic signaling (ligand-independent) to BCR output. See Irish J M, J Immunol. 2006; 177:1581-1589; Monroe J G. Nat Rev Immunol. 2006; 6:283-294; and Wienands JProc Natl Acad Sci USA. 1996; 93:7865-7870. Samples were treated with anti-μ alone, H₂O₂ alone (3.3 mM) or the combination for 10 minutes to recognize differences in BCR signaling between CLL and healthy B cells. The 10-minute time point was chosen based on kinetic analyses in order to see robust, but not necessarily maximal phosphorylation, of all the BCR pathway signaling molecules under study. H₂O₂ titrations were performed and the concentration chosen was one in which minimal effects were seen on canonical signaling in healthy B cells. The millimolar concentration requirement for H₂O₂ is consistent with its intracellular millisecond half-life (See Reth M. Nat Immunol. 2002; 3:1129-1134).

TABLE 2
BCR and apoptosis responses in CLL and healthy samples. Numbers are
percentages of CLL B cells within a sample that undergo peroxide
mediated phoshphorylation of intracellular signaling molecules.

| Sample | CLL | | | | | | | | |
|---|---|---|---|---|---|---|---|---|---|
| Group | Sample | p-Lyn | p-Syk | p-BLNK | p-STAT 5 | p-PLC-γ | p-Akt | p-Erk | Apoptosis |
| Group 1 | CLL014 | 43 | 52 | 52 | 36 | 67 | 59 | 57 | ✓ |
| Group 1 | CLL003 | 73 | 74 | 64 | 60 | 81 | 74 | 86 | ✓ |
| Group 1 | CLL016 | 27 | 26 | 17 | 23 | 39 | 56 | 41 | ✓ |
| Group 1 | CLL008 | 57 | 57 | 47 | 36 | 77 | 67 | 84 | ✓ |
| Group 1 | CLL024 | 80 | 65 | 73 | 47 | 77 | 46 | 88 | ✓ |
| Group 1 | CLL013 | 19 | 25 | 19 | 13 | 37 | 26 | 27 | ✓ |
| Group 1 | CLL010 | 56 | 54 | 39 | 30 | 85 | 80 | 90 | ✓ |
| Group 1 | CLL002 | 39 | 39 | 36 | 21 | 56 | 40 | 70 | ✓ |
| Group 1 | CLL001 | 40 | 41 | 20 | 34 | 51 | 43 | 52 | ✓ |
| Group 1 | CLL004 | 20 | 23 | 19 | 13 | 45 | 31 | 76 | ✓ |
| Group 1 | CLL018 | 20 | 21 | 18 | 15 | 33 | 45 | 61 | ✓ |
| Group 1 | Mean | 43 | 43 | 37 | 30 | 59 | 52 | 67 | |
| Group 1 | SD | 21 | 18 | 20 | 15 | 19 | 17 | 21 | |
| Group 2 | CLL009 | 47 | 63 | 54 | 29 | 79 | 56 | 69 | — |
| Group 2 | CLL019 | 24 | 29 | 19 | 12 | 40 | 66 | 53 | — |
| Group 2 | CLL020 | 10 | 5 | 5 | 7 | 33 | 40 | 44 | — |
| Group 2 | CLL023 | 10 | 16 | 14 | 10 | 43 | 87 | 83 | — |
| Group 2 | CLL011 | 5 | 7 | 5 | 3 | 28 | 68 | 19 | — |
| Group 2 | CLL012 | 17 | 32 | 23 | 7 | 48 | 61 | 34 | — |
| Group 2 | CLL021 | 6 | 8 | 9 | 6 | 7 | 8 | 10 | — |
| Group 2 | CLL005 | 15 | 18 | 10 | 9 | 35 | 74 | 41 | — |
| Group 2 | CLL007 | 8 | 7 | 0 | 4 | 19 | 66 | 40 | — |
| Group 2 | Mean | 12 | 15 | 11 | 7 | 32 | 59 | 41 | |
| Group 2 | SD | 6 | 10 | 7 | 3 | 13 | 23 | 21 | |
| Healthy | CON219 | 1 | 3.5 | 3 | 0.6 | 25 | 44 | 26 | ✓ |
| Healthy | CON196 | 3 | 5 | 6 | 0.16 | 12 | 48 | 15 | ✓ |
| Healthy | CON195 | 4 | 6 | 10 | 3.4 | 18 | 51 | 21 | ✓ |
| Healthy | CON240 | 1 | 1 | 3 | 0.6 | 4.5 | 38 | 10 | ✓ |
| Healthy | CON202 | 0.6 | 3 | 5 | 0.6 | 5 | 45 | 17 | ✓ |
| Healthy | CON228 | 1 | 4 | 5 | 1 | 4 | 30.5 | 29 | ✓ |
| Healthy | CON 203 | 0.5 | 1.2 | 2 | 4 | 0.3 | 33 | 6 | |
| Healthy | Mean | 2 | 3 | 5 | 1 | 10 | 41 | 17 | |
| Healthy | SD | 1 | 1 | 2 | 1 | 7 | 7 | 7 | |
| | Ramos | 50 | 53 | 88 | 38 | 77 | 85 | 64 | ✓ |

Consistent with previous reports, anti-μ-mediated BCR signaling was further potentiated by H₂O₂ in B cells from healthy donors. See Irish, J. Immunol., 2006.

Analysis of the signaling responses showed that the CLL sample cohort could be broadly segregated into two patient groups. In Group 1a significant subpopulation of cells was responsive to H₂O₂ (for example the mean percentage of a cell subset with a responsive Lyn, Syk or BLNK population was 43%, 43% and 37% respectively, Table 2 and FIG. 2(A)). In all but three cases, the addition of anti-μ did not mediate a further increase in downstream signaling responses, consistent with the notion that aberrant phosphatase activity might be regulating BCR activity in CLL signaling. (The numbers in Table 2 are derived from 2D contour plots, such as those shown in FIGS. 2 A, B, and C. Gates are manually set and then applied to all samples. The numbers in Table 2 can vary based on slightly different gate placements.)

In Group II there was a reduced number of such cells after exposure to H₂O₂. For example, the mean percentage of cells with activated Lyn, Syk or BLNK was 12%, 15% and 11% respectively (Table 2 and FIG. 2(B)). On the lower side, CLL021 showed 5-6% and CLL007 showed approximately 2% B cells with activated Syk and BLNK.

Signaling in patient CLL samples was coordinated in that all the measured components of the canonical B cell receptor network, (p-Lyn, p-Syk, p-BLNK, p-PLCγ2) were augmented, in concert. In Group II, although the H₂O₂-mediated signaling response of the proximal BCR effectors was severely abrogated the p-Akt response was similar between the two groups (52% for Group 1 and 59% for Group III, Table 2). The activation of Erk in Group II was less than in Group I (41% and 67% respectively). In healthy B cells, all signaling molecules except Akt were minimally responsive to H₂O₂ treatment alone (Table 2). Given that H₂O₂ is a known inhibitor of phosphatase activity, and that phosphatase activation is a physiological regulator of proximal BCR signaling activities, (J Irish Blood, 2006, Reth Nat. Immunol. 2002, Singh D K, Cell. 2005; 121:281-293, J Irish J. Immunol, 2002, Monroe J G. Nat Rev Immunol. 2006; 6:283-294, Wienands J., Proc Natl Acad Sci USA. 1996; 93:7865-7870 and Rolli V, Mol Cell. 2002; 10:1057-1069) these data suggest that deregulation of phosphatase activity could explain some of the differences observed between CLL and healthy B cell signaling responses.

Unexpectedly, in 14/23 CLL samples there was an increase in phosphorylated STAT5 in response to H₂O₂ within a subset of cells in individual samples (FIG. 2(C)(left hand panels). In 7/23 CLL samples as well as in healthy B cells a minimal number of cells exhibited an increase in phosphorylated STAT5 in response to H₂O₂ (FIG. 2(C)(right hand panels). This observation suggests either that there is a significant re-wiring event downstream of tonic BCR signaling or that an alternative pathway is activated, and either could be connected to STAT5 activity.

Samples that showed an H₂O₂-mediated p-STAT5 response were the same as those in which the canonical components of the BCR network were activated in response to H₂O₂ (Table 2). Interestingly, in many patient samples at least two prominent CLL cell populations with unique and definable signaling responses were observed. For example, a sample in which a dominant cell subset demonstrated augmented signaling in response to H₂O₂, other subsets could be identified with marginal responses (FIG. 2(B-C)). No such distinctions were observed using basal phosphorylation states, underscoring that activation of BCR signaling molecules highlights the differences in pathway biology between and within samples.

Lack of responsiveness of the Lyn/Syk/BLNK/PLCγ2 signaling proteins to H₂O₂ treatment was associated with lack of apoptotic response in CLL B cells.

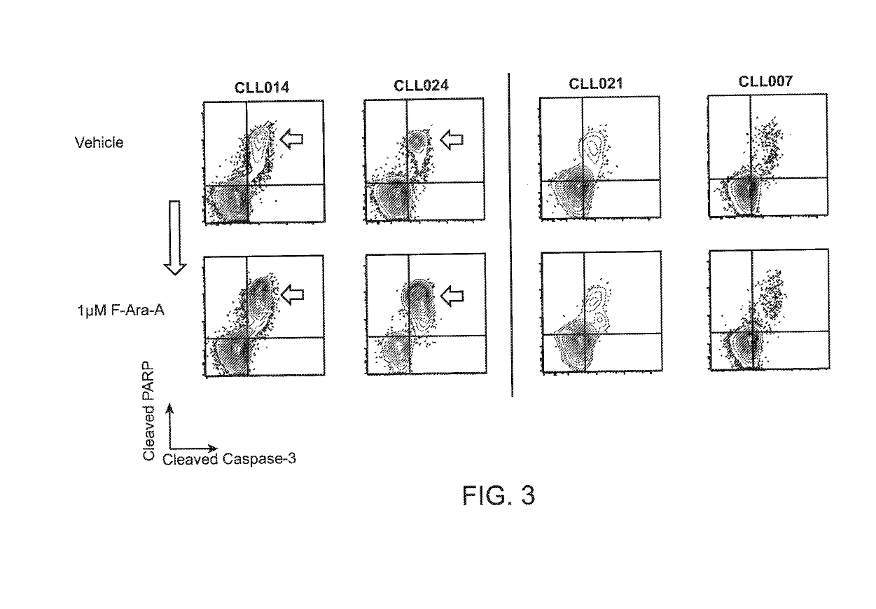

There has long been a presumed link between ligand-induced BCR signaling, tonic BCR signaling, and B cell survival. (See Kraus M, et al Cell 2004; 117:787-800 and Kraus M J Exp Med. 2001; 194:455-469). If such links are critical, then it might be further postulated that in CLL and other B cell malignancies, associations might be present between the observed signaling potential downstream of the BCR. To test this, apoptotic responses of CLL samples and healthy donors were enumerated by SCNP after in vitro exposure to F-ara-A for 48 hours. Representative CLL samples that were responsive or refractory to in vitro F-ara-A exposure are depicted by correlated measurement of cleaved caspase 3 and cleaved PARP in each cell (FIG. 3) Measurements of loss of mitochondrial cytochrome C in the same cells are consistent with the apoptotic responses.

Within responsive samples there were at least two cell subpopulations, with a second cell subset that was refractory to in vitro F-ara-A exposure (FIG. 3 (Left hand panels). This is reminiscent of the signaling data described above in which cell subsets with heterogeneous signal transduction responses were seen within the same sample (FIG. 2(A)). DNA damage was assessed using antibodies against the phospho-threonine 68 epitope on Chk2, the ATM phosphorylation site (F-ara-A incorporation into DNA results in a damaged product and activation of cell cycle checkpoint kinases). (See Antoni L, Nat Rev Cancer. 2007; 7:925-936). Although differences in p-Chk2 levels were seen in cell subsets within F-ara-A responsive and refractory samples, these differences were not statistically significant.

Given the pro-survival role BCR signaling plays in healthy and tumorigenic B cell biology (See Irish Blood 2006, Brerski R J, Monroe J G. Bioscience; 2008; Irish J. Immunol 2006; and Jumaa H, Annu Rev Immunol. 2005; 23:415-445) the data were analyzed for any associations between H₂O₂-modulated BCR signaling and apoptotic response to in vitro F-ara-A exposure. To evaluate the CLL cohort for trends, all cell events from the gated B cells of all CLL samples and, separately, all healthy samples were combined into respective ‘virtual’ samples that represented a composite of signaling for each modulated signaling molecule (FIG. 4(A) healthy B cells (pink-narrow peak) and CLL B cells (cyan-broad peak)). On the assumption that at least two subpopulations of cells could be driving the distribution of expression in the combined samples, the underlying “subpopulations” were decomposed via mixture modeling for the CLL samples to represent the underlying probability distributions (FIG. 4(B)).

Identifying Cell Populations with Distinct Signaling by Mixture Models

SCNP measures signaling for each cell individually, allowing characterization of a spectrum of cell signaling responses. Cells were gated on light scatter characteristics and then evaluated for viability by exclusion of Amine Aqua. Live cells were gated as CD3−/CD20+ and then evaluated for CD5 expression. Metrics including median fluorescent intensity (MFI), percentage of positive cells, and mixture-model derived population content (see below), were extracted from CD3−/CD20+ cells. FCS files were analyzed in FlowJo (Treestar, Ashland, Or) version 8.8.2 Plotting a histogram of the distribution of fluorescence intensities of all cells across all samples suggests that there are often distinct populations of cells with different signaling characteristics (FIG. 1). Expectation maximization methods (Hastie, Tibshirani, and Friedman, The Elements of Statistical Learning, pp 236-243, 2001). were applied to histograms of arcsinh transformed fluorescence intensities to generate mixture models (See Efroni S, Schaefer C F, Buetow K H (2007) Identification of Key Processes Underlying Cancer Phenotypes Using Biologic Pathway Analysis. PLoS ONE 2(5): e425. doi:10.1371/journal.pone.000042) comprised of two normal distributions using the mixdist package (see Peter Macdonald and with contributions from Juan Du (2008). mixdist: Finite Mixture Distribution Models. R package version 0.5-2. http: (double slash)www.r-project.org, http: (double slash)www(dot)math(dot)mcmaster.ca/peter/mix/mix.html) for R (see R Development Core Team (2009). R: A language and environment for statistical computing. R Foundation for Statistical Computing, Vienna, Austria. ISBN 3-900051-07-0, URL http: (double slash)www(dot)R-project.org). The population distributions observed in the H₂O₂-treated samples are evidence of heterogeneity in the phosphatase activity that regulates tonic BCR signaling among different CLL samples. Metrics were defined to characterize each patient sample as to the extent to which it contains cells in each population by computing the area under the curve for the fluorescent intensities from that sample with respect to a random sampling of 50000 events representing each mixture-model derived distribution. These metrics were termed ‘MixMod1’ and ‘MixMod2’ representing the areas under the curve for the distributions with lower and higher mean fluorescent intensities, respectively.

Striking differences were observed in the population distributions between healthy versus CLL B cell populations after treatment with H₂O₂ alone (see arrows in FIG. 4(A)). The histograms show a greater spread in the fluorescence intensities in CLL versus healthy B cells for the measured BCR signaling molecules (FIG. 4(A) CLL B cells (cyan-broad peak) versus (healthy B cells (pink-narrow peak)). Combining H₂O₂ with anti-μ did not produce additional substantial changes in the B cell population distribution of CLL B cells, suggesting that H₂O₂-mediated phosphatase inhibition was defining the signaling potential of these CLL B cell populations. This is surprising and contrasted with healthy B cells in which the combination of H₂O₂ and anti-μ□ resulted in an enhanced population distribution based on signaling, compared to each of these modulators alone (FIG. 4(A), fourth column, see Irish Blood 2006). A comparison of the histograms for healthy B cells versus CLL B cells in the absence of a modulator show that there were only minor differences in basal levels of phosphorylation for each BCR signaling molecule (this differs from the bar chart in FIG. 1 in which differences in basal phosphorylation between CLL and healthy B cells were computed on a per patient basis).

The trends in the mixture models emphasize the patterns (as expected) of the individual patient samples: the presence of an H2O2 de-repressed cell subpopulation and quiescent cell subset. The mixture model has the benefit of showing, at least for this cohort of patients, the averaged boundaries of where such subpopulations of cells lay on the histograms. The metrics that defined these curves were next used to develop classifiers (see below) for responses that might be linked to the presence of absence of these observed cell subsets.

Receiver operating characteristic (ROC) curves were generated to determine whether presence of either or both of the two populations defined by the mixture models was associated with response or lack of response to in vitro exposure to F-ara-A (FIG. 5(A). No such associations could be determined for healthy B cells, as expected, since the H₂O₂ concentration was selected to give no response in healthy B cells as previously reported. (See Irish J Immunol 2006).

Area under the ROC curves (AUC of ROC curve) (See Hanley J A, Radiology. 1982; 143:29-36) for signaling induced by H₂O₂ treatment showed that p-Lyn (AUC 0.84), p-Syk (AUC 0.75), p-BLNK (AUC 0.79), p-PLCγ2 (AUC 0.81), p-Erk (AUC 0.77) and p-STAT5 (AUC 0.84) signaling stratifies patient samples according to their apoptotic pathway response (FIG. 5(A). Using the metrics derived from these mixture models, the AUC plots demonstrated that samples in which signaling was revealed by H₂O₂ exposure were more likely to undergo F-ara-A mediated apoptosis (FIG. 4(B), 5(A)). By contrast, samples in which H₂O₂ failed to induce signaling were largely non-responsive to F-ara-A (FIGS. 2(A-C), FIG. 3 and Table 2). An un-scaled mixture model of H₂O₂-induced phosphorylation of STAT5, (see FIG. 4(B), row 5 from the top, 3rd column), (AUC 0.84 from FIG. 5(A)) was established for the cohort of CLL samples (FIG. 5(B), top panels). Of note, the range of expression observed for SHP-1, SHP-2 and CD45 tyrosine phosphatases was greater in CLL compared to healthy B cells. However, there was no association with the expression levels of these markers with either the magnitude of induced signaling or apoptotic responsiveness. Thus, levels of these phosphatases alone were not surrogates for these pathway functions.

The ROC curves (FIG. 5(A)) demonstrated significant associations between H₂O₂-mediated signaling and apoptotic proficiency. The samples could be divided into two predominant response phenotypes. First, samples CLL007 and CLL021 are exemplary of patients that showed a single major H₂O₂ non-responsive population of cells (FIGS. 5B and 2B). As noted, these patient samples were refractory to F-ara-A exposure in vitro and had a reduced H₂O₂-mediated activation of Lyn, Syk, BLNK, PLCγ, or STAT5. A second phenotypic response group, represented by samples CLL014, and CLL024 were responsive to F-ara-A and had significant activation of Lyn, Syk, BLNK, PLCγ, and STAT5 and whose expression profiles overlapped areas defined by the individual distributions of the mixture model, and in some cases (CLL014 being representative) demonstrate a clear bimodal phenotype (FIG. 5B, 2A). There were two outliers for which this association did not hold. CLL009 exhibited a robust H₂O₂-mediated signaling response for all measured signaling molecules and yet failed to undergo apoptosis (FIG. 5(B) Table 2). These data suggest that in these samples a different biology may be driving CLL, indicating that despite the broad associations observed in signaling responses including STAT5 downstream of the BCR to the apoptotic response, there remain additional linkages between these signaling systems that can vary independently. SCNP Improves In Vitro Fludarabine Response Prediction in CLL Cells that are ZAP-70 positive and IgV_{H} Unmutated Cells when Analyzed Separately from all CLL Cells

No associations could be made between the IgV_{H} mutational status or ZAP-70 expression status and in vitro response to F-ara-A (AUC value for ZAP-70 and apoptotic response 0.53 and Fisher's exact test for association between IgV_{H} status and apoptosis (F-ara-A responder/F-ara-A refractory, p value=1, odds ratio=0.1.2). See Table 3 below.

TABLE 3
ZAP-70 and IgV_{H} mutational status do not discriminate
in vitro fludarabine responders from non-responders

| | Fludarabine responders | Fludarabine non-responders |
|---|---|---|
| ZAP+ | 4 | 4 |
| ZAP− | 5 | 6 |

Fisher exact test:
p = 1,
odds-ratio = 1.2,
data is same for IgV_{H} mutational status

However, SCNP improved in vitro fludarabine response prediction when applied to CLL patient cells that were ZAP-70 positive or IgV_{H} unmutated. ZAP-70 and IgV_{H} mutational status are used to classify patients to inform clinical decisions. Splitting patients according to their ZAP-70 status, as defined by ZAP-70 measured using flow cytometry being >20% (that is, ZAP-70>20% is ZAP positive), or IgV_{H} mutational status improves in vitro fludarabine response prediction using SCNP in the ZAP-70 positive or IgV_{H} unmutated group, as measured by increase in AUC values in an ROC curve generated from fold change analysis. Compare the AUC values for p-Lyn stimulated by H₂O₂ (0.81 split/0.79 unsplit), p-Syk (0.88 split/0.76 unsplit), p-BLNK (0.88 split/0.81 unsplit), p-PLCg2 (0.88 split/0.76 unsplit), and p-STAT5 (1.0 split/0.88 unsplit). See Table 4 below.

TABLE 4
SCNP improves in vitro fludarabine response prediction in ZAP+ and
IgVH unmutated CLL patient cells measured by H202 modulation

| | | AUC for in vitro |
|---|---|---|
| | AUC for in vitro fludarabine | fludarabine response in |
| | response in ZAP+/IgVH | entire (unsplit) patient |
| Node | unmutated patients | group |
| H₂O₂/p-Lyn | 0.81 | 0.79 |
| H₂O₂/p-Syk | 0.88 | 0.76 |
| H₂O₂/p-BLNK | 0.88 | 0.81 |
| H₂O₂/p-PLCg2 | 0.88 | 0.76 |
| H₂O₂/p-STAT5 | 1.0 | 0.88 |

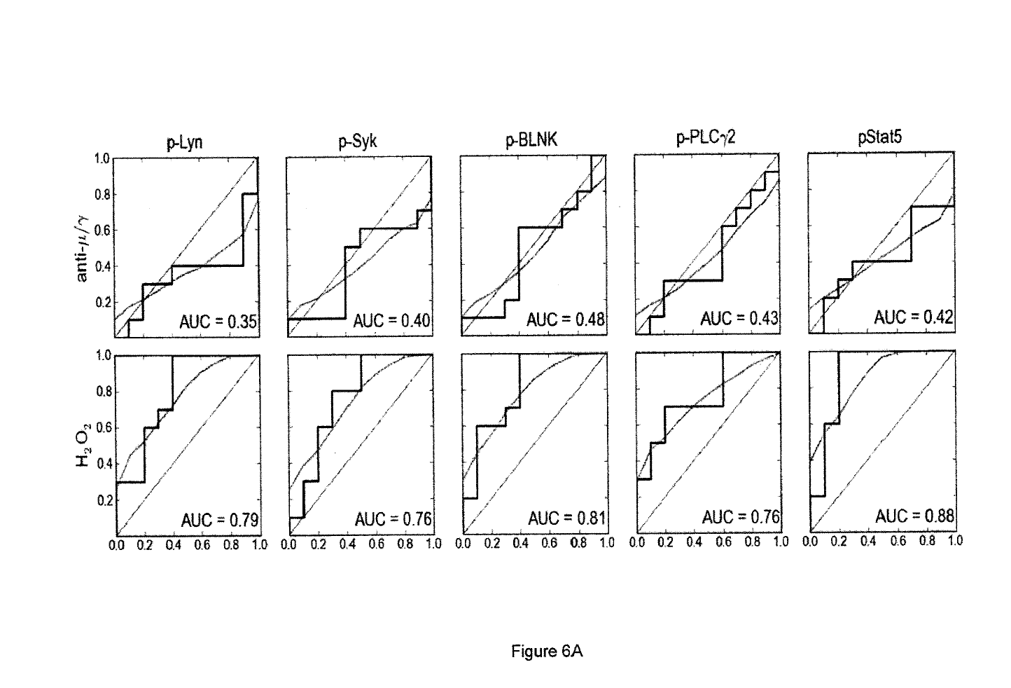

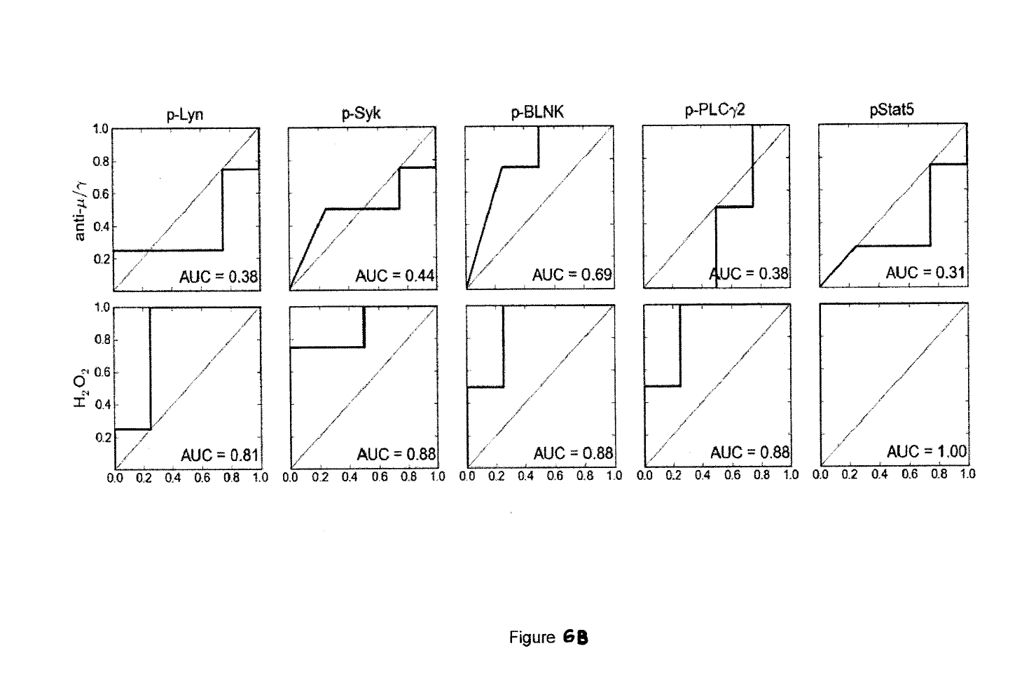

FIG. 6 shows statistical association between H₂O₂-mediated signaling and apoptosis induction by F-ara-A (Fludarabine) in the group comprised of all CLL cells regardless of ZAP-70 or IgV_{H} mutational status compared with the group comprised of ZAP-70 positive or IgV_{H} unmutated status. (A) ROC curves from a fold change model were expressed in order to evaluate how statistically significant H₂O₂-induced signaling is in predicting an in vitro apoptotic response to F-ara-A for all CLL cells, regardless of ZAP-70 or IgV_{H} mutational status (that is, prediction of apoptotic response is based on H₂O₂-induced nodes). The fold change metric for H₂O₂-mediated signaling was used to calculate whether there was an association with response or lack of response to in vitro exposure to F-ara-A. A value of 0.5 for the ROC plots indicates that the association is due to chance. A value of 1.0 indicates that there is a perfect association. (B) ROC curves from a fold change model were expressed with 95% confidence limits to evaluate how statistically significant H₂O₂-induced signaling is in predicting in vitro apoptotic response to F-ara-A for cells with ZAP-70 positive or IgV_{H} unmutated status (that is, prediction of apoptotic response is based on H₂O₂-induced nodes in combination with ZAP-70 or IgVH status). Discussion

Recently, several molecular and cytogenetic lesions have emerged as potential prognostic indicators for CLL. However, there are many disparities and confounding issues limiting their clinical utility. (See Hallek M, Guidelines for the diagnosis and treatment of chronic lymphocytic leukemia: a report from the International Workshop on Chronic Lymphocytic Leukemia updating the National Cancer Institute-Working Group 1996 guidelines. Blood. 2008; 111:5446-5456; Hamblin T J. Best Pract Res Clin Haematol. 2007; 20:455-468; Hamblin T J, Blood. 1999; 94:1848-1854; and Kay N E, Leukemia. 2007; 21:1885-1891). For example, although primary resistance to fludarabine has been shown to occur in patients harboring p53 deletions, a recent study reported that treatment-naïve patients with p53 deletions exhibit clinical heterogeneity with some patients experiencing an indolent course. (See Tam C S, Blood. 2009; 114:957-964 and Dohner H, Blood. 1995; 85:1580-1589). These published clinical studies suggest that there are underlying differences in CLL biology, which if understood, could provide more reliable prognostic information in individual patients.

The data in this study have highlighted a link between H₂O₂-induced changes in phosphorylation of BCR signaling proteins and F-ara-A-mediated apoptosis in CLL B cells. H₂O₂, a second messenger acts by oxidizing cysteines with pKa values below 5.0, such as are found in protein tyrosine phosphatases to sulfenic acid (See Reth, 2002). As an oxidant, H₂O₂ has other activities, these data potentially support a mechanism whereby deregulation of the kinase/phosphatase equilibrium results in activation of signaling proteins within the BCR network. Regardless of its exact mechanism of action, H₂O₂ was able to reveal differential signaling within CLL samples and these signaling differences appear to be associated with a signaling posture that either drives, or is driven by the ability of these cells to respond to apoptotic induction, in this case F-ara-A.

By analyzing signaling on a cell by cell basis, single cell network profiling (SCNP) allowed characterization of a spectrum of cell signaling responses. Single cell analysis, combined with mixture modeling identified at least two phenotypes for CLL B cells in human patients based on their response or lack of response (for proximal BCR signaling molecules) to H₂O₂ (FIG. 3(A), (B)). In samples where signaling is revealed by H₂O₂ a deregulated phosphatase near the BCR and/or other tyrosine kinase receptor signaling system(s) could be dampening signaling of BCR signaling molecules. Notably, some patients demonstrated simultaneous presence of both cell subsets, suggesting co-evolution of signaling phenotypes, a common precursor of these cell subsets, or a lineage relationship between the two subpopulations of cells (FIG. 2(A), (B), (C)). For most of the samples in which H₂O₂-mediated signaling was observed there was an association with an apoptotic response to in vitro F-ara-A exposure with AUCs of 0.8 as strong predictors for F-ara-A-induced apoptosis using H₂O₂-mediated increases in p-Lyn, p-PLCγ-2 and p-STAT5 as a surrogate (FIG. 5(A). Interestingly, and in contrast to studies where the presence of ZAP-70 and unmutated IgV_{H} correlated with greater anti-μ-mediated-BCR signaling (See Chen L, Blood. 2008; 111:2685-2692 and Efremov D G, Autoimmun Rev. 2007; 7:102-108), the signaling responses described here were unrelated to the IgV_{H} mutational status or to ZAP-70 expression and spanned a range of cytogenetic abnormalities. It is important to note that the above studies (Chen Blood 2008, Gobessi S, Leukemia. 2009; 23:686-697, and Efremov Autoimmun Rev. 2007) were accomplished via indirect assay of total phosphotyrosine on signaling proteins in each report. In our study, we undertook direct assay of phosphorylation sites using antibodies directed against known, functional, epitopes.

No associations were observed between either CD22 or CD45 expression levels with H₂O₂-mediated signaling. The contribution of phosphatases to tonic BCR signaling is further substantiated by the global inactivation of tyrosine phosphatases by sodium pervanadate or H₂O₂. These agents conferred de novo phosphorylation of BCR effector molecules that would normally be phosphorylated by ligand-dependent BCR aggregation. (See Reth M. Nat Immunol. 2002; 3:1129-1134 and Wienands J, Proc Natl Acad Sci USA. 1996; 93:7865-7870). An independent study showed that in CLL B cells where Lyn protein is over-expressed its inhibition by small molecule inhibitors in vitro in the absence of a BCR ligand, induced apoptosis. (See Contri A, J Clin Invest. 2005; 115:369-378). Corroborating these findings, in vitro treatment of CLL cells with R406 a small molecule inhibitor of Syk (a substrate of Lyn) also mediated an apoptotic response. (See Buchner M, Cancer Res. 2009; 69:5424-5432 and Gobessi S, Leukemia. 2009; 23:686-697). Both sets of data support a pivotal role for tonic signaling in CLL B-cell survival. Further insights into the relationship between BCR signaling, tonic signaling, phosphatase activity, and apoptotic response could be determined by measuring apoptosis in the presence of specific tyrosine phosphatase inhibitors specifically targeting SHP-1 and/or CD45. A priori, such inhibitors would be predicted to promote survival. Consistent with this hypothesis, ectopic expression of protein tyrosine phosphatase, PTPRO, (silenced in CLL by DNA methylation) increased growth inhibition in response to F-ara-A. See Motiwala T, Clin Cancer Res. 2007; 13:3174-3181.

Although not a member of the canonical BCR signaling network, the increase seen in p-STAT5 (AUC 0.84, FIG. 5 (A) (B)) could be due to a bystander effect resulting from phosphatase inhibition with consequent increase in kinase activities for which STAT5 is a substrate. Interestingly, Sattler et al, showed the importance of H₂O₂ generation with consequent increases in p-STAT5 in several hematopoietic growth factor cascades in cell lines. See Sattler M, Blood. 1999; 93:2928-2935. A pivotal role was also demonstrated for activated STAT5 in hematopoietic stem cell self renewal and expansion of multipotential progenitors in myeloid disease. It is tempting to speculate about such roles for activated STAT5 in CLL. See Kato Y, J Exp Med. 2005; 202:169-179. Furthermore, the dependence of hematological malignancies on p-STAT5 was shown in a recent study where phospholipase C-β3 was shown to be a tumor suppressor by acting as a scaffold for simultaneous interaction with p-STAT5 and SHP-1 and in so doing promoted the dephosphorylation of p-STAT5. See Xiao W, Cancer Cell. 2009; 16:161-171. Whether phospholipase C-β3 plays a similar role in regulating p-STAT5 in CLL awaits further study.

The clinical complexity (and unpredictability) of CLL as well as the many components governing cell proliferation and survival mechanisms, suggest a diversity of mechanisms that give rise to CLL, Remarkably, however the current studies demonstrate a convergence of signaling patterns in CLL that lead to a remarkably limited set of phenotypic cell signaling outcomes. These regularized phenotypes, and the relationships between markers of their activity insofar as they exist in a self-regulating network, can be used to predict drug responses in vitro from signaling data within a ‘heterogeneous’ set of patient samples. This suggests there are only a limited number of pathway variations, despite the underlying heterogeneity, that maintain cellular homeostasis in CLL, and that numerous surrogates exist for pathway structures that ultimately drive apoptotic outcomes in response to therapy. Further Examples show that signaling profiles measured by this technology for individual samples can predict treatment outcome and stratify patients who might gain the most benefit from treatment regimens.

### Example 2

Single Cell Network Profiling (SCNP) Defines Prognosis beyond IGHV Mutational Status in CLL.

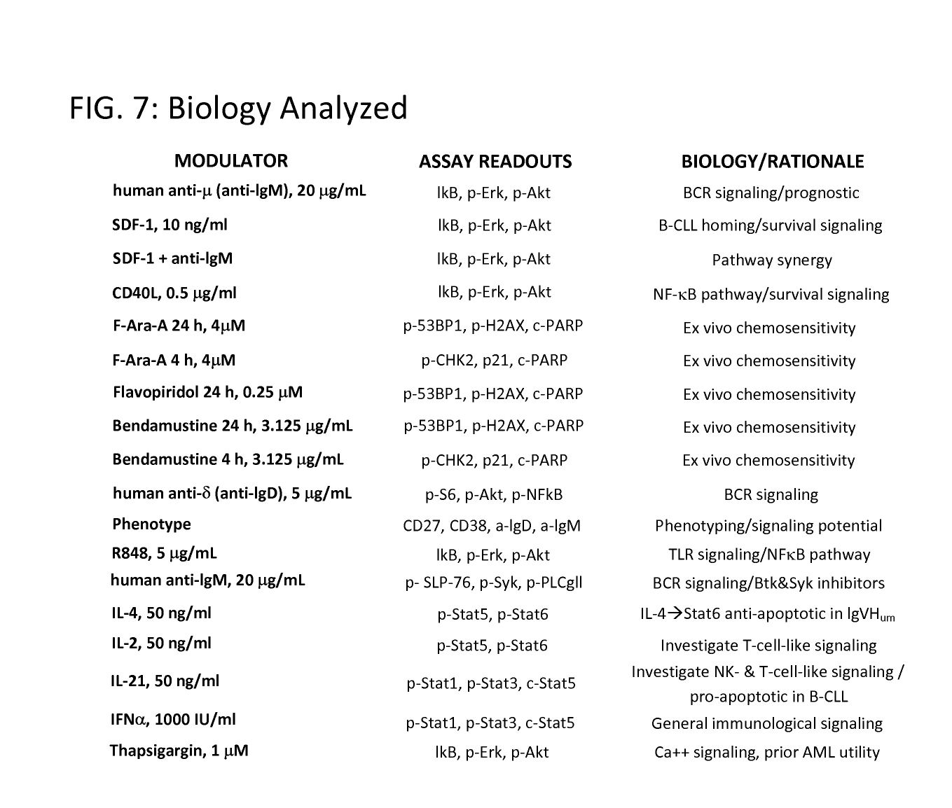

In order to assess the correlation of B-CLL biology (measured by SCNP) and clinical course in a clinically homogeneous population, samples collected as part of a Phase II clinical trial from elderly patients with previously untreated B-CLL prior to therapy initiation were assessed. See FIG. 7 and FIG. 27 for the biology that was analyzed.

B-cell chronic lymphocytic leukemia (B-CLL or CLL) is a disorder that with a highly variable clinical course. Some patients experience indolent disease and don't require treatment for several years, often surviving for over a decade, while others have a more aggressive form that requires early treatment. Current prognostic factors available to stratify patients include IGHV mutational status, ZAP70 expression, cytogenetic risk profile, and CD38 expression. While these can help assess disease risk, no reliable method currently exists to predict when treatment will be needed (time to first treatment, TTFT) or to guide clinical management of individual patients.

Patients with CLL that carry p53 mutations represent a small, but therapeutically challenging patient subgroup. These mutations are found in B-CLL cells in 5 to 8% of patients receiving first line treatment, and patients with disease cells carrying these mutations respond poorly to conventional fludarabine or alkylating agent-based chemotherapy regimens. Without being bound by theory, this may be due to the fact that both these chemotherapeutic drugs require functional p53-dependent pathways in order to induce cell death, although some reports suggest a p53-independent induced death by the more recently approved alkylating agent bendamustine. Mutations in the p53 gene are commonly acquired during the course of disease through clonal evolution and expand under therapeutic pressure, to an approximate incidence of 20% of all B-CLL at disease relapse and of 40% to 50% of fludarabine-refractory B-CLL. Progression free and overall survival are significantly decreased in patients with B-CLL carrying p53 mutations and p53 mutations have been identified as the strongest prognostic marker for overall survival in B-CLL patients.

The mechanisms behind variability in disease course among patients are not yet fully characterized. Microarray studies that compare the gene expression patterns of CLL versus healthy B cells have demonstrated that the expression patterns of IGHV-mutated (M-CLL) B cells share much more genetic similarity with IGHV-unmutated (U-CLL) B cells than they do with normal B cells. This indicates that the reason for differences in disease course are likely downstream of genetics and cannot be defined by simple genetic analyses.

B-cell receptor (BCR) activation has recently emerged as a potential driving force behind progression of B-CLL and BCR activation differs in patients with CLL. IGHV mutation, a widely accepted prognostic marker in CLL, correlates with BCR responsiveness to some degree. M-CLL cells have BCRs that respond weakly to stimuli versus U-CLL and are found in patients who are less likely to require treatment. U-CLL cells display a higher degree of BCR activity and are often found in patients with aggressive disease. Additionally, BCR signaling in U-CLL cells has been shown to activate telomerase and enhance cell survival, consistent with B lymphocyte accumulation. Despite the importance of the above mentioned indicators in determining the B-CLL prognosis, necessary improvements in the accuracy of prognosis and therapeutic selection/prediction of response requires a greater understanding of the functional biology, considering genetics, protein expression levels, and signaling pathways.

Single Cell Network Profiling (SCNP) is a multiparametric flow-cytometry based assay that can quantitatively measure both extracellular surface markers and changes in intracellular signaling proteins in response to external modulators. Using SCNP we have previously reported the functional characterization of the BCR signaling pathway and an association of αIgM→p-ERK with patient prognosis in CLL near the time of diagnosis. Here, we determine whether the association of αIgM→p-ERK with patient prognosis holds true when CLL samples are analyzed at the time of but prior to first treatment. We next examine functional BCR signaling and the environmental milieu (e.g. cytokines, chemokines, and surface receptor) signaling for association with TTFT. Last we use SCNP to explore chemoresistance and introduce a functional assay to detect defects in the p53 signaling pathway.

### Materials and Methods

### Patients and Samples

Peripheral blood mononuclear cells (PBMCs) were obtained from 29 patients with CLL treated on a clinical trial at “La Sapienza” University of Rome between November 2008 and January 2010 at the time of but prior to the initiation of first treatment. All patients provided informed consent for research purposes. SCNP assays were performed blinded to clinical outcomes. Diagnosis and initiation of treatment for B-CLL were based on 1996 National Cancer Institute-Working Group (NCI-WG)/IWCLL 2008 Guidelines for Diagnosis and Treatment of CLL. Clinical and biological disease characteristics at diagnosis are summarized in FIG. 28 IGHV mutational status and expression of CD38 and ZAP-70 were determined. IGHV sequencing utilized a 2% cutoff to delineate unmutated from mutated IGHV, and a cutoff of 30% and 20% were used for CD38 and ZAP-70 determination, respectively. SCNP analysis was performed to quantitatively measure 18 intracellular signaling proteins within CD19+CD5+ CLL cells using a panel of 14 disease-relevant modulators (FIG. 27) as follows:

### Single-Cell Network Profiling

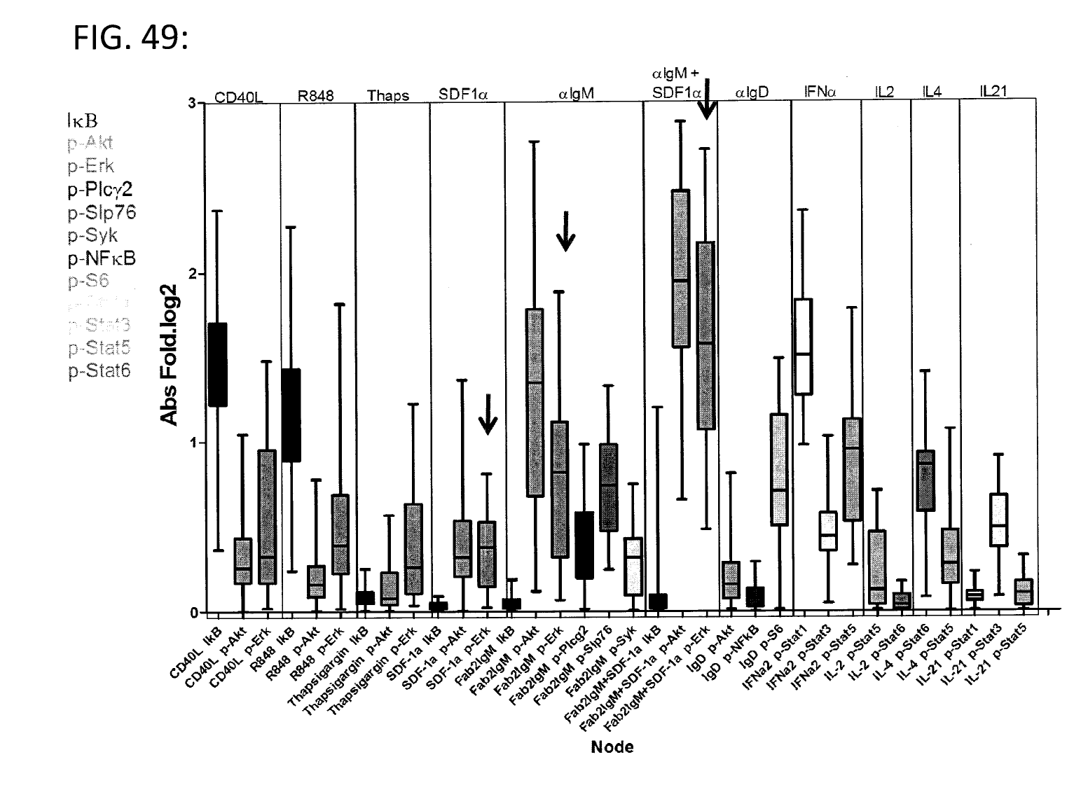

SCNP analysis was performed to quantitatively measure intracellular signaling proteins within CD19+CD5+ CLL cells using a panel of disease-relevant modulators. See FIG. 49. First, the cryopreserved PBMC samples were thawed at 37° C., stained for viability with Amine Aqua (Invitrogen, Carlsbad, Calif.), resuspended in RPMI with 10% FBS and aliquoted at 100,000 cells per well 96-deepwell plates. Cells were rested for 2 hours at 37° C. followed by modulation. Short duration modulation was performed for 10 minutes at 37° C. with the following modulators: 20 μg/mL polyclonal goat F(ab′)2 anti-human IgM (Southern Biotech, Birmingham, Ala.), 5 μg/mL monoclonal anti-human IgD (BD Biosciences, San Jose, Calif.), 10 ng/mL of SDF1□ (R&D Systems, Minneapolis, Minn.), and the combination of 20 μg/mL polyclonal goat F(ab′)2 anti-human IgM and 10 ng/mL of SDF1□□□15 minute modulations were performed for the following: 0.5 μg/mL CD40L (R&D Systems, Minneapolis, Minn.), 50 ng/mL IL-2 (R&D Systems, Minneapolis, Minn.), 50 ng/mL IL-4 (BD Biosciences, San Jose, Calif.), 50 ng/mL IL-21 (Peprotech, Rocky Hill, N.J.), 1000 IU/mL of IFN□ (PBL InterferonSource, Piscataway, N.J.), 5 μg/mL R848 (Invivogen, San Diego, Calif.), and 1 μM thapsigargin (EMD Millipore, Billerica, Mass.). Drug modulations were performed by incubating cells for 4 and 24 hours with a single clinically relevant dose, ranging between the individual agent's Cmaxand trough levels as reported in published pharmacokinetic studies. Bendamustine (Sigma-Aldrich, St. Louis, Mo.) was used at 3.125 μg/mL and the active metabolite of fludarabine, 2-Fluoroadenine-9-β-D-arabinofuranoside (F-ara-A), was used at 4 μM (Sigma-Aldrich, St. Louis, Mo.). Following modulation, cells were fixed with paraformaldehyde at a final concentration of 1.6% for 10 minutes at 37° C. The cells were pelleted, resuspended and permeabilized with 100% methanol, then stored at −80° C. overnight. The permeabilized cells were washed with FACS buffer, pelleted, and stained with a cocktail of fluorochrome-conjugated antibodies (Table 5 gives a partial list of antibodies used). Flow cytometry data were acquired using FACS Diva software (BD Biosciences) on three Canto II flow cytometers (BD Biosciences).

TABLE 5
Representative Antibodies used in Example 2

| | Species and | | |
|---|---|---|---|
| Antibody | [symbol] | Manufacturer | Clone |
| CD3 | Mouse IgG1, κ | Becton Dickson | UCTH1 |
| CD20 | Mouse IgG2a, κ | Becton Dickson | H1 |
| CD5 | Mouse IgG1, κ | Biolegend | UCHT2 |
| p-ERK | Rabbit IgG | Cell Signaling | D13.14.4E |
| (T202/Y204) | | Technology | |
| p-SYK (Y352) | Mouse IgG1 | Becton Dickson | 17A/P-ZAP70 |

[symbol]

indicates data missing or illegible when filed

Flow cytometry data were gated using WinList (Verity House Software, Topsham, Me.). Dead cells and debris were excluded by forward scatter (FSC), side scatter (SSC), and Amine Aqua viability dye. All analyses were gated on B-CLL cells, which were identified as CD3 negative cells exhibiting CD5 and co-expression CD19. The raw instrument fluorescence intensities were converted to calibrated intensity metrics (ERFs, Equivalent Number of Reference Fluorophores). Rainbow calibration particles were included on each plate allowed for the calibration on a plate-by-plate basis. This correction ensures that data across the plate and between plates are calibrated to the same values, regardless of the instrument used for acquisition. The SCNP assay incorporates a number of standardization procedures and process controls that include instrument standardization and calibration, reagent qualification and quality control testing, consistent sample processing, and assay performance monitoring. A cell line control row (Ramos; Burkitt lymphoma cell line) was included on each of the 96-well plates that were processed in this study. The cell line control was used to monitor the reproducibility of the assay performance both during the reported study and to enable comparison with previous studies (data not shown).

### SCNP Terminology and Metrics

The term “signaling node” is used to refer to a proteomic readout in the presence or absence of a specific modulator. For example, the response to anti-IgM modulation can be measured using p-ERK as a readout. That signaling node is designated “anti-IgM→p-ERK”. The term “metric” is used to refer to the quantification method used to evaluate the functional response of signaling proteins. The log 2Fold metric measures the magnitude of the responsiveness of a cell population to modulation relative to the same cell population in the reference well (e.g., isotype or unmodulated) by comparing the median fluorescence values of the responsive cell population to that of the reference population on a log 2 scale. A value of zero would indicate overlapping populations and a value different from zero indicates the responsive population has shifted to higher fluorescence (positive values) or to lower fluorescence (negative values). The log 2Fold metric is calculated as log 2(ERF modulated/ERF unmodulated). The Uu metric is the Mann-Whitney U statistic that compares the ERF values of the modulated and unmodulated wells that have been scaled to the unit interval (0,1) for a given donor and quantifies the fraction of cells responding to a specific modulation{Cesano, 2012 #86}.

When combined, a node-metric is a quantified change in signal and is used to interpret the functionality and biology of each signaling node. It is annotated as “node|metric”, e.g. “anti-IgM→p-ERK|log 2Fold”.

### Statistical Analysis

To evaluate the prognostic significance of the SCNP-defined patient grouping, TTFT curves estimated using the Kaplan-Meier method for the respective patient groups were compared using the log-rank test. Further the SCNP-based prognostic groups were compared (using the log rank test) to their respective prognostic groups defined by IGHV, ZAP-70, or CD38 statuses. For these comparisons as well as the modeling described in following sections, TTFT was calculated from the date of diagnosis to the date of initial therapy. Cases were censored when date of diagnosis were unavailable. Median TTFT and follow-up times were estimated using the Kaplan-Meier method.

Univariate and bivariate models for TTFT were generated using Cox proportional hazards regression implemented in the rms package version 3.1-0 of the R software. Inputs to the models were the change in phosphorylation for each signaling protein in response to modulation (expressed as log 2Fold change or Uu), standard of practice prognostic markers, and clinical covariates. Categorical covariates were coded as 0 or 1 as follows: IGHV mutated=0, IGHV unmutated=1; ZAP-70 negative=0, ZAP-70 positive=1; CD38 negative=0, CD38 positive=1. Bivariate analysis included all possible pairs of inputs to the univariate models. Operating characteristics of the time to event models were summarized using both the likelihood ratio χ2 (LR) and Harrell's concordance index (C), which assesses how well a model orders patients in terms of TTFT. All statistical analyses were performed using the R statistical programming package.

### Model Verification Analysis

The association between increased anti-IgM→p-ERK signaling and shorter TTFT was tested by constructing a Cox proportional hazards model for TTFT using the anti-IgM→p-ERK→log 2Fold or anti-IgM→p-ERK|Uu change as a predictor of TTFT. The association was considered significant if the p-value for the LR chi-square for the model was <0.05.

To implement the anti-IgM→p-ERK finding as a prognostic classifier an optimal cutpoint for separating patients into favorable and unfavorable prognostic groups was identified by linear search of all possible cutoffs with the p-value of the log rank test for difference in Kaplan-Meier TTFT estimates as the objective. That is, a cutoff which yielded the lowest log rank p-value was selected. See Table 6 for modulators, antibodies, sources, concentrations, and modulation times.

TABLE 6
Modulators and incubation times

| | | | Modulation | |
|---|---|---|---|---|
| Modulator | Concentration | | Time (min) | Manufacturer |
| polyclonal goat F(ab′)2 anti- | 20 | ug/mL | 10 | Southern Biotech, |
| human IgM | | | | Birmingham, AL |
| monoclonal anti-human IgD | 5 | ug/mL | 10 | BD Biosciences, San Jose, |
| | | | | CA |
| of SDF1α | 10 | ng/mL | 10 | R&D Systems, Minneapolis, |
| | | | | MN |
| polyclonal goat F(ab′)2 anti- | 20 | ug/mL | 10 | above |
| human IgM and SDF1α | 10 | ng/mL | | |
| CD40L | 0.5 | ug/mL | 15 | R&D Systems, Minneapolis, |
| | | | | MN |
| IL-2 | 50 | ng/mL | 15 | R&D Systems, Minneapolis, |
| | | | | MN |
| IL-4 | 50 | ng/mL | 15 | BD Biosciences, San Jose, |
| | | | | CA |
| IL-21 | 50 | ng/mL | 15 | Peprotech, Rocky Hill, NJ |
| IFNα ( | 1000 | IU/mL | 15 | PBL InterferonSource, |
| | | | | Piscataway, NJ |
| R848 | 5 | ug/mL | 15 | Invivogen, San Diego, CA |
| thapsigargin | 1 | uM | 15 | EMD Millipore, Billerica, |
| | | | | MA |

### Results

### Patient Characteristics

The patient population was predominantly male and representative of the CLL population for cytogenetics and age See FIG. 28. U-CLL and ZAP70 were over-represented (70% and 66% respectively). All patients required treatment; samples were collected prior to initiation of treatment.

αIgM→p-ERK is associated with time to first treatment

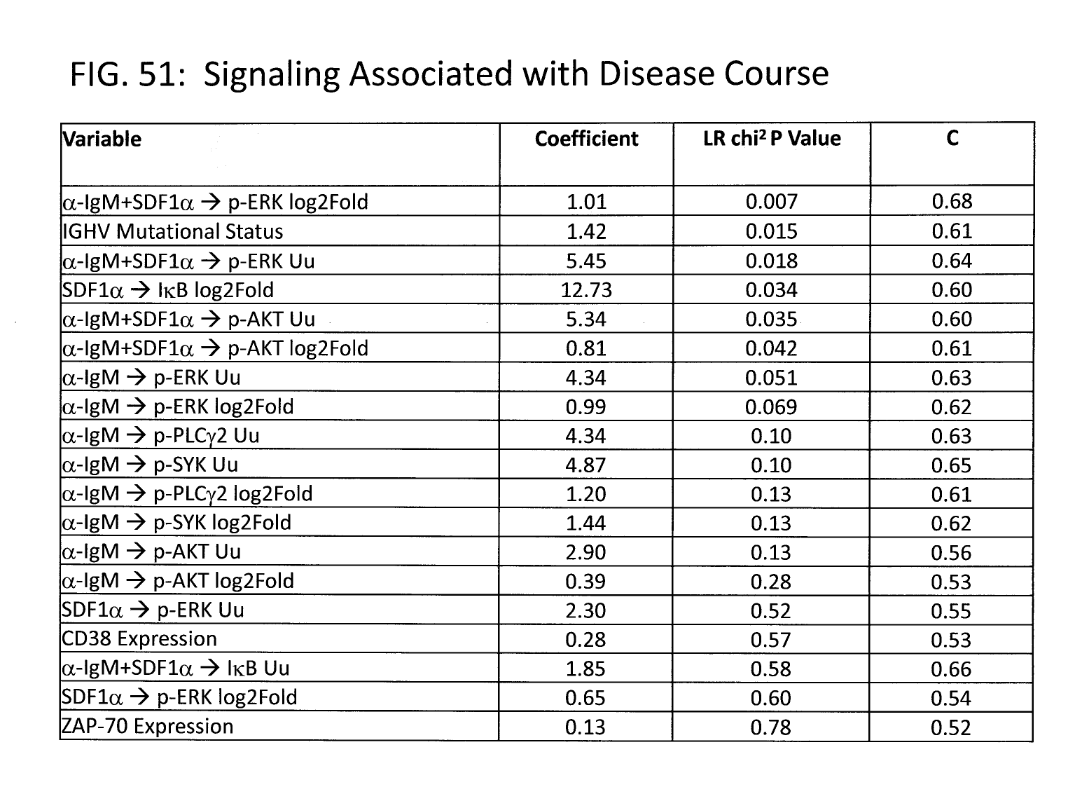

Consistent with prior studies, F(ab)₂IgM→p-ERK signaling was associated with TTFT (p=0.05, likelihood ratio test). Previous studies showed association of αIgM→p-ERK with TTFT in samples collected previously untreated and prior to disease progression requiring the initiation of therapy from patients with Binet stage A CLL. Herein we examined whether BCR engagement using αIgM would correlate with TTFT in samples collected at the time of disease progression from patients with Binet stage A or B CLL. Consistent with prior studies, the trend of greater αIgM→p-ERK signaling with TTFT was observed (Uu metric, p=0.05, likelihood ratio (LR) χ2 test test; log 2Fold metric p=0.07). See FIG. 51. Importantly, these data demonstrate that the association of αIgM→p-ERK signaling to TTFT is consistent throughout the disease course of CLL, even at the time of disease progression requiring the initiation of therapy. That is, previous data was taken at various timepoints in disease progression and showed the association, and the present data, taken just prior to initiation of treatment, show the same association and this indicates a consistent association throughout disease course, making this a useful predictor of TTFT.

Association of Signaling Pathways with TTFT

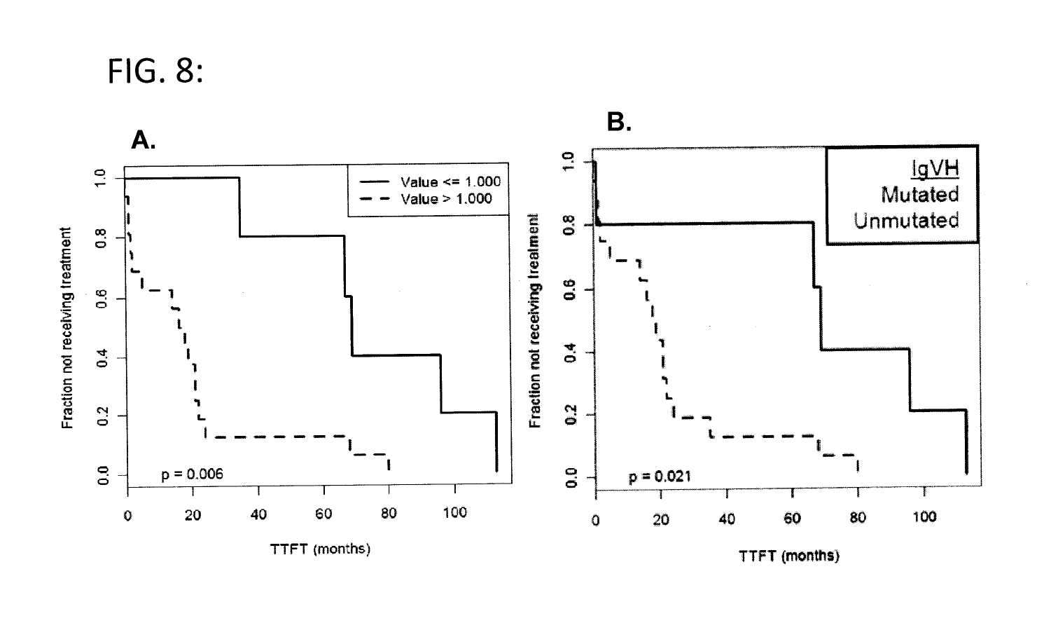

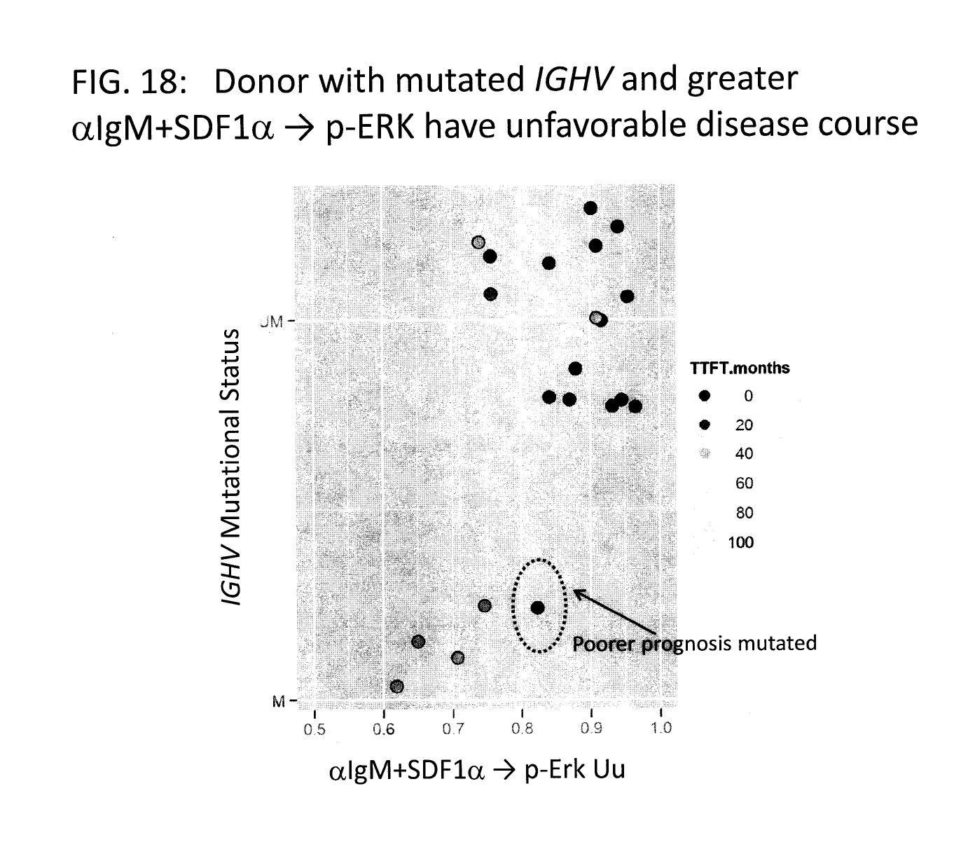

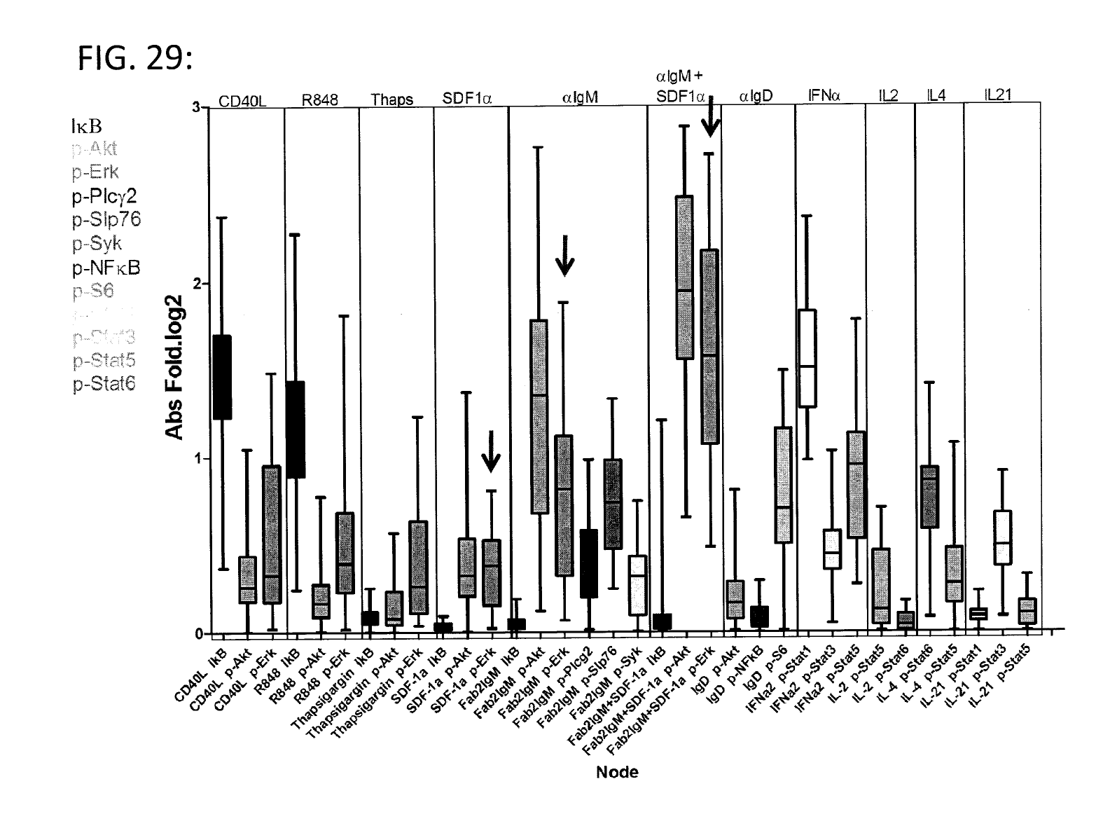

Noteworthy, the combination of SDF1α and F(ab)₂IgM modulation induced greater p-ERK signaling than observed with either agent alone and displayed stronger association with TTFT (p=0.02) (FIG. 8A, FIG. 18). The simultaneous modulation of CLL cells with αIgM plus the chemokine SDF1α (CXCL12) produced a stronger p-ERK signal (1.57 median log 2Fold) than either αIgM (0.82 median log 2Fold) or SDF1α (0.38 median log 2Fold) (FIG. 29). Furthermore, the association αIgM+SDF1α→p-ERK with TTFT was greater than αIgM alone (log 2Fold metric: p=0.007 vs p=0.07, Uu metric: p=0.02 vs p=0.05, respectively) (FIG. 51). Similar improvements in p-AKT association with TTFT were observed with the combined modulation. SDF1α→p-AKT and SDF1α→p-ERK, both produced moderate signals (log 2Fold≧0.25), and did not associate with TTFT.

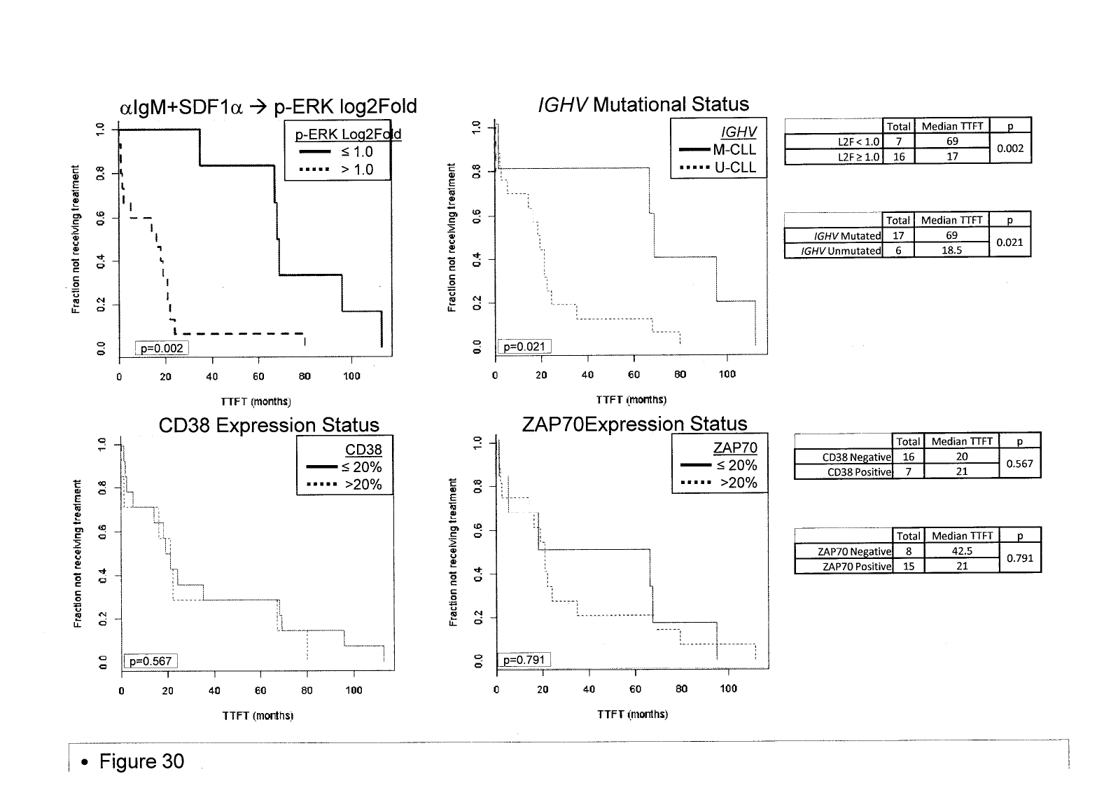

A cut-point of 1.0 (log 2Fold metric) was determined using the linear search method described above to yield the best separation in Kaplan-Meier TTFT estimates for favorable (αIgM→p-ERK|log 2Fold<1.0) and unfavorable (αIgM+SDF1α→p-ERK|log 2Fold≧1.0) prognostic groups (FIG. 30). The median TTFT for patients with high signaling was 17 months compared to more than 60 months for those with lower signaling (p=0.002). Similarly, IGHV mutational status associated with TTFT; however CD38 and ZAP-70 expression did not correlate with TTFT (FIG. 51, FIG. 30).

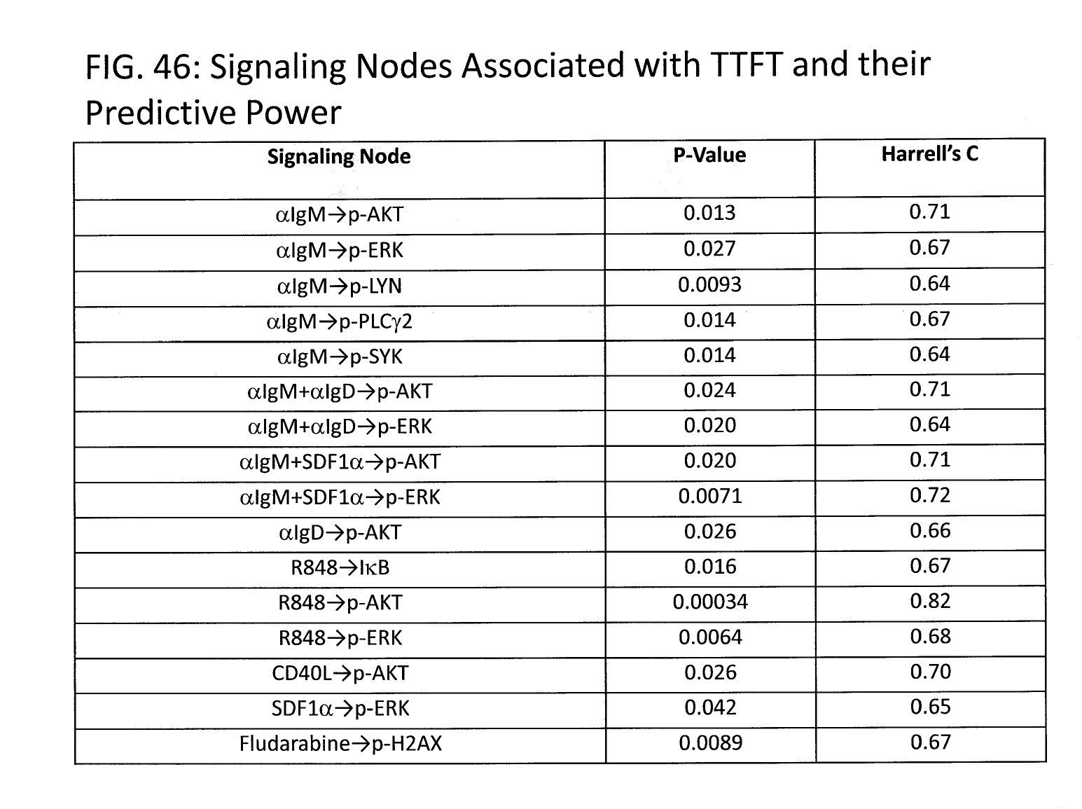

Signaling beyond αIgM→p-ERK was evaluated for clinical utility in predicting unfavorable disease course (FIG. 27). Signaling proteins more proximal to the receptor (LYN, SYK, and PLCγ2) and AKT showed a trend of increased signaling in samples from donors with a more aggressive disease course (FIG. 46). Other signaling pathways including the JAK/STAT, CD40L, or TLR7/8 pathways did not associate with TTFT in this sample set.

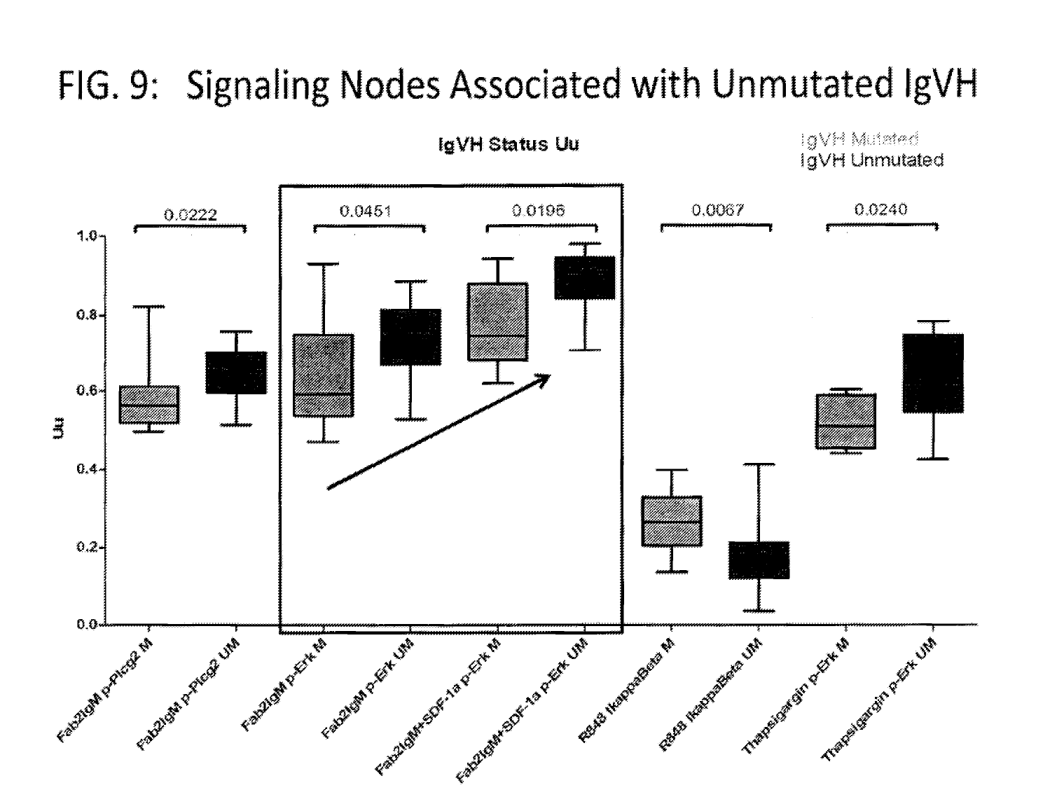

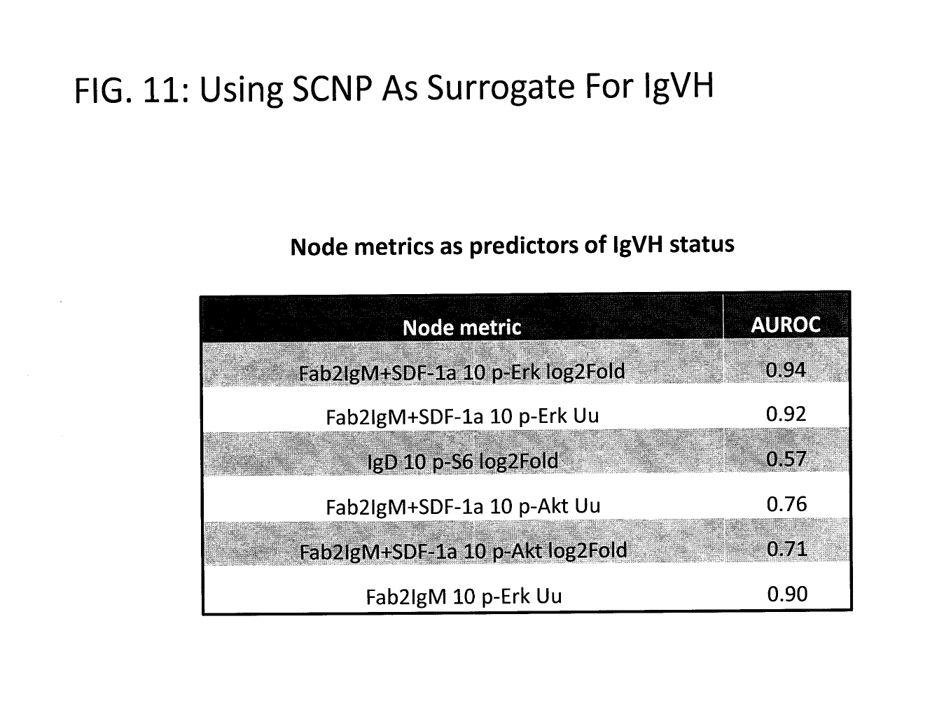

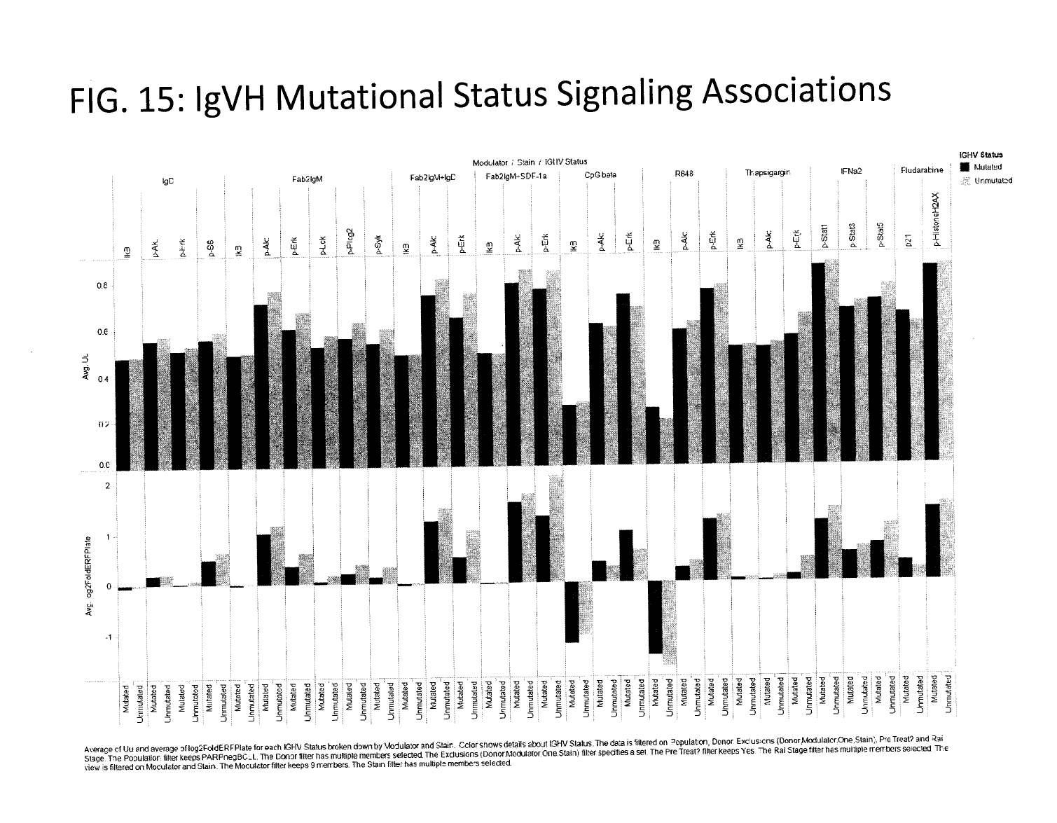

In addition, combining IgVH with F(ab)₂IgM→p-ERK did not improve prediction as the results were already so similar. Significant associations to IgVH unmutated status (FIG. 9) included multiple nodes modulated by F(ab)₂IgM (p-ERK, p-PLCgamma2, p-SYK). See FIGS. 11 and 15. The strength of this relationship was greater using concurrent stimulation with F(ab)₂IgM+SDF1□. R848 (TLR7/8 agonist) and thapsigargin (Ca2+ influx) signaling were also increased in the unmutated samples. See also Tables 5 and 6 below. FIG. 11 shows node metric associated with using SCNP as a predictor of IgVH status.

### SCNP Provides Prognostic Information Beyond IGHV Mutational Status

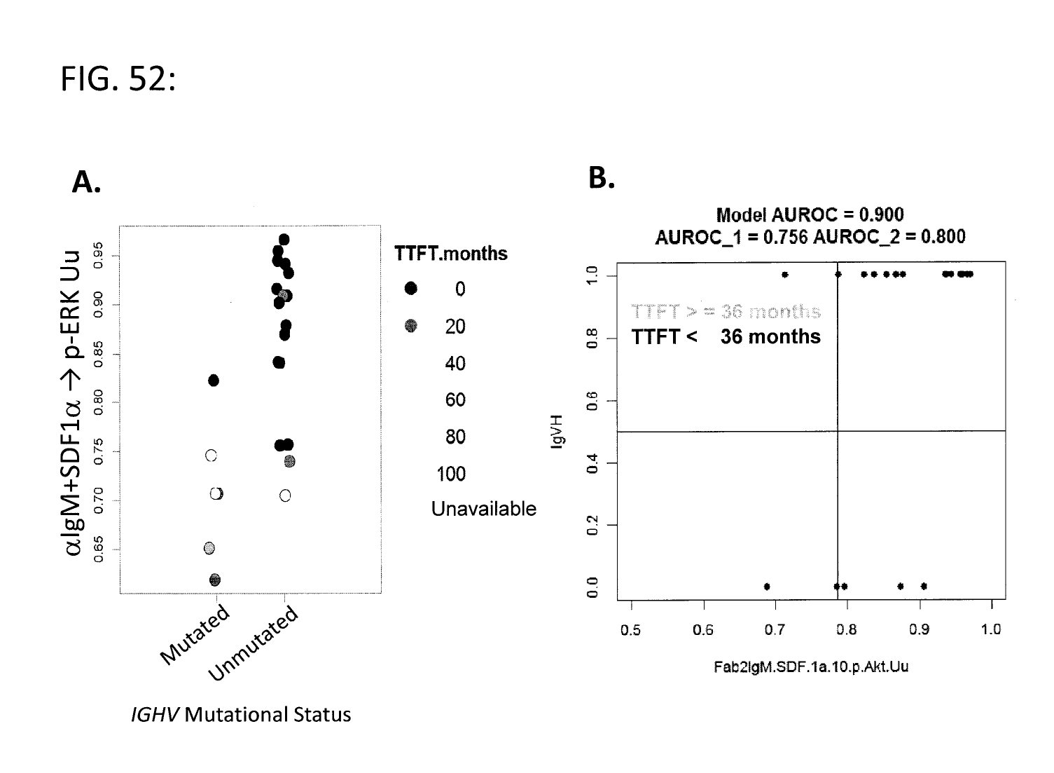

IGHV mutational status (P=0.021, FIG. 30, FIG. 8B) but not CD38 nor ZAP-70 associated with TTFT for this cohort of patients requiring treatment. Having shown that both IGHV mutational status and induced signaling (αIgM→p-ERK, αIgM+SDF1α→p-ERK, αIgM+SDF1α→p-AKT) associated with TTFT, we sought to determine if the information gained from the functional characterization of the cells' signaling potential provided data that could improve modeling disease progression. Anti-IgM+SDF1α→p-ERK|Uu was plotted in IGHV mutated and unmutated samples and TTFT was depicted by color (FIG. 52). Of the five M-CLL samples with TTFT data, the sample with the greatest p-ERK signal came from the donor that had the shortest TTFT. Conversely, of the U-CLL, the sample with the lowest p-ERK signal originated from a donor that had a relatively longer TTFT. Similarly, combining IGHV status and αIgM+SDF1α→p-AKT produces a decision tree model with better performance (AUROC=0.90, p<0.0001) than either variable alone. These data show that disease-relevant stimuli provides data that is independent of IGHV mutational status.

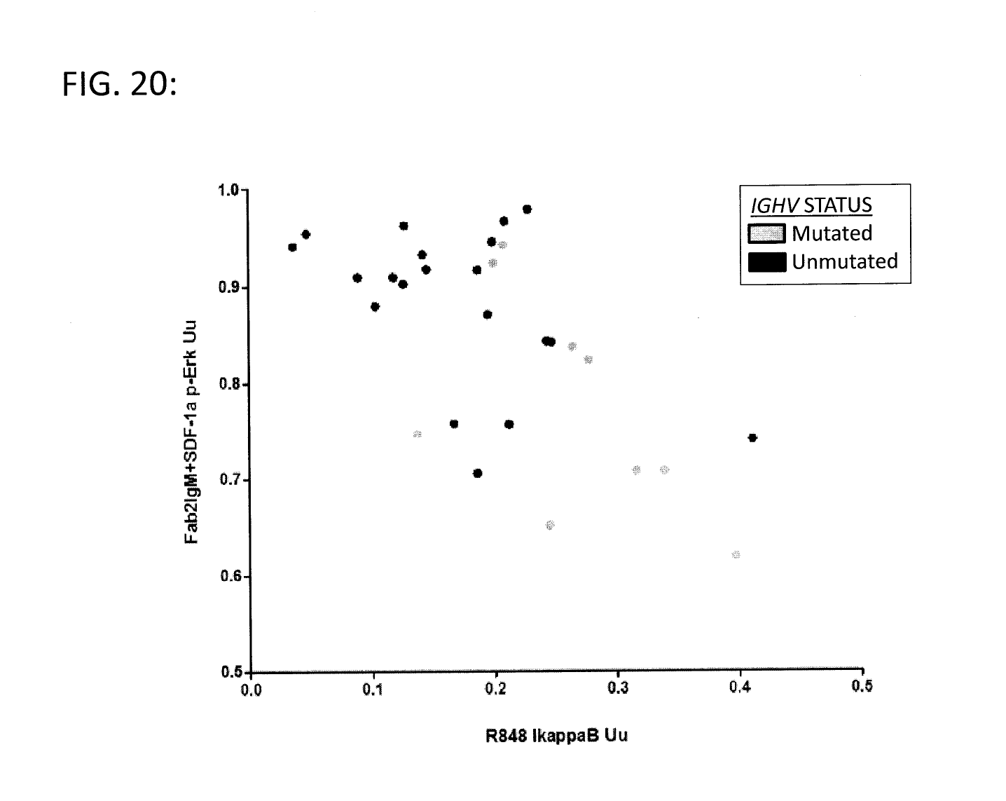

Additionally, SCNP has the potential to define prognosis beyond IGVH. For example, we confirmed that F(ab)₂IgM>p-Erk signaling associates with unmutated IGVH (AUROC=0.90). Patients with unmutated IGVH had greater basal p-Erk and p-H2AX signaling and greater R848/TLR7>NFkB (IkB) signaling. We also observed a weaker drug induced signaling in IGVH unmutated donors. See FIG. 20.

### Characterization of Drug-Induced Signaling Informs on Resistance Mechanisms

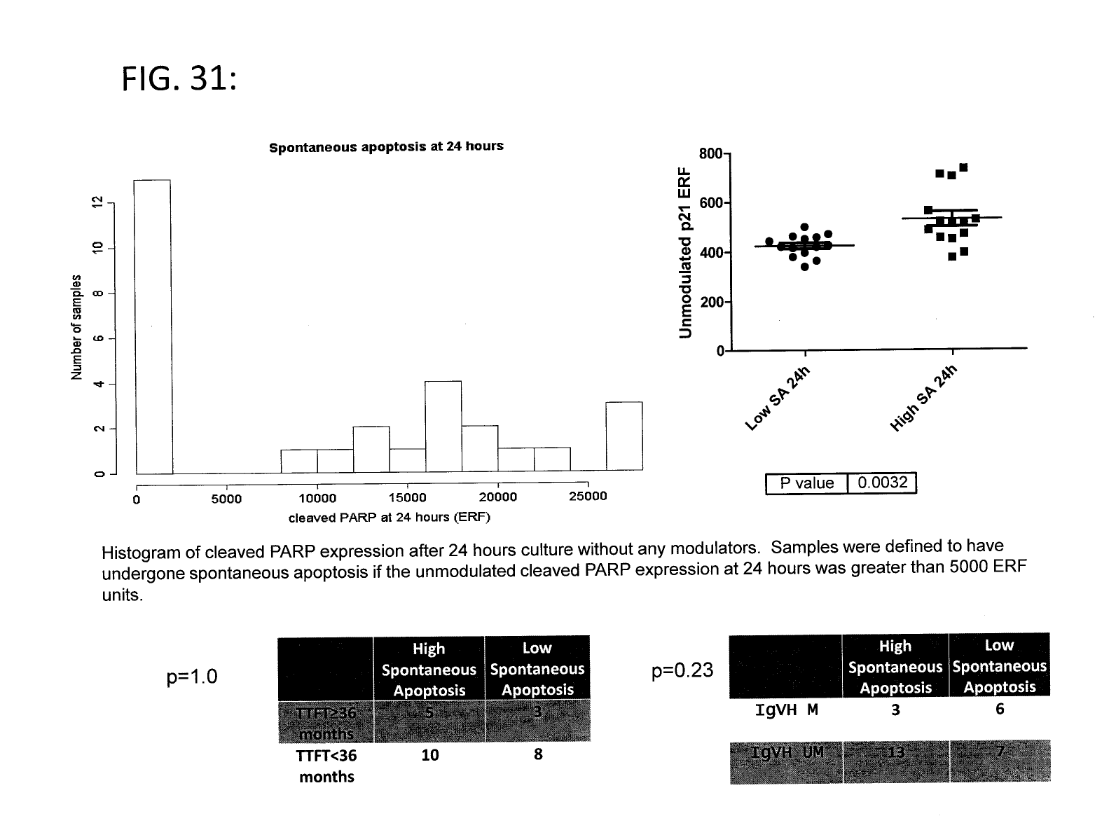

To understand chemosensitivity in samples taken from donors at time of treatment initiation, samples were incubated with either bendamustine, an alkylating agent, or the active metabolite of fludarabine (F-ara-A or fludarabine des-phosphate), a DNA synthesis inhibitor, for 4 and 24 hours. Importantly, cells were stained with cleaved PARP, an early marker of apoptosis, in addition to markers of DNA damage (p-CHK2, p-H2AX, and p-53BP1) or cell cycle arrest (p21). This enabled the characterization of cells capable of signaling by excluding cleaved PARP positive “dying” cells that may have lost their ability to signal. Additionally cleaved PARP by itself can provide a measure of chemosensitivity and a measure of spontaneous apoptosis. Indeed, 16 of the 29 samples had high levels of spontaneous apoptosis at 24 hours cultured in the absence of drug (FIG. 31). Spontaneous apoptosis was not associated with disease course or IGHV mutational status. For the purposes of these analyses, the effects of drug on induced signaling were performed with data from the 13 samples with low spontaneous apoptosis.

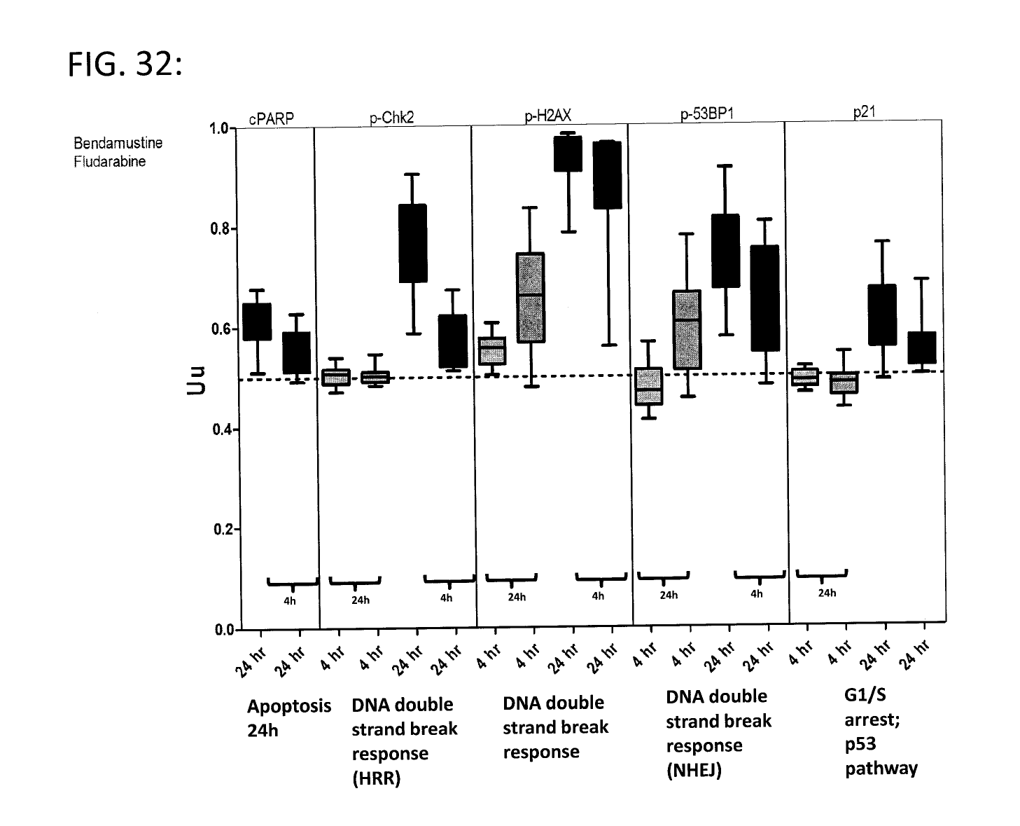

Fludarabine-induced p-H2AX and p-53BP1 signaling was greater than bendamustine signaling at 4 hours (FIG. 32). However this effect was lost at 24 hours with equivalent p-H2AX signaling and greater bendamustine→p-53BP1 signaling. Except for p-H2AX, bendamustine induced greater signaling likely because of greater DNA damage caused by bendamustine that is less dependent than fludarabine on DNA replication. Cell cycle arrest as identified by p21 expression was apparent in cells cultured for 24 hours with drug.

p53 activation induces p21 expression, a protein that inhibits the cell cycle at G1 through inhibition of CDK2. To investigate p53 functional status, p21 levels were measured under conditions that activate p53. When cultured in the presence of DNA-damaging agents, cells with wild type p53 are expected to respond by inducing p53 activity resulting in up-regulation of p21 expression; conversely p21 induction is expected to be absent in p53 mutant cells under the same activating conditions. For both bendamustine and fludarabine, cytotoxicity depends on functional p53 and as a result, the consequent induction of p21 can be considered a marker for drug activity.

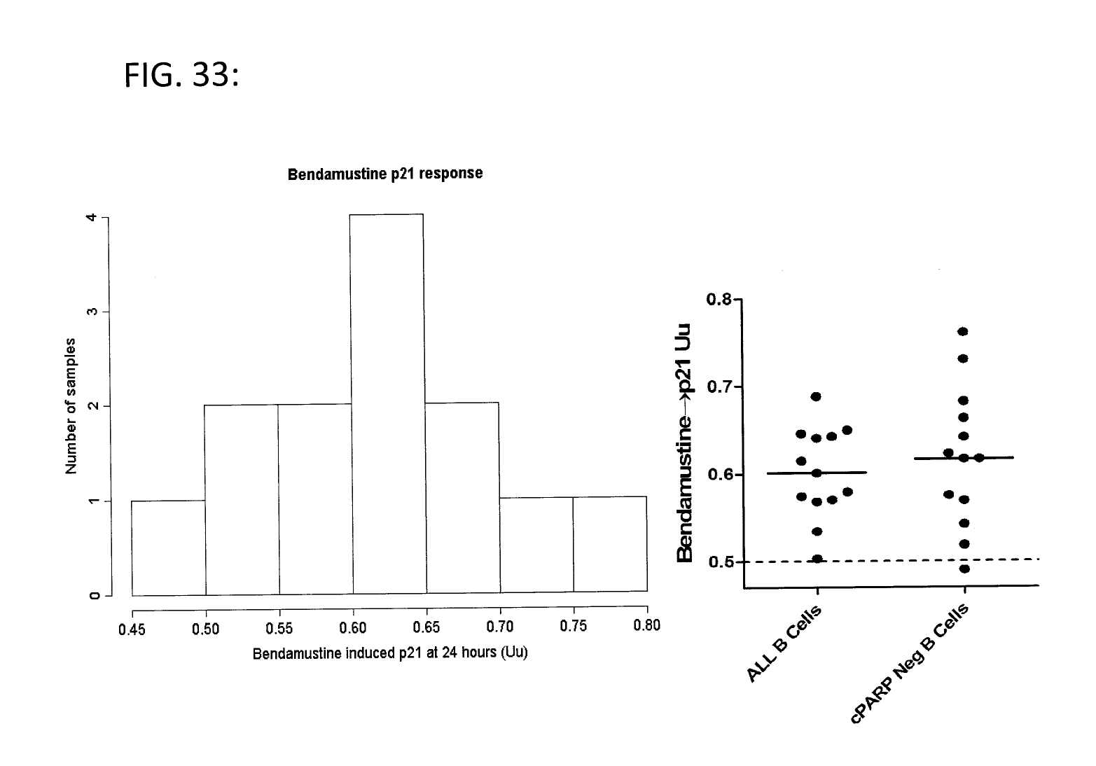

Therefore, we hypothesized that CLL cells that carry p53 alterations (structural i.e. mutations or functional i.e epigenetic regulation) will fail to respond with increased levels of p21 when exposed to bendamustine for 24 hours whereas cells competent for p53 function will induce p21 expression on drug treatment. FIG. 33 shows the distribution of p21 induction by bendamustine in cleaved PARP negative B cells from eligible samples after culturing for 24 hours. Samples showing reduced p21 induction by bendamustine are predicted to have a higher likelihood of having a p53 pathway defect; which is to say samples with increased p21 induction are more likely to carry an intact p53 pathway.

To test the hypothesis that p21 induction would predict p53 (TP53) mutational status, a logistic regression was performed with p53 mutational status as the dependent variable and p21 induction by bendamustine quantified in cleaved PARP negative CLL cells using the Uu metric as the predictor; in other words, the following logistic regression model was built:
p53 molecular status˜bendamustine→p21|Uu

The G test statistic was used to assess the significance of the relationship between p53 mutational status and p21 induction by bendamustine. Prior to unblinding the clinical data, including the mutational status of p53, the p-value for the G test was prespecified as needing to be less than or equal to 0.05 for the relationship between p53 mutational status and p21 induction by bendamustine to be considered significant. Further, the regression coefficient must have a positive sign.

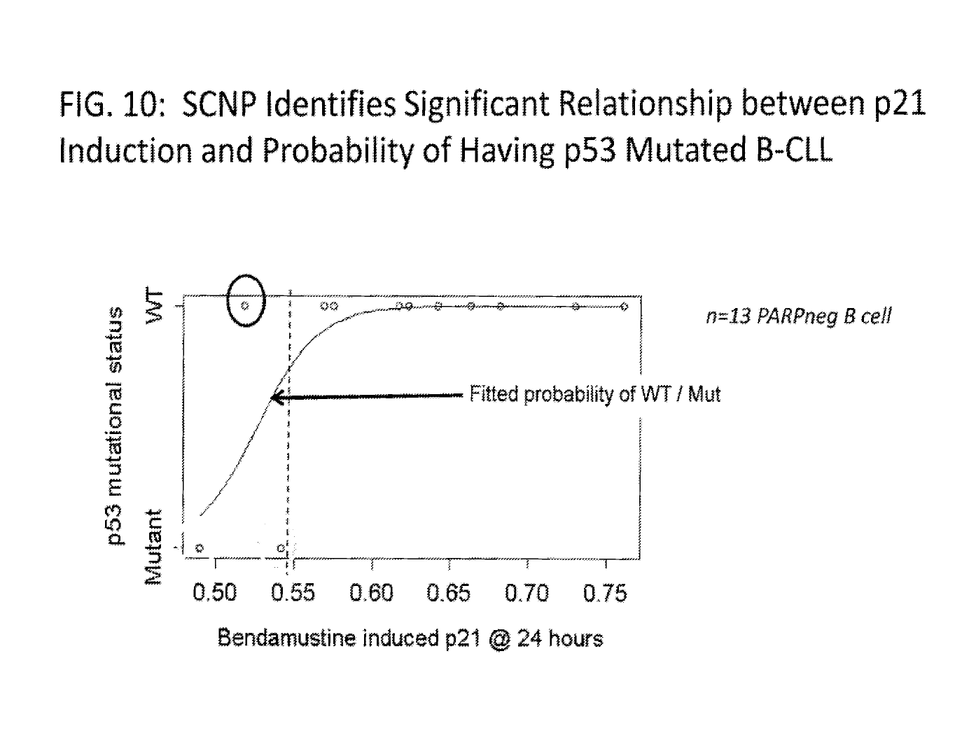

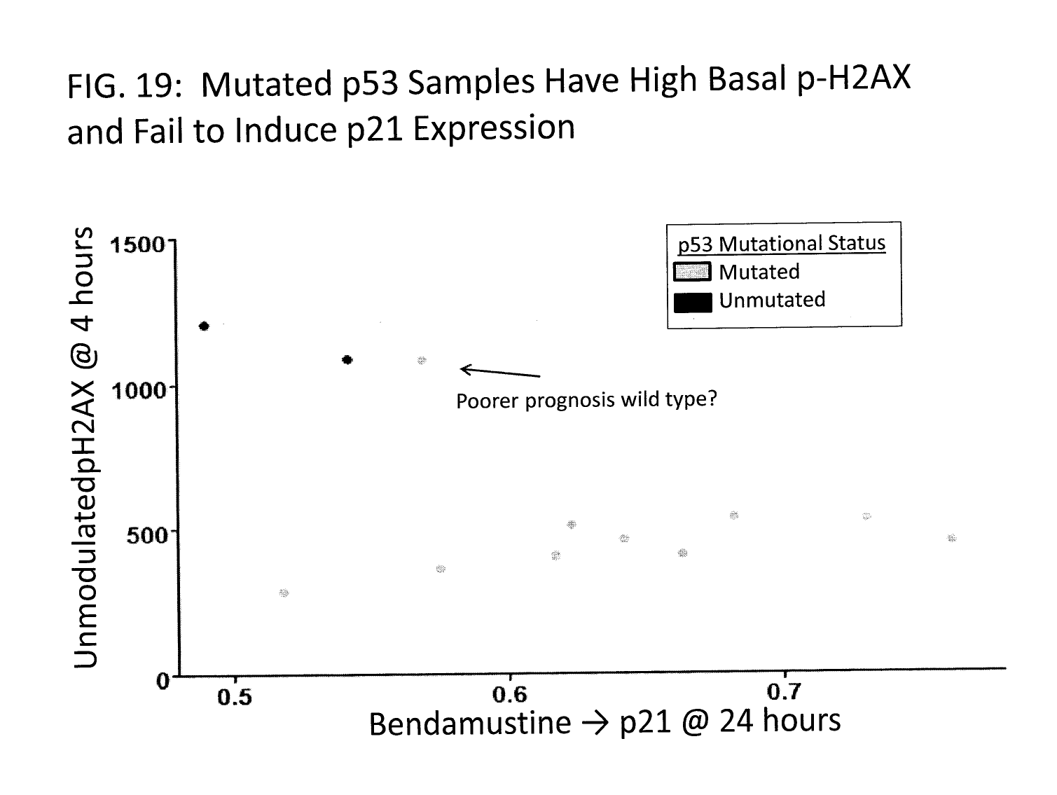

The model correctly predicted (p=0.0125) the two donors which had cells positive for p53 mutations (FIG. 10). Alternatively, testing by Fishers test using a Uu value of 0.55 as a boundary between mutated and unmutated produced a p value of 0.045. An additional third donor with wild-type TP53 was predicted as being mutant. See FIG. 19. Mutated p53 samples have a high basal p-H2AX and fail to induce p21 expression. There are several possible explanations including differences in cellular drug uptake and expulsion via drug efflux pumps or mutations in related p53 pathway mediators. Interestingly, the donor sample that was positive for p53 mutation and also positive for del17p had the lowest p21 induction.

An important advantage of this functional assay is the ability to quantify signaling only in competent, cleaved PARP negative cells. This excludes unhealthy cells initiating apoptosis that may have other activities, such as caspases, that would impede measurements of a drug's effect on signaling. The dynamic range of the assay is greater in cleaved PARP negative cells, providing greater stratification of the samples (FIG. 33).

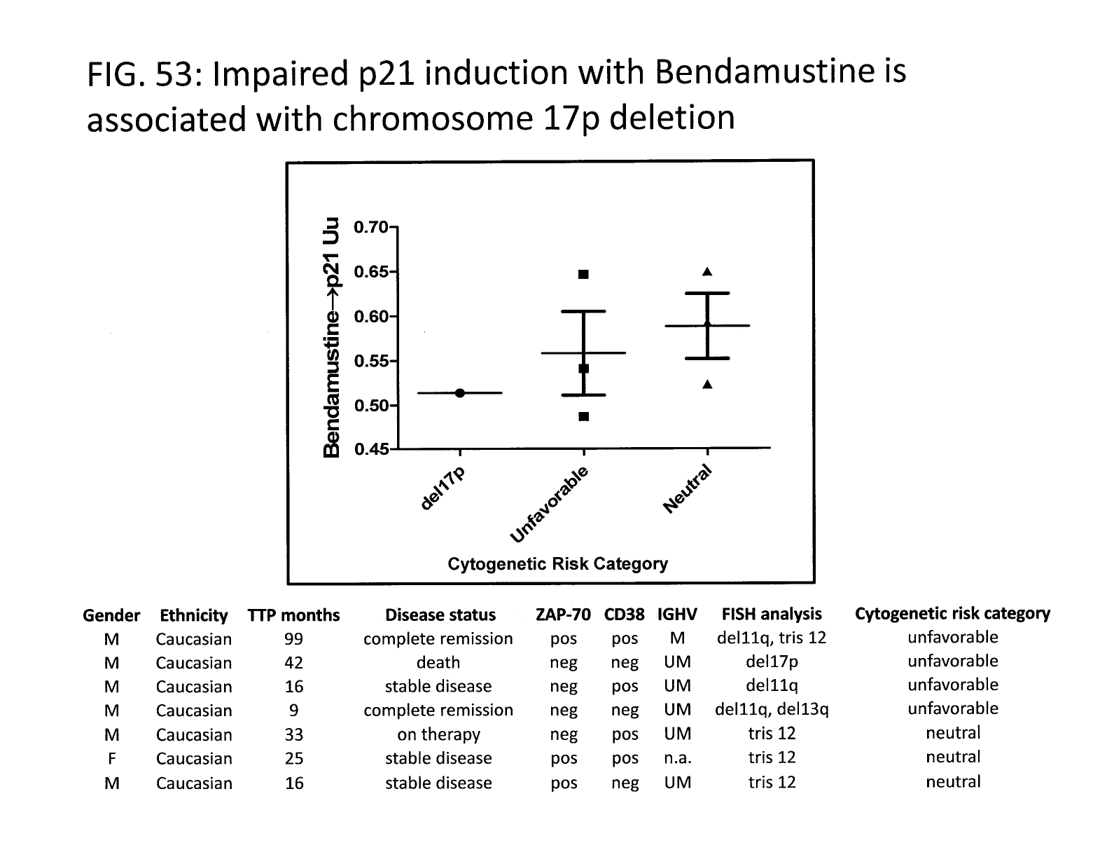

To further characterize the relationship of p21 to p53 mutational status in CLL, an additional small cohort of 7 CLL samples with cytogenetic data was analyzed by SCNP. Using these samples, we examined whether a reduced/absent p21 induction at 24 hour post treatment with bendamustine is associated with the presence of a chromosome 17p deletion, and conversely whether p21 induction is associated with lack of chromosome 17p deletion. In agreement with the earlier results, the samples with 17p deletion had impaired p21 induction in response to culturing in the presence of bendamustine (FIG. 53). Though the small sample size precludes statistical measurement of the association, it lends support for testing the prognostic value of bendamustine induced p21 expression.

This Example confirms the association of BCR and BCR+SDF1alpha signaling in B-CLL disease progression, and the potential for SCNP to identify those patients more likely to require early treatment. This Example demonstrates that αIgM→ERK and αIgM+SDF1α→ERK are prognostic (TTFT) for CLL even at the time just before initiation of therapy, and suggests that the signaling is hard wired into the cells and present throughout the disease. αIgM+SDF1α provided an even more robust prognosis of TTFT. In addition, high levels of BCR induced p-ERK are also associated with IGHV mutational status but can provide independent prognostic information within these molecularly defined CLL subsets. The SCNP assay can provide an independent indication of p53 mutation, and likelihood of a patient to respond to therapy requiring an intact p53. Thus, the SCNP assay can (1) identify in one assay those patients with a more aggressive form of B-CLL, including both unmutated IgVH and p53 pathway alterations, and (2) identify patients with signaling profiles that may be more likely to respond to targeted therapies.

### Example 3

In this Example, patients with CLL at various timepoints before treatment and healthy controls were analyzed to 1) map SCNP signaling profiles in early-stage B-CLL and to 2) identify signaling associations with clinical subgroups defining B-CLL prognosis (IgVH mutational status, cytogenetic risk, CD38/ZAP70 expression).

### Patients and Samples

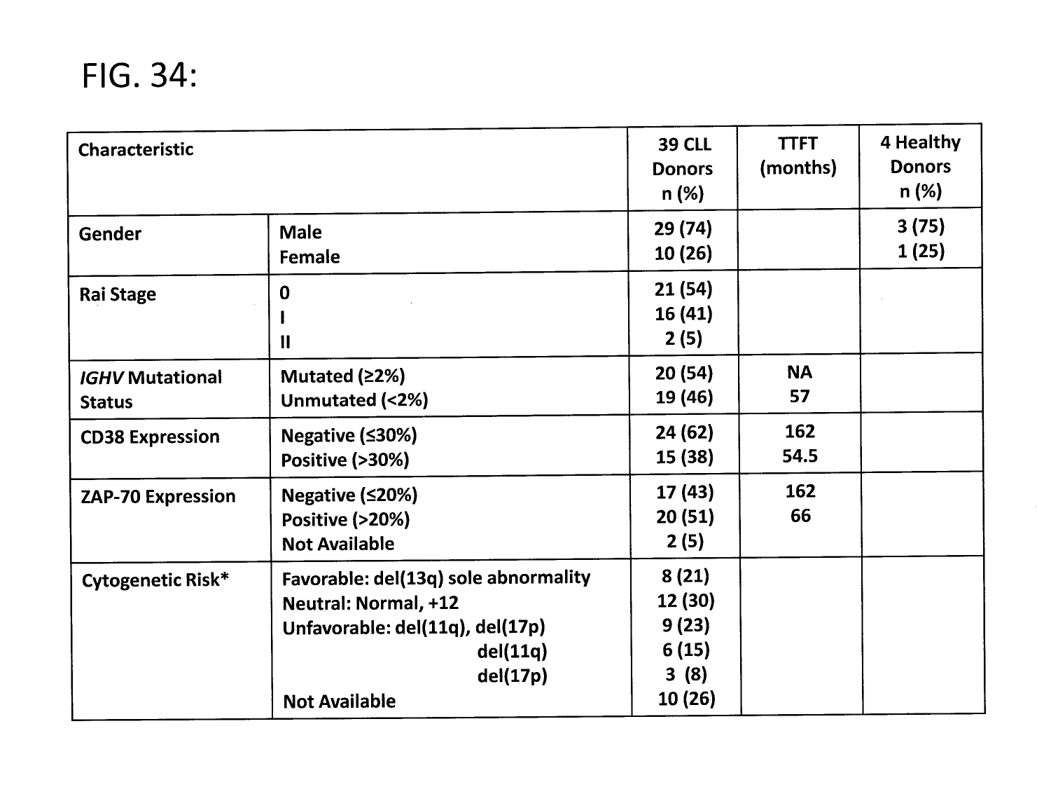

Peripheral blood mononuclear cells (PBMCs) were obtained from 39 B-CLL patients between 2006 and 2007, Rai stage 0-II, at different time points during their clinical course but prior to the initiation of treatment. PBMCs from four age-matched healthy donors were collected at the Stanford Blood Center. All donors provided informed consent for research purposes. SCNP assays were performed blinded to clinical data. Diagnosis and initiation of treatment for B-CLL were based on 1996 National Cancer Institute-Working Group (NCI-WG)/IWCLL 2008 Guidelines for Diagnosis and Treatment of CLL. Clinical and biological disease characteristics at diagnosis are summarized in FIG. 34. The median age of CLL patients was slightly lower than the typical CLL population (58 years vs 65-70). Of the 39 samples evaluated, 15 expressed CD38 (≧30% of cells) and 20 expressed ZAP-70 (≧20% of cells); 19 were IGHV unmutated (98% cutoff); and the cytogenetic risk groups were evenly represented. IGHV mutational status and expression of CD38 and ZAP-70 were determined. IGHV sequencing utilized a 2% cutoff to delineate unmutated from mutated IGHV, and a cutoff of 30% and 20% were used for CD38 and ZAP-70 determination, respectively.

### Single-Cell Network Profiling

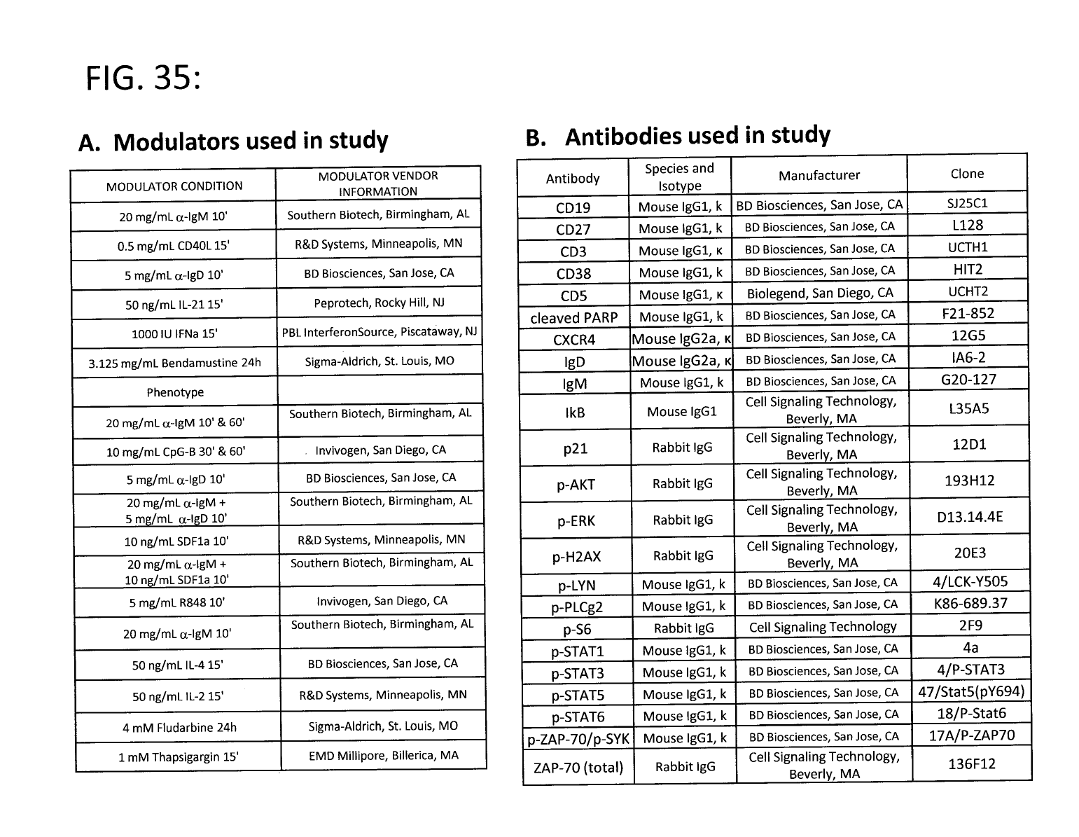

SCNP analysis quantitatively measured intracellular signaling proteins within CD19+CD5+B-CLL cells using a panel of disease-relevant modulators (FIG. 27, FIG. 35A, B). First, the cryopreserved PBMC samples were thawed at 37° C., stained for viability with Amine Aqua (Invitrogen, Carlsbad, Calif.), resuspended in RPMI with 10% FBS and aliquoted at 100,000 cells per well in 96-deepwell plates. Cells were rested for 2 hours at 37° C. followed by modulation. Short duration modulation was performed for 10 minutes at 37° C. with the modulators listed in FIG. 35A. Drug modulations were performed by incubating cells for 24 hours with a single clinically relevant dose, ranging between the individual agent's Cmaxand trough levels as reported in published pharmacokinetic studies. Following modulation, cells were fixed with paraformaldehyde at a final concentration of 1.6% for 10 minutes at 37° C. The cells were pelleted, resuspended and permeabilized with 100% methanol, then stored at −80° C. overnight. The permeabilized cells were washed with FACS buffer, pelleted, and stained with a cocktail of fluorochrome-conjugated antibodies. FIG. 35B. Flow cytometry data were acquired using FACS Diva software (BD Biosciences) on three Canto II flow cytometers (BD Biosciences).

Flow cytometry data were gated using WinList (Verity House Software, Topsham, Me.). Dead cells and debris were excluded by forward scatter (FSC), side scatter (SSC), and Amine Aqua viability dye. All analyses were gated on B-CLL cells, which were identified as CD3 negative cells exhibiting CD5 and co-expression CD19. The raw instrument fluorescence intensities were converted to calibrated intensity metrics (ERFs, Equivalent Number of Reference Fluorophores).

Comparisons of signaling between the ZAP-70- and ZAP-70+ subset of B-CLL cells were performed by using the sample's T cells to set the ZAP-70 cutoff.

### SCNP Terminology and Metrics

SCNP terminology and metrics were as described in Example 2.

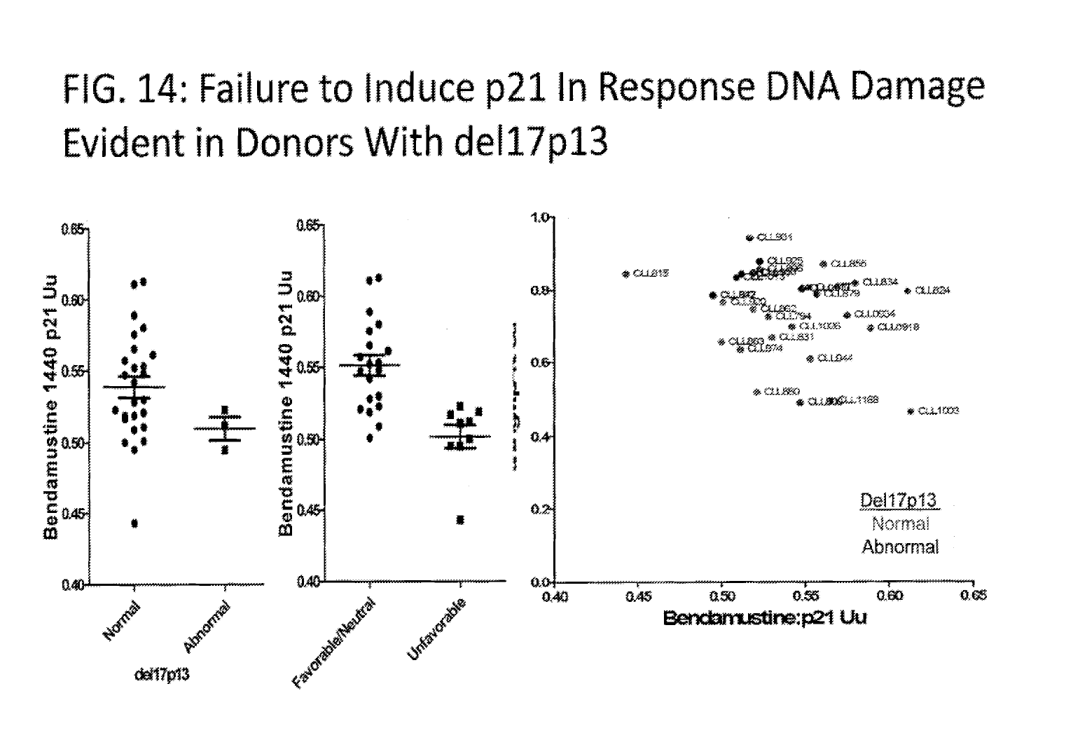

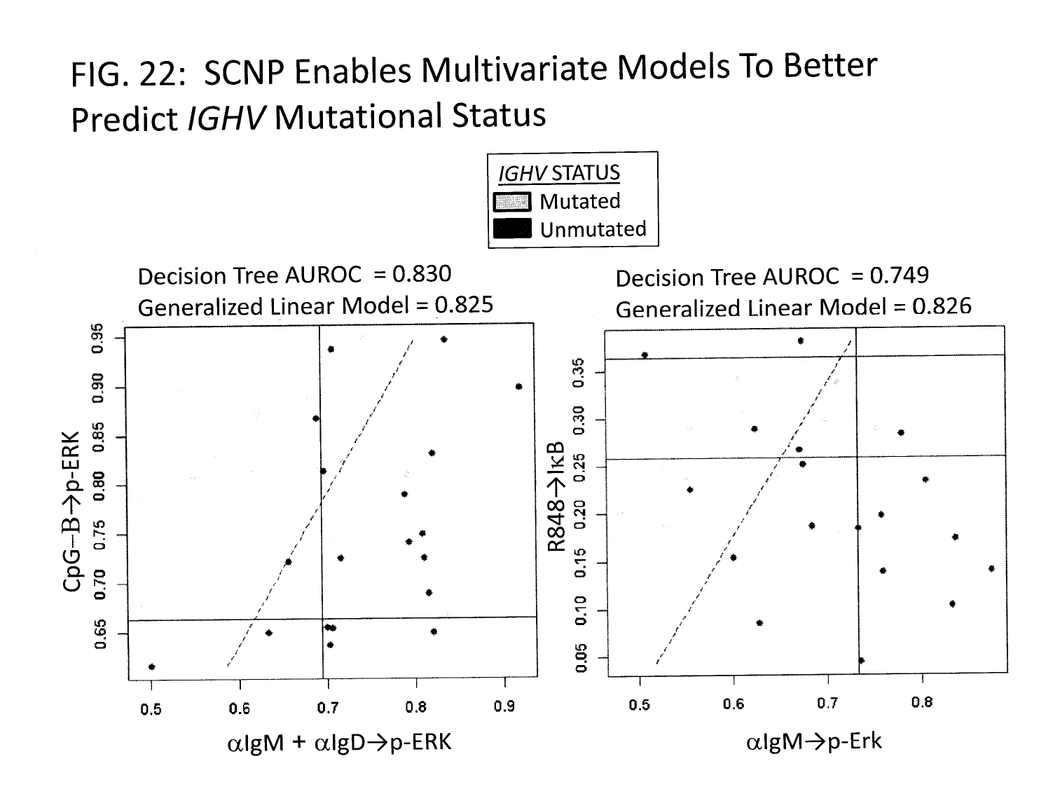

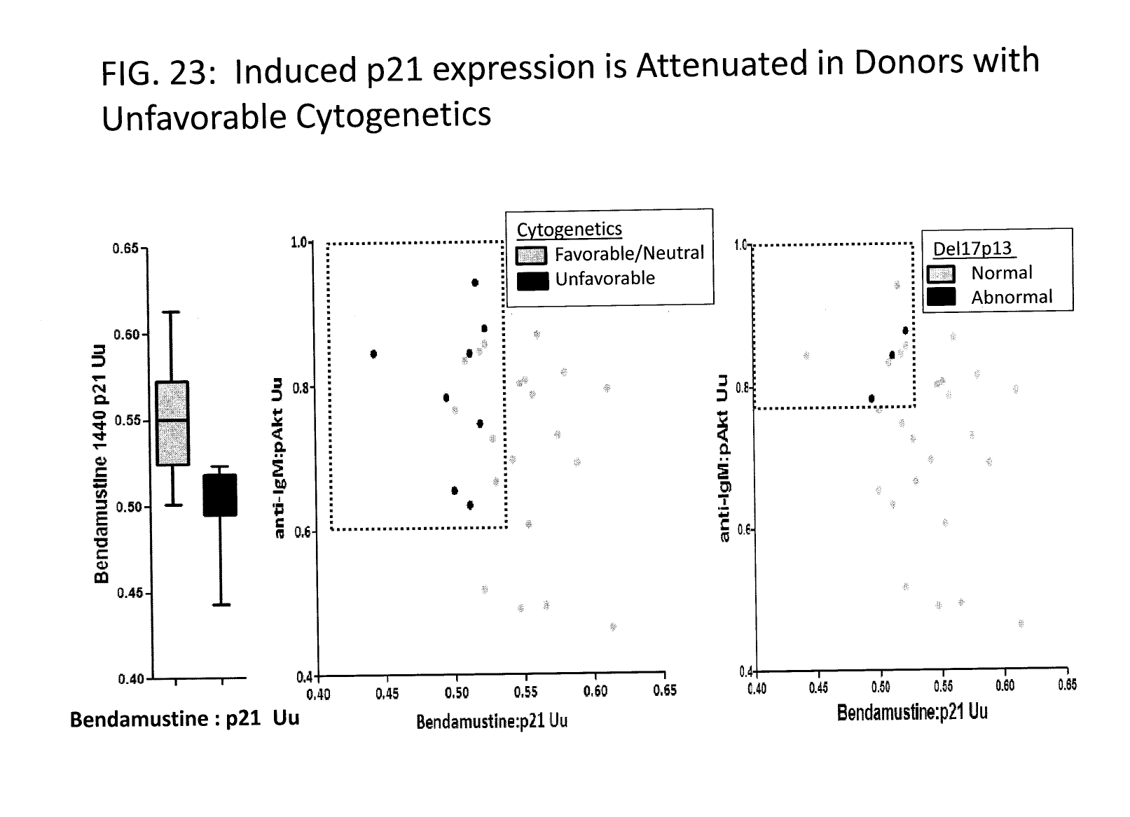

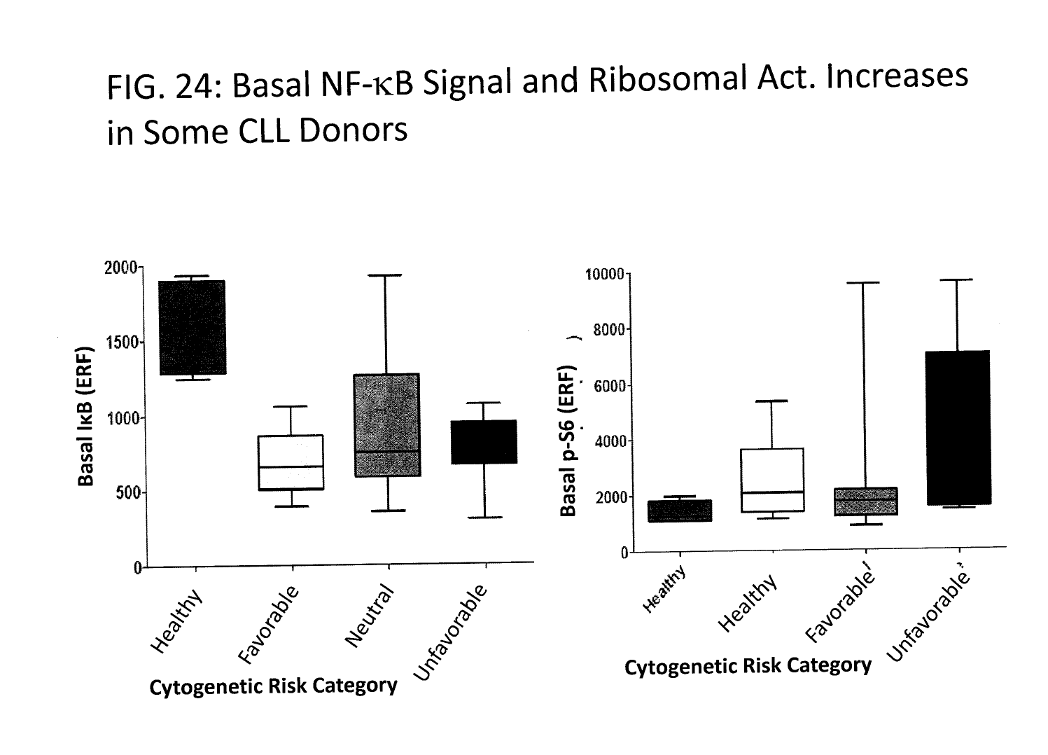

Results: Significant associations with patient risk categories and signaling are summarized in Table 5. IgVH unmutated and ZAP70 samples showed elevated F(ab)₂IgM or anti-IgD induced BCR signaling, either alone or in combination, decreased CpGbeta→p-ERK levels and increased Thapsigargin (Ca²⁺ signaling) signaling. Of note CD38 samples did not show the same associations but shared with IgVH samples higher responsiveness to IFN alpha and weaker induction of p21 in response to bendamustine. The unfavorable cytogenetic group samples showed increased F(ab)₂IgM→p-ERK and had higher basal p-S6 that further increased with anti-IgD crosslinking Lack of p21 induction was also associated with unfavorable cytogenetics, which includes deletion of p53 (del17p), a regulator of p21 expression. See FIGS. 14 and 23. (FIG. 23 shows the bar chart on the left with a Y axis having a scale from 0.40 to 0.65 in 0.05 increments. It is labeled with the Bendamustine 1440 p21 Uu. The left graph shows favorable and unfavorable cytogenetics and the right graph shows normal and abnormal Del17p13 status) SCNP enables multivariate models to better predict IGVH mutational status. See FIG. 22. Also, it shows that basal NF-kB signaling and ribosomal activity increased in some CLL donors. We also found that basal levels of p-S6, a marker of ribosomal activity, observed in donors with unfavorable cytogenetics. See FIG. 24.

TABLE 7
Signaling Associations to Patient Risk Groups (Wilcoxon p-value)

| | | | | Unfavorable | |
|---|---|---|---|---|---|
| | | | | CGX | |
| | | | | (del11q22.3 | Favorable |
| | CD38 ≧ | IGHV | ZAP-70 ≧ | and/or | CGX |
| Signaling Node | 30% | Unmutated | 20% | del17p13) | (13q14.3) |
| ↑ αIgM → p-ERK | | 0.013 | 0.0059 | | |
| ↑ αIgM → p-LYN | | 0.003 | 0.0060 | | |
| ↑ αIgM → p-PLCγ2 | | 0.014 | 0.0244 | | |
| ↑ αIgM → p-SYK | | 0.024 | | | |
| ↑ αIgM → p-ERK* | | 0.018 | 0.0485 | 0.013 | |
| ↑ αIgM + αIgD → p- | | | 0.043 | | |
| AKT | | | | | |
| ↑ αIgM + αIgD → p- | | 0.0039 | 0.0018 | | |
| ERK | | | | | |
| ↑ αIgM + SDF1α → p- | | 0.0050 | 0.0074 | | |
| ERK | | | | | |
| ↑ αIgD → p-S6 | | | 0.0119 | 0.034 | |
| ↓ CpG□ → IκB | | | 0.030 | | |
| ↓ CpG□ → p-ERK | | 0.017 | 0.0355 | | |
| ↑ R848 → IκB | 0.0055 | | | | |
| ↓ R848 → IκB | | | | | 0.031 |
| ↑ IFNα → p-STAT1 | 0.0026 | | | | |
| ↑ IFNα → p-STAT3 | 0.0159 | | | | |
| ↑ IFNα → p-STAT5 | 0.0024 | 0.0047 | | | |
| ↑ IL2 → p-STAT5 | | | | 0.008 | |
| ↑ Thapsigargin → p- | | 0.047 | 0.0102 | | |
| AKT | | | | | |
| ↑ Thapsigargin → p- | | 0.0092 | 0.0018 | | |
| ERK | | | | | |
| ↓ Thapsigargin → p- | 0.0448 | | | | |
| ERK | | | | | |
| ↓ Bendamustine → p21 | 0.0021 | 0.025 | | 0.0003 | |
| ↑ Fludarabine → p- | 0.0073 | | | | |
| H2AX | | | | | |

*Measured at 60 minutes; all other αIgM modulations measured at 10 minutes.

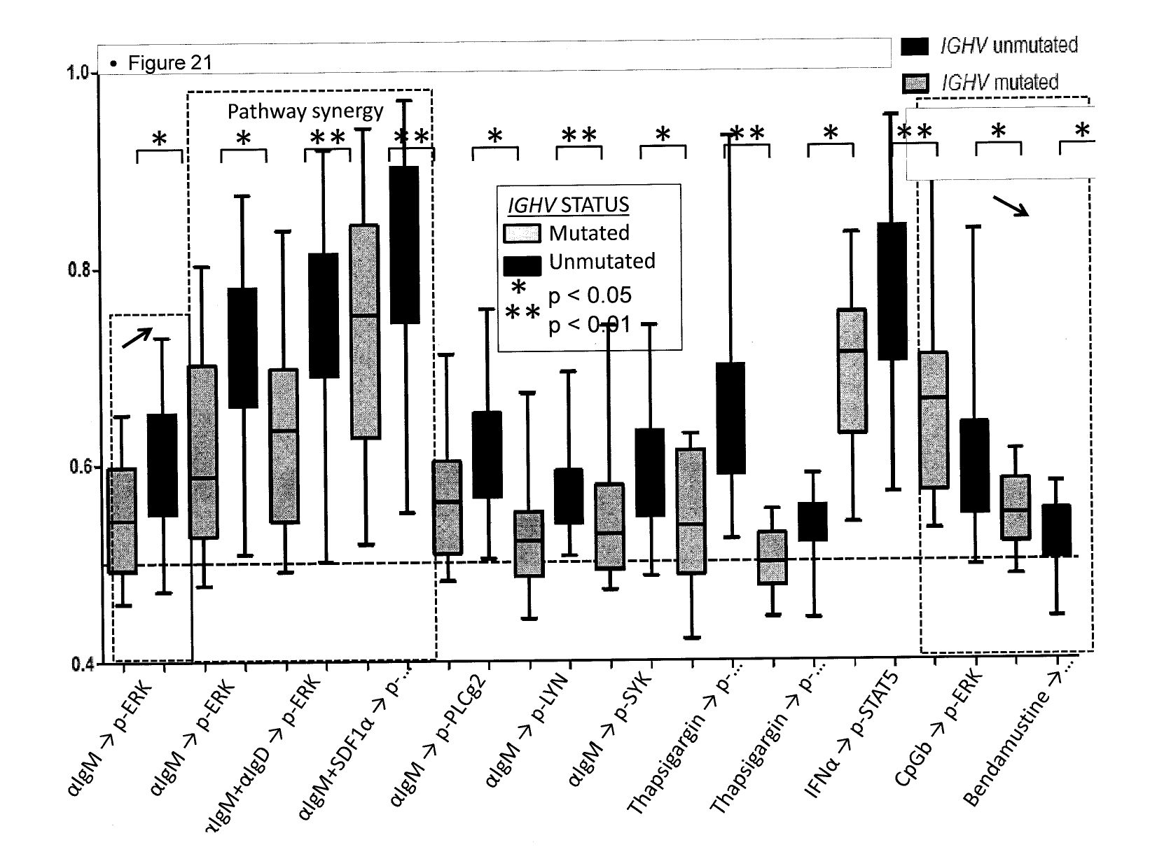

See also FIG. 21 for the above Table 7.

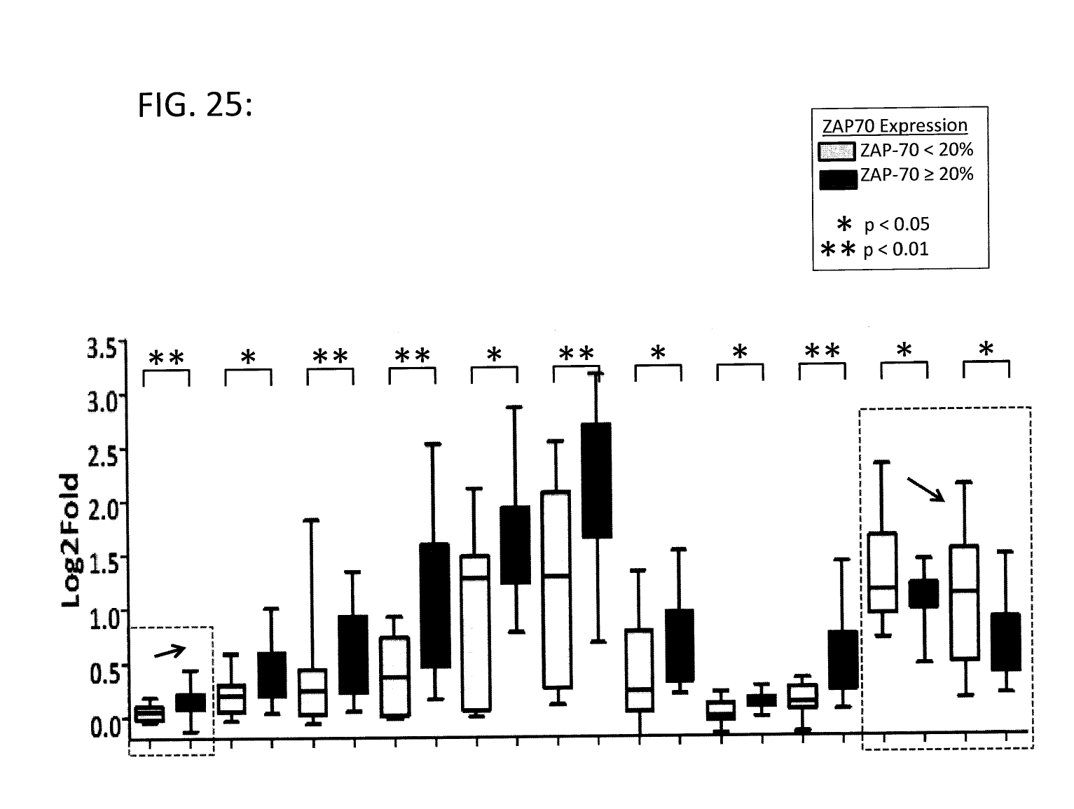

ZAP-70 expression and not CD38 associates with BCR signaling. Donors with ZAP-70 expression show greater BCR signaling, either induced alone with F(ab)₂IgM or anti IgD alone or in combination; stronger thapsigargin/Ca2+ signaling; and lower CpG-B/TLR7 signaling. Donors with CD38 expression on CLL cells showed no measurable difference in BCR signaling; greater responseiveness to IFNα; stronger R848/TLR7 signaling; and lower p21 induction and higher induction of p-H2AX. See FIGS. 25 and 26. In FIG. 25, the nodes for the pairs going from left to right (in a similar manner to FIG. 26) are anti IgM (also known as F(ab)₂IgM)>p-Lyn; anti IgM>p-PLCg2; anti IgM>p-Erk; anti IgM+anti IgD>p-Erk; anti IgM+anti IgD>p-Akt; anti IgM+SDF1a>p-Erk; anti IgD>p-56; Thapsigargin>p-Akt; Thapsigargin>p-Erk; CpGb>IkB; and CpGb>p-Erk.

### Altered Signaling in CLL Cells

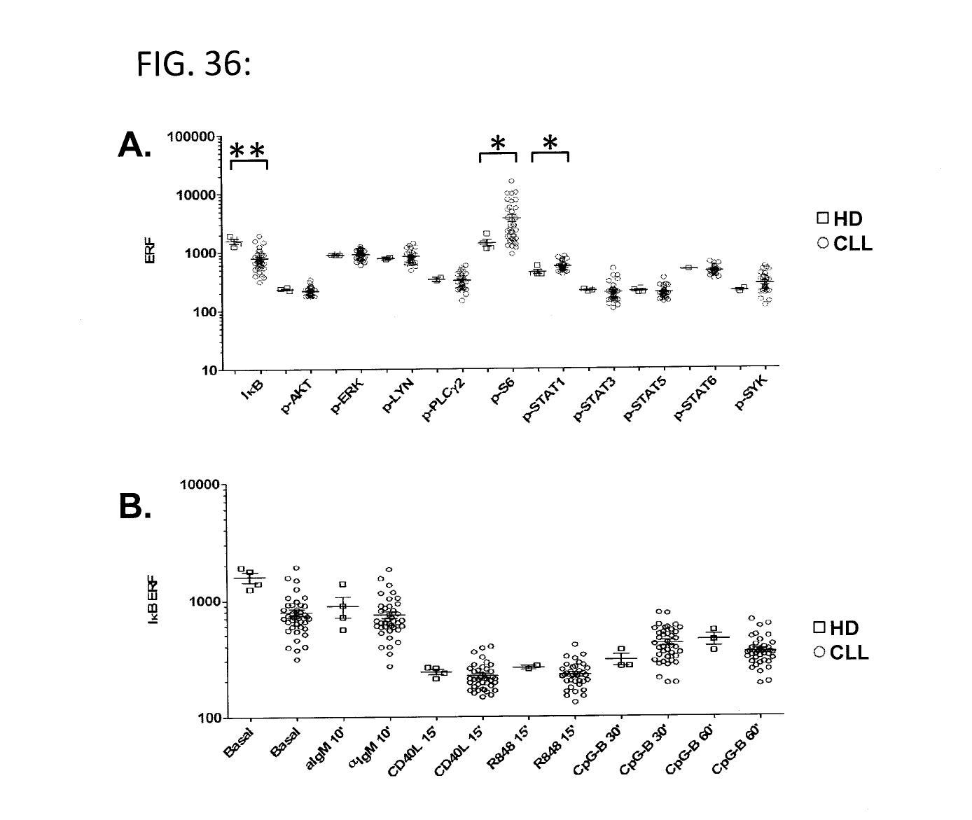

A broad sampling of functional signaling capabilities of B-CLL thought to be associated with disease pathogenesis (FIG. 27) was examined by comparing basal and modulated signaling in CD5⁺CD19⁺ B-CLL cells from 39 patients to CD19⁺ B cells from four age and gender matched healthy donors. While most signaling proteins showed similar basal levels of activation as measured by the normalized MFI metric (ERF) (FIG. 36A) basal levels of p-S6, indicative of ribosomal activity, and p-STAT 1 were significantly higher in the CLL samples. Interestingly, basal ID B levels in CLL samples were similar to levels achieved only after modulation with αIgM in healthy controls, suggesting tonic BCR signaling in CLL. See FIG. 36B.

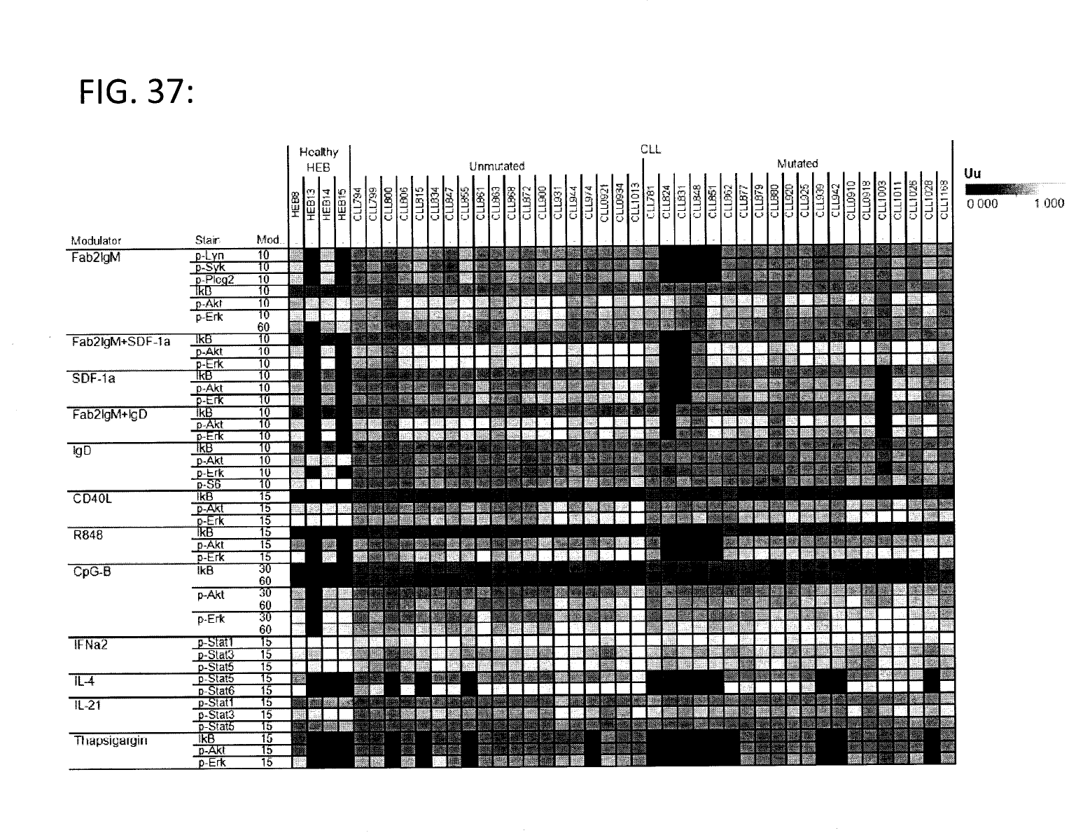

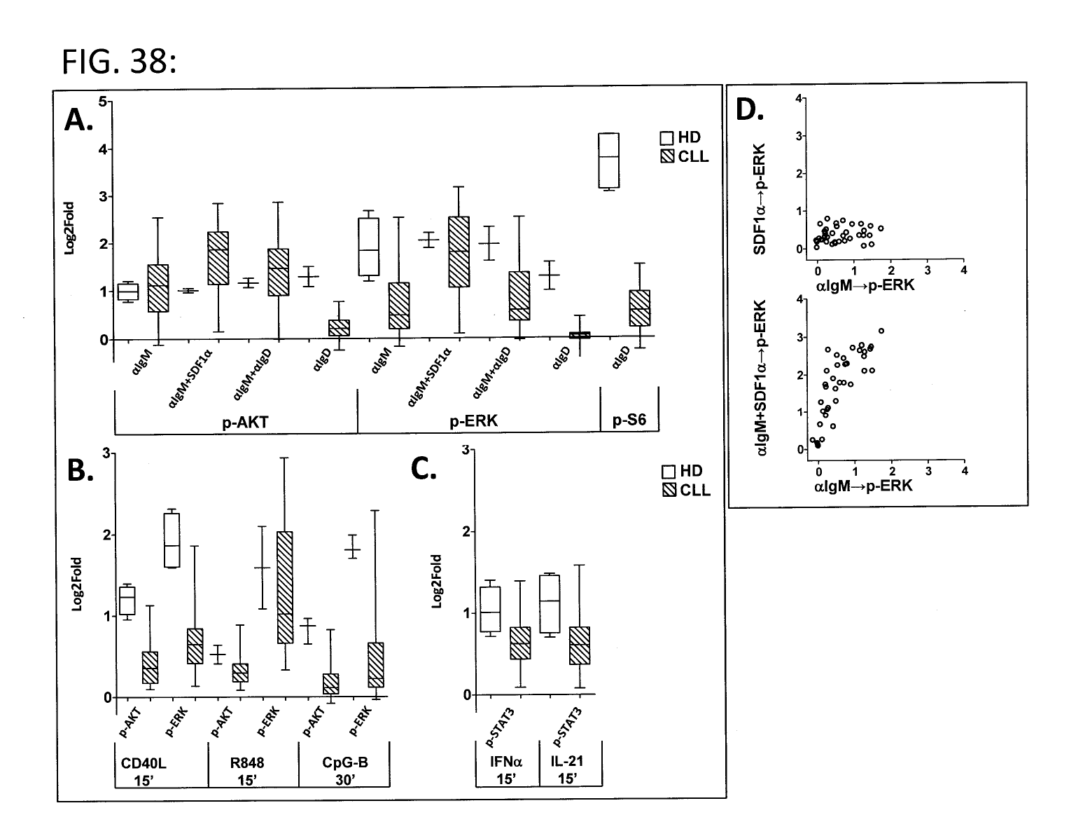

Modulated levels of phosphoproteins demonstrated dysregulated signaling in multiple pathways in B-CLL cells versus healthy B cells including growth factor, cytokine, BCR, CD40L-mediated NFKB, TLR and DNA damage response signaling (FIG. 37, FIG. 38). αIgM modulation identified attenuated activation of proximal signaling proteins LYN, SYK, and PLCγ2 in B-CLL cells relative to the B cells of healthy controls indicative of broad dysfunctional signaling in CLL (FIG. 37). Downstream signaling pathways mediated through ERK and AKT diverged in their alignment with healthy signaling. ERK signaling was attenuated in most CLL samples; in contrast, AKT activation was maintained in CLL with many samples exhibiting greater αIgM→p-AKT than the healthy samples (FIG. 38A, FIG. 37). SDF1α→p-AKT modulation as a single stimulus was weaker in nearly all CLL samples compared to the healthy controls. However, cells are likely exposed to multiple stimuli in vivo and the context in which a stimulus is presented may have a significant effect on the response. Indeed, when cells were modulated with the combination of αIgM and SDF1α induced a greater than additive response was observed in the induction of p-ERK and to a lesser extent p-AKT from the CLL samples (FIG. 38B). Furthermore, CLL samples showed equivalent p-ERK and nearly double the AKT activation (Log 2Fold) observed in healthy samples when modulated with αIgM and SDF1α.

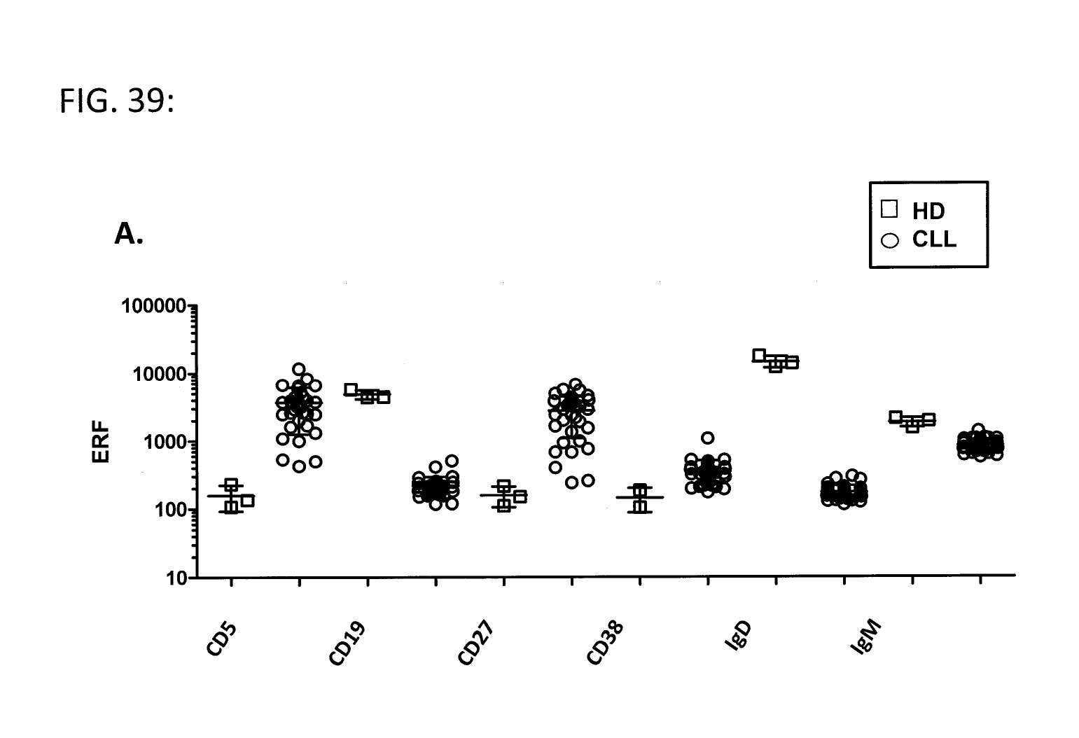

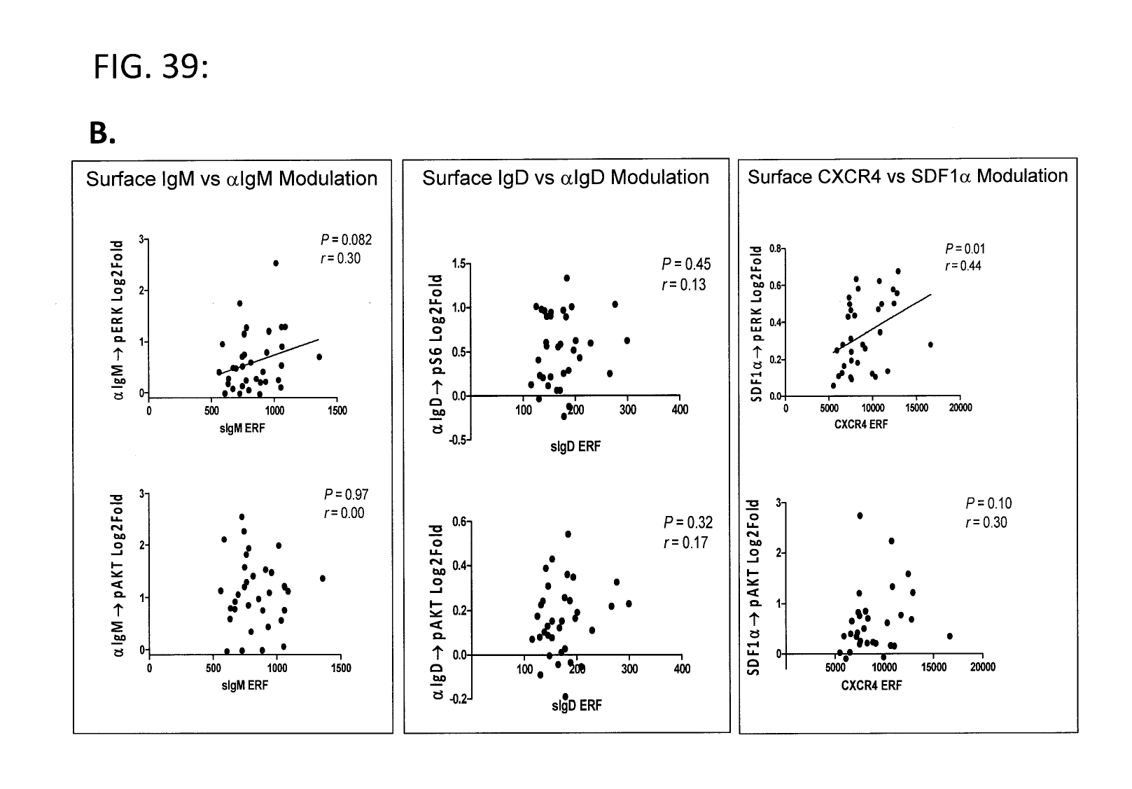

To determine whether the observed attenuated signaling through surface receptors could be explained by lower receptor levels in B-CLL cells, surface expression of IgD, IgM, and CXCR4, the receptor for SDF1α, was measured. Significantly lower expression of IgD and IgM (ERF, p=0.005, Mann-Whitney), no difference in CXCR4 levels, and higher CD27 and CD38 expression was observed in CLL samples compared to healthy samples (FIG. 39). Collectively these results indicate that receptor expression levels do not represent a correlate of signaling capacity or magnitude.

SCNP Identifies Signaling Profiles Associated with IGHV

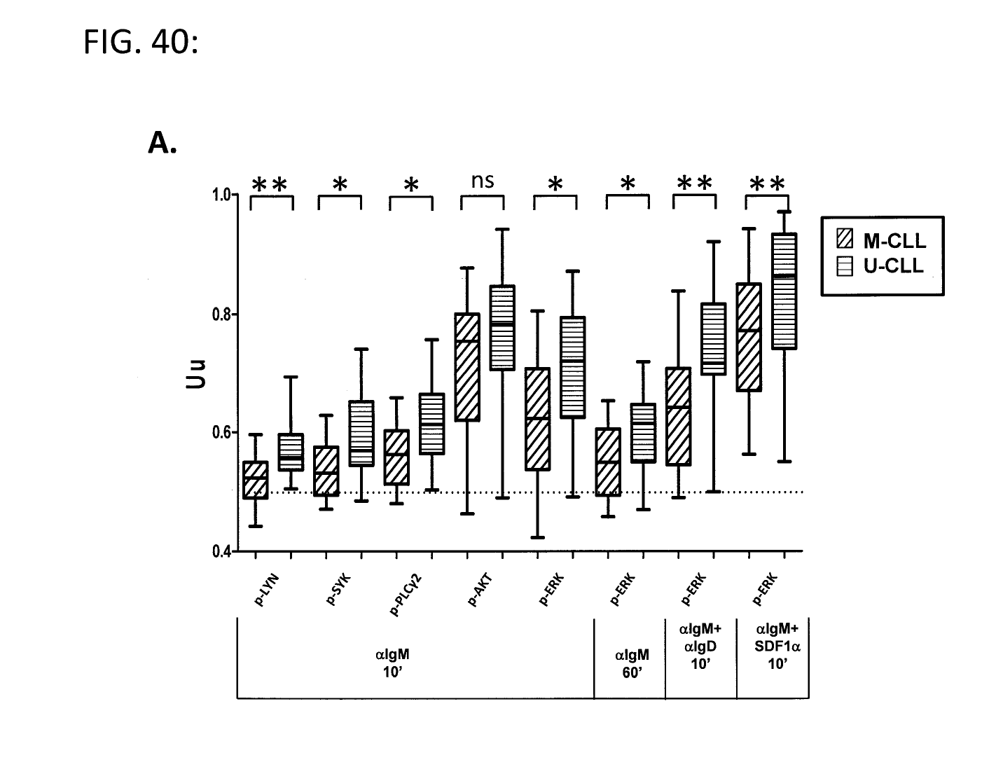

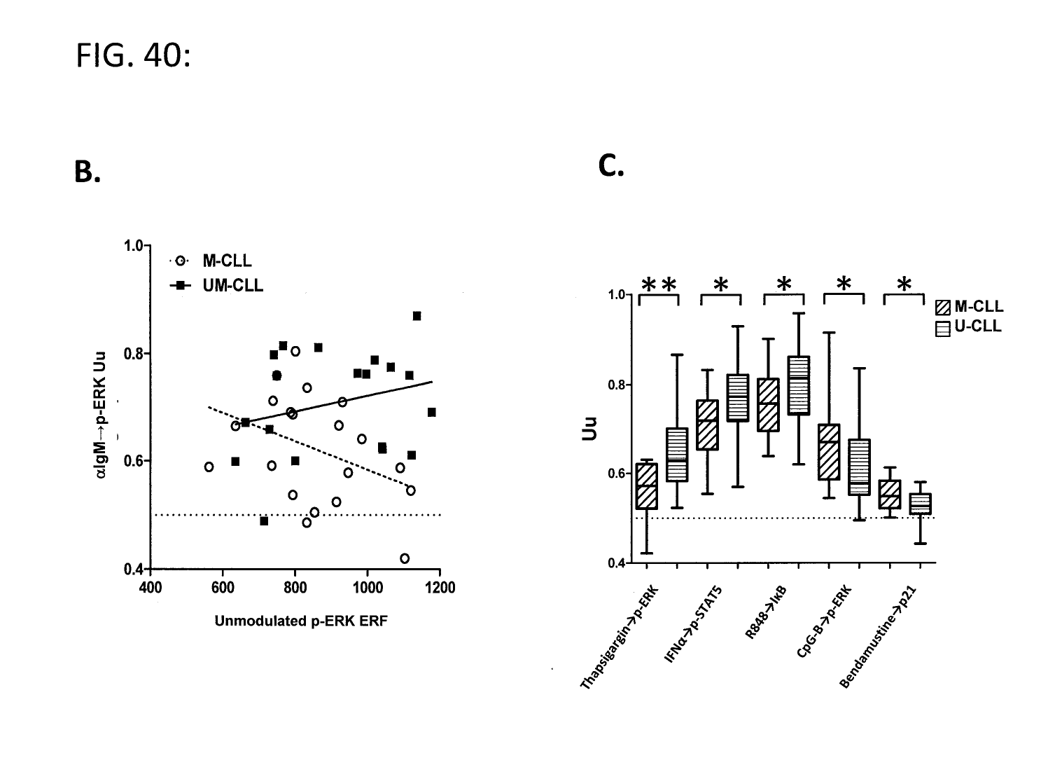

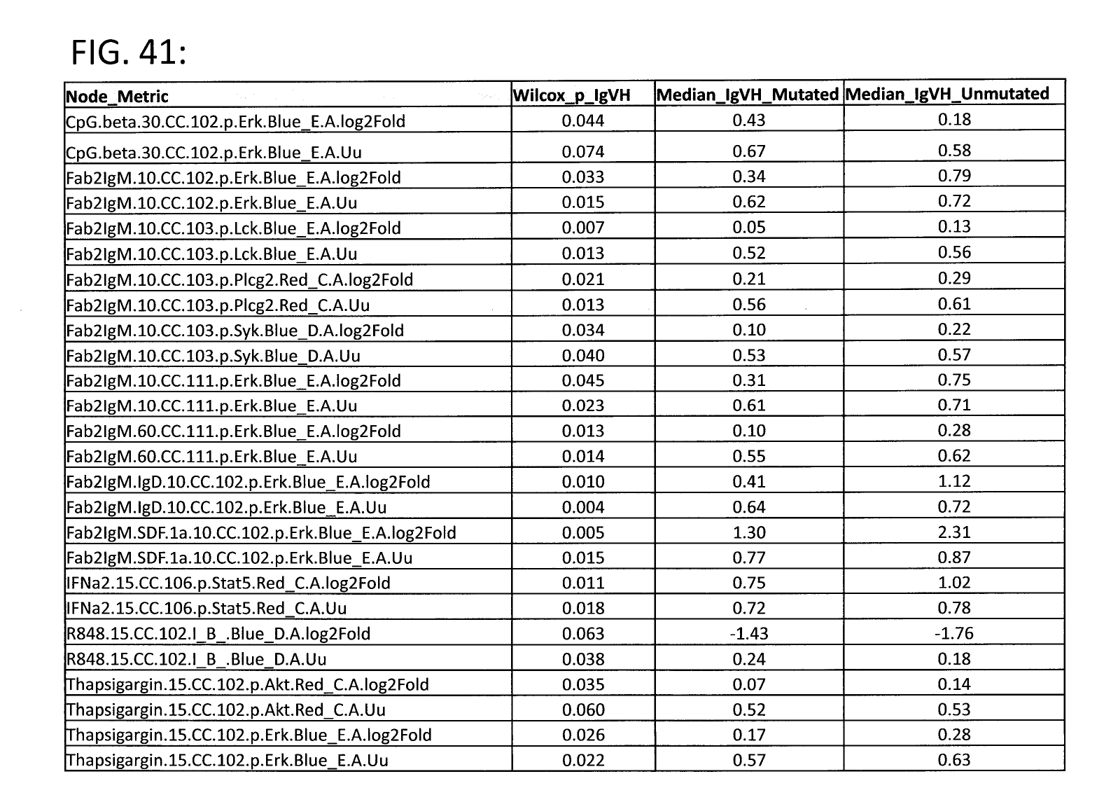

The correlate of elevated signaling via αIgM→p-ERK|Log 2Fold and unmutated IGHV (U-CLL) was previously shown in these Examples. In this Example, we sought to confirm and strengthen this association via broadening of the scope of biology analyzed. BCR modulated signaling across multiple downstream signaling proteins (p-LYN, p-SYK, p-PLCγ2, p-ERK) showed a positive correlation to unmutated IGHV as measured by both the population-based Uu metric and magnitude (Log 2Fold) (FIG. 40A, FIG. 41). Simultaneous modulation of p-ERK by αIgD and αIgM showed a stronger association than either modulation alone (Uu, P=0.0037 for the combination, P=0.015 for αIgM alone, αIgD not significant). Analysis of unmodulated signaling did not reveal differences between samples based on IGHV mutational status. Interestingly, a trend of reduced αIgM→p-ERK was identified in the M-CLL samples which had higher basal p-ERK, though this did not reach significance (p=0.11); U-CLL samples did not show an association between basal and modulated p-ERK (FIG. 40B).

Also showing a trend of increased signaling in U-CLL were TLR 7/8 (R848)→IκB degradation, thapsigargin modulated (intracellular calcium-mediated) p-ERK signaling, and IFNα→p-STAT5. In contrast, TLR9→p-ERK signaling induced by CpG-B was greater in the IGHV mutated samples, in agreement with a recent report showing greater CD86 induction by TLR9 stimulation in M-CLL vs U-CLL (FIG. 40c). The induction of the cell cycle inhibitor, p21, induced in response to p53 checkpoint activation, was weakest in the U-CLL samples. For all node associations both the Uu and Log 2Fold metric were evaluated and similar significance was observed for both metrics (FIG. 41).

### ZAP-70 and CD38 Prognostic Markers and Associated Signaling

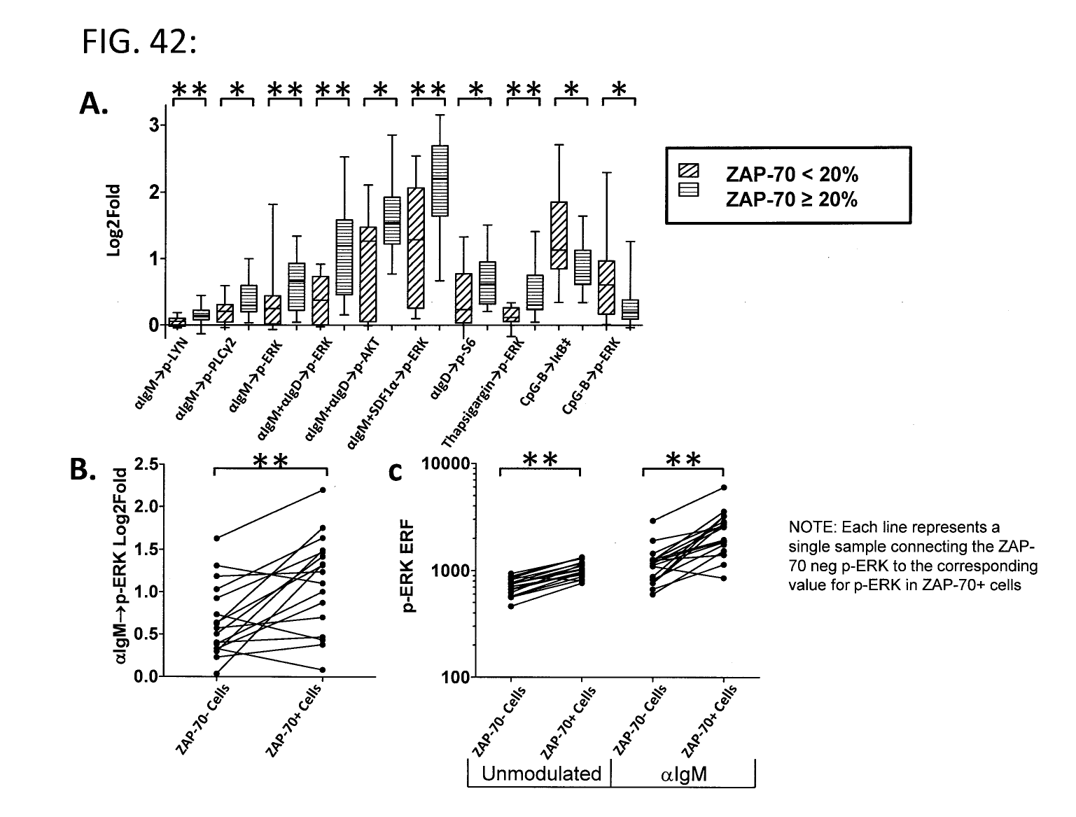

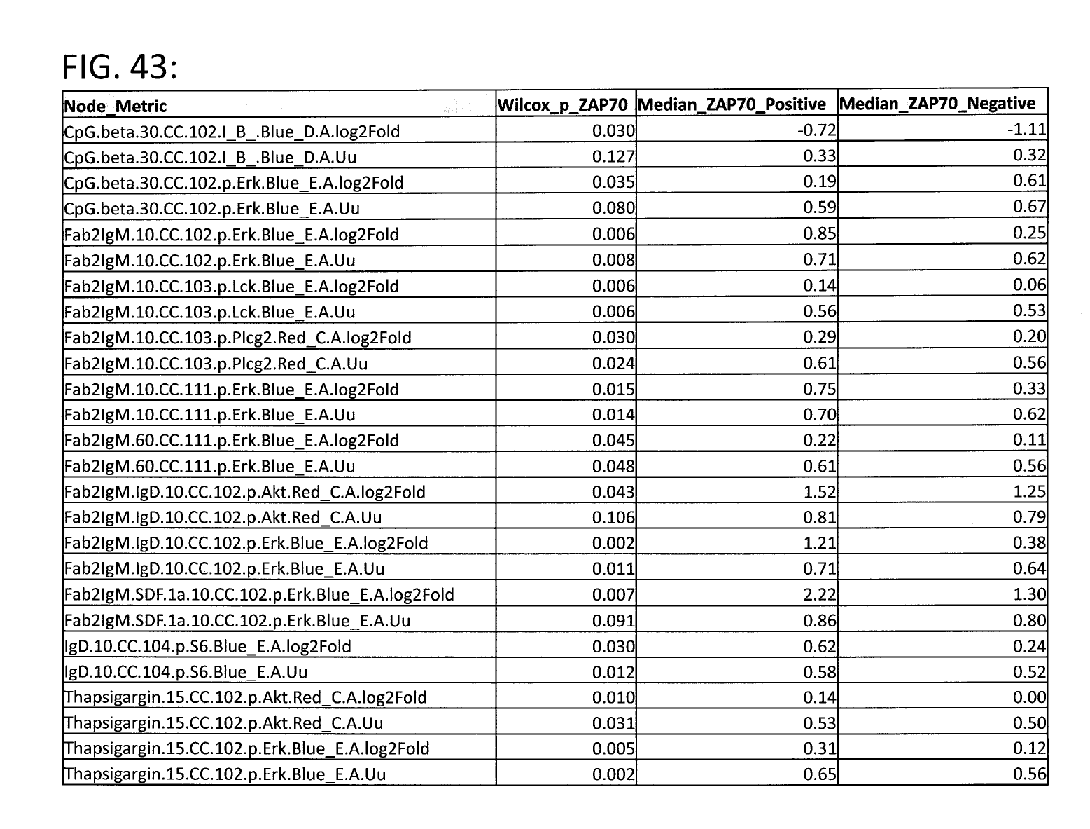

Aberrant expression of ZAP-70 and upregulation of CD38 on CLL cells have been shown to occur most frequently in patients with worse clinical outcome. To better identify and understand pathway dysfunctions driving a more aggressive phenotype, the SCNP data was analyzed for associations to both markers. Greater αIgM modulated signaling (p-LYN, p-PLCγ2, p-ERK) and thapsigargin modulated signaling (p-AKT, p-ERK) were identified in samples with greater than 20% ZAP-70⁺ cells, similar to the trends observed with U-CLL. (FIG. 42a, FIG. 43). In contrast, in these same samples reduced signaling through TLR9 (CpG-B→p-ERK|Log 2Fold and CpG-B→IκB|Log 2Fold) was also observed. No differences in TLR9AKT signaling was observed between the two molecularly defined sample sets.

Analysis of this differential signaling at the level of individual cells simultaneously stained for ZAP-70 positivity, determined by ZAP-70 expression within T cells, and p-ERK identified a continuum of ZAP-70 levels in the 24 samples with at least 100 cells in both ZAP-70 cell subsets. Increased fold activation of at least 20% for αIgM→p-ERK in the ZAP-70⁺ fraction of cells was identified for a majority of αIgM-responsive samples; 12/17 samples with a Log 2Fold p-ERK≧0.3 in the entire B-CLL population irrespective of ZAP-70 expression had a greater response in the ZAP-70+ cells. This trend, identified at the single cell level between ZAP-70 expression and modulated signaling, was significant (p<0.01, (FIG. 42b). Further analysis revealed greater p-ERK levels in both the modulated and unmodulated levels in the ZAP-70⁺ cells (ERF, p<0.001, FIG. 42c.

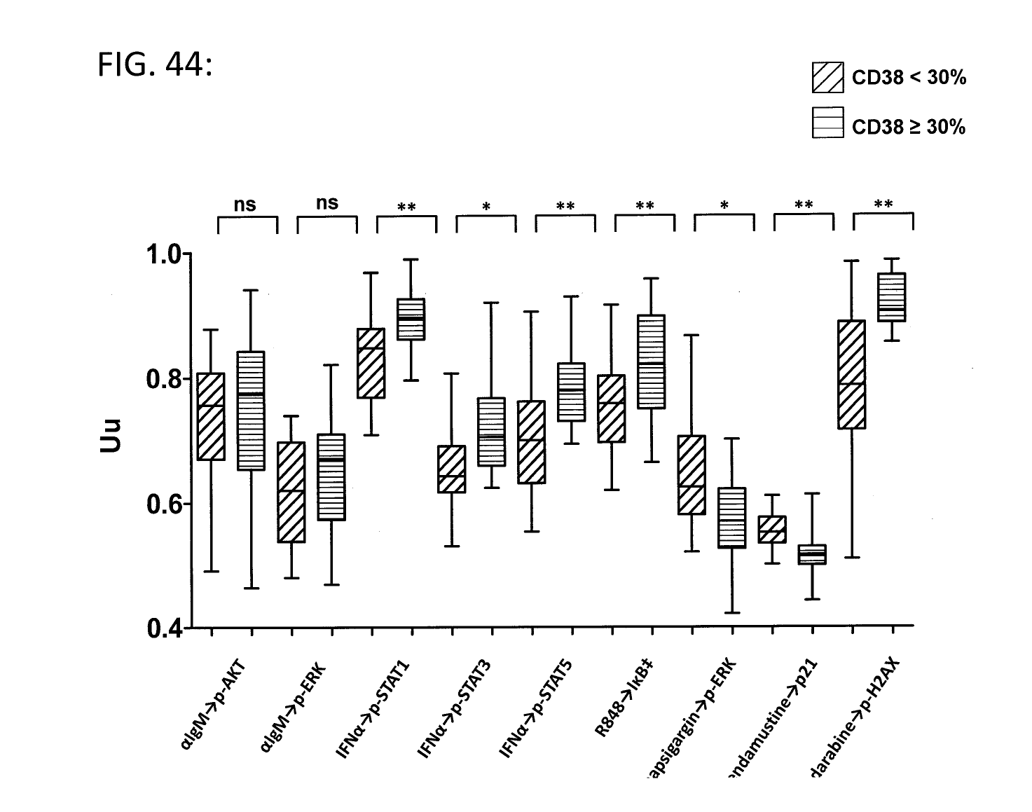

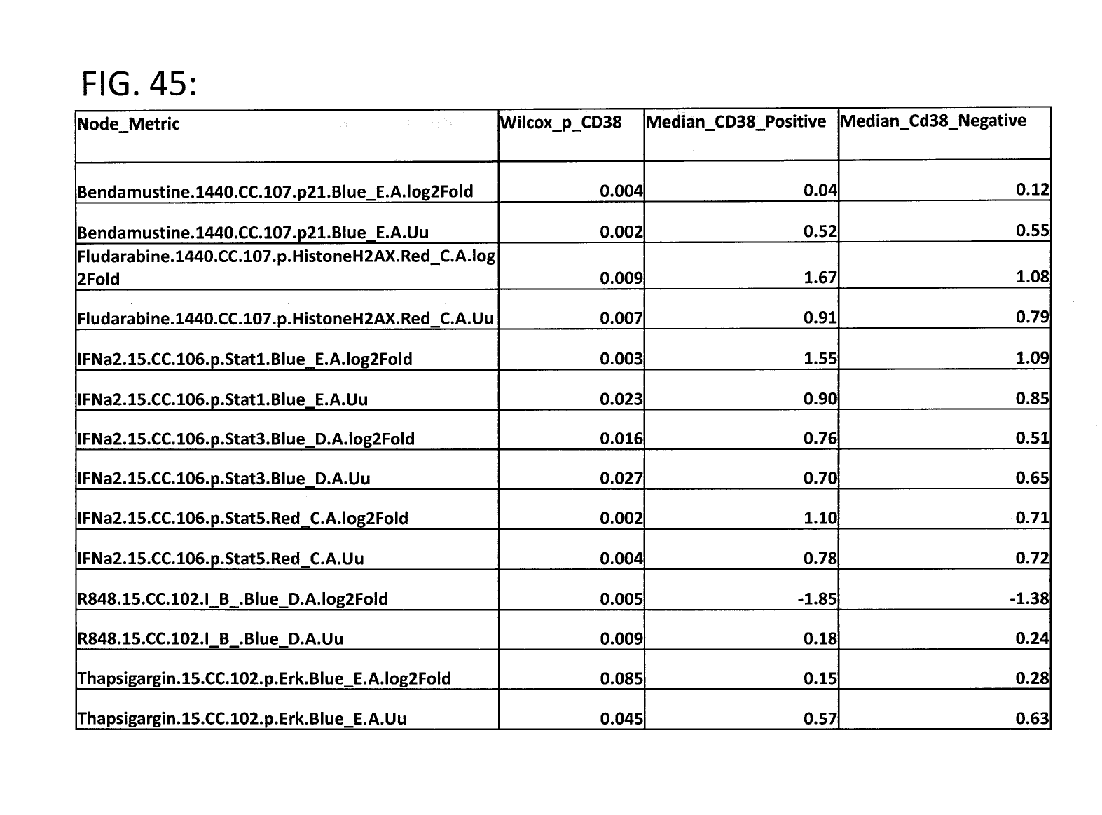

Consistent with the U-CLL and ZAP-70 positive samples, CD38 positive samples showed a trend of increasing BCR signaling capacity, although these associations did not reach significance (FIG. 44, FIG. 45). Unlike ZAP-70+/− subsets, co-staining cells with antibodies that identified CD38 and p-ERK did not show greater levels of p-ERK in □IgM modulated CD38⁺ cells compared to CD38⁻ cells for 34 samples where both subsets were detectable. However, significantly greater responsiveness to IFN□ (p-STAT1, p-STAT3, p-STAT5, Uu and Log 2Fold) was observed in CD38 samples. Additionally, these samples were more sensitive to fludarabine, an inhibitor of DNA synthesis, as measured by the increase in p-H2AX (Log 2Fold, Uu).

Association of CLL Signaling with Clinical Progression

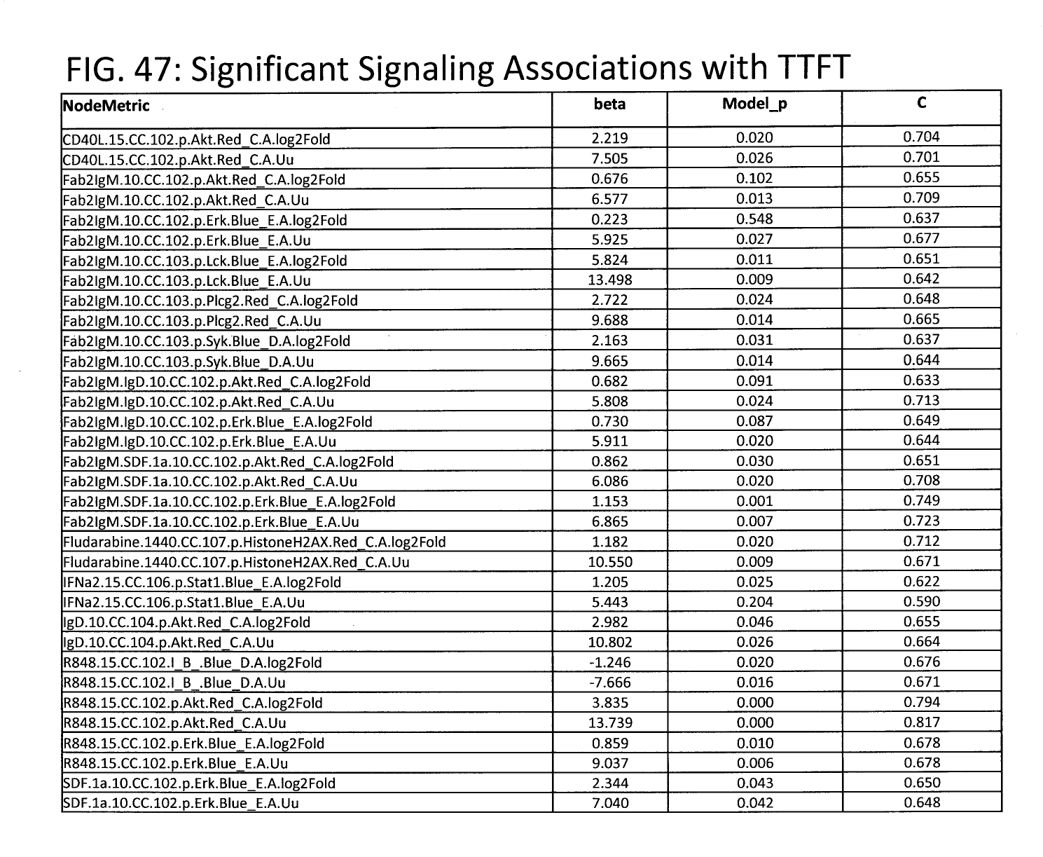

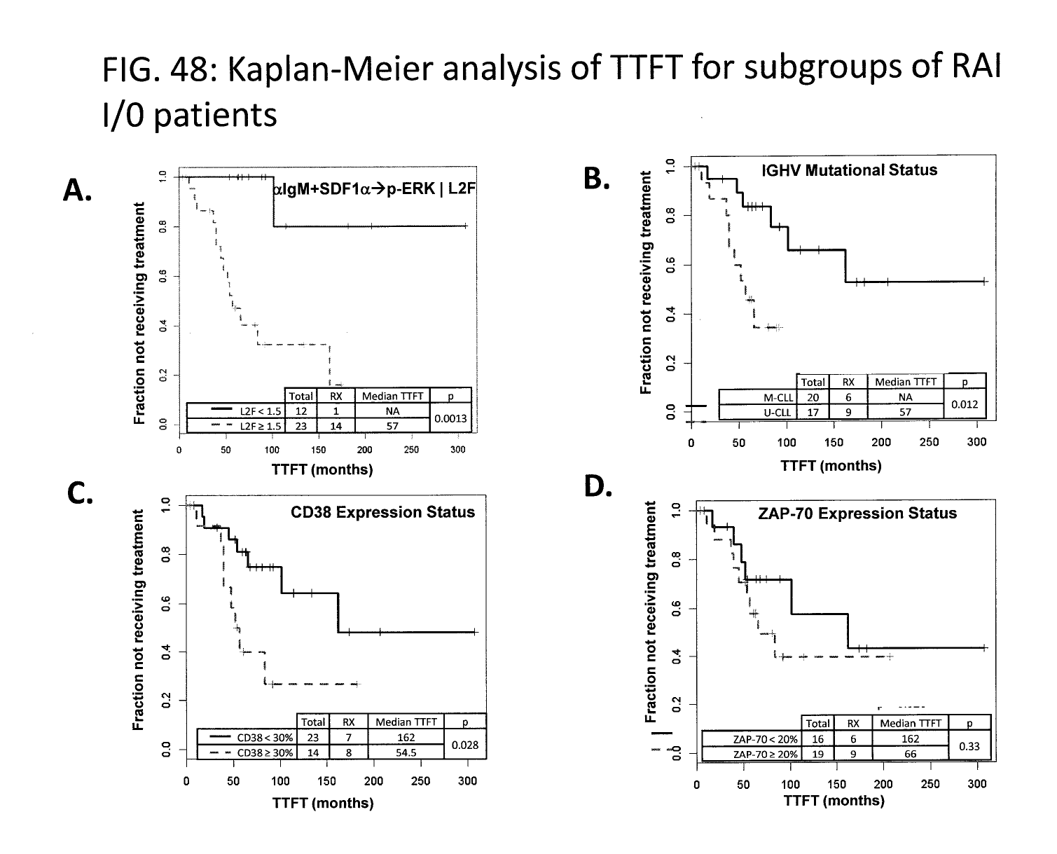

Correlations of signaling with disease course were investigated. Several significant univariate associations between signaling and TTFT were identified (FIG. 46, FIG. 47). Of note, patients could be stratified into two groups using a combination of αIgM and SDF1α together to induce p-ERK using the L2F metric. Examination of Kaplan-Meier curves found αIgM+SDF1α→p-ERK1L2F (p=0.0013) to be comparable to that of IGHV mutational status (p=0.012) and CD38 (p=0.028) and better than ZAP-70 (p=0.33), (FIG. 48).

We first developed models that associated with IGHV mutational status using multivariate logistic regression modeling analysis. For example, the combination of CpG-B→p-ERK and αIgD+αIgM→p-ERK or of R848→IκB and αIgM→p-ERK produced models of association with IGHV mutational status with an AUC greater than 0.80 and with a significance of p<0.01, displaying greater prognostic significance than either alone and exemplifying the increased power of prognostication when combining different signaling pathways that may be associated with disease pathology (FIG. 40A).

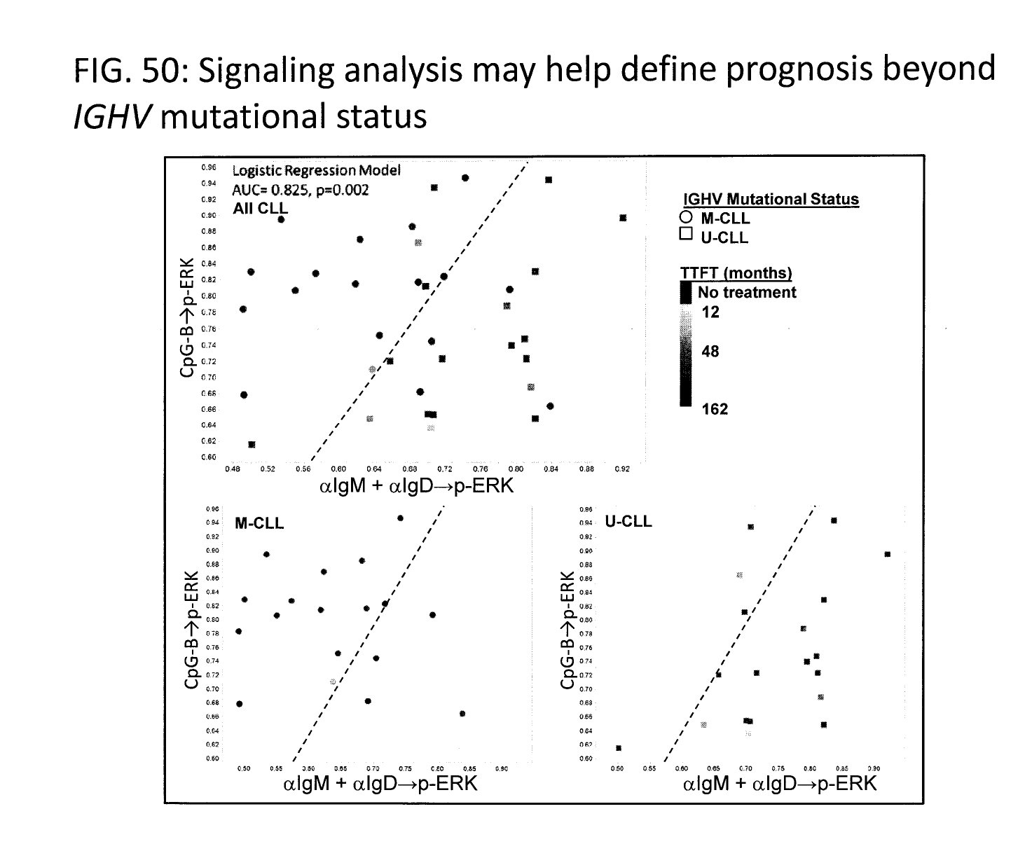

To determine whether SCNP signaling analysis could define prognosis beyond IGHV mutational status we examined the model that combined CpG-B→p-ERK with αIgD+αIgM→p-ERK in the context of available TTFT data. There were 18 samples analyzed from donors with M-CLL and of these 6 donors required treatment after the time of sampling. The boundary of the model of IGHV mutatational status grouped the majority (14/18) of M-CLL samples together based on their signaling profile (FIG. 50). Most of the donors with aggressive disease requiring treatment, including those with poorer prognosis based on IGHV mutational status, had signaling within the boundary of the model. Of the 4 M-CLL that grouped with the U-CLL samples on the basis of their induced signaling, half came from donors with disease that progressed. In contrast, of the 14 M-CLL donors with a signaling profile distinct from U-CLL, only 4 of these donors had progressive disease and 2 of these donors had signaling near the boundary predicting U-CLL. The time of follow up for this cohort is a factor in these analysis. However, M-CLL donors had a median follow-up time 69 months versus 40 months for U-CLL donors. Therefore, the lack of progressive disease in donors with a signaling phenotype of stronger CpG-B→p-ERK signaling and weaker αIgD+αIgM→p-ERK signaling cannot be attributed to lack of patient follow up time but instead to a disease with a distinct signaling profile and associated clinical course.

Conclusions:

This is an independent SCNP analysis of B-CLL signaling showing decreased bendamustine→p21 and increased F(ab)₂IgM→p-ERK in samples with unfavorable cytogenetics. Further associations with IgVH unmutated status included increased BCR signaling in multiple nodes, altered TLR9 responsiveness and decreased drug-induced p21. These data support the utility of SCNP in: (1) identifying in one assay those patients with a more aggressive form of B-CLL, including both unmutated IgVH and p53 pathway alteration, and (2) identification of patients with signaling profiles that may be more likely to respond to targeted therapies.

### Discussion

Intracellular signaling networks are a primary information processing system by which cells interpret their environment. External environmental cues, in the form of cell-cell contact, cytokine engagement via receptors, or therapeutic intervention, lead a normal or cancerous hematologic cell to initiate the phosphorylation or dephosphorylation of intracellular proteins. These changes drive differential outcomes, depending on the cues, in cellular differentiation, homeostasis, and survival. Understanding how cancer cells process information that is carefully linked to clinical outcomes and therapeutic responsiveness will ultimately lead to better disease classification, diagnostics, and treatment selection. Herein multiparameter flow cytometry was used and aberrant growth factor, drug, TLR ligand and BCR-induced modulation of signaling networks was revealed. Receptor levels were found to be necessary but not sufficient for responding to environmental stimuli as samples with similar levels of receptor expression can have different functional outcomes to ligands targeting the receptor. Prognostic markers in CLL were found to be associated with specific activation levels of signaling networks. Based on these signaling data predictive models for TTFT were developed.

BCR signaling is known to be a driving event in CLL disease onset and progression. Reports from our group and others have shown differences between healthy and CLL BCR signaling by measuring Ca²⁺ mobilization, p-SYK, p-ERK, NFAT and NFκB activation. The current Example confirms and extends findings detailed in other Examples of altered BCR signaling in CLL patients. Whereas proximal BCR signaling (LYN, SYK, and PLCγ2) and ERK were reduced, AKT signaling was maintained at the same level as healthy B cells in most samples. AKT signaling has been shown to be a major determinant of cell survival in CLL. NF-□B signaling appeared to be present prior to αIgM modulation as CLL cells' basal I□B were at levels obtained in healthy B cells after αIgM modulation. BCR signaling can provide both survival and apoptotic signals, and it is likely the context in which the BCR modulation is presented that dictates the functional outcome. Surprisingly, although CLL cells in the periphery are largely viewed as being quiescent we found that through modulation, such as with αIgM and the chemokine SDF1α an additive and potentially synergistic signaling was observed for p-ERK and p-AKT and the majority of CLL samples had greater p-AKT signaling than healthy B cells. The tissue microenvironment and extent of survival stimuli present may give the CLL cells an advantage since the role of AKT in cell survival, via MCL1 induction, is well-documented and ERK signaling also contributes to survival signaling in addition to having a role in proliferation.

Importantly, distinct signaling nodes and pathways were found to be associated with current molecular markers of unfavorable prognosis, both the hardwired mutational status of IGHV and the more variable markers of CD38 and ZAP-70 expression. Analysis of gene expression of U-CLL and M-CLL revealed surprisingly few differences in expression between the two risk categories. However, at the level of signaling, U-CLL had not only a greater BCR modulated signaling, as reported previously, but also had increased TLR7 signaling and decreased TLR9 signaling. CLL survival signals have been reported to originate in part from apoptotic cells. TLR7, highly expressed within CLL cells, is localized adjacent to phagosomes containing apoptotic cell particles. Greater sensitivity to auto-antigens mediated through TLR7 modulation could result in a more robust survival or proliferation signal, and TLR7 signaling has been associated with resistance to apoptosis by CLL cells. Our data indicate that SCNP analysis can be applied to better define functional biology and thus patient prognosis subgroups within the IGHV M and U subsets. This is of particular need, for ˜30% of M-CLL patients will likely have a disease course requiring treatment.

ZAP-70 samples were characterized by greater BCR signaling, similar to samples with U-CLL. Furthermore, the results extend prior observations of increased αIgM signaling in ZAP-70 samples by showing for the first time at single cell resolution that the increased signal is indeed originating from the ZAP-70 expressing cells and ZAP-70 is not merely a surrogate or indirect marker for a more signaling-competent clone. Both U-CLL and ZAP-70 cells showed weaker CpG-B→p-ERK than their respective counterparts. This is surprising given CpG-B is a potent B cell factor and is used to induce metaphase for cytogenetics. However, CpG-B has also been shown to induce apoptosis in M-CLL samples. While CD38+ and U-CLL samples shared increased R848→IκB degradation, CD38 samples were notable for their responsiveness to IFNα. This is in agreement with earlier reports that suggested CD38⁺ cells represent recently divided cells more capable of responding to stimuli.

A long-term challenge in CLL is understanding why molecularly homogenous patients have different disease courses. Using the landscaping of CLL signaling biology, functional differences that associated with a shorter TTFT were identified. BCR signaling contributes to CLL cell survival and this survival advantage is thought to contribute to a more aggressive disease. Consistent with this hypothesis, samples from donors with a shorter TTFT showed increased signaling in multiple pathways in response to BCR modulation. In addition, TLR7 signaling through AKT, IκB, and ERK showed an association with TTFT. Similarly, CD40 ligand modulation of p-AKT was strongest in donors with more aggressive disease. Interestingly, and consistent with the more proliferative nature of short TTFT, fludarabine modulation of p-H2AX, a marker of double-strand breaks and DNA damage, was greatest in samples acquired from donors that had a more rapid disease progression. This suggests that CLL cells from donors with relatively more aggressive disease have cells more poised for DNA replication making them more sensitive to fludarabine's DNA-damaging effects. Collectively, these associations are consistent with the biological roles of the signaling nodes examined and provide combinations of multiple signaling pathways that may be involved in CLL pathology, to better define the disease biology and individual patient prognosis.

Specifically, donors with a shorter TTFT had increased signaling with TLR7 and within multiple nodes downstream of the BCR including BCR+SDF1α modulated signaling proteins. These results support the theory that the sensitivity to BCR modulation help tip the balance towards increased cell survival and proliferation and predisposes to more aggressive disease. Donors with a greater activation of AKT by CD40 engagement also tended to have a shorter TTFT. The fact that CD40 modulation of p-ERK and I□B, while robust, did not associate with TTFT further supports the primary effect of CD40 signaling in CLL cells is through AKT, as CD40L protection from spontaneous apoptosis is blocked by inhibition of AKT phosphorylation by the PI3K□ inhibitor (CAL-101/GS-1101). Constitutive engagement of CD40 on CLL cells may facilitate malignant cell growth and resistance to apoptosis through upregulation of anti-apoptotic factors such as Bcl-XL, TNF□-induced protein 3 (A20), survivin, and cFLIP. The data in this Example demonstrate the use of SCNP to better identify those with more aggressive disease who may benefit from early therapeutic intervention.

### Example 4

This example shows the following: Functional Pathway Analysis by Single Cell Network Profiling (SCNP) Provides Insight Into B-cell Chronic Lymphocytic Leukemia (B-CLL) Pathology.

Objectives:

1) Verify the association of greater F(ab)₂IgM induced p-ERK signaling with shorter TTFT in a cohort of untreated donors with Rai Stage 0 or 1 B-CLL. 2) Explore additional signaling biology for associations with TTFT.

Methods:

Peripheral blood mononuclear cells (PBMCs) were collected and cryopreserved from a cohort of 37 untreated B-CLL patients between 2006 and 2007 at different points during their clinical follow up (these sample are from Example 3). At the time of SCNP analysis, 15 (41%) had progressed, requiring treatment. Median follow-up was 102 months (range 11-162 months). SCNP analysis was performed to quantitatively measure 22 intracellular signaling proteins within CD19⁺CD5⁺ B-CLL cells, using a panel of 14 disease-relevant modulators (BCR crosslinkers, chemokines, DNA damaging agents, interferons, interleukins, TLR ligands, etc.) to induce B-CLL cell signaling. Signaling was quantified using 1) the log₂ fold change in signal, and 2) a Uu metric that is a rank-based method with a defined range that represents the percentage of responsive cells within a population. Cox Proportional Hazards regression and Kaplan-Meier curves were used to test for signaling associations with TTFT.

Results:

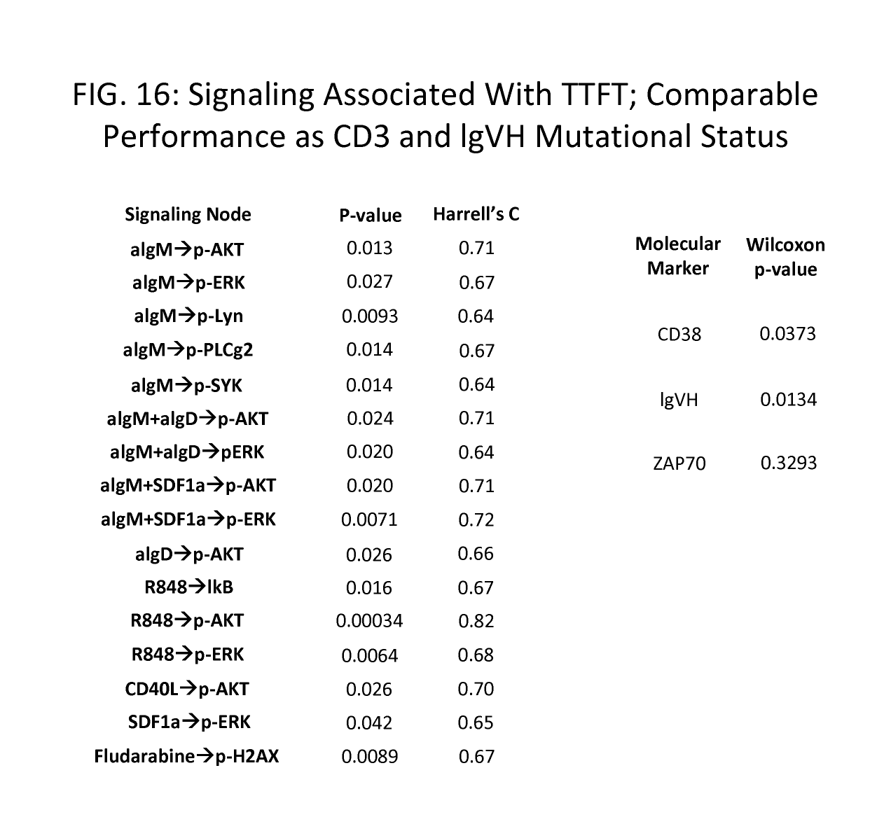

Sixteen signaling nodes were identified as being associated with TTFT (Table 8). Signaling proteins downstream of the BCR receptor, either modulated with F(ab)₂IgM or anti-IgD crosslinking for 10 minutes, were activated to a greater degree in samples that were IgVH unmutated. R848 (TLR7 agonist), CD40L, and SDF1a induced signaling were also observed to be increased in IgVH unmumated. Sustained BCR signaling (F(ab)₂IgM→p-ERK signaling at 60 minutes of modulation) did not significantly associate with TTFT (shorter time points were better). The combination of SDF1□ and F(ab)₂IgM induced greater p-ERK signaling at 10 mins than observed with either agent alone and displayed greater association with TTFT (p=0.007). Additional BCR-modulated nodes also showed an association with TTFT, including p-LYN (p=0.009), p-SYK (p=0.014), p-PLCgamma2 (p=0.014), p-AKT (p=0.013) but not IκB. Modulation of TLR7/8 (using R848), and examination of induced p-ERK, p-AKT, and I□B showed that increased signaling capacity associated with more rapid disease progression (or shorter TTFT). Of the current clinical molecular prognostic markers of TTFT, IgVH mutational status (p=0.013) and CD38 (p=0.037) but not ZAP70 showed a significant association with TTFT. See FIG. 16.

TABLE 8

| Signaling Node | beta | p | C |
|---|---|---|---|
| Fab2IgM (BCR)→p-Akt | 6.58 | 0.013 | 0.71 |
| Fab2IgM→p-Erk | 5.92 | 0.027 | 0.67 |
| Fab2IgM→p-Lck | 13.50 | 0.0093 | 0.64 |
| Fab2IgM→p-Plcg2 | 9.69 | 0.014 | 0.67 |
| Fab2IgM→p-Syk | 9.66 | 0.014 | 0.64 |
| Fab2IgM + IgD→p-Akt | 5.81 | 0.024 | 0.71 |
| Fab2IgM + IgD→p-Erk | 5.91 | 0.020 | 0.64 |
| Fab2IgM + SDF1a→p-Akt | 6.09 | 0.020 | 0.71 |
| Fab2IgM + SDF1a→p-Erk | 6.87 | 0.0071 | 0.72 |
| IgD→p-Akt | 10.80 | 0.026 | 0.66 |
| R848(TLR7)→IkB | −7.67 | 0.016 | 0.67 |
| R848→p-Akt | 13.74 | 0.00034 | 0.82 |
| R848→p-Erk | 9.04 | 0.0064 | 0.68 |
| CD40L→p-Akt | 7.51 | 0.026 | 0.70 |
| SDF1a→p-Erk | 7.04 | 0.042 | 0.65 |
| Fludarabine→p- | 10.55 | 0.0089 | 0.67 |
| HistoneH2AX | | | |

Conclusions:

These data confirm the association with TTFT of BCR signaling in B-CLL cells in early stage patients in an independent cohort. Increased BCR signaling was significantly associated with shorter TTFT in multiple nodes, strengthening the biological data of the role of BCR signaling in disease course. See FIG. 15. In addition to BCR signaling, B-CLL cells also likely receive concomitant signaling in vivo through CD40 and TLRs, and both CD40L and TLR7/8 agonist, R848, showed greater responsiveness in samples obtained from donors with shorter TTFT. Further, the combination of SDF1alpha and F(ab)₂IgM strengthened the association between signaling and disease course. These data are examples of the use of SCNP in: (1) identifying patients with a more aggressive form of B-CLL that may benefit from early intervention, and (2) identification of patients with signaling profiles that may be more likely to respond to targeted therapies currently being developed.

In summary, the results show the following. The use of F(ab)₂IgM+SDF-1a→p-Erk (as a node) and F(ab)₂IgM→p-Erk are good predictors of IgVH status (0.94 and 0.9). F(ab)₂IgM+SDF-1a→p-Erk seems to perform better than IgVH status in modeling TTFT for Binet stages A+B (not out-of-bag however). Cox modeling of TTFT: Harrell c index of 0.61 (IgVH) vs 0.68 (F(ab)₂IgM+SDF-1a→p-Erk log 2Fold). Splitting TTFT into two bins with a cutoff at 36 months: AUROC of 0.8 (IgVH) vs 0.9 (F(ab)₂IgM+SDF-1a→p-Erk log 2Fold/Uu).

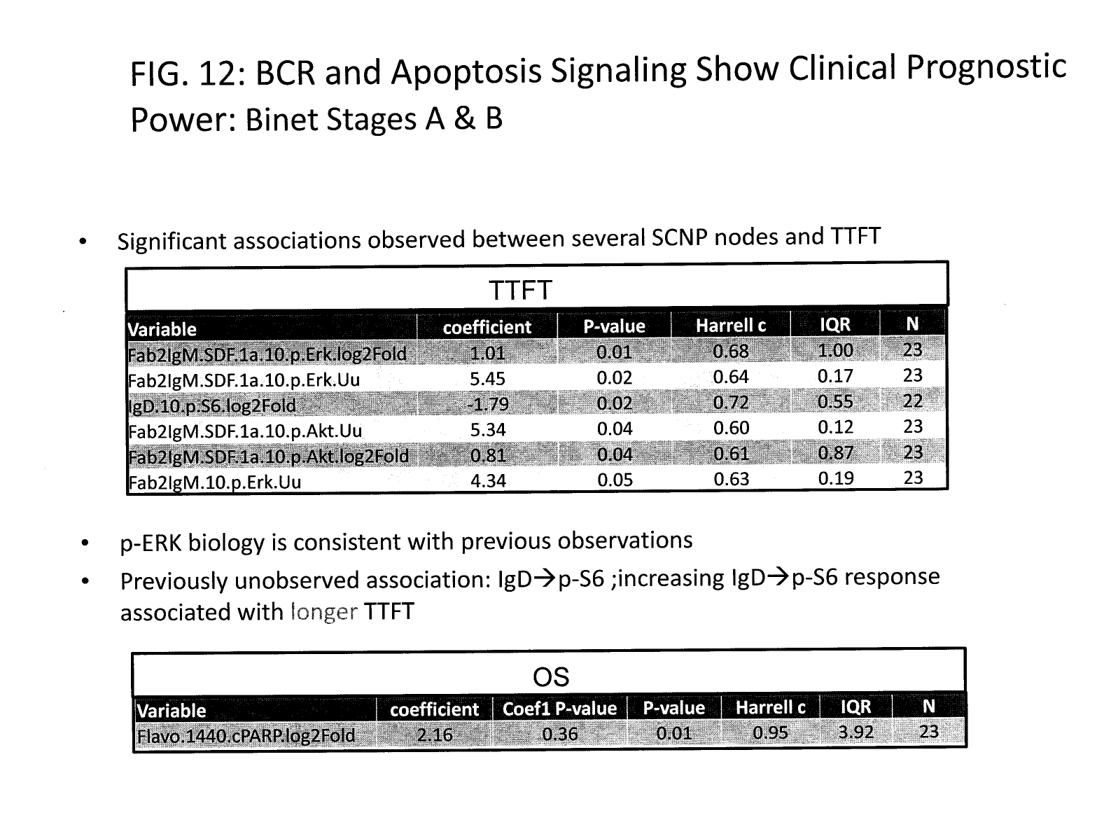

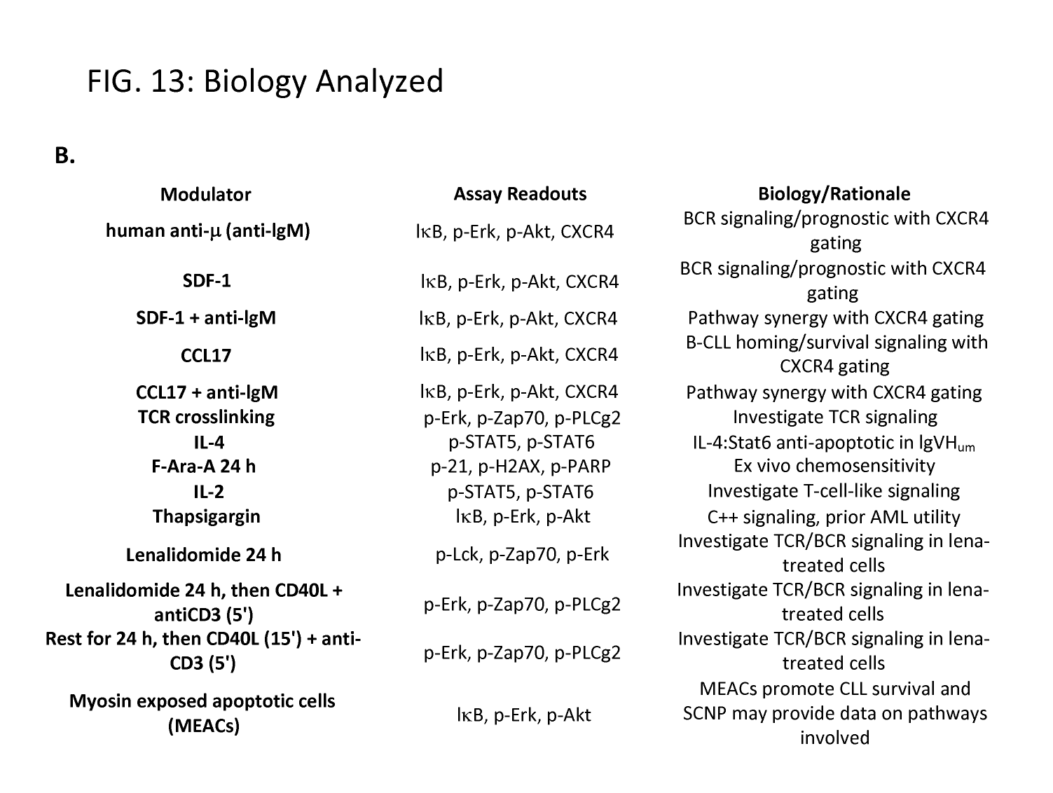

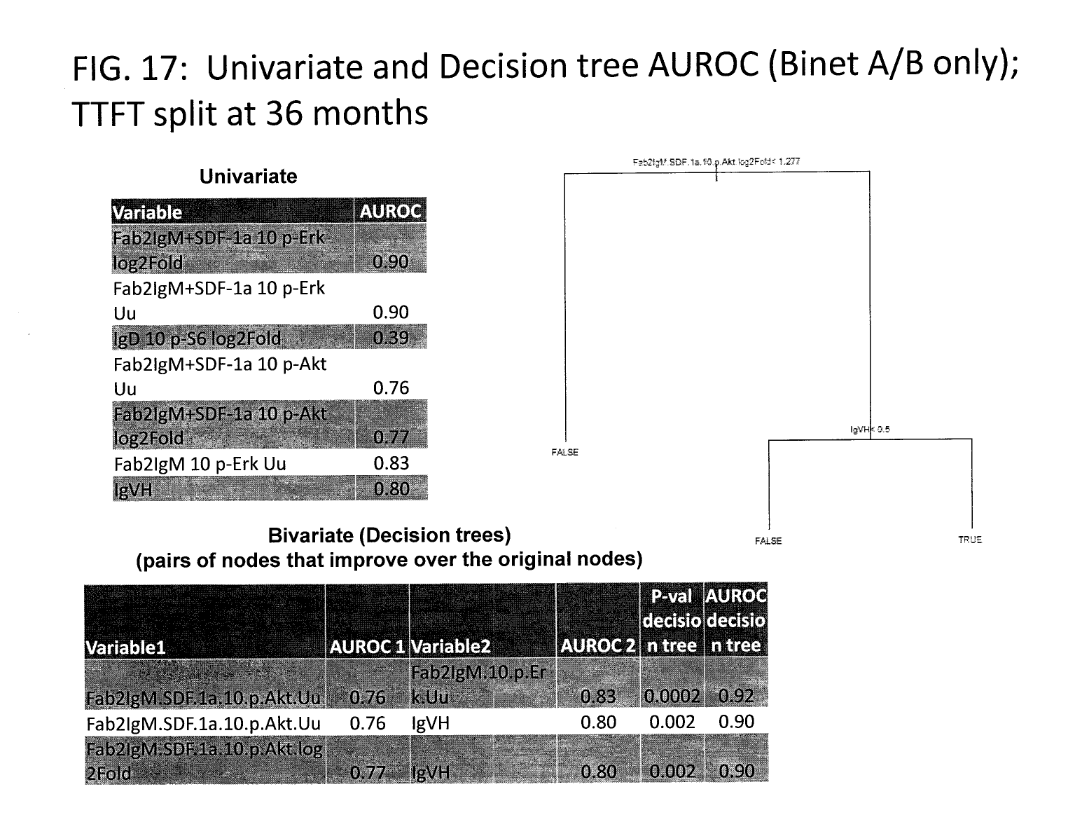

The nodes that perform best in Cox modeling of TTFT are the following. All Binet stages: F(ab)₂IgM+SDF-1a→p-Erk, F(ab)₂IgM→p-Erk, F(ab)₂IgM+SDF-1a→p-Akt, Fludarabine/Bendamustine→cPARP, Bendamustine→p21. Only Binet stages A+B: anti-IgD→p-S6, F(ab)₂IgM+SDF-1a→p-Erk, F(ab)₂IgM→p-Erk, F(ab)₂IgM+SDF-1a→p-Akt. anti-IgD→p-S6 performs well for Binet stages A+B and is part of all bivariate models. F(ab)₂IgM+SDF-1a→p-Erk is better than F(ab)₂IgM modulation alone (Harrell c index of 0.68 vs 0.63 and AUROC from binning of 0.9 vs 0.83). See also FIGS. 12, 16, and 17.

Additonally, the above examples show that SCNP can be an equivalent to IgVH analysis. See also FIGS. 15 and 16 which show the comparable performance between the two methods.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of determining time to first treatment (TTFT) in a subject suffering from or suspected of suffering from Chronic Lymphocytic Leukemia (CLL) comprising
(i) exposing cells from a sample obtained from the subject to at least two modulators;
(ii) measuring, on a single cell basis, the level of an activated form of at least one activatable element in the cells; and
(iii) determining a TTFT for the subject based on the information obtained in step (ii).

2. (canceled)

3. The method of claim 1 wherein the two modulators comprise a BCR crosslinker and a chemokine.

4. The method of claim 3 wherein the BCR crosslinker comprises an anti-IgG antibody or antibody fragment, or an anti-IgD antibody or antibody fragment.

5. The method of claim 3 wherein the BCR crosslinker comprises F(Ab)₂IgM.

6. (canceled)

7. The method of claim 3 wherein the chemokine is SDF 1□.

8. (canceled)

9. The method of claim 1 wherein the activated form of the activatable element is selected from the group consisting of cPARP, p-AKT, p-ERK, p-LYN, p-PLCg2, p-SYK, p-H2AX, p-STAT1, p-STAT3, p-STAT5, p-STAT6, pZAP-70/pSYK, p-Lck and any combination thereof.

10. The method of claim 1 wherein the activated form of the activatable element is selected from the group consisting of p-AKT, p-ERK, p-LYN, p-PLCg2, p-SYK, p-H2AX, and any combination thereof.

11. (canceled)

12. The method of claim 1 further comprising determining basal levels in cells from the sample not exposed to modulator of an intracellular element.

13. The method of claim 1 further comprising gating the assay so that only healthy cells are used in the determination of step (iii).

14. The method of claim 13 wherein the gating comprises exposing the cell to a detectable binding element specific for an activated form of an activatable element in the apoptosis pathway, detecting the level of the activated form of the activatable element in the cell, then gating the cell as either healthy or not healthy based on the level of the activated form of the activatable element detected.

15. The method of claim 14 wherein the activated form of the activatable element is cPARP.

16. The method of claim 1 further comprising taking an action based at least in part on the TTFT determined.

17. (canceled)

18. A method of determining the functional status of a p53 pathway in cells from a subject comprising
(i) exposing cells from a sample obtained from the subject to an agent whose activity depends, at least in part, on a functional p53 pathway;
(ii) measuring on a single cell basis the level of an intracellular protein whose levels increase upon induction of the p53 pathway; and
(iii) from the levels measured in step (ii), determine the functional status of the p53 pathway in the cells.

19. The method of claim 18 wherein the subject suffers from or is suspected of suffering from CLL.

20. The method of claim 19 wherein the intracellular protein is p21.

21. The method of claim 18 further comprising gating the assay so that only healthy cells are used in the determination of step (iii).

22. The method of claim 21 wherein the gating comprises exposing the cell to a detectable binding element specific for an activated form of an activatable element in the apoptosis pathway, detecting the level of the activated form of the activatable element in the cell, then gating the cell as either healthy or not healthy based on the level of the activated form of the activatable element detected.

23. The method of claim 22 wherein the activated form of the activatable element is cPARP.

24. The method of claim 18 wherein the agent is an alkylating agent.

25. The method of claim 18 wherein the agent is selected from the group consisting of bendamustine and fludarabine.

26. (canceled)

27. The method of claim 18 further comprising administering a drug to the subject, wherein the drug is a drug whose activity is dependent, at least in part, on a functional p53 pathway.

28. (canceled)

29. A system for informing a decision by a subject and/or healthcare provider for the subject involving diagnosing, prognosing, evaluating status of, or determining a method of treatment for a condition from which the subject is suffering or is suspected of suffering, wherein the system comprises
(i) the subject and the healthcare provider;
(ii) a unit for analyzing a biological sample obtained from the subject by a method of analysis comprising
(a) exposing cells from the sample to one or modulators, or no modulator,
(b) exposing the cells to a detectable binding element that binds to a form of an activatable element in the cell, and
(c) determining on a single cell basis the levels of the detectable binding element in the cell; and
(iii) a unit for communicating the results of the analysis of the sample to the subject and/or healthcare provider so that a decision may be made regarding diagnosis, prognosis, state of, or treatment of the condition that the subject suffers from or is suspected of suffering from.

30-31. (canceled)

32. The system of claim 29 wherein the condition is a B-Cell or B Cell lineage derived condition.

33. The system of claim 32 wherein the condition comprises CLL.

34. (canceled)

35. The system of claim 29 wherein the analysis unit comprises a flow cytometer or mass spectrometer for determining on a single cell basis the levels of the detectable binding element in the cell.

36. The system of claim 29 wherein the form of the activatable element detected is an activated form, and wherein the activatable element is activated by cleavage or phosphorylation.

37. The system of 33 wherein the modulator comprises a BCR crosslinker.

38. The system of claim 37 further comprising a second modulator comprising a chemokine.

39. The system of claim 33 wherein the form of the activatable element to which the detectable binding element binds is selected from the group consisting of cPARP, p-AKT, p-ERK, p-LYN, p-PLCg2, p-SYK, p-H2AX, p-STAT1, p-STAT3, p-STAT5, p-STAT6, pZAP-70/pSYK, and combinations thereof.

40. The system of claim 29 wherein the analytical unit is configured to gate data from healthy vs. unhealthy cells.

41. The system of claim 40 wherein the gating comprises determining cPARP levels in cells and gating the cells at least in part based on their cPARP levels

42-47. (canceled)
